SEQUENCE LISTING (10 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

(19) United States
(12) Patent Application Publication
(10) Pub. No.: US 2024/0410013 A1
(43) Pub. Date: Dec. 12, 2024

(54) METHODS AND COMPOSITIONS FOR CLASSIFYING AND TREATING KIDNEY CANCER

(71) Applicant: Genentech, Inc., South San Francisco, CA (US)

(72) Inventors: Habib HAMIDI, Fremont, CA (US); Mahrukh Huseni, Union City, CA (US); Romain Francois Banchereau, San Mateo, CA (US)

(21) Appl. No.: 18/653,413

(22) Filed: May 2, 2024

Related U.S. Application Data
(63) Continuation of application No. PCT/US2021/058362, filed on Nov. 5, 2021.

(51) Int. Cl. C12Q 1/6886; A61K 39/00; A61K 39/395

(52) U.S. Cl. CPC C12Q 1/6886; A61K 39/39558; A61K 2039/507; C12Q 2600/106; C12Q 2600/156; C12Q 2600/158

(57) ABSTRACT

The invention provides methods and compositions for classifying kidney cancer (e.g., RCC, e.g., an inoperable, locally advanced, or metastatic RCC); methods and compositions for treating kidney cancer in a patient, for example, by administering a treatment regimen that includes a PD-1 axis binding antagonist (e.g., atezolizumab) and a VEGF antagonist (e.g., bevacizumab) to the patient. Also provided are compositions, pharmaceutical compositions, kits, and articles of manufacture for use in classifying and treating kidney cancer in a patient.

### SEQUENCE LISTING

The instant application contains a Sequence Listing which has been submitted electronically in XML format and is hereby incorporated by reference in its entirety. Said XML copy, created on Apr. 29, 2024, is named 50474-241002_Sequence_Listing_4_29_24 and is 10,418 bytes in size.

### FIELD OF THE INVENTION

This invention relates to methods and compositions for use in classifying and treating kidney cancer (e.g., renal cell carcinoma (RCC)) in a patient.

### BACKGROUND OF THE INVENTION

RCC was diagnosed in more than 400,000 people and associated with approximately 175,000 deaths worldwide in 2018. Approximately 25% of patients present with metastatic disease at initial diagnosis. Clear-cell carcinoma (ccRCC) is the most common histologic subtype (75%) in RCC. About 20% of tumors from patients with advanced RCC contain sarcomatoid elements. RCC tumors that include a sarcomatoid component are highly aggressive and lead to rapid metastasis and poor clinical prognosis.

Inactivation of the VHL gene function by deletion of chromosome 3p, mutation, and/or promoter methylation is a predominant feature of ccRCC and leads to abnormal accumulation of hypoxia inducible factors (HIF) and activation of the angiogenesis program. However, VHL loss alone is insufficient for tumorigenesis, and additional genomic aberrations have been implicated in disease progression and degree of aggressiveness. ccRCC is also characterized as a highly inflamed tumor type, with one of the highest immune infiltration scores in pan-cancer analysis and high expression of immune checkpoints, such as PD-L1 and CTLA-4.

Given the distinct but variable hyper-vascularity, immune cell infiltration and PD-L1 expression in ccRCC, inhibitors of the VEGF pathway and PD-(L)1 axis as monotherapy or in combination have resulted in significant improvement in clinical outcomes in patients with advanced RCC. However, not all patients respond and these treatments can produce significant toxicities. Thus, a better understanding of the molecular basis of clinical heterogeneity in patients with advanced RCC is needed to inform treatment selection strategies and delineate resistance mechanisms. Moreover, improved methods of patient classification and treatment are needed.

### SUMMARY OF THE INVENTION

The present disclosure provides, inter alia, methods of classifying kidney cancer (e.g., RCC, e.g., an inoperable, locally advanced, or metastatic RCC), methods of treating kidney cancer, and related kits, compositions for use, and uses.

In one aspect, the invention features a method of classifying an inoperable, locally advanced, or metastatic RCC in a human patient, wherein the inoperable, locally advanced, or metastatic RCC is previously untreated, the method comprising (a) assaying mRNA in a tumor sample from the patient to provide a transcriptional profile of the patient's tumor; and (b) assigning the patient's tumor sample into one of the following seven clusters based on the transcriptional profile of the patient's tumor: (1) angiogenic/stromal; (2) angiogenic; (3) complement/Ω-oxidation; (4) T-effector/proliferative; (5) proliferative (6) stromal/proliferative; and (7) snoRNA, thereby classifying the previously untreated inoperable, locally advanced, or metastatic RCC in the patient.

In another aspect, the invention features a method of treating an inoperable, locally advanced, or metastatic RCC in a human patient, the method comprising: classifying the previously untreated inoperable, locally advanced, or metastatic RCC in the patient according to any one of the methods disclosed herein; and administering an anti-cancer therapy to the patient based on the classification.

In another aspect, the invention features an anti-cancer therapy for use in treating an inoperable, locally advanced, or metastatic RCC in a human patient, wherein the previously untreated inoperable, locally advanced, or metastatic RCC in the patient has been classified according to any one of the methods disclosed herein.

In another aspect, the invention features the use of an anti-cancer therapy in the preparation of a medicament for treating an inoperable, locally advanced, or metastatic RCC in a human patient, wherein the previously untreated inoperable, locally advanced, or metastatic RCC in the patient has been classified according to any one of the methods disclosed herein.

In some aspects, the anti-cancer therapy includes a PD-1 axis binding antagonist (e.g., an anti-PD-L1 antibody, e.g., atezolizumab). In some aspects, the anti-cancer therapy includes a VEGF antagonist (e.g., an anti-VEGF antibody, e.g., bevacizumab). In some aspects, the anti-cancer therapy includes a PD-1 axis binding antagonist and an anti-angiogenesis agent. In some aspects, the anti-cancer therapy includes atezolizumab and bevacizumab.

In another aspect, the invention features a method of treating a previously untreated inoperable, locally advanced, or metastatic RCC in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, the method comprising administering to the patient an anti-cancer therapy comprising atezolizumab and bevacizumab.

In another aspect, the present invention features a kit for classifying an inoperable, locally advanced, or metastatic RCC in a human patient, wherein the inoperable, locally advanced, or metastatic RCC is previously untreated, the kit comprising: (a) reagents for assaying mRNA in a tumor sample from the patient to provide a transcriptional profile of the patient's tumor; and (b) instructions for assigning the patient's tumor sample into one of the following seven clusters based on the transcriptional profile of the patient's tumor: (1) angiogenic/stromal; (2) angiogenic; (3) complement/Q-oxidation; (4) T-effector/proliferative; (5) proliferative; (6) stromal/proliferative; and (7) snoRNA, thereby classifying the previously untreated inoperable, locally advanced, or metastatic RCC in the patient.

In another aspect, the invention features a kit for identifying a human patient suffering from an inoperable, locally advanced, or metastatic RCC who may benefit from treatment with an anti-cancer therapy comprising atezolizumab and bevacizumab, wherein the inoperable, locally advanced, or metastatic RCC is previously untreated, the kit comprising: (a) reagents for determining the presence of a somatic alteration in one or more of the following genes: PBRM1, CDKN2A, CDK2NB, TP53, ARID1A, and KMT2C in a tumor sample obtained from the patient; and (b) instructions for using the reagents to identify the patient as one who may benefit from a treatment with an anti-cancer therapy comprising atezolizumab and bevacizumab.

### BRIEF DESCRIPTION OF THE DRAWINGS

FIG. 1A is a consensus matrix depicting clusters (k=7) identified by non-negative matrix factorization (NMF) clustering of 823 patient tumors. Clusters 1-7 are shown (top, horizontal axis). The number of patient tumors in each cluster are shown in parentheses.

FIG. 1B is a heatmap representing MSigDb hallmark gene set QuSAGE enrichment scores for each NMF patient cluster compared to all other patients. Black cells represent non-significant enrichment after false discovery rate (FDR) correction.

FIG. 1C is heatmap of genes comprised in transcriptional signatures. Z-scores were calculated for each gene. Samples are grouped by NMF cluster. MSKCC, Memorial-Sloan Kettering Cancer Center clinical risk score; TMB, tumor mutation burden; FAO, fatty acid oxidation; FAS, fatty acid synthesis.

FIG. 1D is a dot plot summarizing the heatmap in FIG. 1C. Samples were aggregated by NMF group using the mean across samples for each gene, and the median z-score for each signature was calculated, resulting in one z-score per signature per NMF cluster. The horizontal bar plot on the right depicts the −log 10(p-value) obtained from Kruskal-Wallis test for each signature across NMF clusters.

FIG. 1E is a bar plot representing PD-L1 expression (dark grey or light grey) by immunohistochemistry in each NMF cluster. The p-value was obtained from Pearson's Chi-squared test.

FIG. 2A is a volcano plot depicting differentially expressed genes between responders (CR/PR) and non-responders (PD) in the sunitinib arm. Genes with FDR-corrected p<0.05 and absolute log-fold change ≥0.25 are shown. CR, complete response; PR, partial response; PD, progressive disease.

FIG. 2B is a bar plot representing pathway enrichment scores for the top upregulated or downregulated MSigDb hallmark gene sets within the differentially expressed genes identified in FIG. 2A.

FIG. 2C is a volcano plot depicting differentially expressed genes in responders (CR/PR) treated with atezolizumab+bevacizumab or sunitinib. Genes with FDR-corrected p<0.05 and absolute log-fold change ≥0.25 are shown.

FIG. 2D is a bar plot representing pathway enrichment scores for the top upregulated or downregulated MSigDb hallmark gene sets within the differentially expressed genes identified in FIG. 2C.

FIG. 3A is a workflow depicting the validation strategy for Angiogenesis and T-effector signatures established in IMmotion150.

FIG. 3B are a series of Kaplan-Meier curves of progression free survival (PFS) by treatment arm (left panel, atezolizumab+bevacizumab; right panel, sunitinib) in patients with angiogenesis low (dotted line) or high (continuous line) tumors. HR, hazard ratio.

FIG. 3C are a series of Kaplan-Meier curves of PFS by treatment arm (dark grey, atezolizumab+bevacizumab; grey, sunitinib) in patients with Angiogenesis low or high and patients with T-effector low or high tumors.

FIG. 4A is a diagram showing the selection of cluster number based on consensus matrices for k=2 to k=8, and measure of cophenetic coefficient stability at various values of k. k=7, with a cophenetic coefficient of 0.90, was chosen.

FIG. 4B is a series of boxplots showing transcriptional z-scores for the 10 signatures presented in the dot plot in FIG. 1D by patient cluster.

FIG. 4C is a heatmap showing hierarchical clustering of deconvolution z-scores obtained from xCell. Samples are ordered by NMF cluster.

FIG. 4D is a graph showing the distribution of primary and metastatic tumors in NMF clusters.

FIG. 4E is a diagram showing correlations between transcriptional signatures across the IMmotion151 data set. Signature z-scores were computed for each of the 823 samples from IMmotion151 and Pearson correlations between signatures were calculated in a pairwise fashion. Positive and negative correlations are shown. The diameter of the circles is proportional to the absolute Pearson R value, which is also numerically displayed in the circles.

FIG. 4F is a bar plot representing the distribution of NMF clusters in tumors with or without TFE fusions. Fusions in TFE3 and TFEB were grouped together. Tumors from 12 patients had TFE3 fusions and 3 patients had TFEB fusions.

FIG. 4G is a Kaplan-Meier curve of PFS by treatment arm (dark grey, atezolizumab+bevacizumab; grey, sunitinib) in patients with TFE-fusions.

FIG. 5A is a series of heatmaps showing the IMmotion151 heatmap (left panel) in FIG. 1D which was then used to derive the IMmotion150 heatmap (right panel), following a model that was applied to assign patients from IMmotion150 into each cluster. Signature patterns across patient clusters were highly conserved between IMmotion151 and IMmotion150 datasets.

FIG. 5B is a series of X-Y graphs representing the mean aggregate z-score for the ten transcriptional signatures in IMmotion151 (x-axis) and IMmotion150 (y-axis) for each NMF group. The Pearson R value is represented on each plot.

FIG. 6A is a series of bar plots representing NMF cluster distribution by Memorial-Sloan Kettering Cancer Center (MSKCC, left panel) or International Metastatic Renal Cell Carcinoma Database Consortium (IMDC, right panel) clinical risk categories. P-values were obtained from Pearson's Chi-squared test.

FIG. 6B is a series of Kaplan-Meier curves of PFS in NMF clusters of patients treated with atezolizumab+bevacizumab or sunitinib.

FIG. 6C is a bar plot representing objective response rate by treatment arm in each NMF cluster. P-value was obtained using Pearson's Chi-squared test. NE, not evaluable; PD, progressive disease; SD, stable disease; PR, partial response; CR, complete response; n.s., not statistically significant (p-value>0.05); A/B, atezolizumab+bevacizumab; Sun., sunitinib.

FIG. 6D is a series of forest plots for PFS hazard ratios in patients treated with atezolizumab+bevacizumab (A/B) vs. sunitinib, by NMF cluster. mPFS=median PFS.

FIG. 7A is an oncoprint of genes with somatic alterations in at least 10% of 715 advanced RCC tumors. Tumor mutation burden (TMB) is represented for individual samples as a bar plot above the oncoprint.

FIG. 7B is a series of oncoprints displaying somatic alterations in NMF clusters. The horizontal bar plots to the right of each oncoprint represent the number of patients with alterations for each gene. P-values were obtained using the Pearson's Chi-squared test (**: p<0.01; ***: p<0.001).

FIG. 7C is a bar plot showing the NMF cluster distribution in patients with somatic alterations in PBRM1, KDM5C, CDKN2A/B, TP53, and BAP1

FIG. 7D is a heatmap (left panel) and a series of boxplots (right panel). Left panel: Hierarchical cluster depicting the ratio of transcriptional signature z-scores (columns) between altered and non-altered tumor samples for each gene considered (rows). Only genes with somatic alterations in ≥10% of patients and significant differences (p<0.05) between altered and non-altered tumors as measured by the two-side Mann-Whitney test for at least one of the transcriptional signatures considered are displayed. Right panel: Boxplots representing the z-scores of gene signatures in samples with genomic alterations in PBRM1 (n=328), KDM5C (n=100), TP53 (n=107) and/or CDKN2A/B (n=116). P-values represent the statistical significance of the comparison of signature z-scores between patients with PBRM1 and/or KDM5C alterations vs. patients with TP53 and/or CDKN2A/B alterations using the two-side Mann-Whitney test.

FIG. 8A is an oncoprint depicting the top 50 most frequently somatically altered genes in tumors from IMmotion151.

FIG. 8B is a heatmap representing the overlap proportion between pairs of the most common somatic alterations in this dataset. Proportion was calculated as the ratio of overlap between two groups over the size of the smaller group. The heatmap highlights minimal overlap between PBRM1 mutations and BAP1/CDKN2A/B alterations.

FIG. 8C is a Venn diagram representing the overlap between tumors somatically altered in PBRM1, CDKN2/B and TP53.

FIG. 8D is an oncoprint depicting somatic alterations in PBRM1, CDKN2A/B, TP53 and KDM5C.

FIG. 8E is a forest plot depicting PFS hazard ratios comparing patients treated with atezolizumab+bevacizumab vs. sunitinib by somatic alteration status for each gene. Whiskers represent 95% confidence intervals.

FIG. 9A is a series of Kaplan-Meier curves of PFS by treatment arm in patients with somatically altered or non-altered tumors for patients treated with atezolizumab+bevacizumab (dark grey) vs. sunitinib (grey).

FIG. 9B is a series of bar plots depicting objective response (OR) by arm and by somatic alteration status for the same genes as FIG. 9A. P-values were obtained from Pearson's Chi-squared test. NE, not evaluable; PD, progressive disease; SD, stable disease; PR, partial response; CR, complete response; n.s., not statistically significant (p-value>0.05); A/B, atezolizumab+bevacizumab; Sun, sunitinib.

FIG. 9C is a forest plot representing PFS hazard ratios in patients with somatically altered vs. non-altered tumors, by gene and treatment arm.

FIG. 10A is a volcano plot depicting differentially expressed genes between clear cell renal cell carcinoma-sarcomatoid (ccRCC-Sarc) and ccRCC-non-sarcomatoid (ccRCC-NonSarc) tumors. Genes with FDR-corrected p<0.05 and absolute log-fold change ≥0.25 are shown.

FIG. 10B is a bar plot representing pathway enrichment scores for the top upregulated or downregulated MSigDb hallmark gene sets within the differentially expressed genes identified in FIG. 10A.

FIG. 10C is a volcano plot depicting differentially expressed genes between ccRCC-Sarc and non-ccRCC-Sarc tumors. Genes with FDR-corrected p<0.05 and absolute log-fold change ≥0.25 are shown.

FIG. 10D is a bar plot representing pathway enrichment scores for the top upregulated or downregulated MSigDb hallmark gene sets within the differentially expressed genes identified in FIG. 10C.

FIG. 10E is a bar plot representing the distribution of PD-L1 expression by immunohistochemistry (IHC) in ccRCC-Sarc, non-ccRCC-sarcomatoid (non-ccRCC-Sarc) and ccRCC-NonSarc tumors. P-values were obtained from Pearson's Chi-squared test conducted between each pair of conditions.

FIG. 10F is a bar plot representing distribution of NMF clusters in ccRCC-Sarc, non-ccRCC-Sarc and ccRCC-NonSarc tumors.

FIG. 11A is a volcano plot representing differentially expressed genes between sarcomatoid RCC (sRCC) and non-sarcomatoid RCC (non-sRCC) tumors. Genes with FDR-corrected p<0.05 and absolute log-fold change ≥0.25 are shown.

FIG. 11B is a bar plot representing pathway enrichment scores for the top 15 upregulated or downregulated MSigDb hallmark gene sets within the differentially expressed genes identified in FIG. 11A.

FIG. 11C is a bar plot representing the distribution of NMF defined transcriptomic subgroups.

FIG. 11D is a series of bar plots representing transcriptional signature z-scores, with p-values obtained from two-sided Mann-Whitney test.

FIG. 11E is a bar plot depicting prevalence of PD-L1 expression by immunohistochemistry.

FIG. 11F is a series of pie charts representing the distribution of somatic alterations for select genes in sRCC vs. non-sRCC tumors, with p-values obtained from Pearson's Chi-squared test.

FIG. 11G is a series of Kaplan-Meier curves of PFS in sRCC patients treated with atezolizumab+bevacizumab (dark grey) or sunitinib (grey).

FIG. 11H is a series of waterfall plots depicting the best percent reduction from baseline in sum of longest diameters (SLD). The bars indicate objective response defined by Response Evaluation Criteria in Solid Tumors (RECIST) 1.1. Objective response rate was 49% in sRCC patients treated with atezolizumab+bevacizumab, and 14% in sRCC patients treated with sunitinib, p=7.7e-05 with Pearson's Chi-squared test. CR, complete response; PR, partial response; SD, stable disease; PD, progressive disease.

FIG. 12 is a schematic diagram showing a summary of molecular characteristics in transcriptomic subsets in tumors from advanced RCC patients. Radar charts in the RNA profile panel represent mean z-scores for each gene signature in the respective cluster. “DNA alts”, somatic alterations.

FIG. 13A is a series of heatmaps showing gene expression comprised in transcriptional signatures from the IMmotion151 (left panel) and JAVELIN 101 (right panel) studies. Z-scores were calculated for each gene. Samples are grouped by NMF cluster. “n” indicates the number of patient tumors and “%” indicates the percentage of patient tumors in each cluster.

FIG. 13B is a series of pie charts showing the percentage of patient tumors in each NMF cluster from the IMmotion151 and JAVELIN 101 studies.

FIG. 14A is a series of Kaplan-Meier curves of PFS in NMF clusters of patients treated with sunitinib or atezolizumab+bevacizumab in the IMmotion151 study, or with sunitinib or avelumab+axitinib in the JAVELIN 101 study.

FIG. 14B is a series of forest plots for PFS hazard ratios in patients treated with atezolizumab+bevacizumab (A/B) vs. sunitinib in the IMmotion151 study (top panel) or avelumab+axitinib (Ave+Axi) or sunitinib (Sun) in the JAVELIN 101 study (bottom panel). The PFS hazard ratios for each NMF cluster are shown. mPFS=median PFS.

### DETAILED DESCRIPTION OF THE INVENTION

The present invention provides diagnostic and therapeutic methods and compositions for cancer, for example, kidney cancer (e.g., RCC, e.g., an inoperable, locally advanced, or metastatic RCC). The invention is based, at least in part, on the discovery that the methods of classification described herein identify patient subgroups that have unexpectedly favorable response to anti-cancer therapies, including anti-cancer therapies that include a PD-1 axis binding antagonist (e.g., an anti-PD-L1 antibody, e.g., atezolizumab) and a VEGF antagonist (e.g., an anti-VEGF antibody, e.g., bevacizumab), as shown in Example 1. Moreover, Example 2 demonstrates that the methods of classification herein also are effective for identifying patient subgroups for other anti-cancer therapies, such as an anti-cancer therapy that includes the anti-PD-L1 antibody avelumab and the tyrosine kinase inhibitor axitinib. Based on these data, it is expected that the methods of classification described herein can also identify patient subgroups with favorable response to other anti-cancer therapies, e.g., anti-cancer therapies including an immunotherapy agent, a cytotoxic agent, a growth inhibitory agent, a stromal inhibitor, a metabolism inhibitor, a complement antagonist, a radiation therapy agent, an anti-angiogenic agent, or a combination thereof.

### 1. Definitions

The term “anti-cancer therapy” refers to a therapy useful in treating cancer. An anti-cancer therapy may include a treatment regimen with one or more anti-cancer therapeutic agents. Examples of anti-cancer therapeutic agents include, but are limited to, an immunotherapy agent (e.g., a PD-1 axis binding antagonist), a cytotoxic agent, a growth inhibitory agent, a stromal inhibitor, a metabolism inhibitor, a complement antagonist, a radiation therapy agent, an anti-angiogenic agent (e.g., a VEGF antagonist), and other agents to treat cancer. Combinations thereof are also included in the invention.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-88, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

A “stromal inhibitor” refers to any molecule that partially or fully blocks, inhibits, or neutralizes a biological activity and/or function of a gene or gene product associated with stroma (e.g., tumor-associated stroma). In some embodiments, the stromal inhibitor partially or fully blocks, inhibits, or neutralizes a biological activity and/or function of a gene or gene product associated with fibrotic tumors. In some embodiments, treatment with a stromal inhibitor results in the reduction of stroma, thereby resulting in an increased activity of an immunotherapy; for example, by increasing the ability of activating immune cells (e.g., proinflammatory cells) to infiltrate a fibrotic tissue (e.g., a fibrotic tumor). Targets for stromal gene antagonists are known in the art; for example, see Turley et al., Nature Reviews Immunology 15:669-682, 2015 and Rosenbloom et al., Biochimica et Biophysica Acta 1832:1088-1103, 2013. In some embodiments, the stromal inhibitor is a transforming growth factor beta (TGF-β), podoplanin (PDPN), leukocyte-associated immunoglobulin-like receptor 1 (LAIR1), SMAD, anaplastic lymphoma kinase (ALK), connective tissue growth factor (CTGF/CCN2), endothelial-1 (ET-1), AP-1, interleukin (IL)-13, lysyl oxidase homolog 2 (LOXL2), endoglin (CD105), fibroblast activation protein (FAP), vascular cell adhesion protein 1 (CD106), thymocyte antigen 1 (THY1), beta 1 integrin (CD29), platelet-derived growth factor (PDGF), PDGF receptor A (PDGFRα), PDGF receptor B (PDGFRβ), vimentin, smooth muscle actin alpha (ACTA2), desmin, endosialin (CD248), or S100 calcium-binding protein A4 (S100A4) antagonist.

A “TGF-β antagonist” refers to any molecule that decreases, blocks, inhibits, abrogates or interferes with signal transduction resulting from the interaction of TGF-β with one or more of its interaction partners, such as a TGF-β cellular receptor. In some embodiments, a “TGF-β binding antagonist” is a molecule that inhibits the binding of TGF-β to its binding partners. In some embodiments, the TGF-β antagonist inhibits the activation of TGF-β. In some embodiments, the TGF-β antagonist includes an anti-TGF-β antibody, antigen binding fragments thereof, an immunoadhesin, a fusion protein, an oligopeptide, and other molecules that decrease, block, inhibit, abrogate or interfere with signal transduction resulting from the interaction of TGF-β with one or more of its interaction partners. In some embodiments, the TGF-β antagonist is a polypeptide, a small molecule, or a nucleic acid. In some embodiments, the TGF-β antagonist (e.g., the TGF-β binding antagonist) inhibits TGF-β 1, TGF-β2, and/or TGF-β3. In some embodiments, the TGF-β antagonist (e.g., the TGF-β binding antagonist) inhibits TGF-β receptor-1 (TGFBR1), TGF-β receptor-2 (TGFBR2), and/or TGF-β receptor-3 (TGFBR3).

The terms “anti-TGF-β antibody” and “an antibody that binds to TGF-β” refer to an antibody that is capable of binding TGF-β with sufficient affinity such that the antibody is useful as a diagnostic and/or therapeutic agent in targeting TGF-β. In one embodiment, the extent of binding of an anti-TGF-β antibody to an unrelated, non-TGF-β protein is less than about 10% of the binding of the antibody to TGF-β as measured, for example, by a RIA. In certain embodiments, an anti-TGF-β antibody binds to an epitope of TGF-β that is conserved among TGF-β from different species. In some embodiments, the anti-TGF-β antibody inhibits TGF-β 1, TGF-β2, and/or TGF-β3. In some embodiments, the anti-TGF-β antibody inhibits TGF-β 1, TGF-β2, and TGF-β3. In some embodiments, the anti-TGF-β antibody is a pan-specific anti-TGF-β antibody. In some embodiments, the anti-TGF-β antibody may be any anti-TGF-β antibody disclosed in, for example, U.S. Pat. No. 5,571,714 or in International Patent Application Nos. WO 92/00330, WO 92/08480, WO 95/26203, WO 97/13844, WO 00/066631, WO 05/097832, WO 06/086469, WO 05/010049, WO 06/116002, WO 07/076391, WO 12/167143, WO 13/134365, WO 14/164709, or WO 16/201282, each of which is incorporated herein by reference in its entirety. In particular embodiments, the anti-TGF-β antibody is fresolimumab, metelimumab, lerdelimumab, 1 D11, 2G7, or a derivative thereof.

An “angiogenesis inhibitor” or “anti-angiogenesis agent” refers to a small molecular weight substance (including tyrosine kinase inhibitors), a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or the VEGF-A receptor (e.g., KDR receptor or Flt-1 receptor), anti-PDGFR inhibitors such as GLEEVEC™ (imatinib mesylate). Anti-angiogenesis agents also include native angiogenesis inhibitors, e.g., angiostatin, endostatin, etc. See, for example, Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) and, Sato Int. J. Clin. Oncol., 8:200-206 (2003).

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors, VEGF signaling, and VEGF mediated angiogenesis and endothelial cell survival or proliferation. For example, a molecule capable of neutralizing, blocking, inhibiting, abrogating, reducing, or interfering with VEGF biological activities can exert its effects by binding to one or more VEGF receptor (VEGFR) (e.g., VEGFR1, VEGFR2, VEGFR3, membrane-bound VEGF receptor (mbVEGFR), or soluble VEGF receptor (sVEGFR)). Such antagonists are also referred to herein as “VEGFR inhibitors.” Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. VEGF antagonists also include polypeptides that bind to VEGFR, anti-VEGFR antibodies, and antigen-binding fragments thereof, and derivatives which bind to VEGFR thereby blocking, inhibiting, abrogating, reducing, or interfering with VEGF biological activities (e.g., VEGF signaling), or fusions proteins. VEGF-specific antagonists also include nonpeptide small molecules that bind to VEGF or VEGFR and are capable of blocking, inhibiting, abrogating, reducing, or interfering with VEGF biological activities. Thus, the term “VEGF activities” specifically includes VEGF mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF. In some embodiments, the VEGF inhibited by the VEGF-specific antagonist is VEGF (8-109), VEGF (1-109), or VEGF₁₆₅.

As used herein VEGF antagonists can include, but are not limited to, anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), bispecific VEGF antibodies (e.g., MP-0250, vanucizumab (VEGF-ANG2), and bispecific antibodies disclosed in US 2001/0236388), bispecific antibodies including combinations of two of anti-VEGF, anti-VEGFR1, and anti-VEGFR2 arms, anti-VEGFA antibodies (e.g., bevacizumab, sevacizumab), anti-VEGFB antibodies, anti-VEGFC antibodies (e.g., VGX-100), anti-VEGFD antibodies, and nonpeptide small molecule VEGF antagonists (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, and tivozanib). In some examples, the VEGF antagonist may be a tyrosine kinase inhibitor, including a receptor tyrosine kinase inhibitors (e.g., a multi-targeted receptor tyrosine kinase inhibitor such as sunitinib or axitinib).

An “anti-VEGF antibody” is an antibody that binds to VEGF with sufficient affinity and specificity. In certain embodiments, the antibody will have a sufficiently high binding affinity for VEGF, for example, the antibody may bind hVEGF with a Kd value of between 100 nM-1 pM. Antibody affinities may be determined, e.g., by a surface plasmon resonance based assay (such as the BIAcore® assay as described in PCT Application Publication No. WO2005/012359); enzyme-linked immunoabsorbent assay (ELISA); and competition assays (e.g. radioimmunoassays (RIAs)).

In certain embodiments, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PIGF, PDGF, or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (Cancer Res. 57:4593-4599, 1997), including but not limited to the antibody known as bevacizumab (BV; AVASTIN®).

The anti-VEGF antibody “bevacizumab (BV),” also known as “rhuMAb VEGF” or “AVASTIN®,” is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

The term “immunotherapy agent” refers the use of a therapeutic agent that modulates an immune response. Exemplary, non-limiting immunotherapy agents include a PD-1 axis binding antagonist, a CTLA-4 antagonist (e.g., an anti-CTLA-4 antibody (e.g., ipilimumab)), a TIGIT antagonist (e.g., an anti-TIGIT antibody (e.g., tiragolumab)), PD1-IL2v (a fusion of an anti-PD-1 antibody and modified IL-2), PD1-LAG3, IL-15, anti-CCR8 (e.g., an anti-CCR8 antibody, e.g., FPA157), FAP-4-1 BBL (fibroblast activation protein-targeted 4-1 BBL agonist), or a combination thereof. In some examples, the immunotherapy agent is an immune checkpoint inhibitor. In some examples, the immunotherapy agent is a CD28, OX40, GITR, CD137, CD27, ICOS, HVEM, NKG2D, MICA, or 2B4 agonist or a CTLA-4, PD-1 axis, TIM-3, BTLA, VISTA, LAG-3, B7H4, CD96, TIGIT, or CD226 antagonist. Other particular immunotherapy agents include anti-TIGIT antibodies and antigen-binding fragments thereof, anti-CTLA-4 antibodies or antigen-binding fragments thereof, anti-CD27 antibodies or antigen-binding fragments thereof, anti-CD30 antibodies or antigen-binding fragments thereof, anti-CD40 antibodies or antigen-binding fragments thereof, anti-4-1 BB antibodies or antigen-binding fragments thereof, anti-GITR antibodies or antigen-binding fragments thereof, anti-OX40 antibodies or antigen-binding fragments thereof, anti-TRAILR1 antibodies or antigen-binding fragments thereof, anti-TRAILR2 antibodies or antigen-binding fragments thereof, anti-TWEAK antibodies or antigen-binding fragments thereof, anti-TWEAKR antibodies or antigen-binding fragments thereof, anti-BRAF antibodies or antigen-binding fragments thereof, anti-MEK antibodies or antigen-binding fragments thereof, anti-CD33 antibodies or antigen-binding fragments thereof, anti-CD20 antibodies or antigen-binding fragments thereof, anti-CD52 antibodies or antigen-binding fragments thereof, anti-A33 antibodies or antigen-binding fragments thereof, anti-GD3 antibodies or antigen-binding fragments thereof, anti-PSMA antibodies or antigen-binding fragments thereof, anti-Ceacan 1 antibodies or antigen-binding fragments thereof, anti-Galedin 9 antibodies or antigen-binding fragments thereof, anti-HVEM antibodies or antigen-binding fragments thereof, anti-VISTA antibodies or antigen-binding fragments thereof, anti-B7 H4 antibodies or antigen-binding fragments thereof, anti-HHLA2 antibodies or antigen-binding fragments thereof, anti-CD155 antibodies or antigen-binding fragments thereof, anti-CD80 antibodies or antigen-binding fragments thereof, anti-BTLA antibodies or antigen-binding fragments thereof, anti-CD160 antibodies or antigen-binding fragments thereof, anti-CD28 antibodies or antigen-binding fragments thereof, anti-CD226 antibodies or antigen-binding fragments thereof, anti-CEACAM1 antibodies or antigen-binding fragments thereof, anti-TIM3 antibodies or antigen-binding fragments thereof, anti-CD96 antibodies or antigen-binding fragments thereof, anti-CD70 antibodies or antigen-binding fragments thereof, anti-CD27 antibodies or antigen-binding fragments thereof, anti-LIGHT antibodies or antigen-binding fragments thereof, anti-CD137 antibodies or antigen-binding fragments thereof, anti-DR4 antibodies or antigen-binding fragments thereof, anti-CR5 antibodies or antigen-binding fragments thereof, anti-FAS antibodies or antigen-binding fragments thereof, anti-CD95 antibodies or antigen-binding fragments thereof, anti-TRAIL antibodies or antigen-binding fragments thereof, anti-DR6 antibodies or antigen-binding fragments thereof, anti-EDAR antibodies or antigen-binding fragments thereof, anti-NGFR antibodies or antigen-binding fragments thereof, anti-OPG antibodies or antigen-binding fragments thereof, anti-RANKL antibodies or antigen-binding fragments thereof, anti-LTPR antibodies or antigen-binding fragments thereof, anti-BCMA antibodies or antigen-binding fragments thereof, anti-TACI antibodies or antigen-binding fragments thereof, anti-BAFFR antibodies or antigen-binding fragments thereof, anti-EDAR2 antibodies or antigen-binding fragments thereof, anti-TROY antibodies or antigen-binding fragments thereof, and anti-RELT antibodies or antigen-binding fragments thereof.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1 LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The term “cancer” refers to a disease caused by an uncontrolled division of abnormal cells in a part of the body. In one instance, the cancer is kidney cancer e.g., an inoperable, locally advanced, or metastatic RCC. The cancer may be locally advanced or metastatic. In some instances, the cancer is locally advanced. In other instances, the cancer is metastatic. In some instances, the cancer may be unresectable (e.g., unresectable locally advanced or metastatic cancer). In some embodiments, the kidney cancer is sarcomatoid kidney cancer (e.g., sarcomatoid RCC (e.g., sarcomatoid advanced or mRCC)). In some embodiments, the kidney cancer is non-sarcomatoid kidney cancer (e.g., non-sarcomatoid RCC (e.g., non-sarcomatoid advanced or mRCC)). In some embodiments, the kidney cancer is clear cell kidney cancer (e.g., clear cell RCC (ccRCC) (e.g., advanced or metastatic ccRCC)). In some embodiments, the kidney cancer is non-clear cell kidney cancer (e.g., non-clear cell RCC (e.g., non-clear cell advanced or mRCC)).

As used herein, “cluster” refers to a subtype of a cancer (e.g., kidney cancer (e.g., inoperable, locally advanced, or metastatic RCC)) that is defined, e.g., transcriptionally (e.g., as assessed by RNA-seq or other techniques described herein) and/or by evaluation of somatic alterations. Cluster analysis can be used to identify subtypes of cancer by clustering samples (e.g., tumor samples) from patients having similar gene expression patterns and to find groups of genes that have similar expression profiles across different samples. A patient's sample (e.g., tumor sample) can be assigned into a cluster as described herein. In some examples, clusters are identified by non-negative matrix factorization (NMF); however, other clustering approaches are described herein and known in the art. In some examples, a patient's tumor sample is assigned into one of the following seven clusters based on the transcriptional profile of the patient's tumor: (1) angiogenic/stromal; (2) angiogenic; (3) complement/Q-oxidation; (4) T-effector/proliferative; (5) proliferative; (6) stromal/proliferative; and (7) snoRNA.

The term “sarcomatoid” refers to a cancer (e.g., kidney cancer (e.g., inoperable, locally advanced, or metastatic RCC)) that is characterized by sarcomatoid morphology, for example, as assessed by histology. Sarcomatoid kidney cancer (e.g., sarcomatoid RCC) is associated with aggressive behavior and poor prognosis. In some embodiments, a sarcomatoid kidney cancer includes or consists of atypical spindle-shaped cells and/or resembles any form of sarcoma. See, e.g., El Mouallem et al. Urol. Oncol. 36:265-271, 2018, which is incorporated herein by reference in its entirety. Sarcomatoid RCC can occur in any subtype of RCC, including clear cell RCC, chromophobe RCC, collecting duct carcinoma, renal medullary carcinoma, fumarate hydratase (FH)-deficient RCC, and succinate dehydrogenase (SDH)-deficient RCC. The incidence of sarcomatoid RCC varies among subtypes, but is typically higher in clear cell RCC (approximately 5-8%) and chromophobe RCC (approximately 8-10%). The histology of the sarcomatoid component can be variable, and may include a fibrosarcoma-like pattern, a pleomorphic undifferentiated sarcoma-like pattern, or other heterologous sarcomatoid patterns (e.g., osteosarcoma-, chondrosarcoma-, or rhabdomyosarcoma-like patterns). Necrosis is typically present in a large majority (about 90%) of cases. In some embodiments, there is no minimum amount or percentage of sarcomatoid differentiation for an individual's kidney cancer to be classified as sarcomatoid. Sarcomatoid RCC may be assessed as described in Example 1 of U.S. Patent Application Publication No. 2021/0253710, which is incorporated by reference herein in its entirety. In other embodiments, sarcomatoid RCC may be characterized as described by the 2012 International Society of Urological Pathology (ISUP) Vancouver consensus (see Srigley et al. Am. J. Surg. Pathol. 37:1469-89, 2013, which is incorporated herein by reference in its entirety).

The term “Memorial Sloan Kettering Cancer Center (MSKCC) risk score” refers to a scoring system based on set of prognostic factors associated with survival in kidney cancer (e.g., RCC, e.g., mRCC) patients. See, e.g., Motzer et al. J. Clin. Oncol. 17(8):2530-2540, 1999 and Motzer et al. J. Clin. Oncol. 20(1):289-296, 2002, which are incorporated herein by reference in their entirety. In some embodiments, a MSKCC risk score can be calculated based on the following factors: (i) a time from nephrectomy to treatment (e.g., systemic treatment) of less than one year, a lack of a nephrectomy, or an initial diagnosis with metastatic disease; (ii) a hemoglobin level less than the lower limit of normal (LLN), optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8(4−serum albumin (g/dL)); (iv) a serum lactate dehydrogenase (LDH) level greater than 1.5 times the upper limit of normal (ULN), optionally wherein the ULN is 140 U/L; and/or (v) a Karnofsky Performance Status (KPS) score of <80. In some embodiments, an individual has a favorable MSKCC risk score if the individual has zero of the preceding characteristics. In some embodiments, an individual has an intermediate MSKCC risk score if the individual has one or two of the preceding characteristics. In some embodiments, an individual has a poor MSKCC risk score if the individual has three or more of the preceding characteristics. In some examples, an individual's MSKCC risk score may be used to identify whether the individual may benefit from an anti-cancer therapy, e.g., an anti-cancer therapy that includes a PD-L1 axis binding antagonist (e.g., an anti-PD-L1 antibody such as atezolizumab) and a VEGF antagonist (e.g., an anti-VEGF antibody such as bevacizumab), e.g., as described in U.S. Patent Application Publication No. 2021/0253710.

As used herein, “treating” comprises effective cancer treatment with an effective amount of a therapeutic agent (e.g., a PD-1 axis binding antagonist (e.g., atezolizumab) or combination of therapeutic agents (e.g., a PD-1 axis antagonist and one or more additional therapeutic agents, e.g., a VEGF antagonist). Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the patient may be previously untreated or not have received prior systemic therapy), or second line or later treatment. In particular examples, the treatment may be first-line treatment (e.g., the patient may be previously untreated or not have received prior systemic therapy).

Herein, an “effective amount” refers to the amount of a therapeutic agent (e.g., a PD-1 axis binding antagonist (e.g., atezolizumab) or a combination of therapeutic agents (e.g., a PD-1 axis antagonist and one or more additional therapeutic agents, e.g., a VEGF antagonist)), that achieves a therapeutic result. In some examples, the effective amount of a therapeutic agent or a combination of therapeutic agents is the amount of the agent or of the combination of agents that achieves a clinical endpoint of improved overall response rate (ORR), a complete response (CR), a pathological complete response (pCR), a partial response (PR), improved survival (e.g., disease-free survival (DFS), progression-free survival (PFS) and/or overall survival (OS)), and/or improved duration of response (DOR). Improvement (e.g., in terms of response rate (e.g., ORR, CR, and/or PR), survival (e.g., PFS and/or OS), or DOR) may be relative to a suitable reference treatment, for example, treatment that does not include the PD-1 axis binding antagonist and/or treatment that includes a tyrosine kinase inhibitor (e.g., sunitinib). For example, treatment with an anti-cancer therapy that includes atezolizumab and bevacizumab may be compared with a reference treatment which is treatment with sunitinib. In another example, treatment with an anti-cancer therapy that includes avelumab and axitinib may be compared with a reference treatment which is treatment with sunitinib.

As used herein, “complete response” and “CR” refers to disappearance of the cancer. In some examples, tumor response is assessed according to RECIST v 1.1. For example, CR may be the disappearance of all target lesions and non-target lesions and (if applicable) normalization of tumor marker level or reduction in short axis of any pathological lymph nodes to <10 mm.

As used herein, “partial response” and “PR” refers to at least a 30% decrease in the sum of the longest diameters (SLD) of target lesions, taking as reference the baseline SLD prior to treatment. In some examples, tumor response is assessed according to RECIST v 1.1. For example, PR may be a ≥30% decrease in the sum of diameters (SoD) of target lesions (taking as reference the baseline SoD) or persistence of ≥1 non-target lesions(s) and/or (if applicable) maintenance of tumor marker level above the normal limits. In some examples, the SoD may be of the longest diameters for non-nodal lesions, and the short axis for nodal lesions.

As used herein, “disease progression,” “progressive disease,” and “PD” refers to an increase in the size or number of target lesions. For example, PD may be a ≥20% relative increase in the sum of diameters (SoD) of all target lesions, taking as reference the smallest SoD on study, including baseline, and an absolute increase of ≥5 mm; ≥1 new lesion(s); and/or unequivocal progression of existing non-target lesions. In some examples, the SoD may be of the longest diameters for non-nodal lesions, and the short axis for nodal lesions.

As used herein, “overall response rate,” “objective response rate,” and “ORR” refer interchangeably to the sum of CR rate and PR rate. For example, ORR may refer to the percentage of participants with a documented CR or PR.

As used herein, “progression-free survival” and “PFS” refer to the length of time during and after treatment during which the cancer does not get worse. PFS may include the amount of time patients have experienced a CR or a PR, as well as the amount of time patients have experienced stable disease. For example, PFS may be the time from randomization to PD, as determined by the investigator per RECIST v 1.1, or death from any cause, whichever occurred first.

As used herein, “overall survival” and “OS” refer to the length of time from either the date of diagnosis or the start of treatment for a disease (e.g., cancer) that the patient is still alive. For example, OS may be the time from randomization to death due to any cause.

As used herein, the term “duration of response” and “DOR” refer to a length of time from documentation of a tumor response until disease progression or death from any cause, whichever occurs first. For example, DOR may be the time from the first occurrence of CR/PR to PD as determined by the investigator per RECIST v 1.1, or death from any cause, whichever occurred first.

As used herein, the term “chemotherapeutic agent” refers to a compound useful in the treatment of cancer, such as kidney cancer (e.g., RCC, e.g., an inoperable, locally advanced, or metastatic RCC). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “small molecule” refers to any molecule with a molecular weight of about 2000 daltons or less, preferably of about 500 daltons or less.

The term “patient” refers to a human patient. For example, the patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).
   Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and a VEGF antagonist (e.g., bevacizumab). As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3 week cycle. The term “detection” includes any means of detecting, including direct and indirect detection.

The term “biomarker” as used herein refers to an indicator, e.g., predictive, diagnostic, and/or prognostic, which can be detected in a sample, for example, a cluster, gene, or an alteration (e.g., a somatic alteration) disclosed herein. The biomarker may serve as an indicator of a particular subtype of a disease or disorder (e.g., cancer) characterized by certain, molecular, pathological, histological, and/or clinical features. Biomarkers include, but are not limited to, clusters, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers. In some examples, a biomarker is a cluster, e.g., a cluster identified by NMF, e.g., one of the following clusters: (1) angiogenic/stromal; (2) angiogenic; (3) complement/Q-oxidation; (4) T-effector/proliferative; (5) proliferative; (6) stromal/proliferative; and (7) snoRNA. In other examples, a biomarker is a gene. In yet other examples, a biomarker is an alteration (e.g., a somatic alteration).

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker).

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “diagnosis” is used herein to refer to the identification or classification of a molecular or pathological state, disease or condition (e.g., cancer (e.g., kidney cancer (e.g., RCC, e.g., an inoperable, locally advanced, or metastatic RCC))). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)). In some examples, a patient may be diagnosed by classifying the patient's cancer according to the methods disclosed herein, e.g., by assigning the patient's tumor sample into one of the following seven clusters based on the transcriptional profile of the patient's tumor: (1) angiogenic/stromal; (2) angiogenic; (3) complement/Q-oxidation; (4) T-effector/proliferative; (5) proliferative (6) stromal/proliferative; and (7) snoRNA.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor (e.g., a liver tumor) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+T lymphocytes and/or CD4+T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” “control tissue,” or “reference level,” as used herein, refers to a sample, cell, tissue, standard, or level that is used for comparison purposes. In one embodiment, a reference sample, reference cell, reference tissue, control sample, control cell, control tissue, or reference level is obtained from a healthy and/or non-diseased part of the body (e.g., tissue or cells) of the same subject or individual. For example, the reference sample, reference cell, reference tissue, control sample, control cell, control tissue, or reference level may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, control tissue, or reference level is obtained from a healthy and/or non-diseased part of the body (e.g., tissues or cells) of an individual who is not the subject or individual. In even another embodiment, a reference sample, reference cell, reference tissue, control sample, control cell, control tissue, or reference level is obtained from an untreated tissue and/or cell of the body of an individual who is not the subject or individual.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

The phrase “based on” when used herein means that the information about one or more biomarkers is used to inform a treatment decision, information provided on a package insert, or marketing/promotional guidance, and the like. For example, a patient may be selected for an anti-cancer therapy and/or treated with an anti-cancer therapy based on classification of the patient as disclosed herein, e.g., by assignment of the patient's tumor sample into one of the following seven clusters based on the transcriptional profile of the patient's tumor: (1) angiogenic/stromal; (2) angiogenic; (3) complement/Q-oxidation; (4) T-effector/proliferative; (5) proliferative (6) stromal/proliferative; and (7) snoRNA. In another example, a patient may be selected for an anti-cancer therapy and/or treated with an anti-cancer therapy based on (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1.

The term “multiplex-PCR” refers to a single PCR reaction carried out on nucleic acid obtained from a single source (e.g., an individual) using more than one primer set for the purpose of amplifying two or more DNA sequences in a single reaction.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real-time polymerase chain reaction” or “qRT-PCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

The term “RNA-seq,” also called “Whole Transcriptome Shotgun Sequencing (WTSS),” refers to the use of high-throughput sequencing technologies to sequence and/or quantify cDNA to obtain information about a sample's RNA content. Publications describing RNA-seq include: Wang et al. Nature Reviews Genetics 10(1):57-63, 2009; Ryan et al. BioTechniques 45(1):81-94, 2008; and Maher et al. Nature 458(7234):97-101, 2009.

### II. Methods of Classifying Kidney Cancer

Provided herein are methods for classifying kidney cancer (e.g., an inoperable, locally advanced, or metastatic RCC), which may involve assigning a sample (e.g., a tumor sample) from the patient into a cluster as disclosed herein.

In one example, provided herein is a method of classifying a kidney cancer (e.g., RCC, e.g., an inoperable, locally advanced, or metastatic RCC) in a human patient, the method comprising assigning a sample obtained from the patient into one of the following seven clusters based on a transcriptional profile of the patient's sample: (1) angiogenic/stromal; (2) angiogenic; (3) complement/Q-oxidation; (4) T-effector/proliferative; (5) proliferative; (6) stromal/proliferative; and (7) snoRNA, thereby classifying the kidney cancer in the patient. In some examples, the transcriptional profile has been provided by assaying mRNA in a sample (e.g., a tumor sample) from the patient.

In another example, provided herein is a method of classifying a kidney cancer (e.g., RCC, e.g., an inoperable, locally advanced, or metastatic RCC) in a human patient, the method comprising: (a) assaying mRNA in a tumor sample from the patient to provide a transcriptional profile of the patient's tumor; and (b) assigning the patient's tumor sample into one of the following seven clusters based on the transcriptional profile of the patient's tumor: (1) angiogenic/stromal; (2) angiogenic; (3) complement/Q-oxidation; (4) T-effector/proliferative; (5) proliferative; (6) stromal/proliferative; and (7) snoRNA, thereby classifying the kidney cancer in the patient.

In some examples, the kidney cancer is previously untreated.

In one example, provided herein is a method of classifying a kidney cancer (e.g., RCC, e.g., an inoperable, locally advanced, or metastatic RCC) in a human patient, wherein the kidney cancer is previously untreated, the method comprising assigning the patient's tumor sample into one of the following seven clusters based on a transcriptional profile of the patient's tumor: (1) angiogenic/stromal; (2) angiogenic; (3) complement/Q-oxidation; (4) T-effector/proliferative; (5) proliferative; (6) stromal/proliferative; and (7) snoRNA, thereby classifying the kidney cancer in the patient. In some examples, the transcriptional profile has been provided by assaying mRNA in a sample (e.g., a tumor sample) from the patient.

In another example, provided herein is a method of classifying a kidney cancer (e.g., RCC, e.g., an inoperable, locally advanced, or metastatic RCC) in a human patient, wherein the kidney cancer is previously untreated, the method comprising: (a) assaying mRNA in a tumor sample from the patient to provide a transcriptional profile of the patient's tumor; and (b) assigning the patient's tumor sample into one of the following seven clusters based on the transcriptional profile of the patient's tumor: (1) angiogenic/stromal; (2) angiogenic; (3) complement/Ω-oxidation; (4) T-effector/proliferative; (5) proliferative; (6) stromal/proliferative; and (7) snoRNA, thereby classifying the kidney cancer in the patient.

Any suitable approach for assaying mRNA may be used. In some examples, assaying mRNA in the tumor sample from the patient comprises RNA sequencing (RNA-seq), reverse transcription-quantitative polymerase chain reaction (RT-qPCR), qPCR, multiplex qPCR or RT-qPCR, microarray analysis, serial analysis of gene expression (SAGE), MassARRAY technique, in situ hybridization (ISH), or a combination thereof. In some particular examples, assaying mRNA in the tumor sample from the patient comprises RNA-seq.

Any suitable approach can be used to identify clusters into which a patient's sample (e.g., tumor sample) may be assigned. For example, in some examples, clusters are identified by non-negative matrix factorization (NMF; see, e.g., Lee et al. Nature 401(6755):788-791, 1999 and Brunet et al. Proc. Nat'l Acad. Sci. USA 101:4164-4169, 2004), hierarchical clustering (see, e.g., Eisen et al. Proc. Nat'l Acad. Sci. USA 95(25):14863-8, 1998), partition clustering (e.g., K-means clustering, K-mediods clustering, or partitioning around medioids (PAM, see, e.g., Kaufman et al. Finding Groups in Data: John Wiley and Sons, Inc. 2008, pages 68-125)), model-based clustering (e.g., gaussian mixture models), principal component analysis, clustering with deep learning (see, e.g., Li et al. Nat. Commun. 11:2338, 2020), self-organizing map (see, e.g., Kohonen et al. Biol. Cybernet. 43(1):59-69, 1982), density-based spatial clustering of applications with noise (DBSCAN, see, e.g., Ester et al. Proceedings of the Second International Conference on Knowledge Discovery and Data Mining; Portland, Oregon: 3001507: AAAI Press; 1996. p. 226-31), and the like. In some examples, hierarchical clustering may include single-linkage, average-linkage, or complete-linkage hierarchical clustering algorithms. Reviews of exemplary clustering approaches are provided, e.g., in Oyalade et al. Bioinform. And Biol. Insights 10:237-253, 2016; Vidman et al. PLoS One 14(12)e0219102, 2019; and Jamail and Moussa, IntechOpen (DOI: 10.5772/intechopen.94069). In particular examples, clusters are identified by non-negative NMF, e.g., as described herein in Example 1.

In some examples, RNA-seq count data may be transformed prior to cluster analysis. Any suitable transformation approach can be used, e.g., logarithmic transformation (e.g., log 2-transformation), variance stabilizing transformation, eight data transformation, and the like.

In some examples, the seven clusters are identified by NMF. In some examples, the seven clusters identified by NMF are based on a set of genes representing the top 10% most variable genes in a population of patients having previously untreated kidney cancer (e.g., an inoperable, locally advanced, or metastatic RCC). In some examples, the set of genes is set forth in Table 1.

TABLE 1
Genes Representing Top 10% Most Variable Transcripts in Previously Untreated Kidney Cancer

| Gene ID | Symbol | Gene Name | Gene ID | Symbol | Gene Name |
|---|---|---|---|---|---|
| 100 | ADA | adenosine deaminase | 4329 | ALDH6A1 | aldehyde dehydrogenase 6 family |
| | | | | | member A1 |
| 100033413 | SNORD116-1 | small nucleolar RNA, C/D box | 4332 | MNDA | myeloid cell nuclear differentiation |
| | | 116-1 | | | antigen |
| 100033414 | SNORD116-2 | small nucleolar RNA, C/D box | 4337 | MOCS1 | molybdenum cofactor synthesis 1 |
| | | 116-2 | | | |
| 100033418 | SNORD116-6 | small nucleolar RNA, C/D box | 4345 | CD200 | CD200 molecule |
| | | 116-6 | | | |
| 100033420 | SNORD116-8 | small nucleolar RNA, C/D box | 4360 | MRC1 | mannose receptor C-type 1 |
| | | 116-8 | | | |
| 100033423 | SNORD116-11 | small nucleolar RNA, C/D box | 439921 | MXRA7 | matrix remodeling associated 7 |
| | | 116-11 | | | |
| 100033425 | SNORD116-13 | small nucleolar RNA, C/D box | 440050 | KRTAP5-7 | keratin associated protein 5-7 |
| | | 116-13 | | | |
| 100033426 | SNORD116-14 | small nucleolar RNA, C/D box | 440270 | GOLGA8B | golgin A8 family member B |
| | | 116-14 | | | |
| 100033427 | SNORD116-15 | small nucleolar RNA, C/D box | 440348 | NPIPB15 | nuclear pore complex interacting |
| | | 116-15 | | | protein family member B15 |
| 100033428 | SNORD116-16 | small nucleolar RNA, C/D box | 440482 | ANKRD20A5P | ankyrin repeat domain 20 family |
| | | 116-16 | | | member A5, pseudogene |
| 100033431 | SNORD116-20 | small nucleolar RNA, C/D box | 440567 | UQCRHL | ubiquinol-cytochrome c reductase |
| | | 116-20 | | | hinge protein like |
| 100033432 | SNORD116-21 | small nucleolar RNA, C/D box | 440585 | FAM183A | family with sequence similarity |
| | | 116-21 | | | 183 member A |
| 100033433 | SNORD116-22 | small nucleolar RNA, C/D box | 440689 | HIST2H2BF | histone cluster 2 H2B family |
| | | 116-22 | | | member f |
| 100033434 | SNORD116-23 | small nucleolar RNA, C/D box | 440712 | RHEX | regulator of hemoglobinization |
| | | 116-23 | | | and erythroid cell expansion |
| 100033435 | SNORD116-24 | small nucleolar RNA, C/D box | 441027 | TMEM150C | transmembrane protein 150C |
| | | 116-24 | | | |
| 100033436 | SNORD116-25 | small nucleolar RNA, C/D box | 441054 | C4orf47 | chromosome 4 open reading |
| | | 116-25 | | | frame 47 |
| 100033438 | SNORD116-26 | small nucleolar RNA, C/D box | 441124 | GTF2IP20 | general transcription factor IIi |
| | | 116-26 | | | pseudogene 20 |
| 100033439 | SNORD116-27 | small nucleolar RNA, C/D box | 441168 | CALHM6 | calcium homeostasis modulator |
| | | 116-27 | | | family member 6 |
| 100033804 | SNORD115-30 | small nucleolar RNA, C/D box | 441294 | CTAGE15 | CTAGE family member 15 |
| | | 115-30 | | | |
| 100033806 | SNORD115-32 | small nucleolar RNA, C/D box | 441528 | NA | NA |
| | | 115-32 | | | |
| 100033807 | SNORD115-33 | small nucleolar RNA, C/D box | 442213 | PTCHD4 | patched domain containing 4 |
| | | 115-33 | | | |
| 100033812 | SNORD115-38 | small nucleolar RNA, C/D box | 442319 | ZNF727 | zinc finger protein 727 |
| | | 115-38 | | | |
| 100033818 | SNORD115-44 | small nucleolar RNA, C/D box | 443 | ASPA | aspartoacylase |
| | | 115-44 | | | |
| 100033821 | SNORD116-29 | small nucleolar RNA, C/D box | 445 | ASS1 | argininosuccinate synthase 1 |
| | | 116-29 | | | |
| 100049587 | SIGLEC14 | sialic acid binding Ig like | 445347 | TARP | TCR gamma alternate reading |
| | | lectin 14 | | | frame protein |
| 10008 | KCNE3 | potassium voltage-gated | 4485 | MST1 | macrophage stimulating 1 |
| | | channel subfamily E | | | |
| | | regulatory subunit 3 | | | |
| 1001 | CDH3 | cadherin 3 | 4489 | MT1A | metallothionein 1A |
| 100113393 | SNORD12B | small nucleolar RNA, C/D box | 4493 | MT1E | metallothionein 1E |
| | | 12B | | | |
| 100124536 | SNORA38B | small nucleolar RNA, H/ACA | 4494 | MT1F | metallothionein 1F |
| | | box 38B | | | |
| 100124539 | SNORA11B | small nucleolar RNA, H/ACA | 4495 | MT1G | metallothionein 1G |
| | | box 11B | | | |
| 100126299 | VTRNA2-1 | vault RNA 2-1 | 4496 | MT1H | metallothionein 1H |
| 100127983 | C8orf88 | chromosome 8 open reading | 4499 | MT1M | metallothionein 1M |
| | | frame 88 | | | |
| 100129543 | ZNF730 | zinc finger protein 730 | 4500 | MT1L | metallothionein 1L, pseudogene |
| 100129697 | LOC100129697 | uncharacterized | 4501 | MT1X | metallothionein 1X |
| | | LOC100129697 | | | |
| 100132116 | ACTA2-AS1 | ACTA2 antisense RNA 1 | 4502 | MT2A | metallothionein 2A |
| 100132287 | LOC100132287 | uncharacterized | 4504 | MT3 | metallothionein 3 |
| | | LOC100132287 | | | |
| 100132417 | FCGR1CP | Fc fragment of IgG receptor | 4508 | ATP6 | ATP synthase F0 subunit 6 |
| | | Ic, pseudogene | | | |
| 100151683 | RNU4ATAC | RNA, U4atac small nuclear | 4509 | ATP8 | ATP synthase F0 subunit 8 |
| | | (U12-dependent splicing) | | | |
| 100151684 | RNU6ATAC | RNA, U6atac small nuclear | 4512 | COX1 | cytochrome c oxidase subunit I |
| | | (U12-dependent splicing) | | | |
| 100192204 | PPIAP30 | peptidylprolyl isomerase A | 4513 | COX2 | cytochrome c oxidase subunit II |
| | | pseudogene 30 | | | |
| 1002 | CDH4 | cadherin 4 | 4514 | COX3 | cytochrome c oxidase III |
| 100233156 | LOC100233156 | tektin 4 pseudogene | 4515 | MTCP1 | mature T-cell proliferation 1 |
| 10024 | TROAP | trophinin associated protein | 4519 | CYTB | cytochrome b |
| 100240734 | LOC100240734 | uncharacterized | 4535 | ND1 | NADH dehydrogenase, subunit 1 |
| | | LOC100240734 | | | (complex I) |
| 100271927 | RASA4B | RAS p21 protein activator 4B | 4536 | ND2 | MTND2 |
| 100272147 | CMC4 | C-X9-C motif containing 4 | 4537 | ND3 | NADH dehydrogenase, subunit 3 |
| | | | | | (complex I) |
| 100287171 | WASHC1 | WASH complex subunit 1 | 4538 | ND4 | NADH dehydrogenase, subunit 4 |
| | | | | | (complex I) |
| 100287569 | LINC00173 | long intergenic non-protein | 4539 | ND4L | NADH dehydrogenase, subunit 4L |
| | | coding RNA 173 | | | (complex I) |
| 100288152 | SLC9A3-AS1 | SLC9A3 antisense RNA 1 | 4540 | ND5 | NADH dehydrogenase, subunit 5 |
| | | | | | (complex I) |
| 100288332 | NPIPA5 | nuclear pore complex | 4541 | ND6 | NADH dehydrogenase, subunit 6 |
| | | interacting protein family | | | (complex I) |
| | | member A5 | | | |
| 100288778 | LOC100288778 | WASH complex subunit 1 | 4543 | MTNR1A | melatonin receptor 1A |
| | | pseudogene | | | |
| 100289333 | LOC100289333 | uncharacterized | 4547 | MTTP | microsomal triglyceride transfer |
| | | LOC100289333 | | | protein |
| 100293211 | NA | NA | 4564 | TRNH | tRNA |
| 100294362 | LOC100294362 | uncharacterized | 4569 | TRNM | tRNA |
| | | LOC100294362 | | | |
| 1003 | CDH5 | cadherin 5 | 4582 | MUC1 | mucin 1, cell surface associated |
| 100302743 | SNORA80B | small nucleolar RNA, H/ACA | 4584 | MUC3A | mucin 3A, cell surface associated |
| | | box 80B | | | |
| 100303491 | ZEB2-AS1 | ZEB2 antisense RNA 1 | 4605 | MYBL2 | MYB proto-oncogene like 2 |
| 100313769 | MIR320B2 | microRNA 320b-2 | 4616 | GADD45B | growth arrest and DNA damage |
| | | | | | inducible beta |
| 1004 | CDH6 | cadherin 6 | 4629 | MYH11 | myosin heavy chain 11 |
| 100423062 | IGLL5 | immunoglobulin lambda like | 4634 | MYL3 | myosin light chain 3 |
| | | polypeptide 5 | | | |
| 10050 | SLC17A4 | solute carrier family 17 | 4645 | MYO5B | myosin VB |
| | | member 4 | | | |
| 100505679 | UBE2Q2L | ubiquitin conjugating enzyme | 4647 | MYO7A | myosin VIIA |
| | | E2 Q2 like | | | |
| 100506658 | OCLN | occludin | 4648 | MYO7B | myosin VIIB |
| 100506736 | SLFN12L | schlafen family member 12 | 467 | ATF3 | activating transcription factor 3 |
| | | like | | | |
| 100506755 | MIR497HG | mir-497-195 cluster host | 4674 | NAP1L2 | nucleosome assembly protein 1 |
| | | gene | | | like 2 |
| 100506898 | MAGOH2P | mago homolog 2, | 4684 | NCAM1 | neural cell adhesion molecule 1 |
| | | pseudogene | | | |
| 100507203 | SMLR1 | small leucine rich protein 1 | 4688 | NCF2 | neutrophil cytosolic factor 2 |
| 100507421 | TMEM178B | transmembrane protein 178B | 4689 | NCF4 | neutrophil cytosolic factor 4 |
| 100509457 | NA | NA | 4703 | NEB | Nebulin |
| 100510710 | LOC100510710 | glucosylceramidase-like | 4739 | NEDD9 | neural precursor cell expressed, |
| | | | | | developmentally down-regulated 9 |
| 10053 | AP1M2 | adaptor related protein | 4741 | NEFM | neurofilament medium |
| | | complex 1 mu 2 subunit | | | |
| 100652781 | SNX29P1 | sorting nexin 29 pseudogene | 4747 | NEFL | neurofilament light |
| | | 1 | | | |
| 10071 | MUC12 | mucin 12, cell surface | 4751 | NEK2 | NIMA related kinase 2 |
| | | associated | | | |
| 10076 | PTPRU | protein tyrosine phosphatase, | 4753 | NELL2 | neural EGFL like 2 |
| | | receptor type U | | | |
| 10083 | USH1C | USH1 protein network | 477 | ATP1A2 | ATPase Na₊/K₊ transporting |
| | | component harmonin | | | subunit alpha 2 |
| 10085 | EDIL3 | EGF like repeats and | 478 | ATP1A3 | ATPase Na₊/K₊ transporting |
| | | discoidin domains 3 | | | subunit alpha 3 |
| 100874323 | HOXA10-AS | HOXA10 antisense RNA | 4803 | NGF | nerve growth factor |
| 1009 | CDH11 | cadherin 11 | 4804 | NGFR | nerve growth factor receptor |
| 100996809 | NA | NA | 481 | ATP1B1 | ATPase Na₊/K₊ transporting |
| | | | | | subunit beta 1 |
| 10100 | TSPAN2 | tetraspanin 2 | 4818 | NKG7 | natural killer cell granule protein 7 |
| 10103 | TSPAN1 | tetraspanin 1 | 482 | ATP1B2 | ATPase Na₊/K₊ transporting |
| | | | | | subunit beta 2 |
| 101059918 | GOLGA8R | golgin A8 family member R | 4828 | NMB | neuromedin B |
| 101060026 | NA | NA | 4837 | NNMT | nicotinamide N-methyltransferase |
| 101060789 | NA | NA | 4854 | NOTCH3 | notch 3 |
| 101060846 | NA | NA | 4855 | NOTCH4 | notch 4 |
| 10107 | TRIM10 | tripartite motif containing 10 | 4856 | NOV | nephroblastoma overexpressed |
| 10110 | SGK2 | SGK2, serine/threonine | 4857 | NOVA1 | NOVA alternative splicing |
| | | kinase 2 | | | regulator 1 |
| 10112 | KIF20A | kinesin family member 20A | 486 | FXYD2 | FXYD domain containing ion |
| | | | | | transport regulator 2 |
| 10117 | ENAM | enamelin | 487 | ATP2A1 | ATPase |
| | | | | | sarcoplasmic/endoplasmic |
| | | | | | reticulum Ca2₊ transporting 1 |
| 1012 | CDH13 | cadherin 13 | 4881 | NPR1 | natriuretic peptide receptor 1 |
| 10123 | ARL4C | ADP ribosylation factor like | 4883 | NPR3 | natriuretic peptide receptor 3 |
| | | GTPase 4C | | | |
| 10125 | RASGRP1 | RAS guanyl releasing protein | 4885 | NPTX2 | neuronal pentraxin 2 |
| | | 1 | | | |
| 10129 | FRY | FRY microtubule binding | 4886 | NPY1R | neuropeptide Y receptor Y1 |
| | | protein | | | |
| 1014 | CDH16 | cadherin 16 | 4888 | NPY6R | neuropeptide Y receptor Y6 |
| | | | | | (pseudogene) |
| 10141 | LINC01587 | long intergenic non-protein | 4897 | NRCAM | neuronal cell adhesion molecule |
| | | coding RNA 1587 | | | |
| 10144 | FAM13A | family with sequence | 4907 | NT5E | 5′-nucleotidase ecto |
| | | similarity 13 member A | | | |
| 10149 | ADGRG2 | adhesion G protein-coupled | 4908 | NTF3 | neurotrophin 3 |
| | | receptor G2 | | | |
| 10158 | PDZK1IP1 | PDZK1 interacting protein 1 | 491 | ATP2B2 | ATPase plasma membrane Ca2₊ |
| | | | | | transporting 2 |
| 10178 | TENM1 | teneurin transmembrane | 4915 | NTRK2 | neurotrophic receptor tyrosine |
| | | protein 1 | | | kinase 2 |
| 10186 | LHFPL6 | LHFPL tetraspan subfamily | 4916 | NTRK3 | neurotrophic receptor tyrosine |
| | | member 6 | | | kinase 3 |
| 101927594 | NA | NA | 4920 | ROR2 | receptor tyrosine kinase like |
| | | | | | orphan receptor 2 |
| 101927733 | NA | NA | 492307 | PPDPFL | pancreatic progenitor cell |
| | | | | | differentiation and proliferation |
| | | | | | factor like |
| 101927746 | LOC101927746 | uncharacterized | 4929 | NR4A2 | nuclear receptor subfamily 4 |
| | | LOC101927746 | | | group A member 2 |
| 101927905 | LINC02449 | long intergenic non-protein | 4935 | GPR143 | G protein-coupled receptor 143 |
| | | coding RNA 2449 | | | |
| 101927960 | LOC101927960 | uncharacterized | 4948 | OCA2 | OCA2 melanosomal |
| | | LOC101927960 | | | transmembrane protein |
| 101927999 | LOC101927999 | putative uncharacterized | 4958 | OMD | osteomodulin |
| | | protein FLJ44672 | | | |
| 101928149 | LOC101928149 | nascent polypeptide- | 4969 | OGN | osteoglycin |
| | | associated complex alpha | | | |
| | | subunit pseudogene | | | |
| 101928281 | NA | NA | 497190 | CLEC18B | C-type lectin domain family 18 |
| | | | | | member B |
| 101928706 | NA | NA | 4973 | OLR1 | oxidized low density lipoprotein |
| | | | | | receptor 1 |
| 101929206 | NA | NA | 4982 | TNFRSF11B | TNF receptor superfamily member |
| | | | | | 11b |
| 101929335 | ADAMTS9-AS1 | ADAMTS9 antisense RNA 1 | 5003 | SLC22A18AS | solute carrier family 22 member |
| | | | | | 18 antisense |
| 101929560 | LOC101929560 | uncharacterized | 5004 | ORM1 | orosomucoid 1 |
| | | LOC101929560 | | | |
| 101929773 | LOC101929773 | UDP-glucuronosyltransferase | 5010 | CLDN11 | claudin 11 |
| | | 2B10-like | | | |
| 101930013 | LOC101930013 | polycystin-1-like | 5046 | PCSK6 | proprotein convertase |
| | | | | | subtilisin/kexin type 6 |
| 101930662 | NA | NA | 50486 | GOS2 | G0/G1 switch 2 |
| 101930669 | NA | NA | 50507 | NOX4 | NADPH oxidase 4 |
| 10203 | CALCRL | calcitonin receptor like | 50509 | COL5A3 | collagen type V alpha 3 chain |
| | | receptor | | | |
| 10216 | PRG4 | proteoglycan 4 | 50512 | PODXL2 | podocalyxin like 2 |
| 10225 | CD96 | CD96 molecule | 5053 | PAH | phenylalanine hydroxylase |
| 10231 | RCAN2 | regulator of calcineurin 2 | 5054 | SERPINE1 | serpin family E member 1 |
| 10234 | LRRC17 | leucine rich repeat containing | 50614 | GALNT9 | polypeptide N- |
| | | 17 | | | acetylgalactosaminyltransferase 9 |
| 10246 | SLC17A2 | solute carrier family 17 | 5063 | PAK3 | p21 (RAC1) activated kinase 3 |
| | | member 2 | | | |
| 102465485 | MIR6809 | microRNA 6809 | 5071 | PRKN | parkin RBR E3 ubiquitin protein |
| | | | | | ligase |
| 102467147 | LINC01948 | long intergenic non-protein | 5076 | PAX2 | paired box 2 |
| | | coding RNA 1948 | | | |
| 10247 | RIDA | reactive intermediate imine | 50852 | TRAT1 | T-cell receptor associated |
| | | deaminase A homolog | | | transmembrane adaptor 1 |
| 10249 | GLYAT | glycine-N-acyltransferase | 50861 | STMN3 | stathmin 3 |
| 10252 | SPRY1 | sprouty RTK signaling | 5087 | PBX1 | PBX homeobox 1 |
| | | antagonist 1 | | | |
| 10256 | CNKSR1 | connector enhancer of kinase | 5091 | PC | pyruvate carboxylase |
| | | suppressor of Ras 1 | | | |
| 1026 | CDKN1A | cyclin dependent kinase | 50937 | CDON | cell adhesion associated, |
| | | inhibitor 1A | | | oncogene regulated |
| 10261 | IGSF6 | immunoglobulin superfamily | 50940 | PDE11A | phosphodiesterase 11A |
| | | member 6 | | | |
| 10265 | IRX5 | iroquois homeobox 5 | 5099 | PCDH7 | protocadherin 7 |
| 10266 | RAMP2 | receptor activity modifying | 5104 | SERPINA5 | serpin family A member 5 |
| | | protein 2 | | | |
| 10268 | RAMP3 | receptor activity modifying | 5105 | PCK1 | phosphoenolpyruvate |
| | | protein 3 | | | carboxykinase 1 |
| 10272 | FSTL3 | follistatin like 3 | 51084 | CRYL1 | crystallin lambda 1 |
| 102723407 | LOC102723407 | putative V-set and | 51085 | MLXIPL | MLX interacting protein like |
| | | immunoglobulin domain- | | | |
| | | containing-like protein | | | |
| | | IGHV4OR15-8 | | | |
| 102723493 | LOC102723493 | uncharacterized | 51087 | YBX2 | Y-box binding protein 2 |
| | | LOC102723493 | | | |
| 102723647 | RPL23AP97 | ribosomal protein L23a | 51090 | PLLP | plasmolipin |
| | | pseudogene 97 | | | |
| 102724058 | LOC102724058 | uncharacterized | 51129 | ANGPTL4 | angiopoietin like 4 |
| | | LOC102724058 | | | |
| 102724343 | NA | NA | 51162 | EGFL7 | EGF like domain multiple 7 |
| 102724424 | NA | NA | 51171 | HSD17B14 | hydroxysteroid 17-beta |
| | | | | | dehydrogenase 14 |
| 102724436 | NA | NA | 51176 | LEF1 | lymphoid enhancer binding factor |
| | | | | | 1 |
| 102724660 | LOC102724660 | uncharacterized | 51179 | HAO2 | hydroxyacid oxidase 2 |
| | | LOC102724660 | | | |
| 102724668 | DPY19L1P2 | DPY19L1 pseudogene 2 | 5118 | PCOLCE | procollagen C-endopeptidase |
| | | | | | enhancer |
| 102724788 | LOC102724788 | proline dehydrogenase 1, | 51200 | CPA4 | carboxypeptidase A4 |
| | | mitochondrial | | | |
| 102724850 | LOC102724850 | uncharacterized | 51206 | GP6 | glycoprotein VI platelet |
| | | LOC102724850 | | | |
| 102724880 | LOC102724880 | uncharacterized | 51232 | CRIM1 | cysteine rich transmembrane BMP |
| | | LOC102724880 | | | regulator 1 |
| 102725001 | NA | NA | 51233 | DRICH1 | aspartate rich 1 |
| 102725018 | NA | NA | 51237 | MZB1 | marginal zone B and B1 cell |
| | | | | | specific protein |
| 102725414 | NA | NA | 5125 | PCSK5 | proprotein convertase |
| | | | | | subtilisin/kexin type 5 |
| 10276 | NET1 | neuroepithelial cell | 51268 | PIPOX | pipecolic acid and sarcosine |
| | | transforming 1 | | | oxidase |
| 10288 | LILRB2 | leukocyte immunoglobulin | 51284 | TLR7 | toll like receptor 7 |
| | | like receptor B2 | | | |
| 10319 | LAMC3 | laminin subunit gamma 3 | 5129 | CDK18 | cyclin dependent kinase 18 |
| 10326 | SIRPB1 | signal regulatory protein beta | 51294 | PCDH12 | protocadherin 12 |
| | | 1 | | | |
| 1033 | CDKN3 | cyclin dependent kinase | 51299 | NRN1 | neuritin 1 |
| | | inhibitor 3 | | | |
| 10331 | B3GNT3 | UDP-GIcNAc:betaGal beta- | 51302 | CYP39A1 | cytochrome P450 family 39 |
| | | 1,3-N- | | | subfamily A member 1 |
| | | acetylglucosaminyltransferase | | | |
| | | 3 | | | |
| 10335 | MRVI1 | murine retrovirus integration | 51305 | KCNK9 | potassium two pore domain |
| | | site 1 homolog | | | channel subfamily K member 9 |
| 10350 | ABCA9 | ATP binding cassette | 51310 | SLC22A17 | solute carrier family 22 member |
| | | subfamily A member 9 | | | 17 |
| 10351 | ABCA8 | ATP binding cassette | 51311 | TLR8 | toll like receptor 8 |
| | | subfamily A member 8 | | | |
| 10370 | CITED2 | Cbp/p300 interacting | 51316 | PLAC8 | placenta specific 8 |
| | | transactivator with Glu/Asp | | | |
| | | rich carboxy-terminal domain | | | |
| | | 2 | | | |
| 10371 | SEMA3A | semaphorin 3A | 5133 | PDCD1 | programmed cell death 1 |
| 103752587 | FOXC2-AS1 | FOXC2 antisense RNA 1 | 51330 | TNFRSF12A | TNF receptor superfamily member |
| | | | | | 12A |
| 10381 | TUBB3 | tubulin beta 3 class III | 51338 | MS4A4A | membrane spanning 4-domains |
| | | | | | A4A |
| 10382 | TUBB4A | tubulin beta 4A class IVa | 51339 | DACT1 | dishevelled binding antagonist of |
| | | | | | beta catenin 1 |
| 103908605 | LOC103908605 | uncharacterized | 51351 | ZNF117 | zinc finger protein 117 |
| | | LOC103908605 | | | |
| 10397 | NDRG1 | N-myc downstream regulated | 5136 | PDE1A | phosphodiesterase 1A |
| | | 1 | | | |
| 1040 | CDS1 | CDP-diacylglycerol synthase | 51361 | HOOK1 | hook microtubule tethering protein |
| | | 1 | | | 1 |
| 10406 | WFDC2 | WAP four-disulfide core | 51365 | PLA1A | phospholipase A1 member A |
| | | domain 2 | | | |
| 10409 | BASP1 | brain abundant membrane | 5137 | PDE1C | phosphodiesterase 1C |
| | | attached signal protein 1 | | | |
| 10411 | RAPGEF3 | Rap guanine nucleotide | 5138 | PDE2A | phosphodiesterase 2A |
| | | exchange factor 3 | | | |
| 10417 | SPON2 | spondin 2 | 5139 | PDE3A | phosphodiesterase 3A |
| 10418 | SPON1 | spondin 1 | 5140 | PDE3B | phosphodiesterase 3B |
| 1043 | CD52 | CD52 molecule | 51411 | BIN2 | bridging integrator 2 |
| 10437 | IFI30 | IFI30, lysosomal thiol | 51421 | AMOTL2 | angiomotin like 2 |
| | | reductase | | | |
| 10439 | OLFM1 | olfactomedin 1 | 51435 | SCARA3 | scavenger receptor class A |
| | | | | | member 3 |
| 10449 | ACAA2 | acetyl-CoA acyltransferase 2 | 51454 | GULP1 | GULP, engulfment adaptor PTB |
| | | | | | domain containing 1 |
| 10457 | GPNMB | glycoprotein nmb | 51471 | NAT8B | N-acetyltransferase 8B (putative, |
| | | | | | gene/pseudogene) |
| 10462 | CLEC10A | C-type lectin domain | 51473 | DCDC2 | doublecortin domain containing 2 |
| | | containing 10A | | | |
| 1047 | CLGN | calmegin | 51513 | ETV7 | ETS variant 7 |
| 10489 | LRRC41 | leucine rich repeat containing | 5152 | PDE9A | phosphodiesterase 9A |
| | | 41 | | | |
| 1050 | CEBPA | CCAAT/enhancer binding | 51559 | NT5DC3 | 5′-nucleotidase domain containing |
| | | protein alpha | | | 3 |
| 10509 | SEMA4B | semaphorin 4B | 5156 | PDGFRA | platelet derived growth factor |
| | | | | | receptor alpha |
| 1051 | CEBPB | CCAAT/enhancer binding | 51560 | RAB6B | RAB6B, member RAS oncogene |
| | | protein beta | | | family |
| 10512 | SEMA3C | semaphorin 3C | 5157 | PDGFRL | platelet derived growth factor |
| | | | | | receptor like |
| 10516 | FBLN5 | fibulin 5 | 5158 | PDE6B | phosphodiesterase 6B |
| 10529 | NEBL | nebulette | 5159 | PDGFRB | platelet derived growth factor |
| | | | | | receptor beta |
| 10536 | P3H3 | prolyl 3-hydroxylase 3 | 5164 | PDK2 | pyruvate dehydrogenase kinase 2 |
| 10537 | UBD | ubiquitin D | 51655 | RASD1 | ras related dexamethasone |
| | | | | | induced 1 |
| 10538 | BATF | basic leucine zipper ATF-like | 51659 | GINS2 | GINS complex subunit 2 |
| | | transcription factor | | | |
| 10563 | CXCL13 | C-X-C motif chemokine | 5166 | PDK4 | pyruvate dehydrogenase kinase 4 |
| | | ligand 13 | | | |
| 10568 | SLC34A2 | solute carrier family 34 | 5167 | ENPP1 | ectonucleotide |
| | | member 2 | | | pyrophosphatase/phosphodiesterase |
| | | | | | 1 |
| 10578 | GNLY | granulysin | 51673 | TPPP3 | tubulin polymerization promoting |
| | | | | | protein family member 3 |
| 10579 | TACC2 | transforming acidic coiled-coil | 51678 | MPP6 | membrane palmitoylated protein 6 |
| | | containing protein 2 | | | |
| 10580 | SORBS1 | sorbin and SH3 domain | 5168 | ENPP2 | ectonucleotide |
| | | containing 1 | | | pyrophosphatase/phosphodiesterase |
| | | | | | 2 |
| 10590 | SCGN | secretagogin, EF-hand | 5169 | ENPP3 | ectonucleotide |
| | | calcium binding protein | | | pyrophosphatase/phosphodiester |
| | | | | | ase 3 |
| 10610 | ST6GALNAC2 | ST6 N-acetylgalactosaminide | 51700 | CYB5R2 | cytochrome b5 reductase 2 |
| | | alpha-2,6-sialyltransferase 2 | | | |
| 10615 | SPAG5 | sperm associated antigen 5 | 51703 | ACSL5 | acyl-CoA synthetase long chain |
| | | | | | family member 5 |
| 1062 | CENPE | centromere protein E | 51704 | GPRC5B | G protein-coupled receptor class |
| | | | | | C group 5 member B |
| 1063 | CENPF | centromere protein F | 51705 | EMCN | endomucin |
| 10630 | PDPN | podoplanin | 51733 | UPB1 | beta-ureidopropionase 1 |
| 10631 | POSTN | periostin | 5174 | PDZK1 | PDZ domain containing 1 |
| 10642 | IGF2BP1 | insulin like growth factor 2 | 51751 | HIGD1B | HIG1 hypoxia inducible domain |
| | | mRNA binding protein 1 | | | family member 1B |
| 10643 | IGF2BP3 | insulin like growth factor 2 | 5176 | SERPINF1 | serpin family F member 1 |
| | | mRNA binding protein 3 | | | |
| 10644 | IGF2BP2 | insulin like growth factor 2 | 51760 | SYT17 | synaptotagmin 17 |
| | | mRNA binding protein 2 | | | |
| 10647 | SCGB1D2 | secretoglobin family 1D | 5187 | PER1 | period circadian regulator 1 |
| | | member 2 | | | |
| 1066 | CES1 | carboxylesterase 1 | 5197 | PF4V1 | platelet factor 4 variant 1 |
| 10663 | CXCR6 | C-X-C motif chemokine | 5222 | PGA5 | pepsinogen 5, group I |
| | | receptor 6 | | | (pepsinogen A) |
| 10669 | CGREF1 | cell growth regulator with EF- | 5224 | PGAM2 | phosphoglycerate mutase 2 |
| | | hand domain 1 | | | |
| 10673 | TNFSF13B | TNF superfamily member 13b | 5228 | PGF | placental growth factor |
| 10687 | PNMA2 | PNMA family member 2 | 5239 | PGM5 | phosphoglucomutase 5 |
| 1071 | CETP | cholesteryl ester transfer | 5243 | ABCB1 | ATP binding cassette subfamily B |
| | | protein | | | member 1 |
| 10718 | NRG3 | neuregulin 3 | 5244 | ABCB4 | ATP binding cassette subfamily B |
| | | | | | member 4 |
| 10742 | RAI2 | retinoic acid induced 2 | 5255 | PHKA1 | phosphorylase kinase regulatory |
| | | | | | subunit alpha 1 |
| 10752 | CHL1 | cell adhesion molecule L1 | 5265 | SERPINA1 | serpin family A member 1 |
| | | like | | | |
| 10763 | NES | nestin | 5266 | PI3 | peptidase inhibitor 3 |
| 10786 | SLC17A3 | solute carrier family 17 | 5270 | SERPINE2 | serpin family E member 2 |
| | | member 3 | | | |
| 108 | ADCY2 | adenylate cyclase 2 | 5274 | SERPINI1 | serpin family I member 1 |
| 10819 | OR7E14P | olfactory receptor family 7 | 5284 | PIGR | polymeric immunoglobulin |
| | | subfamily E member 14 | | | receptor |
| | | pseudogene | | | |
| 10826 | FAXDC2 | fatty acid hydroxylase domain | 5307 | PITX1 | paired like homeodomain 1 |
| | | containing 2 | | | |
| 10840 | ALDH1L1 | aldehyde dehydrogenase 1 | 5313 | PKLR | pyruvate kinase L/R |
| | | family member L1 | | | |
| 10841 | FTCD | formimidoyltransferase | 5314 | PKHD1 | PKHD1, fibrocystin/polyductin |
| | | cyclodeaminase | | | |
| 10846 | PDE10A | phosphodiesterase 10A | 5317 | PKP1 | plakophilin 1 |
| 10870 | HCST | hematopoietic cell signal | 5318 | PKP2 | plakophilin 2 |
| | | transducer | | | |
| 10874 | NMU | neuromedin U | 5319 | PLA2G1B | phospholipase A2 group IB |
| 10878 | CFHR3 | complement factor H related | 5320 | PLA2G2A | phospholipase A2 group IIA |
| | | 3 | | | |
| 10882 | C1QL1 | complement C1q like 1 | 5327 | PLAT | plasminogen activator, tissue type |
| 10891 | PPARGC1A | PPARG coactivator 1 alpha | 5328 | PLAU | plasminogen activator, urokinase |
| 10893 | MMP24 | matrix metallopeptidase 24 | 5329 | PLAUR | plasminogen activator, urokinase |
| | | | | | receptor |
| 10894 | LYVE1 | lymphatic vessel endothelial | 5332 | PLCB4 | phospholipase C beta 4 |
| | | hyaluronan receptor 1 | | | |
| 10903 | MTMR11 | myotubularin related protein | 5334 | PLCL1 | phospholipase C like 1 (inactive) |
| | | 11 | | | |
| 10911 | UTS2 | urotensin 2 | 53345 | TM6SF2 | transmembrane 6 superfamily |
| | | | | | member 2 |
| 10924 | SMPDL3A | sphingomyelin | 53347 | UBASH3A | ubiquitin associated and SH3 |
| | | phosphodiesterase acid like | | | domain containing A |
| | | 3A | | | |
| 10936 | GPR75 | G protein-coupled receptor | 53354 | PANK1 | pantothenate kinase 1 |
| | | 75 | | | |
| 10954 | PDIA5 | protein disulfide isomerase | 5341 | PLEK | pleckstrin |
| | | family A member 5 | | | |
| 10964 | IFI44L | interferon induced protein 44 | 5345 | SERPINF2 | serpin family F member 2 |
| | | like | | | |
| 10974 | ADIRF | adipogenesis regulatory | 5347 | PLK1 | polo like kinase 1 |
| | | factor | | | |
| 10990 | LILRB5 | leukocyte immunoglobulin | 5348 | FXYD1 | FXYD domain containing ion |
| | | like receptor B5 | | | transport regulator 1 |
| 10993 | SDS | serine dehydratase | 5350 | PLN | phospholamban |
| 11001 | SLC27A2 | solute carrier family 27 | 5352 | PLOD2 | procollagen-lysine,2-oxoglutarate |
| | | member 2 | | | 5-dioxygenase 2 |
| 11004 | KIF2C | kinesin family member 2C | 5355 | PLP2 | proteolipid protein 2 |
| 11005 | SPINK5 | serine peptidase inhibitor, | 5357 | PLS1 | plastin 1 |
| | | Kazal type 5 | | | |
| 11006 | LILRB4 | leukocyte immunoglobulin | 5360 | PLTP | phospholipid transfer protein |
| | | like receptor B4 | | | |
| 11013 | TMSB15A | thymosin beta 15a | 53616 | ADAM22 | ADAM metallopeptidase domain |
| | | | | | 22 |
| 11015 | KDELR3 | KDEL endoplasmic reticulum | 53630 | BCO1 | beta-carotene oxygenase 1 |
| | | protein retention receptor 3 | | | |
| 11040 | PIM2 | Pim-2 proto-oncogene, | 5365 | PLXNB3 | plexin B3 |
| | | serine/threonine kinase | | | |
| 11065 | UBE2C | ubiquitin conjugating enzyme | 5367 | PMCH | pro-melanin concentrating |
| | | E2 C | | | hormone |
| 11067 | DEPP1 | DEPP1, autophagy regulator | 5380 | PMS2P2 | PMS1 homolog 2, mismatch |
| | | | | | repair system component |
| | | | | | pseudogene 2 |
| 11069 | RAPGEF4 | Rap guanine nucleotide | 53829 | P2RY13 | purinergic receptor P2Y13 |
| | | exchange factor 4 | | | |
| 11078 | TRIOBP | TRIO and F-actin binding | 53833 | IL20RB | interleukin 20 receptor subunit |
| | | protein | | | beta |
| 11082 | ESM1 | endothelial cell specific | 53841 | CDHR5 | cadherin related family member 5 |
| | | molecule 1 | | | |
| 111 | ADCY5 | adenylate cyclase 5 | 53904 | MYO3A | myosin IIIA |
| 11113 | CIT | citron rho-interacting | 5396 | PRRX1 | paired related homeobox 1 |
| | | serine/threonine kinase | | | |
| 11117 | EMILIN1 | elastin microfibril interfacer 1 | 54 | ACP5 | acid phosphatase 5, tartrate |
| | | | | | resistant |
| 11118 | BTN3A2 | butyrophilin subfamily 3 | 540 | ATP7B | ATPase copper transporting beta |
| | | member A2 | | | |
| 11136 | SLC7A9 | solute carrier family 7 | 54039 | PCBP3 | poly(rC) binding protein 3 |
| | | member 9 | | | |
| 11148 | HHLA2 | HERV-H LTR-associating 2 | 54101 | RIPK4 | receptor interacting |
| | | | | | serine/threonine kinase 4 |
| 11151 | CORO1A | coronin 1A | 54102 | CLIC6 | chloride intracellular channel 6 |
| 11155 | LDB3 | LIM domain binding 3 | 5414 | SEPT4 | septin 4 |
| 1116 | CHI3L1 | chitinase 3 like 1 | 5420 | PODXL | podocalyxin like |
| 11167 | FSTL1 | follistatin like 1 | 54206 | ERRFI1 | ERBB receptor feedback inhibitor |
| | | | | | 1 |
| 1117 | CHI3L2 | chitinase 3 like 2 | 54209 | TREM2 | triggering receptor expressed on |
| | | | | | myeloid cells 2 |
| 1118 | CHIT1 | chitinase 1 | 54210 | TREM1 | triggering receptor expressed on |
| | | | | | myeloid cells 1 |
| 11184 | MAP4K1 | mitogen-activated protein | 54345 | SOX18 | SRY-box 18 |
| | | kinase kinase kinase kinase 1 | | | |
| 11185 | INMT | indolethylamine N- | 54360 | CYTL1 | cytokine like 1 |
| | | methyltransferase | | | |
| 11227 | GALNT5 | polypeptide N- | 54437 | SEMA5B | semaphorin 5B |
| | | acetylgalactosaminyltransferase | | | |
| | | 5 | | | |
| 112399 | EGLN3 | egl-9 family hypoxia inducible | 54443 | ANLN | anillin actin binding protein |
| | | factor 3 | | | |
| 11240 | PADI2 | peptidyl arginine deiminase 2 | 5446 | PON3 | paraoxonase 3 |
| 11247 | NXPH4 | neurexophilin 4 | 54463 | RETREG1 | reticulophagy regulator 1 |
| 11259 | FILIP1L | filamin A interacting protein 1 | 54507 | ADAMTSL4 | ADAMTS like 4 |
| | | like | | | |
| 11262 | SP140 | SP140 nuclear body protein | 54509 | RHOF | ras homolog family member F, |
| | | | | | filopodia associated |
| 112724 | RDH13 | retinol dehydrogenase 13 | 54510 | PCDH18 | protocadherin 18 |
| 112817 | HOGA1 | 4-hydroxy-2-oxoglutarate | 54538 | ROBO4 | roundabout guidance receptor 4 |
| | | aldolase 1 | | | |
| 113026 | PLCD3 | phospholipase C delta 3 | 54541 | DDIT4 | DNA damage inducible transcript |
| | | | | | 4 |
| 113146 | AHNAK2 | AHNAK nucleoprotein 2 | 54546 | RNF186 | ring finger protein 186 |
| 113220 | KIF12 | kinesin family member 12 | 5455 | POU3F3 | POU class 3 homeobox 3 |
| 11326 | VSIG4 | V-set and immunoglobulin | 54550 | NECAB2 | N-terminal EF-hand calcium |
| | | domain containing 4 | | | binding protein 2 |
| 113278 | SLC52A3 | solute carrier family 52 | 54567 | DLL4 | delta like canonical Notch ligand 4 |
| | | member 3 | | | |
| 11346 | SYNPO | synaptopodin | 54575 | UGT1A10 | UDP glucuronosyltransferase |
| | | | | | family 1 member A10 |
| 113835 | ZNF257 | zinc finger protein 257 | 54576 | UGT1A8 | UDP glucuronosyltransferase |
| | | | | | family 1 member A8 |
| 1140 | CHRNB1 | cholinergic receptor nicotinic | 54577 | UGT1A7 | UDP glucuronosyltransferase |
| | | beta 1 subunit | | | family 1 member A7 |
| 114088 | TRIM9 | tripartite motif containing 9 | 54578 | UGT1A6 | UDP glucuronosyltransferase |
| | | | | | family 1 member A6 |
| 114569 | MAL2 | mal, T-cell differentiation | 54579 | UGT1A5 | UDP glucuronosyltransferase |
| | | protein 2 (gene/pseudogene) | | | family 1 member A5 |
| 114757 | CYGB | cytoglobin | 54587 | MXRA8 | matrix remodeling associated 8 |
| 114800 | CCDC85A | coiled-coil domain containing | 5460 | POU5F1 | POU class 5 homeobox 1 |
| | | 85A | | | |
| 114804 | RNF157 | ring finger protein 157 | 54600 | UGT1A9 | UDP glucuronosyltransferase |
| | | | | | family 1 member A9 |
| 114827 | FHAD1 | forkhead associated | 5462 | POU5F1B | POU class 5 homeobox 1B |
| | | phosphopeptide binding | | | |
| | | domain 1 | | | |
| 114836 | SLAMF6 | SLAM family member 6 | 54657 | UGT1A4 | UDP glucuronosyltransferase |
| | | | | | family 1 member A4 |
| 114897 | C1QTNF1 | C1q and TNF related 1 | 54658 | UGT1A1 | UDP glucuronosyltransferase |
| | | | | | family 1 member A1 |
| 1152 | CKB | creatine kinase B | 54659 | UGT1A3 | UDP glucuronosyltransferase |
| | | | | | family 1 member A3 |
| 115265 | DDIT4L | DNA damage inducible | 54660 | PCDHB18P | protocadherin beta 18 |
| | | transcript 4 like | | | pseudogene |
| 115273 | RAB42 | RAB42, member RAS | 54661 | PCDHB17P | protocadherin beta 17 |
| | | oncogene family | | | pseudogene |
| 115290 | FBXO17 | F-box protein 17 | 5468 | PPARG | peroxisome proliferator activated |
| | | | | | receptor gamma |
| 115352 | FCRL3 | Fc receptor like 3 | 54682 | MANSC1 | MANSC domain containing 1 |
| 115361 | GBP4 | guanylate binding protein 4 | 5473 | PPBP | pro-platelet basic protein |
| 115362 | GBP5 | guanylate binding protein 5 | 54757 | FAM20A | FAM20A, golgi associated |
| | | | | | secretory pathway pseudokinase |
| 115677 | NOSTRIN | nitric oxide synthase | 54762 | GRAMD1C | GRAM domain containing 1C |
| | | trafficking | | | |
| 115701 | ALPK2 | alpha kinase 2 | 54768 | HYDIN | HYDIN, axonemal central pair |
| | | | | | apparatus protein |
| 115908 | CTHRC1 | collagen triple helix repeat | 54769 | DIRAS2 | DIRAS family GTPase 2 |
| | | containing 1 | | | |
| 1160 | CKMT2 | creatine kinase, mitochondrial | 54798 | DCHS2 | dachsous cadherin-related 2 |
| | | 2 | | | |
| 116085 | SLC22A12 | solute carrier family 22 | 54810 | GIPC2 | GIPC PDZ domain containing |
| | | member 12 | | | family member 2 |
| 116159 | CYYR1 | cysteine and tyrosine rich 1 | 54825 | CDHR2 | cadherin related family member 2 |
| 116238 | TLCD1 | TLC domain containing 1 | 54829 | ASPN | asporin |
| 116362 | RBP7 | retinol binding protein 7 | 54830 | NUP62CL | nucleoporin 62 C-terminal like |
| 1164 | CKS2 | CDC28 protein kinase | 54843 | SYTL2 | synaptotagmin like 2 |
| | | regulatory subunit 2 | | | |
| 116441 | TM4SF18 | transmembrane 4 L six family | 54845 | ESRP1 | epithelial splicing regulatory |
| | | member 18 | | | protein 1 |
| 116449 | CLNK | cytokine dependent | 54848 | ARHGEF38 | Rho guanine nucleotide exchange |
| | | hematopoietic cell linker | | | factor 38 |
| 116832 | RPL39L | ribosomal protein L39 like | 54852 | PAQR5 | progestin and adipoQ receptor |
| | | | | | family member 5 |
| 116842 | LEAP2 | liver enriched antimicrobial | 54855 | FAM46C | family with sequence similarity 46 |
| | | peptide 2 | | | member C |
| 116844 | LRG1 | leucine rich alpha-2- | 54866 | PPP1R14D | protein phosphatase 1 regulatory |
| | | glycoprotein 1 | | | inhibitor subunit 14D |
| 116937 | SNORD83A | small nucleolar RNA, C/D box | 54869 | EPS8L1 | EPS8 like 1 |
| | | 83A | | | |
| 116938 | SNORD83B | small nucleolar RNA, C/D box | 54873 | PALMD | palmdelphin |
| | | 83B | | | |
| 116966 | WDR17 | WD repeat domain 17 | 54900 | LAX1 | lymphocyte transmembrane |
| | | | | | adaptor 1 |
| 117153 | NA | NA | 54922 | RASIP1 | Ras interacting protein 1 |
| 117177 | RAB3IP | RAB3A interacting protein | 54923 | LIME1 | Lck interacting transmembrane |
| | | | | | adaptor 1 |
| 117247 | SLC16A10 | solute carrier family 16 | 5493 | PPL | periplakin |
| | | member 10 | | | |
| 117248 | GALNT15 | polypeptide N- | 54972 | TMEM132A | transmembrane protein 132A |
| | | acetylgalactosaminyltransferase | | | |
| | | 15 | | | |
| 117283 | IP6K3 | inositol hexakisphosphate | 54979 | HRASLS2 | HRAS like suppressor 2 |
| | | kinase 3 | | | |
| 117289 | TAGAP | T-cell activation RhoGTPase | 54988 | ACSM5 | acyl-CoA synthetase medium |
| | | activating protein | | | chain family member 5 |
| 1184 | CLCN5 | chloride voltage-gated | 54996 | MTARC2 | mitochondrial amidoxime reducing |
| | | channel 5 | | | component 2 |
| 118471 | PRAP1 | proline rich acidic protein 1 | 54997 | TESC | tescalcin |
| 118663 | BTBD16 | BTB domain containing 16 | 55001 | TTC22 | tetratricopeptide repeat domain 22 |
| 1187 | CLCNKA | chloride voltage-gated | 5502 | PPP1R1A | protein phosphatase 1 regulatory |
| | | channel Ka | | | inhibitor subunit 1A |
| 118788 | PIK3AP1 | phosphoinositide-3-kinase | 55026 | TMEM255A | transmembrane protein 255A |
| | | adaptor protein 1 | | | |
| 1188 | CLCNKB | chloride voltage-gated | 55034 | MOCOS | molybdenum cofactor sulfurase |
| | | channel Kb | | | |
| 118932 | ANKRD22 | ankyrin repeat domain 22 | 55036 | CCDC40 | coiled-coil domain containing 40 |
| 1191 | CLU | clusterin | 55064 | SPATA6L | spermatogenesis associated 6 like |
| 119385 | AGAP11 | ArfGAP with GTPase domain, | 5507 | PPP1R3C | protein phosphatase 1 regulatory |
| | | ankyrin repeat and PH | | | subunit 3C |
| | | domain 11 | | | |
| 119391 | GSTO2 | glutathione S-transferase | 55073 | LRRC37A4P | leucine rich repeat containing 37 |
| | | omega 2 | | | member A4, pseudogene |
| 119467 | CLRN3 | clarin 3 | 55076 | TMEM45A | transmembrane protein 45A |
| 119587 | CPXM2 | carboxypeptidase X, M14 | 55083 | KIF26B | kinesin family member 26B |
| | | family member 2 | | | |
| 12 | SERPINA3 | serpin family A member 3 | 55084 | SOBP | sine oculis binding protein |
| | | | | | homolog |
| 120071 | LARGE2 | LARGE xylosyl- and | 55086 | CXorf57 | chromosome X open reading |
| | | glucuronyltransferase 2 | | | frame 57 |
| 120224 | TMEM45B | transmembrane protein 45B | 55107 | ANO1 | anoctamin 1 |
| 120376 | COLCA2 | colorectal cancer associated | 55118 | CRTAC1 | cartilage acidic protein 1 |
| | | 2 | | | |
| 120425 | JAML | junction adhesion molecule | 55138 | FAM90A1 | family with sequence similarity 90 |
| | | like | | | member A1 |
| 120892 | LRRK2 | leucine rich repeat kinase 2 | 55143 | CDCA8 | cell division cycle associated 8 |
| 121551 | BTBD11 | BTB domain containing 11 | 55151 | TMEM38B | transmembrane protein 38B |
| 121601 | ANO4 | anoctamin 4 | 55165 | CEP55 | centrosomal protein 55 |
| 122402 | TDRD9 | tudor domain containing 9 | 55195 | CCDC198 | coiled-coil domain containing 198 |
| 122481 | AK7 | adenylate kinase 7 | 55214 | P3H2 | prolyl 3-hydroxylase 2 |
| 122618 | PLD4 | phospholipase D family | 55224 | ETNK2 | ethanolamine kinase 2 |
| | | member 4 | | | |
| 122622 | ADSSL1 | adenylosuccinate synthase | 55228 | PNMA8A | PNMA family member 8A |
| | | like 1 | | | |
| 122970 | ACOT4 | acyl-CoA thioesterase 4 | 55240 | STEAP3 | STEAP3 metalloreductase |
| 123 | PLIN2 | perilipin 2 | 55244 | SLC47A1 | solute carrier family 47 member 1 |
| 1230 | CCR1 | C-C motif chemokine | 55247 | NEIL3 | nei like DNA glycosylase 3 |
| | | receptor 1 | | | |
| 1233 | CCR4 | C-C motif chemokine | 55258 | THNSL2 | threonine synthase like 2 |
| | | receptor 4 | | | |
| 1234 | CCR5 | C-C motif chemokine | 55259 | CASC1 | cancer susceptibility 1 |
| | | receptor 5 | | | |
| | | (gene/pseudogene) | | | |
| 1235 | CCR6 | C-C motif chemokine | 55282 | LRRC36 | leucine rich repeat containing 36 |
| | | receptor 6 | | | |
| 123872 | DNAAF1 | dynein axonemal assembly | 55286 | C4orf19 | chromosome 4 open reading |
| | | factor 1 | | | frame 19 |
| 123876 | ACSM2A | acyl-CoA synthetase medium | 55304 | SPTLC3 | serine palmitoyltransferase long |
| | | chain family member 2A | | | chain base subunit 3 |
| 1244 | ABCC2 | ATP binding cassette | 55329 | MNS1 | meiosis specific nuclear structural |
| | | subfamily C member 2 | | | 1 |
| 124872 | B4GALNT2 | beta-1,4-N-acetyl- | 55349 | CHDH | choline dehydrogenase |
| | | galactosaminyltransferase 2 | | | |
| 124976 | SPNS2 | sphingolipid transporter 2 | 55351 | STK32B | serine/threonine kinase 32B |
| 125 | ADH1B | alcohol dehydrogenase 1B | 55355 | HJURP | Holliday junction recognition |
| | | (class I), beta polypeptide | | | protein |
| 125050 | RN7SK | RNA, 7SK small nuclear | 55365 | TMEM176A | transmembrane protein 176A |
| 125206 | SLC5A10 | solute carrier family 5 | 55423 | SIRPG | signal regulatory protein gamma |
| | | member 10 | | | |
| 126 | ADH1C | alcohol dehydrogenase 1C | 554236 | DPY19L2P1 | DPY19L2 pseudogene 1 |
| | | (class I), gamma polypeptide | | | |
| 126353 | MISP | mitotic spindle positioning | 55450 | CAMK2N1 | calcium/calmodulin dependent |
| | | | | | protein kinase II inhibitor 1 |
| 126393 | HSPB6 | heat shock protein family B | 5549 | PRELP | proline and arginine rich end |
| | | (small) member 6 | | | leucine rich repeat protein |
| 1264 | CNN1 | calponin 1 | 55504 | TNFRSF19 | TNF receptor superfamily member |
| | | | | | 19 |
| 126433 | FBXO27 | F-box protein 27 | 5551 | PRF1 | perforin 1 |
| 126868 | MAB21L3 | mab-21 like 3 | 55510 | DDX43 | DEAD-box helicase 43 |
| 126969 | SLC44A3 | solute carrier family 44 | 5553 | PRG2 | proteoglycan 2, pro eosinophil |
| | | member 3 | | | major basic protein |
| 127069 | OR2T10 | olfactory receptor family 2 | 55540 | IL17RB | interleukin 17 receptor B |
| | | subfamily T member 10 | | | |
| 127077 | OR2T11 | olfactory receptor family 2 | 55553 | SOX6 | SRY-box 6 |
| | | subfamily T member 11 | | | |
| | | (gene/pseudogene) | | | |
| 1272 | CNTN1 | contactin 1 | 55559 | HAUS7 | HAUS augmin like complex |
| | | | | | subunit 7 |
| 127294 | MYOM3 | myomesin 3 | 55586 | MIOX | myo-inositol oxygenase |
| 127435 | PODN | podocan | 55612 | FERMT1 | fermitin family member 1 |
| 1277 | COL1A1 | collagen type I alpha 1 chain | 55616 | ASAP3 | ArfGAP with SH3 domain, ankyrin |
| | | | | | repeat and PH domain 3 |
| 127707 | KLHDC7A | kelch domain containing 7A | 55620 | STAP2 | signal transducing adaptor family |
| | | | | | member 2 |
| 1278 | COL1A2 | collagen type I alpha 2 chain | 5563 | PRKAA2 | protein kinase AMP-activated |
| | | | | | catalytic subunit alpha 2 |
| 127845 | GOLT1A | golgi transport 1A | 55638 | SYBU | syntabulin |
| 1281 | COL3A1 | collagen type III alpha 1 chain | 55655 | NLRP2 | NLR family pyrin domain |
| | | | | | containing 2 |
| 1282 | COL4A1 | collagen type IV alpha 1 | 55679 | LIMS2 | LIM zinc finger domain containing |
| | | chain | | | 2 |
| 128239 | IQGAP3 | IQ motif containing GTPase | 55713 | ZNF334 | zinc finger protein 334 |
| | | activating protein 3 | | | |
| 128312 | HIST3H2BB | histone cluster 3 H2B family | 55714 | TENM3 | teneurin transmembrane protein 3 |
| | | member b | | | |
| 128344 | PIFO | primary cilia formation | 55748 | CNDP2 | carnosine dipeptidase 2 |
| 128346 | C1orf162 | chromosome 1 open reading | 55753 | OGDHL | oxoglutarate dehydrogenase like |
| | | frame 162 | | | |
| 128414 | NKAIN4 | sodium/potassium | 55765 | C1orf106 | chromosome 1 open reading |
| | | transporting ATPase | | | frame 106 |
| | | interacting 4 | | | |
| 1285 | COL4A3 | collagen type IV alpha 3 | 55786 | ZNF415 | zinc finger protein 415 |
| | | chain | | | |
| 128553 | TSHZ2 | teashirt zinc finger homeobox | 5579 | PRKCB | protein kinase C beta |
| | | 2 | | | |
| 1286 | COL4A4 | collagen type IV alpha 4 | 55790 | CSGALNACT1 | chondroitin sulfate N- |
| | | chain | | | acetylgalactosaminyltransferase 1 |
| 1287 | COL4A5 | collagen type IV alpha 5 | 55799 | CACNA2D3 | calcium voltage-gated channel |
| | | chain | | | auxiliary subunit alpha2delta 3 |
| 1289 | COL5A1 | collagen type V alpha 1 chain | 55825 | PECR | peroxisomal trans-2-enoyl-CoA |
| | | | | | reductase |
| 1290 | COL5A2 | collagen type V alpha 2 chain | 5583 | PRKCH | protein kinase C eta |
| 129049 | SGSM1 | small G protein signaling | 55867 | SLC22A11 | solute carrier family 22 member |
| | | modulator 1 | | | 11 |
| 1292 | COL6A2 | collagen type VI alpha 2 | 55872 | PBK | PDZ binding kinase |
| | | chain | | | |
| 1293 | COL6A3 | collagen type VI alpha 3 | 5588 | PRKCQ | protein kinase C theta |
| | | chain | | | |
| 1294 | COL7A1 | collagen type VII alpha 1 | 55893 | ZNF395 | zinc finger protein 395 |
| | | chain | | | |
| 1295 | COL8A1 | collagen type VIII alpha 1 | 5592 | PRKG1 | protein kinase, cGMP-dependent, |
| | | chain | | | type I |
| 129530 | LYG1 | lysozyme g1 | 5593 | PRKG2 | protein kinase, cGMP-dependent, |
| | | | | | type II |
| 129804 | FBLN7 | fibulin 7 | 55937 | APOM | apolipoprotein M |
| 129881 | CCDC173 | coiled-coil domain containing | 55959 | SULF2 | sulfatase 2 |
| | | 173 | | | |
| 130 | ADH6 | alcohol dehydrogenase 6 | 55966 | AJAP1 | adherens junctions associated |
| | | (class V) | | | protein 1 |
| 1300 | COL10A1 | collagen type X alpha 1 chain | 55971 | BAIAP2L1 | BAI1 associated protein 2 like 1 |
| 130013 | ACMSD | aminocarboxymuconate | 56062 | KLHL4 | kelch like family member 4 |
| | | semialdehyde decarboxylase | | | |
| 130075 | OR9A4 | olfactory receptor family 9 | 56099 | PCDHGB7 | protocadherin gamma subfamily |
| | | subfamily A member 4 | | | B, 7 |
| 1301 | COL11A1 | collagen type XI alpha 1 | 56100 | PCDHGB6 | protocadherin gamma subfamily |
| | | chain | | | B, 6 |
| 130106 | CIB4 | calcium and integrin binding | 56101 | PCDHGB5 | protocadherin gamma subfamily |
| | | family member 4 | | | B, 5 |
| 130132 | RFTN2 | raftlin family member 2 | 56102 | PCDHGB3 | protocadherin gamma subfamily |
| | | | | | B, 3 |
| 130271 | PLEKHH2 | pleckstrin homology, MyTH4 | 56103 | PCDHGB2 | protocadherin gamma subfamily |
| | | and FERM domain containing | | | B, 2 |
| | | H2 | | | |
| 1303 | COL12A1 | collagen type XII alpha 1 | 56104 | PCDHGB1 | protocadherin gamma subfamily |
| | | chain | | | B, 1 |
| 130340 | AP1S3 | adaptor related protein | 56106 | PCDHGA10 | protocadherin gamma subfamily |
| | | complex 1 sigma 3 subunit | | | A, 10 |
| 1306 | COL15A1 | collagen type XV alpha 1 | 56107 | PCDHGA9 | protocadherin gamma subfamily |
| | | chain | | | A, 9 |
| 1307 | COL16A1 | collagen type XVI alpha 1 | 56108 | PCDHGA7 | protocadherin gamma subfamily |
| | | chain | | | A, 7 |
| 130749 | CPO | carboxypeptidase O | 56109 | PCDHGA6 | protocadherin gamma subfamily |
| | | | | | A, 6 |
| 130752 | MDH1B | malate dehydrogenase 1B | 56110 | PCDHGA5 | protocadherin gamma subfamily |
| | | | | | A, 5 |
| 130940 | CCDC148 | coiled-coil domain containing | 56111 | PCDHGA4 | protocadherin gamma subfamily |
| | | 148 | | | A, 4 |
| 1311 | COMP | cartilage oligomeric matrix | 56112 | PCDHGA3 | protocadherin gamma subfamily |
| | | protein | | | A, 3 |
| 131450 | CD200R1 | CD200 receptor 1 | 56113 | PCDHGA2 | protocadherin gamma subfamily |
| | | | | | A, 2 |
| 131566 | DCBLD2 | discoidin, CUB and LCCL | 56114 | PCDHGA1 | protocadherin gamma subfamily |
| | | domain containing 2 | | | A, 1 |
| 1316 | KLF6 | Kruppel like factor 6 | 56120 | PCDHGB8P | protocadherin gamma subfamily |
| | | | | | B, 8 pseudogene |
| 132430 | PABPC4L | poly(A) binding protein | 56121 | PCDHB15 | protocadherin beta 15 |
| | | cytoplasmic 4 like | | | |
| 132671 | SPATA18 | spermatogenesis associated | 56122 | PCDHB14 | protocadherin beta 14 |
| | | 18 | | | |
| 132864 | CPEB2 | cytoplasmic polyadenylation | 56123 | PCDHB13 | protocadherin beta 13 |
| | | element binding protein 2 | | | |
| 133 | ADM | adrenomedullin | 56124 | PCDHB12 | protocadherin beta 12 |
| 133418 | EMB | embigin | 56125 | PCDHB11 | protocadherin beta 11 |
| 133584 | EGFLAM | EGF like, fibronectin type III | 56126 | PCDHB10 | protocadherin beta 10 |
| | | and laminin G domains | | | |
| 133688 | UGT3A1 | UDP glycosyltransferase | 56127 | PCDHB9 | protocadherin beta 9 |
| | | family 3 member A1 | | | |
| 134147 | CMBL | carboxymethylenebutenolidase | 56128 | PCDHB8 | protocadherin beta 8 |
| | | homolog | | | |
| 134265 | AFAP1L1 | actin filament associated | 56129 | PCDHB7 | protocadherin beta 7 |
| | | protein 1 like 1 | | | |
| 134285 | TMEM171 | transmembrane protein 171 | 56130 | PCDHB6 | protocadherin beta 6 |
| 1346 | COX7A1 | cytochrome c oxidase subunit | 56131 | PCDHB4 | protocadherin beta 4 |
| | | 7A1 | | | |
| 1356 | CP | ceruloplasmin | 56132 | PCDHB3 | protocadherin beta 3 |
| 135656 | DPCR1 | diffuse panbronchiolitis | 56133 | PCDHB2 | protocadherin beta 2 |
| | | critical region 1 | | | |
| 1359 | CPA3 | carboxypeptidase A3 | 56136 | PCDHA13 | protocadherin alpha 13 |
| 135932 | TMEM139 | transmembrane protein 139 | 56137 | PCDHA12 | protocadherin alpha 12 |
| 136 | ADORA2B | adenosine A2b receptor | 56138 | PCDHA11 | protocadherin alpha 11 |
| 1363 | CPE | carboxypeptidase E | 56139 | PCDHA10 | protocadherin alpha 10 |
| 1364 | CLDN4 | claudin 4 | 56140 | PCDHA8 | protocadherin alpha 8 |
| 1365 | CLDN3 | claudin 3 | 56141 | PCDHA7 | protocadherin alpha 7 |
| 1366 | CLDN7 | claudin 7 | 56142 | PCDHA6 | protocadherin alpha 6 |
| 1368 | CPM | carboxypeptidase M | 56143 | PCDHA5 | protocadherin alpha 5 |
| 1373 | CPS1 | carbamoyl-phosphate | 56144 | PCDHA4 | protocadherin alpha 4 |
| | | synthase 1 | | | |
| 1378 | CR1 | complement C3b/C4b | 56154 | TEX15 | testis expressed 15, meiosis and |
| | | receptor 1 (Knops blood | | | synapsis associated |
| | | group) | | | |
| 137872 | ADHFE1 | alcohol dehydrogenase, iron | 56159 | TEX11 | testis expressed 11 |
| | | containing 1 | | | |
| 1379 | CR1L | complement C3b/C4b | 5616 | PRKY | protein kinase, Y-linked, |
| | | receptor 1 like | | | pseudogene |
| 137902 | PXDNL | peroxidasin like | 56171 | DNAH7 | dynein axonemal heavy chain 7 |
| 138162 | C9orf116 | chromosome 9 open reading | 56241 | SUSD2 | sushi domain containing 2 |
| | | frame 116 | | | |
| 139065 | SLITRK4 | SLIT and NTRK like family | 56253 | CRTAM | cytotoxic and regulatory T-cell |
| | | member 4 | | | molecule |
| 139170 | DCAF12L1 | DDB1 and CUL4 associated | 56256 | SERTAD4 | SERTA domain containing 4 |
| | | factor 12 like 1 | | | |
| 1396 | CRIP1 | cysteine rich protein 1 | 56265 | CPXM1 | carboxypeptidase X, M14 family |
| | | | | | member 1 |
| 139728 | PNCK | pregnancy up-regulated | 5627 | PROS1 | protein S |
| | | nonubiquitous CaM kinase | | | |
| 140 | ADORA3 | adenosine A3 receptor | 56271 | BEX4 | brain expressed X-linked 4 |
| 140686 | WFDC3 | WAP four-disulfide core | 563 | AZGP1 | alpha-2-glycoprotein 1, zinc- |
| | | domain 3 | | | binding |
| 140733 | MACROD2 | MACRO domain containing 2 | 56477 | CCL28 | C-C motif chemokine ligand 28 |
| 140738 | TMEM37 | transmembrane protein 37 | 5648 | MASP1 | mannan binding lectin serine |
| | | | | | peptidase 1 |
| 140766 | ADAMTS14 | ADAM metallopeptidase with | 5649 | RELN | reelin |
| | | thrombospondin type 1 motif | | | |
| | | 14 | | | |
| 140862 | ISM1 | isthmin 1 | 5652 | PRSS8 | protease, serine 8 |
| 140876 | RIPOR3 | RIPOR family member 3 | 56521 | DNAJC12 | DnaJ heat shock protein family |
| | | | | | (Hsp40) member C12 |
| 1410 | CRYAB | crystallin alpha B | 5654 | HTRA1 | HtrA serine peptidase 1 |
| 1415 | CRYBB2 | crystallin beta B2 | 56606 | SLC2A9 | solute carrier family 2 member 9 |
| 1428 | CRYM | crystallin mu | 56664 | VTRNA1-1 | vault RNA 1-1 |
| 143425 | SYT9 | synaptotagmin 9 | 56667 | MUC13 | mucin 13, cell surface associated |
| 143872 | ARHGAP42 | Rho GTPase activating | 56670 | SUCNR1 | succinate receptor 1 |
| | | protein 42 | | | |
| 1441 | CSF3R | colony stimulating factor 3 | 56833 | SLAMF8 | SLAM family member 8 |
| | | receptor | | | |
| 144100 | PLEKHA7 | pleckstrin homology domain | 56892 | TCIM | transcriptional and immune |
| | | containing A7 | | | response regulator |
| 144165 | PRICKLE1 | prickle planar cell polarity | 56898 | BDH2 | 3-hydroxybutyrate dehydrogenase |
| | | protein 1 | | | 2 |
| 144193 | AMDHD1 | amidohydrolase domain | 56899 | ANKS1B | ankyrin repeat and sterile alpha |
| | | containing 1 | | | motif domain containing 1B |
| 144406 | WDR66 | WD repeat domain 66 | 56901 | NDUFA4L2 | NDUFA4, mitochondrial complex |
| | | | | | associated like 2 |
| 144455 | E2F7 | E2F transcription factor 7 | 56911 | MAP3K7CL | MAP3K7 C-terminal like |
| 144501 | KRT80 | keratin 80 | 56937 | PMEPA1 | prostate transmembrane protein, |
| | | | | | androgen induced 1 |
| 145200 | LINC00239 | long intergenic non-protein | 56938 | ARNTL2 | aryl hydrocarbon receptor nuclear |
| | | coding RNA 239 | | | translocator like 2 |
| 145270 | PRIMA1 | proline rich membrane | 56944 | OLFML3 | olfactomedin like 3 |
| | | anchor 1 | | | |
| 145864 | HAPLN3 | hyaluronan and proteoglycan | 56969 | RPL23AP32 | ribosomal protein L23a |
| | | link protein 3 | | | pseudogene 32 |
| 1462 | VCAN | versican | 570 | BAAT | bile acid-CoA:amino acid N- |
| | | | | | acyltransferase |
| 1464 | CSPG4 | chondroitin sulfate | 57007 | ACKR3 | atypical chemokine receptor 3 |
| | | proteoglycan 4 | | | |
| 146439 | BICDL2 | BICD family like cargo | 57016 | AKR1B10 | aldo-keto reductase family 1 |
| | | adaptor 2 | | | member B10 |
| 146456 | TMED6 | transmembrane p24 | 57094 | CPA6 | carboxypeptidase A6 |
| | | trafficking protein 6 | | | |
| 1466 | CSRP2 | cysteine and glycine rich | 57101 | ANO2 | anoctamin 2 |
| | | protein 2 | | | |
| 147 | ADRA1B | adrenoceptor alpha 1B | 57105 | CYSLTR2 | cysteinyl leukotriene receptor 2 |
| 1470 | CST2 | cystatin SA | 57110 | HRASLS | HRAS like suppressor |
| 147138 | TMC8 | transmembrane channel like | 57124 | CD248 | CD248 molecule |
| | | 8 | | | |
| 147495 | APCDD1 | APC down-regulated 1 | 57125 | PLXDC1 | plexin domain containing 1 |
| 1475 | CSTA | cystatin A | 57139 | RGL3 | ral guanine nucleotide dissociation |
| | | | | | stimulator like 3 |
| 147686 | ZNF418 | zinc finger protein 418 | 57158 | JPH2 | junctophilin 2 |
| 147798 | TMC4 | transmembrane channel like | 57165 | GJC2 | gap junction protein gamma 2 |
| | | 4 | | | |
| 147968 | CAPN12 | calpain 12 | 57167 | SALL4 | spalt like transcription factor 4 |
| 148229 | ATP8B3 | ATPase phospholipid | 57172 | CAMK1G | calcium/calmodulin dependent |
| | | transporting 8B3 | | | protein kinase IG |
| 148523 | CIART | circadian associated | 57188 | ADAMTSL3 | ADAMTS like 3 |
| | | repressor of transcription | | | |
| 148641 | SLC35F3 | solute carrier family 35 | 57194 | ATP10A | ATPase phospholipid transporting |
| | | member F3 | | | 10A (putative) |
| 148979 | GLIS1 | GLIS family zinc finger 1 | 57211 | ADGRG6 | adhesion G protein-coupled |
| | | | | | receptor G6 |
| 1490 | CTGF | connective tissue growth | 57214 | CEMIP | cell migration inducing hyaluronan |
| | | factor | | | binding protein |
| 149175 | MANEAL | mannosidase endo-alpha like | 57216 | VANGL2 | VANGL planar cell polarity protein |
| | | | | | 2 |
| 1493 | CTLA4 | cytotoxic T-lymphocyte | 57221 | ARFGEF3 | ARFGEF family member 3 |
| | | associated protein 4 | | | |
| 149466 | C1orf210 | chromosome 1 open reading | 5730 | PTGDS | prostaglandin D2 synthase |
| | | frame 210 | | | |
| 149628 | PYHIN1 | pyrin and HIN domain family | 5733 | PTGER3 | prostaglandin E receptor 3 |
| | | member 1 | | | |
| 150468 | CKAP2L | cytoskeleton associated | 57381 | RHOJ | ras homolog family member J |
| | | protein 2 like | | | |
| 151 | ADRA2B | adrenoceptor alpha 2B | 57393 | TMEM27 | transmembrane protein 27 |
| 1510 | CTSE | cathepsin E | 5740 | PTGIS | prostaglandin 12 synthase |
| 1511 | CTSG | cathepsin G | 574042 | SNORA10 | small nucleolar RNA, H/ACA box |
| | | | | | 10 |
| 151126 | ZNF385B | zinc finger protein 385B | 57405 | SPC25 | SPC25, NDC80 kinetochore |
| | | | | | complex component |
| 151258 | SLC38A11 | solute carrier family 38 | 57406 | ABHD6 | abhydrolase domain containing 6 |
| | | member 11 | | | |
| 151295 | SLC23A3 | solute carrier family 23 | 57419 | SLC24A3 | solute carrier family 24 member 3 |
| | | member 3 | | | |
| 1513 | CTSK | cathepsin K | 5742 | PTGS1 | prostaglandin-endoperoxide |
| | | | | | synthase 1 |
| 151507 | MSL3P1 | MSL complex subunit 3 | 5743 | PTGS2 | prostaglandin-endoperoxide |
| | | pseudogene 1 | | | synthase 2 |
| 151651 | EFHB | EF-hand domain family | 5744 | PTHLH | parathyroid hormone like hormone |
| | | member B | | | |
| 151827 | LRRC34 | leucine rich repeat containing | 57447 | NDRG2 | NDRG family member 2 |
| | | 34 | | | |
| 151887 | CCDC80 | coiled-coil domain containing | 5745 | PTH1R | parathyroid hormone 1 receptor |
| | | 80 | | | |
| 152 | ADRA2C | adrenoceptor alpha 2C | 57451 | TENM2 | teneurin transmembrane protein 2 |
| 1520 | CTSS | cathepsin S | 57452 | GALNT16 | polypeptide N- |
| | | | | | acetylgalactosaminyltransferase |
| | | | | | 16 |
| 152078 | PQLC2L | PQ loop repeat containing 2 | 57453 | DSCAML1 | DS cell adhesion molecule like 1 |
| | | like | | | |
| 1521 | CTSW | cathepsin W | 57463 | AMIGO1 | adhesion molecule with Ig like |
| | | | | | domain 1 |
| 152273 | FGD5 | FYVE, RhoGEF and PH | 57464 | STRIP2 | striatin interacting protein 2 |
| | | domain containing 5 | | | |
| 152330 | CNTN4 | contactin 4 | 57502 | NLGN4X | neuroligin 4, X-linked |
| 1524 | CX3CR1 | C-X3-C motif chemokine | 57520 | HECW2 | HECT, C2 and WW domain |
| | | receptor 1 | | | containing E3 ubiquitin protein |
| | | | | | ligase 2 |
| 1525 | CXADR | CXADR, Ig-like cell adhesion | 57530 | CGN | cingulin |
| | | molecule | | | |
| 152573 | SHISA3 | shisa family member 3 | 57537 | SORCS2 | sortilin related VPS10 domain |
| | | | | | containing receptor 2 |
| 152789 | JAKMIP1 | janus kinase and microtubule | 57538 | ALPK3 | alpha kinase 3 |
| | | interacting protein 1 | | | |
| 1528 | CYB5A | cytochrome b5 type A | 5754 | PTK7 | protein tyrosine kinase 7 (inactive) |
| 153218 | SPINK13 | serine peptidase inhibitor, | 57552 | NCEH1 | neutral cholesterol ester hydrolase |
| | | Kazal type 13 (putative) | | | 1 |
| 153562 | MARVELD2 | MARVEL domain containing | 57554 | LRRC7 | leucine rich repeat containing 7 |
| | | 2 | | | |
| 153579 | BTNL9 | butyrophilin like 9 | 57556 | SEMA6A | semaphorin 6A |
| 1536 | CYBB | cytochrome b-245 beta chain | 57561 | ARRDC3 | arrestin domain containing 3 |
| 153643 | FAM81B | family with sequence | 57572 | DOCK6 | dedicator of cytokinesis 6 |
| | | similarity 81 member B | | | |
| 153768 | PRELID2 | PRELI domain containing 2 | 57573 | ZNF471 | zinc finger protein 471 |
| 153769 | SH3RF2 | SH3 domain containing ring | 57575 | PCDH10 | protocadherin 10 |
| | | finger 2 | | | |
| 154 | ADRB2 | adrenoceptor beta 2 | 57586 | SYT13 | synaptotagmin 13 |
| 154043 | CNKSR3 | CNKSR family member 3 | 57593 | EBF4 | early B-cell factor 4 |
| 1545 | CYP1B1 | cytochrome P450 family 1 | 57619 | SHROOM3 | shroom family member 3 |
| | | subfamily B member 1 | | | |
| 154661 | RUNDC3B | RUN domain containing 3B | 57639 | CCDC146 | coiled-coil domain containing 146 |
| 154796 | AMOT | angiomotin | 5764 | PTN | pleiotrophin |
| 154865 | IQUB | IQ motif and ubiquitin domain | 57643 | ZSWIM5 | zinc finger SWIM-type containing |
| | | containing | | | 5 |
| 155368 | METTL27 | methyltransferase like 27 | 57662 | CAMSAP3 | calmodulin regulated spectrin |
| | | | | | associated protein family member |
| | | | | | 3 |
| 1557 | CYP2C19 | cytochrome P450 family 2 | 5768 | QSOX1 | quiescin sulfhydryl oxidase 1 |
| | | subfamily C member 19 | | | |
| 1558 | CYP2C8 | cytochrome P450 family 2 | 57715 | SEMA4G | semaphorin 4G |
| | | subfamily C member 8 | | | |
| 1559 | CYP2C9 | cytochrome P450 family 2 | 57717 | PCDHB16 | protocadherin beta 16 |
| | | subfamily C member 9 | | | |
| 1573 | CYP2J2 | cytochrome P450 family 2 | 57722 | IGDCC4 | immunoglobulin superfamily DCC |
| | | subfamily J member 2 | | | subclass member 4 |
| 157313 | CDCA2 | cell division cycle associated | 57733 | GBA3 | glucosylceramidase beta 3 |
| | | 2 | | | (gene/pseudogene) |
| 1577 | CYP3A5 | cytochrome P450 family 3 | 57761 | TRIB3 | tribbles pseudokinase 3 |
| | | subfamily A member 5 | | | |
| 157869 | SBSPON | somatomedin B and | 5778 | PTPN7 | protein tyrosine phosphatase, |
| | | thrombospondin type 1 | | | non-receptor type 7 |
| | | domain containing | | | |
| 1579 | CYP4A11 | cytochrome P450 family 4 | 57817 | HAMP | hepcidin antimicrobial peptide |
| | | subfamily A member 11 | | | |
| 158067 | AK8 | adenylate kinase 8 | 57823 | SLAMF7 | SLAM family member 7 |
| 158158 | RASEF | RAS and EF-hand domain | 57830 | KRTAP5-8 | keratin associated protein 5-8 |
| | | containing | | | |
| 1582 | CYP8B1 | cytochrome P450 family 8 | 57834 | CYP4F11 | cytochrome P450 family 4 |
| | | subfamily B member 1 | | | subfamily F member 11 |
| 158326 | FREM1 | FRAS1 related extracellular | 57863 | CADM3 | cell adhesion molecule 3 |
| | | matrix 1 | | | |
| 158376 | SPAAR | small regulatory polypeptide | 5787 | PTPRB | protein tyrosine phosphatase, |
| | | of amino acid response | | | receptor type B |
| 158399 | ZNF483 | zinc finger protein 483 | 5788 | PTPRC | protein tyrosine phosphatase, |
| | | | | | receptor type C |
| 158471 | PRUNE2 | prune homolog 2 | 5789 | PTPRD | protein tyrosine phosphatase, |
| | | | | | receptor type D |
| 1586 | CYP17A1 | cytochrome P450 family 17 | 5790 | PTPRCAP | protein tyrosine phosphatase, |
| | | subfamily A member 1 | | | receptor type C associated protein |
| 1589 | CYP21A2 | cytochrome P450 family 21 | 5794 | PTPRH | protein tyrosine phosphatase, |
| | | subfamily A member 2 | | | receptor type H |
| 1590 | CYP21A1P | cytochrome P450 family 21 | 5797 | PTPRM | protein tyrosine phosphatase, |
| | | subfamily A member 1, | | | receptor type M |
| | | pseudogene | | | |
| 1591 | CYP24A1 | cytochrome P450 family 24 | 5806 | PTX3 | pentraxin 3 |
| | | subfamily A member 1 | | | |
| 1593 | CYP27A1 | cytochrome P450 family 27 | 58189 | WFDC1 | WAP four-disulfide core domain 1 |
| | | subfamily A member 1 | | | |
| 159963 | SLC5A12 | solute carrier family 5 | 5827 | PXMP2 | peroxisomal membrane protein 2 |
| | | member 12 | | | |
| 1602 | DACH1 | dachshund family | 5831 | PYCR1 | pyrroline-5-carboxylate reductase |
| | | transcription factor 1 | | | 1 |
| 160364 | CLEC12A | C-type lectin domain family | 5837 | PYGM | glycogen phosphorylase, muscle |
| | | 12 member A | | | associated |
| 160428 | ALDH1L2 | aldehyde dehydrogenase 1 | 58475 | MS4A7 | membrane spanning 4-domains |
| | | family member L2 | | | A7 |
| 160728 | SLC5A8 | solute carrier family 5 | 58494 | JAM2 | junctional adhesion molecule 2 |
| | | member 8 | | | |
| 1610 | DAO | D-amino acid oxidase | 58510 | PRODH2 | proline dehydrogenase 2 |
| 161198 | CLEC14A | C-type lectin domain | 58528 | RRAGD | Ras related GTP binding D |
| | | containing 14A | | | |
| 162417 | NAGS | N-acetylglutamate synthase | 586 | BCAT1 | branched chain amino acid |
| | | | | | transaminase 1 |
| 162461 | TMEM92 | transmembrane protein 92 | 5880 | RAC2 | Rac family small GTPase 2 |
| 162632 | USP32P1 | ubiquitin specific peptidase | 5896 | RAG1 | recombination activating 1 |
| | | 32 pseudogene 1 | | | |
| 162967 | ZNF320 | zinc finger protein 320 | 58985 | IL22RA1 | interleukin 22 receptor subunit |
| | | | | | alpha 1 |
| 163059 | ZNF433 | zinc finger protein 433 | 59 | ACTA2 | actin, alpha 2, smooth muscle, |
| | | | | | aorta |
| 163071 | ZNF114 | zinc finger protein 114 | 590 | BCHE | butyrylcholinesterase |
| 163175 | LGI4 | leucine rich repeat LGI family | 59084 | ENPP5 | ectonucleotide |
| | | member 4 | | | pyrophosphatase/phosphodiesterase |
| | | | | | 5 (putative) |
| 163223 | ZNF676 | zinc finger protein 676 | 5909 | RAP1GAP | RAP1 GTPase activating protein |
| 1634 | DCN | decorin | 5918 | RARRES1 | retinoic acid receptor responder 1 |
| 163404 | PLPPR5 | phospholipid phosphatase | 5919 | RARRES2 | retinoic acid receptor responder 2 |
| | | related 5 | | | |
| 1636 | ACE | angiotensin I converting | 5920 | RARRES3 | retinoic acid receptor responder 3 |
| | | enzyme | | | |
| 164312 | LRRN4 | leucine rich repeat neuronal 4 | 5924 | RASGRF2 | Ras protein specific guanine |
| | | | | | nucleotide releasing factor 2 |
| 1644 | DDC | dopa decarboxylase | 59272 | ACE2 | angiotensin I converting enzyme 2 |
| 1645 | AKR1C1 | aldo-keto reductase family 1 | 59277 | NTN4 | netrin 4 |
| | | member C1 | | | |
| 1646 | AKR1C2 | aldo-keto reductase family 1 | 59341 | TRPV4 | transient receptor potential cation |
| | | member C2 | | | channel subfamily V member 4 |
| 164668 | APOBEC3H | apolipoprotein B mRNA | 59350 | RXFP1 | relaxin/insulin like family peptide |
| | | editing enzyme catalytic | | | receptor 1 |
| | | subunit 3H | | | |
| 1647 | GADD45A | growth arrest and DNA | 5947 | RBP1 | retinol binding protein 1 |
| | | damage inducible alpha | | | |
| 165 | AEBP1 | AE binding protein 1 | 594838 | SNORD100 | small nucleolar RNA, C/D box 100 |
| 165631 | PARP15 | poly(ADP-ribose) polymerase | 594839 | SNORA33 | small nucleolar RNA, H/ACA box |
| | | family member 15 | | | 33 |
| 166824 | RASSF6 | Ras association domain | 595 | CCND1 | cyclin D1 |
| | | family member 6 | | | |
| 1672 | DEFB1 | defensin beta 1 | 5950 | RBP4 | retinol binding protein 4 |
| 1674 | DES | desmin | 595101 | SMG1P5 | SMG1 pseudogene 5 |
| 167465 | ZNF366 | zinc finger protein 366 | 5959 | RDH5 | retinol dehydrogenase 5 |
| 1675 | CFD | complement factor D | 596 | BCL2 | BCL2, apoptosis regulator |
| 168537 | GIMAP7 | GTPase, IMAP family | 5967 | REG1A | regenerating family member 1 |
| | | member 7 | | | alpha |
| 168620 | BHLHA15 | basic helix-loop-helix family | 597 | BCL2A1 | BCL2 related protein A1 |
| | | member a15 | | | |
| 168667 | BMPER | BMP binding endothelial | 5972 | REN | renin |
| | | regulator | | | |
| 1687 | GSDME | gasdermin E | 5973 | RENBP | renin binding protein |
| 1690 | COCH | cochlin | 5996 | RGS1 | regulator of G protein signaling 1 |
| 169044 | COL22A1 | collagen type XXII alpha 1 | 5997 | RGS2 | regulator of G protein signaling 2 |
| | | chain | | | |
| 169611 | OLFML2A | olfactomedin like 2A | 5999 | RGS4 | regulator of G protein signaling 4 |
| 169693 | TMEM252 | transmembrane protein 252 | 6004 | RGS16 | regulator of G protein signaling 16 |
| 169834 | ZNF883 | zinc finger protein 883 | 6029 | RN7SL1 | RNA, 7SL, cytoplasmic 1 |
| 170063 | NA | NA | 6035 | RNASE1 | ribonuclease A family member 1, |
| | | | | | pancreatic |
| 170679 | PSORS1C1 | psoriasis susceptibility 1 | 6036 | RNASE2 | ribonuclease A family member 2 |
| | | candidate 1 | | | |
| 170690 | ADAMTS16 | ADAM metallopeptidase with | 6038 | RNASE4 | ribonuclease A family member 4 |
| | | thrombospondin type 1 motif | | | |
| | | 16 | | | |
| 170692 | ADAMTS18 | ADAM metallopeptidase with | 6044 | SNORA62 | small nucleolar RNA, H/ACA box |
| | | thrombospondin type 1 motif | | | 62 |
| | | 18 | | | |
| 171024 | SYNPO2 | synaptopodin 2 | 606500 | SNORD68 | small nucleolar RNA, C/D box 68 |
| 1718 | DHCR24 | 24-dehydrocholesterol | 60681 | FKBP10 | FK506 binding protein 10 |
| | | reductase | | | |
| 1728 | NQO1 | NAD(P)H quinone | 608 | TNFRSF17 | TNF receptor superfamily member |
| | | dehydrogenase 1 | | | 17 |
| 1731 | SEPT1 | septin 1 | 6083 | SNORD21 | small nucleolar RNA, C/D box 21 |
| 1749 | DLX5 | distal-less homeobox 5 | 6084 | RNY1 | RNA, Ro-associated Y1 |
| 1755 | DMBT1 | deleted in malignant brain | 6086 | RNY4 | RNA, Ro-associated Y4 |
| | | tumors 1 | | | |
| 1756 | DMD | dystrophin | 6090 | RNY5 | RNA, Ro-associated Y5 |
| 1759 | DNM1 | dynamin 1 | 6091 | ROBO1 | roundabout guidance receptor 1 |
| 176 | ACAN | aggrecan | 6092 | ROBO2 | roundabout guidance receptor 2 |
| 1767 | DNAH5 | dynein axonemal heavy chain | 6097 | RORC | RAR related orphan receptor C |
| | | 5 | | | |
| 1768 | DNAH6 | dynein axonemal heavy chain | 6101 | RP1 | RP1, axonemal microtubule |
| | | 6 | | | associated |
| 1776 | DNASE1L3 | deoxyribonuclease 1 like 3 | 6133 | RPL9 | ribosomal protein L9 |
| 1794 | DOCK2 | dedicator of cytokinesis 2 | 6192 | RPS4Y1 | ribosomal protein S4, Y-linked 1 |
| 1800 | DPEP1 | dipeptidase 1 | 619279 | ZNF704 | zinc finger protein 704 |
| 1803 | DPP4 | dipeptidyl peptidase 4 | 619498 | SNORD74 | small nucleolar RNA, C/D box 74 |
| 1805 | DPT | dermatopontin | 619505 | SNORA21 | small nucleolar RNA, H/ACA box |
| | | | | | 21 |
| 1807 | DPYS | dihydropyrimidinase | 619562 | SNORA3A | small nucleolar RNA, H/ACA box |
| | | | | | 3A |
| 1809 | DPYSL3 | dihydropyrimidinase like 3 | 619569 | SNORA41 | small nucleolar RNA, H/ACA box |
| | | | | | 41 |
| 1824 | DSC2 | desmocollin 2 | 619570 | SNORD95 | small nucleolar RNA, C/D box 95 |
| 1829 | DSG2 | desmoglein 2 | 619571 | SNORD96A | small nucleolar RNA, C/D box 96A |
| 183 | AGT | angiotensinogen | 6236 | RRAD | RRAD, Ras related glycolysis |
| | | | | | inhibitor and calcium channel |
| | | | | | regulator |
| 1831 | TSC22D3 | TSC22 domain family | 6241 | RRM2 | ribonucleotide reductase |
| | | member 3 | | | regulatory subunit M2 |
| 1832 | DSP | desmoplakin | 6261 | RYR1 | ryanodine receptor 1 |
| 1837 | DTNA | dystrobrevin alpha | 6262 | RYR2 | ryanodine receptor 2 |
| 1839 | HBEGF | heparin binding EGF like | 6271 | S100A1 | S100 calcium binding protein A1 |
| | | growth factor | | | |
| 1842 | ECM2 | extracellular matrix protein 2 | 6279 | S100A8 | S100 calcium binding protein A8 |
| 1843 | DUSP1 | dual specificity phosphatase | 6280 | S100A9 | S100 calcium binding protein A9 |
| | | 1 | | | |
| 1844 | DUSP2 | dual specificity phosphatase | 6283 | S100A12 | S100 calcium binding protein A12 |
| | | 2 | | | |
| 185 | AGTR1 | angiotensin II receptor type 1 | 6285 | S100B | S100 calcium binding protein B |
| 187 | APLNR | apelin receptor | 6288 | SAA1 | serum amyloid A1 |
| 1879 | EBF1 | early B-cell factor 1 | 6289 | SAA2 | serum amyloid A2 |
| 1880 | GPR183 | G protein-coupled receptor | 629 | CFB | complement factor B |
| | | 183 | | | |
| 1893 | ECM1 | extracellular matrix protein 1 | 6296 | ACSM3 | acyl-CoA synthetase medium |
| | | | | | chain family member 3 |
| 1894 | ECT2 | epithelial cell transforming 2 | 6300 | MAPK12 | mitogen-activated protein kinase |
| | | | | | 12 |
| 1901 | S1PR1 | sphingosine-1-phosphate | 6319 | SCD | stearoyl-CoA desaturase |
| | | receptor 1 | | | |
| 1903 | S1PR3 | sphingosine-1-phosphate | 6324 | SCN1B | sodium voltage-gated channel |
| | | receptor 3 | | | beta subunit 1 |
| 1906 | EDN1 | endothelin 1 | 6326 | SCN2A | sodium voltage-gated channel |
| | | | | | alpha subunit 2 |
| 1907 | EDN2 | endothelin 2 | 6328 | SCN3A | sodium voltage-gated channel |
| | | | | | alpha subunit 3 |
| 1909 | EDNRA | endothelin receptor type A | 633 | BGN | biglycan |
| 1910 | EDNRB | endothelin receptor type B | 6330 | SCN4B | sodium voltage-gated channel |
| | | | | | beta subunit 4 |
| 1917 | EEF1A2 | eukaryotic translation | 6334 | SCN8A | sodium voltage-gated channel |
| | | elongation factor 1 alpha 2 | | | alpha subunit 8 |
| 1942 | EFNA1 | ephrin A1 | 6335 | SCN9A | sodium voltage-gated channel |
| | | | | | alpha subunit 9 |
| 1946 | EFNA5 | ephrin A5 | 6337 | SCNN1A | sodium channel epithelial 1 alpha |
| | | | | | subunit |
| 1948 | EFNB2 | ephrin B2 | 6338 | SCNN1B | sodium channel epithelial 1 beta |
| | | | | | subunit |
| 1950 | EGF | epidermal growth factor | 634 | CEACAM1 | carcinoembryonic antigen related |
| | | | | | cell adhesion molecule 1 |
| 1952 | CELSR2 | cadherin EGF LAG seven- | 6340 | SCNN1G | sodium channel epithelial 1 |
| | | pass G-type receptor 2 | | | gamma subunit |
| 1956 | EGFR | epidermal growth factor | 6347 | CCL2 | C-C motif chemokine ligand 2 |
| | | receptor | | | |
| 1958 | EGR1 | early growth response 1 | 6348 | CCL3 | C-C motif chemokine ligand 3 |
| 1959 | EGR2 | early growth response 2 | 6349 | CCL3L1 | C-C motif chemokine ligand 3 like |
| | | | | | 1 |
| 1962 | EHHADH | enoyl-CoA hydratase and 3- | 635 | BHMT | betaine—homocysteine S- |
| | | hydroxyacyl CoA | | | methyltransferase |
| | | dehydrogenase | | | |
| 196410 | METTL7B | methyltransferase like 7B | 6351 | CCL4 | C-C motif chemokine ligand 4 |
| 196446 | MYRFL | myelin regulatory factor-like | 6352 | CCL5 | C-C motif chemokine ligand 5 |
| 196883 | ADCY4 | adenylate cyclase 4 | 6355 | CCL8 | C-C motif chemokine ligand 8 |
| 197135 | PATL2 | PAT1 homolog 2 | 6356 | CCL11 | C-C motif chemokine ligand 11 |
| 199 | AIF1 | allograft inflammatory factor 1 | 6357 | CCL13 | C-C motif chemokine ligand 13 |
| 199731 | CADM4 | cell adhesion molecule 4 | 6358 | CCL14 | C-C motif chemokine ligand 14 |
| 1999 | ELF3 | E74 like ETS transcription | 6362 | CCL18 | C-C motif chemokine ligand 18 |
| | | factor 3 | | | |
| 200010 | SLC5A9 | solute carrier family 5 | 6363 | CCL19 | C-C motif chemokine ligand 19 |
| | | member 9 | | | |
| 200162 | SPAG17 | sperm associated antigen 17 | 6364 | CCL20 | C-C motif chemokine ligand 20 |
| 2003 | ELK2AP | ELK2A, member of ETS | 6366 | CCL21 | C-C motif chemokine ligand 21 |
| | | oncogene family, | | | |
| | | pseudogene | | | |
| 200373 | CFAP221 | cilia and flagella associated | 6368 | CCL23 | C-C motif chemokine ligand 23 |
| | | protein 221 | | | |
| 200420 | ALMS1P1 | ALMS1, centrosome and | 6372 | CXCL6 | C-X-C motif chemokine ligand 6 |
| | | basal body associated protein | | | |
| | | pseudogene 1 | | | |
| 2006 | ELN | elastin | 6373 | CXCL11 | C-X-C motif chemokine ligand 11 |
| 200634 | KRTCAP3 | keratinocyte associated | 6374 | CXCL5 | C-X-C motif chemokine ligand 5 |
| | | protein 3 | | | |
| 200879 | LIPH | lipase H | 6376 | CX3CL1 | C-X3-C motif chemokine ligand 1 |
| 200916 | RPL22L1 | ribosomal protein L22 like 1 | 6382 | SDC1 | syndecan 1 |
| 200931 | SLC51A | solute carrier family 51 alpha | 6387 | CXCL12 | C-X-C motif chemokine ligand 12 |
| | | subunit | | | |
| 200958 | MUC20 | mucin 20, cell surface | 63895 | PIEZO2 | piezo type mechanosensitive ion |
| | | associated | | | channel component 2 |
| 201161 | CENPV | centromere protein V | 63910 | SLC17A9 | solute carrier family 17 member 9 |
| 2012 | EMP1 | epithelial membrane protein 1 | 63917 | GALNT11 | polypeptide N- |
| | | | | | acetylgalactosaminyltransferase |
| | | | | | 11 |
| 201232 | SLC16A13 | solute carrier family 16 | 63951 | DMRTA1 | DMRT like family A1 |
| | | member 13 | | | |
| 2015 | ADGRE1 | adhesion G protein-coupled | 63982 | ANO3 | anoctamin 3 |
| | | receptor E1 | | | |
| 201501 | ZBTB7C | zinc finger and BTB domain | 640 | BLK | BLK proto-oncogene, Src family |
| | | containing 7C | | | tyrosine kinase |
| 2018 | EMX2 | empty spiracles homeobox 2 | 64005 | MYO1G | myosin IG |
| 202134 | FAM153B | family with sequence | 6402 | SELL | selectin L |
| | | similarity 153 member B | | | |
| 202333 | CMYA5 | cardiomyopathy associated 5 | 6403 | SELP | selectin P |
| 2026 | ENO2 | enolase 2 | 6405 | SEMA3F | semaphorin 3F |
| 2028 | ENPEP | glutamyl aminopeptidase | 64073 | C19orf33 | chromosome 19 open reading |
| | | | | | frame 33 |
| 203100 | HTRA4 | HtrA serine peptidase 4 | 64081 | PBLD | phenazine biosynthesis like |
| | | | | | protein domain containing |
| 203111 | ERICH5 | glutamate rich 5 | 64084 | CLSTN2 | calsyntenin 2 |
| 2034 | EPAS1 | endothelial PAS domain | 64092 | SAMSN1 | SAM domain, SH3 domain and |
| | | protein 1 | | | nuclear localization signals 1 |
| 203562 | TMEM31 | transmembrane protein 31 | 64093 | SMOC1 | SPARC related modular calcium |
| | | | | | binding 1 |
| 203859 | ANO5 | anoctamin 5 | 64094 | SMOC2 | SPARC related modular calcium |
| | | | | | binding 2 |
| 2039 | DMTN | dematin actin binding protein | 64097 | EPB41L4A | erythrocyte membrane protein |
| | | | | | band 4.1 like 4A |
| 2042 | EPHA3 | EPH receptor A3 | 64108 | RTP4 | receptor transporter protein 4 |
| 2043 | EPHA4 | EPH receptor A4 | 64122 | FN3K | fructosamine 3 kinase |
| 2045 | EPHA7 | EPH receptor A7 | 64123 | ADGRL4 | adhesion G protein-coupled |
| | | | | | receptor L4 |
| 2048 | EPHB2 | EPH receptor B2 | 64127 | NOD2 | nucleotide binding oligomerization |
| | | | | | domain containing 2 |
| 204962 | SLC44A5 | solute carrier family 44 | 64129 | TINAGL1 | tubulointerstitial nephritis antigen |
| | | member 5 | | | like 1 |
| 2053 | EPHX2 | epoxide hydrolase 2 | 641371 | ACOT1 | acyl-CoA thioesterase 1 |
| 2064 | ERBB2 | erb-b2 receptor tyrosine | 6414 | SELENOP | selenoprotein P |
| | | kinase 2 | | | |
| 2065 | ERBB3 | erb-b2 receptor tyrosine | 641451 | SNORA19 | small nucleolar RNA, H/ACA box |
| | | kinase 3 | | | 19 |
| 2066 | ERBB4 | erb-b2 receptor tyrosine | 641648 | SNORD87 | small nucleolar RNA, C/D box 87 |
| | | kinase 4 | | | |
| 2070 | EYA4 | EYA transcriptional | 641649 | TMEM91 | transmembrane protein 91 |
| | | coactivator and phosphatase | | | |
| | | 4 | | | |
| 2078 | ERG | ERG, ETS transcription factor | 64167 | ERAP2 | endoplasmic reticulum |
| | | | | | aminopeptidase 2 |
| 2104 | ESRRG | estrogen related receptor | 641700 | ECSCR | endothelial cell surface expressed |
| | | gamma | | | chemotaxis and apoptosis |
| | | | | | regulator |
| 2115 | ETV1 | ETS variant 1 | 64218 | SEMA4A | semaphorin 4A |
| 2124 | EVI2B | ecotropic viral integration site | 6422 | SFRP1 | secreted frizzled related protein 1 |
| | | 2B | | | |
| 213 | ALB | albumin | 642236 | FRG1JP | FSHD region gene 1 family |
| | | | | | member J, pseudogene |
| 2138 | EYA1 | EYA transcriptional | 6423 | SFRP2 | secreted frizzled related protein 2 |
| | | coactivator and phosphatase | | | |
| | | 1 | | | |
| 2139 | EYA2 | EYA transcriptional | 64231 | MS4A6A | membrane spanning 4-domains |
| | | coactivator and phosphatase | | | A6A |
| | | 2 | | | |
| 2147 | F2 | coagulation factor II, thrombin | 6424 | SFRP4 | secreted frizzled related protein 4 |
| 2149 | F2R | coagulation factor II thrombin | 642517 | AGAP9 | ArfGAP with GTPase domain, |
| | | receptor | | | ankyrin repeat and PH domain 9 |
| 2150 | F2RL1 | F2R like trypsin receptor 1 | 64283 | ARHGEF28 | Rho guanine nucleotide exchange |
| | | | | | factor 28 |
| 2152 | F3 | coagulation factor III, tissue | 64284 | RAB17 | RAB17, member RAS oncogene |
| | | factor | | | family |
| 2153 | F5 | coagulation factor V | 64288 | ZSCAN31 | zinc finger and SCAN domain |
| | | | | | containing 31 |
| 2157 | F8 | coagulation factor VIII | 642987 | TMEM232 | transmembrane protein 232 |
| 2159 | F10 | coagulation factor X | 64321 | SOX17 | SRY-box 17 |
| 216 | ALDH1A1 | aldehyde dehydrogenase 1 | 643236 | TMEM72 | transmembrane protein 72 |
| | | family member A1 | | | |
| 2162 | F13A1 | coagulation factor XIII A | 64332 | NFKBIZ | NFKB inhibitor zeta |
| | | chain | | | |
| 2166 | FAAH | fatty acid amide hydrolase | 64333 | ARHGAP9 | Rho GTPase activating protein 9 |
| 2167 | FABP4 | fatty acid binding protein 4 | 644165 | BCRP3 | breakpoint cluster region |
| | | | | | pseudogene 3 |
| 2168 | FABP1 | fatty acid binding protein 1 | 6442 | SGCA | sarcoglycan alpha |
| 2170 | FABP3 | fatty acid binding protein 3 | 644246 | KANSL1-AS1 | KANSL1 antisense RNA 1 |
| 2171 | FABP5 | fatty acid binding protein 5 | 6447 | SCG5 | secretogranin V |
| 2172 | FABP6 | fatty acid binding protein 6 | 645090 | NA | NA |
| 2173 | FABP7 | fatty acid binding protein 7 | 645367 | GGT8P | gamma-glutamyltransferase 8 |
| | | | | | pseudogene |
| 2180 | ACSL1 | acyl-CoA synthetase long | 645432 | ARRDC5 | arrestin domain containing 5 |
| | | chain family member 1 | | | |
| 2184 | FAH | fumarylacetoacetate | 64577 | ALDH8A1 | aldehyde dehydrogenase 8 family |
| | | hydrolase | | | member A1 |
| 2191 | FAP | fibroblast activation protein | 645784 | ANKRD36BP2 | ankyrin repeat domain 36B |
| | | alpha | | | pseudogene 2 |
| 2192 | FBLN1 | fibulin 1 | 64581 | CLEC7A | C-type lectin domain containing |
| | | | | | 7A |
| 219285 | SAMD9L | sterile alpha motif domain | 646023 | ADORA2A- | ADORA2A antisense RNA 1 |
| | | containing 9 like | | AS1 | |
| 219348 | PLAC9 | placenta specific 9 | 646396 | REREP3 | arginine-glutamic acid dipeptide |
| | | | | | repeats pseudogene 3 |
| 219621 | CABCOCO1 | ciliary associated calcium | 64641 | EBF2 | early B-cell factor 2 |
| | | binding coiled-coil 1 | | | |
| 219736 | STOX1 | storkhead box 1 | 64651 | CSRNP1 | cysteine and serine rich nuclear |
| | | | | | protein 1 |
| 2199 | FBLN2 | fibulin 2 | 6469 | SHH | sonic hedgehog |
| 220 | ALDH1A3 | aldehyde dehydrogenase 1 | 64699 | TMPRSS3 | transmembrane protease, serine 3 |
| | | family member A3 | | | |
| 2200 | FBN1 | fibrillin 1 | 6470 | SHMT1 | serine hydroxymethyltransferase 1 |
| 220001 | VWCE | von Willebrand factor C and | 647024 | C6orf132 | chromosome 6 open reading |
| | | EGF domains | | | frame 132 |
| 2201 | FBN2 | fibrillin 2 | 64757 | MTARC1 | mitochondrial amidoxime reducing |
| | | | | | component 1 |
| 2202 | EFEMP1 | EGF containing fibulin | 64762 | GAREM1 | GRB2 associated regulator of |
| | | extracellular matrix protein 1 | | | MAPK1 subtype 1 |
| 2203 | FBP1 | fructose-bisphosphatase 1 | 647859 | LOC647859 | occludin pseudogene |
| 2205 | FCER1A | Fc fragment of IgE receptor | 6480 | ST6GAL1 | ST6 beta-galactoside alpha-2,6- |
| | | Ia | | | sialyltransferase 1 |
| 220594 | USP32P2 | ubiquitin specific peptidase | 64805 | P2RY12 | purinergic receptor P2Y12 |
| | | 32 pseudogene 2 | | | |
| 2206 | MS4A2 | membrane spanning 4- | 64838 | FNDC4 | fibronectin type III domain |
| | | domains A2 | | | containing 4 |
| 2207 | FCER1G | Fc fragment of IgE receptor | 64849 | SLC13A3 | solute carrier family 13 member 3 |
| | | Ig | | | |
| 2209 | FCGR1A | Fc fragment of IgG receptor | 64866 | CDCP1 | CUB domain containing protein 1 |
| | | Ia | | | |
| 220963 | SLC16A9 | solute carrier family 16 | 64901 | RANBP17 | RAN binding protein 17 |
| | | member 9 | | | |
| 220965 | FAM13C | family with sequence | 64902 | AGXT2 | alanine—glyoxylate |
| | | similarity 13 member C | | | aminotransferase 2 |
| 221002 | RASGEF1A | RasGEF domain family | 6492 | SIM1 | single-minded family bHLH |
| | | member 1A | | | transcription factor 1 |
| 2213 | FCGR2B | Fc fragment of IgG receptor | 64922 | LRRC19 | leucine rich repeat containing 19 |
| | | IIb | | | |
| 221395 | ADGRF5 | adhesion G protein-coupled | 64926 | RASAL3 | RAS protein activator like 3 |
| | | receptor F5 | | | |
| 2214 | FCGR3A | Fc fragment of IgG receptor | 650 | BMP2 | bone morphogenetic protein 2 |
| | | IIIa | | | |
| 221416 | C6orf223 | chromosome 6 open reading | 6503 | SLA | Src like adaptor |
| | | frame 223 | | | |
| 221421 | RSPH9 | radial spoke head 9 homolog | 650368 | TSSC2 | tumor suppressing |
| | | | | | subtransferable candidate 2 |
| | | | | | pseudogene |
| 2215 | FCGR3B | Fc fragment of lgG receptor | 6504 | SLAMF1 | signaling lymphocytic activation |
| | | IIIb | | | molecule family member 1 |
| 221806 | VWDE | von Willebrand factor D and | 6505 | SLC1A1 | solute carrier family 1 member 1 |
| | | EGF domains | | | |
| 2219 | FCN1 | ficolin 1 | 6507 | SLC1A3 | solute carrier family 1 member 3 |
| 221935 | SDK1 | sidekick cell adhesion | 65078 | RTN4R | reticulon 4 receptor |
| | | molecule 1 | | | |
| 221981 | THSD7A | thrombospondin type 1 | 6508 | SLC4A3 | solute carrier family 4 member 3 |
| | | domain containing 7A | | | |
| 222223 | KIAA 1324L | KIAA1324 like | 6513 | SLC2A1 | solute carrier family 2 member 1 |
| 222256 | CDHR3 | cadherin related family | 6514 | SLC2A2 | solute carrier family 2 member 2 |
| | | member 3 | | | |
| 222643 | UNC5CL | unc-5 family C-terminal like | 6515 | SLC2A3 | solute carrier family 2 member 3 |
| 222865 | TMEM130 | transmembrane protein 130 | 6517 | SLC2A4 | solute carrier family 2 member 4 |
| 222962 | SLC29A4 | solute carrier family 29 | 6518 | SLC2A5 | solute carrier family 2 member 5 |
| | | member 4 | | | |
| 223117 | SEMA3D | semaphorin 3D | 6519 | SLC3A1 | solute carrier family 3 member 1 |
| 2239 | GPC4 | glypican 4 | 652 | BMP4 | bone morphogenetic protein 4 |
| 224 | ALDH3A2 | aldehyde dehydrogenase 3 | 6523 | SLC5A1 | solute carrier family 5 member 1 |
| | | family member A2 | | | |
| 2243 | FGA | fibrinogen alpha chain | 6526 | SLC5A3 | solute carrier family 5 member 3 |
| 2244 | FGB | fibrinogen beta chain | 65266 | WNK4 | WNK lysine deficient protein |
| | | | | | kinase 4 |
| 2245 | FGD1 | FYVE, RhoGEF and PH | 6527 | SLC5A4 | solute carrier family 5 member 4 |
| | | domain containing 1 | | | |
| 2247 | FGF2 | fibroblast growth factor 2 | 6529 | SLC6A1 | solute carrier family 6 member 1 |
| 2252 | FGF7 | fibroblast growth factor 7 | 6531 | SLC6A3 | solute carrier family 6 member 3 |
| 2256 | FGF11 | fibroblast growth factor 11 | 653113 | FAM86FP | family with sequence similarity 86, |
| | | | | | member A pseudogene |
| 225689 | MAPK15 | mitogen-activated protein | 653190 | ABCC6P1 | ATP binding cassette subfamily C |
| | | kinase 15 | | | member 6 pseudogene 1 |
| 2259 | FGF14 | fibroblast growth factor 14 | 653316 | FAM153C | family with sequence similarity |
| | | | | | 153 member C |
| 2261 | FGFR3 | fibroblast growth factor | 653361 | NCF1 | neutrophil cytosolic factor 1 |
| | | receptor 3 | | | |
| 2263 | FGFR2 | fibroblast growth factor | 6535 | SLC6A8 | solute carrier family 6 member 8 |
| | | receptor 2 | | | |
| 2264 | FGFR4 | fibroblast growth factor | 653604 | HIST2H3D | histone cluster 2 H3 family |
| | | receptor 4 | | | member d |
| 2266 | FGG | fibrinogen gamma chain | 653689 | GSTT2B | glutathione S-transferase theta 2B |
| | | | | | (gene/pseudogene) |
| 2273 | FHL1 | four and a half LIM domains 1 | 653720 | GOLGA8M | golgin A8 family member M |
| 2274 | FHL2 | four and a half LIM domains 2 | 6539 | SLC6A12 | solute carrier family 6 member 12 |
| 22795 | NID2 | nidogen 2 | 6540 | SLC6A13 | solute carrier family 6 member 13 |
| 22797 | TFEC | transcription factor EC | 6542 | SLC7A2 | solute carrier family 7 member 2 |
| 22801 | ITGA11 | integrin subunit alpha 11 | 654321 | SNORA75 | small nucleolar RNA, H/ACA box |
| | | | | | 75 |
| 22806 | IKZF3 | IKAROS family zinc finger 3 | 654322 | SNORA13 | small nucleolar RNA, H/ACA box |
| | | | | | 13 |
| 22822 | PHLDA1 | pleckstrin homology like | 6555 | SLC10A2 | solute carrier family 10 member 2 |
| | | domain family A member 1 | | | |
| 22829 | NLGN4Y | neuroligin 4, Y-linked | 6556 | SLC11A1 | solute carrier family 11 member 1 |
| 22836 | RHOBTB3 | Rho related BTB domain | 6561 | SLC13A1 | solute carrier family 13 member 1 |
| | | containing 3 | | | |
| 22871 | NLGN1 | neuroligin 1 | 6563 | SLC14A1 | solute carrier family 14 member 1 |
| | | | | | (Kidd blood group) |
| 22885 | ABLIM3 | actin binding LIM protein | 6564 | SLC15A1 | solute carrier family 15 member 1 |
| | | family member 3 | | | |
| 2289 | FKBP5 | FK506 binding protein 5 | 6568 | SLC17A1 | solute carrier family 17 member 1 |
| 22899 | ARHGEF15 | Rho guanine nucleotide | 6578 | SLCO2A1 | solute carrier organic anion |
| | | exchange factor 15 | | | transporter family member 2A1 |
| 229 | ALDOB | aldolase, fructose- | 6581 | SLC22A3 | solute carrier family 22 member 3 |
| | | bisphosphate B | | | |
| 22915 | MMRN1 | multimerin 1 | 6582 | SLC22A2 | solute carrier family 22 member 2 |
| 22932 | POMZP3 | POM121 and ZP3 fusion | 6583 | SLC22A4 | solute carrier family 22 member 4 |
| 22936 | ELL2 | elongation factor for RNA | 6584 | SLC22A5 | solute carrier family 22 member 5 |
| | | polymerase II 2 | | | |
| 2294 | FOXF1 | forkhead box F1 | 6586 | SLIT3 | slit guidance ligand 3 |
| 22941 | SHANK2 | SH3 and multiple ankyrin | 6590 | SLPI | secretory leukocyte peptidase |
| | | repeat domains 2 | | | inhibitor |
| 22949 | PTGR1 | prostaglandin reductase 1 | 6591 | SNAI2 | snail family transcriptional |
| | | | | | repressor 2 |
| 2297 | FOXD1 | forkhead box D1 | 65975 | STK33 | serine/threonine kinase 33 |
| 22974 | TPX2 | TPX2, microtubule nucleation | 660 | BMX | BMX non-receptor tyrosine kinase |
| | | factor | | | |
| 22977 | AKR7A3 | aldo-keto reductase family 7 | 66002 | CYP4F12 | cytochrome P450 family 4 |
| | | member A3 | | | subfamily F member 12 |
| 22986 | SORCS3 | sortilin related VPS10 domain | 6614 | SIGLEC1 | sialic acid binding Ig like lectin 1 |
| | | containing receptor 3 | | | |
| 22996 | TTC39A | tetratricopeptide repeat | 6616 | SNAP25 | synaptosome associated protein |
| | | domain 39A | | | 25 |
| 230 | ALDOC | aldolase, fructose- | 6622 | SNCA | synuclein alpha |
| | | bisphosphate C | | | |
| 2300 | FOXL1 | forkhead box L1 | 6623 | SNCG | synuclein gamma |
| 23015 | GOLGA8A | golgin A8 family member A | 6624 | FSCN1 | fascin actin-bundling protein 1 |
| 23024 | PDZRN3 | PDZ domain containing ring | 664 | BNIP3 | BCL2 interacting protein 3 |
| | | finger 3 | | | |
| 2303 | FOXC2 | forkhead box C2 | 6640 | SNTA1 | syntrophin alpha 1 |
| 23037 | PDZD2 | PDZ domain containing 2 | 664701 | ZNF826P | zinc finger protein 826, |
| | | | | | pseudogene |
| 2305 | FOXM1 | forkhead box M1 | 6648 | SOD2 | superoxide dismutase 2 |
| 2307 | FOXS1 | forkhead box S1 | 6662 | SOX9 | SRY-box 9 |
| 23072 | HECW1 | HECT, C2 and WW domain | 6688 | SPI1 | Spi-1 proto-oncogene |
| | | containing E3 ubiquitin | | | |
| | | protein ligase 1 | | | |
| 23086 | EXPH5 | exophilin 5 | 6690 | SPINK1 | serine peptidase inhibitor, Kazal |
| | | | | | type 1 |
| 23089 | PEG10 | paternally expressed 10 | 6692 | SPINT1 | serine peptidase inhibitor, Kunitz |
| | | | | | type 1 |
| 231 | AKR1B1 | aldo-keto reductase family 1 | 6695 | SPOCK1 | SPARC/osteonectin, cwcv and |
| | | member B | | | kazal like domains proteoglycan 1 |
| 23114 | NFASC | neurofascin | 6696 | SPP1 | secreted phosphoprotein 1 |
| 23149 | FCHO1 | FCH domain only 1 | 6712 | SPTBN2 | spectrin beta, non-erythrocytic 2 |
| 23151 | GRAMD4 | GRAM domain containing 4 | 6752 | SSTR2 | somatostatin receptor 2 |
| 2318 | FLNC | filamin C | 6768 | ST14 | suppression of tumorigenicity 14 |
| 23189 | KANK1 | KN motif and ankyrin repeat | 6769 | STAC | SH3 and cysteine rich domain |
| | | domains 1 | | | |
| 2321 | FLT1 | fms related tyrosine kinase 1 | 6772 | STAT1 | signal transducer and activator of |
| | | | | | transcription 1 |
| 23213 | SULF1 | sulfatase 1 | 677679 | SCARNA3 | small Cajal body-specific RNA 3 |
| 23236 | PLCB1 | phospholipase C beta 1 | 677681 | SCARNA20 | small Cajal body-specific RNA 20 |
| 2324 | FLT4 | fms related tyrosine kinase 4 | 677765 | SCARNA18 | small Cajal body-specific RNA 18 |
| 23242 | COBL | cordon-bleu WH2 repeat | 677770 | SCARNA22 | small Cajal body-specific RNA 22 |
| | | protein | | | |
| 23245 | ASTN2 | astrotactin 2 | 677771 | SCARNA4 | small Cajal body-specific RNA 4 |
| 23250 | ATP11A | ATPase phospholipid | 677772 | SCARNA6 | small Cajal body-specific RNA 6 |
| | | transporting 11A | | | |
| 23255 | MTCL1 | microtubule crosslinking | 677773 | SCARNA23 | small Cajal body-specific RNA 23 |
| | | factor 1 | | | |
| 2326 | FMO1 | flavin containing | 677775 | SCARNA5 | small Cajal body-specific RNA 5 |
| | | monooxygenase 1 | | | |
| 23263 | MCF2L | MCF.2 cell line derived | 677776 | SCARNA8 | small Cajal body-specific RNA 8 |
| | | transforming sequence like | | | |
| 2327 | FMO2 | flavin containing | 677780 | SCARNA11 | small Cajal body-specific RNA 11 |
| | | monooxygenase 2 | | | |
| 2328 | FMO3 | flavin containing | 677792 | SNORA1 | small nucleolar RNA, H/ACA box |
| | | monooxygenase 3 | | | 1 |
| 23284 | ADGRL3 | adhesion G protein-coupled | 677793 | SNORA2A | small nucleolar RNA, H/ACA box |
| | | receptor L3 | | | 2A |
| 23286 | WWC1 | WW and C2 domain | 677794 | SNORA2B | small nucleolar RNA, H/ACA box |
| | | containing 1 | | | 2B |
| 23302 | WSCD1 | WSC domain containing 1 | 677796 | SNORA5C | small nucleolar RNA, H/ACA box |
| | | | | | 5C |
| 2331 | FMOD | fibromodulin | 677798 | SNORA9 | small nucleolar RNA, H/ACA box |
| | | | | | 9 |
| 23314 | SATB2 | SATB homeobox 2 | 677799 | SNORA11 | small nucleolar RNA, H/ACA box |
| | | | | | 11 |
| 2335 | FN1 | fibronectin 1 | 677801 | SNORA14A | small nucleolar RNA, H/ACA box |
| | | | | | 14A |
| 23362 | PSD3 | pleckstrin and Sec7 domain | 677802 | SNORA14B | small nucleolar RNA, H/ACA box |
| | | containing 3 | | | 14B |
| 23363 | OBSL1 | obscurin like 1 | 677803 | SNORA15 | small nucleolar RNA, H/ACA box |
| | | | | | 15 |
| 23414 | ZFPM2 | zinc finger protein, FOG | 677806 | SNORA20 | small nucleolar RNA, H/ACA box |
| | | family member 2 | | | 20 |
| 23417 | MLYCD | malonyl-CoA decarboxylase | 677810 | SNORA26 | small nucleolar RNA, H/ACA box |
| | | | | | 26 |
| 23426 | GRIP1 | glutamate receptor interacting | 677811 | SNORA28 | small nucleolar RNA, H/ACA box |
| | | protein 1 | | | 28 |
| 23428 | SLC7A8 | solute carrier family 7 | 677812 | SNORA29 | small nucleolar RNA, H/ACA box |
| | | member 8 | | | 29 |
| 23430 | TPSD1 | tryptase delta 1 | 677813 | SNORA30 | small nucleolar RNA, H/ACA box |
| | | | | | 30 |
| 23452 | ANGPTL2 | angiopoietin like 2 | 677814 | SNORA31 | small nucleolar RNA, H/ACA box |
| | | | | | 31 |
| 2346 | FOLH1 | folate hydrolase 1 | 677815 | SNORA2C | small nucleolar RNA, H/ACA box |
| | | | | | 2C |
| 23460 | ABCA6 | ATP binding cassette | 677818 | SNORA36B | small nucleolar RNA, H/ACA box |
| | | subfamily A member 6 | | | 36B |
| 23462 | HEY1 | hes related family bHLH | 677821 | SNORA71E | small nucleolar RNA, H/ACA box |
| | | transcription factor with | | | 71E |
| | | YRPW motif 1 | | | |
| 2348 | FOLR1 | folate receptor 1 | 677823 | SNORA80E | small nucleolar RNA, H/ACA box |
| | | | | | 80E |
| 23491 | CES3 | carboxylesterase 3 | 677825 | SNORA44 | small nucleolar RNA, H/ACA box |
| | | | | | 44 |
| 23493 | HEY2 | hes related family bHLH | 677826 | SNORA3B | small nucleolar RNA, H/ACA box |
| | | transcription factor with | | | 3B |
| | | YRPW motif 2 | | | |
| 23498 | HAAO | 3-hydroxyanthranilate 3,4- | 677827 | SNORA46 | small nucleolar RNA, H/ACA box |
| | | dioxygenase | | | 46 |
| 2350 | FOLR2 | folate receptor beta | 677828 | SNORA47 | small nucleolar RNA, H/ACA box |
| | | | | | 47 |
| 23500 | DAAM2 | dishevelled associated | 677830 | SNORA50A | small nucleolar RNA, H/ACA box |
| | | activator of morphogenesis 2 | | | 50A |
| 23516 | SLC39A14 | solute carrier family 39 | 677831 | SNORA51 | small nucleolar RNA, H/ACA box |
| | | member 14 | | | 51 |
| 2353 | FOS | Fos proto-oncogene, AP-1 | 677833 | SNORA54 | small nucleolar RNA, H/ACA box |
| | | transcription factor subunit | | | 54 |
| 23532 | PRAME | preferentially expressed | 677834 | SNORA55 | small nucleolar RNA, H/ACA box |
| | | antigen in melanoma | | | 55 |
| 2354 | FOSB | FosB proto-oncogene, AP-1 | 677836 | SNORA58 | small nucleolar RNA, H/ACA box |
| | | transcription factor subunit | | | 58 |
| 23547 | LILRA4 | leukocyte immunoglobulin | 677837 | SNORA60 | small nucleolar RNA, H/ACA box |
| | | like receptor A4 | | | 60 |
| 23551 | RASD2 | RASD family member 2 | 677838 | SNORA61 | small nucleolar RNA, H/ACA box |
| | | | | | 61 |
| 23554 | TSPAN12 | tetraspanin 12 | 677839 | SNORA71C | small nucleolar RNA, H/ACA box |
| | | | | | 71C |
| 2357 | FPR1 | formyl peptide receptor 1 | 677840 | SNORA71D | small nucleolar RNA, H/ACA box |
| | | | | | 71D |
| 23576 | DDAH1 | dimethylarginine | 677842 | SNORA50C | small nucleolar RNA, H/ACA box |
| | | dimethylaminohydrolase 1 | | | 50C |
| 2358 | FPR2 | formyl peptide receptor 2 | 677843 | SNORA77 | small nucleolar RNA, H/ACA box |
| | | | | | 77 |
| 23584 | VSIG2 | V-set and immunoglobulin | 677844 | SNORA78 | small nucleolar RNA, H/ACA box |
| | | domain containing 2 | | | 78 |
| 23594 | ORC6 | origin recognition complex | 677845 | SNORA79 | small nucleolar RNA, H/ACA box |
| | | subunit 6 | | | 79 |
| 23596 | OPN3 | opsin 3 | 677846 | SNORA80A | small nucleolar RNA, H/ACA box |
| | | | | | 80A |
| 23600 | AMACR | alpha-methylacyl-CoA | 677850 | SNORD1C | small nucleolar RNA, C/D box 1C |
| | | racemase | | | |
| 23643 | LY96 | lymphocyte antigen 96 | 6781 | STC1 | stanniocalcin 1 |
| 23704 | KCNE4 | potassium voltage-gated | 6790 | AURKA | aurora kinase A |
| | | channel subfamily E | | | |
| | | regulatory subunit 4 | | | |
| 23705 | CADM1 | cell adhesion molecule 1 | 6799 | SULT1A2 | sulfotransferase family 1A |
| | | | | | member 2 |
| 23743 | BHMT2 | betaine—homocysteine S- | 6812 | STXBP1 | syntaxin binding protein 1 |
| | | methyltransferase 2 | | | |
| 23767 | FLRT3 | fibronectin leucine rich | 6817 | SULT1A1 | sulfotransferase family 1A |
| | | transmembrane protein 3 | | | member 1 |
| 240 | ALOX5 | arachidonate 5-lipoxygenase | 6819 | SULT1C2 | sulfotransferase family 1C |
| | | | | | member 2 |
| 241 | ALOX5AP | arachidonate 5-lipoxygenase | 684959 | SNORA25 | small nucleolar RNA, H/ACA box |
| | | activating protein | | | 25 |
| 24137 | KIF4A | kinesin family member 4A | 6876 | TAGLN | transgelin |
| 24141 | LAMP5 | lysosomal associated | 688 | KLF5 | Kruppel like factor 5 |
| | | membrane protein family | | | |
| | | member 5 | | | |
| 246126 | TXLNGY | taxilin gamma pseudogene, | 6887 | TAL2 | TAL bHLH transcription factor 2 |
| | | Y-linked | | | |
| 246181 | AKR7L | aldo-keto reductase family 7 | 6907 | TBL1X | transducin beta like 1 X-linked |
| | | like (gene/pseudogene) | | | |
| 246721 | POLR2J2 | RNA polymerase II subunit J2 | 6909 | TBX2 | T-box 2 |
| 246744 | STH | saitohin | 6913 | TBX15 | T-box 15 |
| 246777 | SPESP1 | sperm equatorial segment | 6920 | TCEA3 | transcription elongation factor A3 |
| | | protein 1 | | | |
| 247 | ALOX15B | arachidonate 15- | 692053 | SNORD9 | small nucleolar RNA, C/D box 9 |
| | | lipoxygenase, type B | | | |
| 2487 | FRZB | frizzled related protein | 692057 | SNORD12 | small nucleolar RNA, C/D box 12 |
| 249 | ALPL | alkaline phosphatase, | 692063 | SNORA32 | small nucleolar RNA, H/ACA box |
| | | liver/bone/kidney | | | 32 |
| 2528 | FUT6 | fucosyltransferase 6 | 692073 | SNORA16A | small nucleolar RNA, H/ACA box |
| | | | | | 16A |
| 2532 | ACKR1 | atypical chemokine receptor | 692076 | SNORD7 | small nucleolar RNA, C/D box 7 |
| | | 1 (Duffy blood group) | | | |
| 2533 | FYB1 | FYN binding protein 1 | 692084 | SNORD13 | small nucleolar RNA, C/D box 13 |
| 253650 | ANKRD18A | ankyrin repeat domain 18A | 692085 | SNORD45C | small nucleolar RNA, C/D box |
| | | | | | 45C |
| 253738 | EBF3 | early B-cell factor 3 | 692090 | SNORD59B | small nucleolar RNA, C/D box 59B |
| 253982 | ASPHD1 | aspartate beta-hydroxylase | 692106 | SNORD65 | small nucleolar RNA, C/D box 65 |
| | | domain containing 1 | | | |
| 2542 | SLC37A4 | solute carrier family 37 | 692107 | SNORD66 | small nucleolar RNA, C/D box 66 |
| | | member 4 | | | |
| 254295 | PHYHD1 | phytanoyl-CoA dioxygenase | 692108 | SNORD67 | small nucleolar RNA, C/D box 67 |
| | | domain containing 1 | | | |
| 255027 | MPV17L | MPV17 mitochondrial inner | 692109 | SNORD69 | small nucleolar RNA, C/D box 69 |
| | | membrane protein like | | | |
| 255231 | MCOLN2 | mucolipin 2 | 692111 | SNORD71 | small nucleolar RNA, C/D box 71 |
| 255631 | COL24A1 | collagen type XXIV alpha 1 | 692149 | SCARNA14 | small Cajal body-specific RNA 14 |
| | | chain | | | |
| 255743 | NPNT | nephronectin | 692196 | SNORD76 | small nucleolar RNA, C/D box 76 |
| 255877 | BCL6B | B-cell CLL/lymphoma 6B | 692199 | SNORD84 | small nucleolar RNA, C/D box 84 |
| 2562 | GABRB3 | gamma-aminobutyric acid | 692200 | SNORD103C | small nucleolar RNA, C/D box |
| | | type A receptor beta3 subunit | | | 103C |
| 256236 | NAPSB | napsin B aspartic peptidase, | 692204 | SNORD88C | small nucleolar RNA, C/D box |
| | | pseudogene | | | 88C |
| 2563 | GABRD | gamma-aminobutyric acid | 692205 | SNORD89 | small nucleolar RNA, C/D box 89 |
| | | type A receptor delta subunit | | | |
| 2564 | GABRE | gamma-aminobutyric acid | 692206 | SNORD90 | small nucleolar RNA, C/D box 90 |
| | | type A receptor epsilon | | | |
| | | subunit | | | |
| 256691 | MAMDC2 | MAM domain containing 2 | 692208 | SNORD91B | small nucleolar RNA, C/D box 91B |
| 256714 | MAP7D2 | MAP7 domain containing 2 | 692209 | SNORD92 | small nucleolar RNA, C/D box 92 |
| 256764 | WDR72 | WD repeat domain 72 | 692212 | SNORD99 | small nucleolar RNA, C/D box 99 |
| 257019 | FRMD3 | FERM domain containing 3 | 692213 | SNORD110 | small nucleolar RNA, C/D box 110 |
| 2571 | GAD1 | glutamate decarboxylase 1 | 692225 | SNORD94 | small nucleolar RNA, C/D box 94 |
| 257106 | ARHGAP30 | Rho GTPase activating | 692227 | SNORD104 | small nucleolar RNA, C/D box 104 |
| | | protein 30 | | | |
| 257177 | CFAP126 | cilia and flagella associated | 692229 | SNORD105 | small nucleolar RNA, C/D box 105 |
| | | protein 126 | | | |
| 257194 | NEGR1 | neuronal growth regulator 1 | 692233 | SNORD117 | small nucleolar RNA, C/D box 117 |
| 257407 | C2orf72 | chromosome 2 open reading | 6926 | TBX3 | T-box 3 |
| | | frame 72 | | | |
| 25759 | SHC2 | SHC adaptor protein 2 | 6927 | HNF1A | HNF1 homeobox A |
| 257629 | ANKS4B | ankyrin repeat and sterile | 6928 | HNF1B | HNF1 homeobox B |
| | | alpha motif domain | | | |
| | | containing 4B | | | |
| 25787 | DGCR9 | DiGeorge syndrome critical | 693197 | MIR612 | microRNA 612 |
| | | region gene 9 (non-protein | | | |
| | | coding) | | | |
| 25791 | NGEF | neuronal guanine nucleotide | 6943 | TCF21 | transcription factor 21 |
| | | exchange factor | | | |
| 25797 | QPCT | glutaminyl-peptide | 6947 | TCN1 | transcobalamin 1 |
| | | cyclotransferase | | | |
| 25802 | LMOD1 | leiomodin 1 | 6948 | TCN2 | transcobalamin 2 |
| 25805 | BAMBI | BMP and activin membrane | 695 | BTK | Bruton tyrosine kinase |
| | | bound inhibitor | | | |
| 25825 | BACE2 | beta-site APP-cleaving | 699 | BUB1 | BUB1 mitotic checkpoint |
| | | enzyme 2 | | | serine/threonine kinase |
| 25826 | SNORD82 | small nucleolar RNA, C/D box | 6999 | TDO2 | tryptophan 2,3-dioxygenase |
| | | 82 | | | |
| 2583 | B4GALNT1 | beta-1,4-N-acetyl- | 70 | ACTC1 | actin, alpha, cardiac muscle 1 |
| | | galactosaminyltransferase 1 | | | |
| 25840 | METTL7A | methyltransferase like 7A | 701 | BUB1B | BUB1 mitotic checkpoint |
| | | | | | serine/threonine kinase B |
| 25841 | ABTB2 | ankyrin repeat and BTB | 7010 | TEK | TEK receptor tyrosine kinase |
| | | domain containing 2 | | | |
| 25849 | PARM1 | prostate androgen-regulated | 7018 | TF | transferrin |
| | | mucin-like protein 1 | | | |
| 25854 | FAM149A | family with sequence | 7020 | TFAP2A | transcription factor AP-2 alpha |
| | | similarity 149 member A | | | |
| 25878 | MXRA5 | matrix remodeling associated | 7025 | NR2F1 | nuclear receptor subfamily 2 |
| | | 5 | | | group F member 1 |
| 25890 | ABI3BP | ABI family member 3 binding | 7033 | TFF3 | trefoil factor 3 |
| | | protein | | | |
| 25891 | PAMR1 | peptidase domain containing | 7035 | TFPI | tissue factor pathway inhibitor |
| | | associated with muscle | | | |
| | | regeneration 1 | | | |
| 25894 | PLEKHG4 | pleckstrin homology and | 7039 | TGFA | transforming growth factor alpha |
| | | RhoGEF domain containing | | | |
| | | G4 | | | |
| 259 | AMBP | alpha-1-microglobulin/bikunin | 7045 | TGFBI | transforming growth factor beta |
| | | precursor | | | induced |
| 25903 | OLFML2B | olfactomedin like 2B | 7049 | TGFBR3 | transforming growth factor beta |
| | | | | | receptor 3 |
| 2591 | GALNT3 | polypeptide N- | 7051 | TGM1 | transglutaminase 1 |
| | | acetylgalactosaminyltransferase | | | |
| | | 3 | | | |
| 259232 | NALCN | sodium leak channel, non- | 7052 | TGM2 | transglutaminase 2 |
| | | selective | | | |
| 25925 | ZNF521 | zinc finger protein 521 | 7057 | THBS1 | thrombospondin 1 |
| 259266 | ASPM | abnormal spindle microtubule | 7058 | THBS2 | thrombospondin 2 |
| | | assembly | | | |
| 259289 | TAS2R43 | taste 2 receptor member 43 | 7060 | THBS4 | thrombospondin 4 |
| 2593 | GAMT | guanidinoacetate N- | 7066 | THPO | thrombopoietin |
| | | methyltransferase | | | |
| 259307 | IL411 | interleukin 4 induced 1 | 7070 | THY1 | Thy-1 cell surface antigen |
| 25975 | EGFL6 | EGF like domain multiple 6 | 7075 | TIE1 | tyrosine kinase with |
| | | | | | immunoglobulin like and EGF like |
| | | | | | domains 1 |
| 25976 | TIPARP | TCDD inducible poly(ADP- | 7078 | TIMP3 | TIMP metallopeptidase inhibitor 3 |
| | | ribose) polymerase | | | |
| 25987 | TSKU | tsukushi, small leucine rich | 7079 | TIMP4 | TIMP metallopeptidase inhibitor 4 |
| | | proteoglycan | | | |
| 26 | AOC1 | amine oxidase, copper | 7083 | TK1 | thymidine kinase 1 |
| | | containing 1 | | | |
| 26002 | MOXD1 | monooxygenase DBH like 1 | 7089 | TLE2 | transducin like enhancer of split 2 |
| 26011 | TENM4 | teneurin transmembrane | 7092 | TLL1 | tolloid like 1 |
| | | protein 4 | | | |
| 26022 | TMEM98 | transmembrane protein 98 | 7098 | TLR3 | toll like receptor 3 |
| 260293 | CYP4X1 | cytochrome P450 family 4 | 7102 | TSPAN7 | tetraspanin 7 |
| | | subfamily X member 1 | | | |
| 26033 | ATRNL1 | attractin like 1 | 7103 | TSPAN8 | tetraspanin 8 |
| 26050 | SLITRK5 | SLIT and NTRK like family | 7108 | TM7SF2 | transmembrane 7 superfamily |
| | | member 5 | | | member 2 |
| 26053 | AUTS2 | AUTS2, activator of | 7111 | TMOD1 | tropomodulin 1 |
| | | transcription and | | | |
| | | developmental regulator | | | |
| 26084 | ARHGEF26 | Rho guanine nucleotide | 712 | C1QA | complement C1q A chain |
| | | exchange factor 26 | | | |
| 26095 | PTPN20 | protein tyrosine phosphatase, | 7123 | CLEC3B | C-type lectin domain family 3 |
| | | non-receptor type 20 | | | member B |
| 2615 | LRRC32 | leucine rich repeat containing | 713 | C1QB | complement C1q B chain |
| | | 32 | | | |
| 26150 | RIBC2 | RIB43A domain with coiled- | 7130 | TNFAIP6 | TNF alpha induced protein 6 |
| | | coils 2 | | | |
| 26154 | ABCA12 | ATP binding cassette | 7134 | TNNC1 | troponin C1, slow skeletal and |
| | | subfamily A member 12 | | | cardiac type |
| 26167 | PCDHB5 | protocadherin beta 5 | 7138 | TNNT1 | troponin T1, slow skeletal type |
| 261729 | STEAP2 | STEAP2 metalloreductase | 7139 | TNNT2 | troponin T2, cardiac type |
| 26191 | PTPN22 | protein tyrosine phosphatase, | 714 | C1QC | complement C1q C chain |
| | | non-receptor type 22 | | | |
| 2620 | GAS2 | growth arrest specific 2 | 7140 | TNNT3 | troponin T3, fast skeletal type |
| 26207 | PITPNC1 | phosphatidylinositol transfer | 7145 | TNS1 | tensin 1 |
| | | protein, cytoplasmic 1 | | | |
| 26219 | OR1J4 | olfactory receptor family 1 | 7148 | TNXB | tenascin XB |
| | | subfamily J member 4 | | | |
| 26223 | FBXL21 | F-box and leucine rich repeat | 715 | C1R | complement C1r |
| | | protein 21 | | | |
| | | (gene/pseudogene) | | | |
| 26227 | PHGDH | phosphoglycerate | 7153 | TOP2A | DNA topoisomerase II alpha |
| | | dehydrogenase | | | |
| 26247 | OR2L1P | olfactory receptor family 2 | 716 | C1S | complement C1s |
| | | subfamily L member 1 | | | |
| | | pseudogene | | | |
| 2625 | GATA3 | GATA binding protein 3 | 7164 | TPD52L1 | tumor protein D52 like 1 |
| 26253 | CLEC4E | C-type lectin domain family 4 | 717 | C2 | complement C2 |
| | | member E | | | |
| 2627 | GATA6 | GATA binding protein 6 | 718 | C3 | complement C3 |
| 26279 | PLA2G2D | phospholipase A2 group IID | 72 | ACTG2 | actin, gamma 2, smooth muscle, |
| | | | | | enteric |
| 2628 | GATM | glycine amidinotransferase | 720 | C4A | complement C4A (Rodgers blood |
| | | | | | group) |
| 2633 | GBP1 | guanylate binding protein 1 | 721 | C4B | complement C4B (Chido blood |
| | | | | | group) |
| 2635 | GBP3 | guanylate binding protein 3 | 7216 | TRO | trophinin |
| 26353 | HSPB8 | heat shock protein family B | 7225 | TRPC6 | transient receptor potential cation |
| | | (small) member 8 | | | channel subfamily C member 6 |
| 2638 | GC | GC, vitamin D binding protein | 723778 | MIR650 | microRNA 650 |
| 26470 | SEZ6L2 | seizure related 6 homolog | 7253 | TSHR | thyroid stimulating hormone |
| | | like 2 | | | receptor |
| 26499 | PLEK2 | pleckstrin 2 | 7262 | PHLDA2 | pleckstrin homology like domain |
| | | | | | family A member 2 |
| 26508 | HEYL | hes related family bHLH | 7263 | TST | thiosulfate sulfurtransferase |
| | | transcription factor with | | | |
| | | YRPW motif-like | | | |
| 26575 | RGS17 | regulator of G protein | 7272 | TTK | TTK protein kinase |
| | | signaling 17 | | | |
| 26577 | PCOLCE2 | procollagen C-endopeptidase | 7273 | TTN | titin |
| | | enhancer 2 | | | |
| 26579 | MYEOV | myeloma overexpressed | 7275 | TUB | tubby bipartite transcription factor |
| 26585 | GREM1 | gremlin 1, DAN family BMP | 727800 | RNF208 | ring finger protein 208 |
| | | antagonist | | | |
| 2669 | GEM | GTP binding protein | 727936 | GXYLT2 | glucoside xylosyltransferase 2 |
| | | overexpressed in skeletal | | | |
| | | muscle | | | |
| 267010 | RNU12 | RNA, U12 small nuclear | 727956 | SDHAP2 | succinate dehydrogenase |
| | | | | | complex flavoprotein subunit A |
| | | | | | pseudogene 2 |
| 2674 | GFRA1 | GDNF family receptor alpha 1 | 7280 | TUBB2A | tubulin beta 2A class lla |
| 26751 | SH3YL1 | SH3 and SYLF domain | 728053 | NA | NA |
| | | containing 1 | | | |
| 26762 | HAVCR1 | hepatitis A virus cellular | 728233 | PI4KAP1 | phosphatidylinositol 4-kinase |
| | | receptor 1 | | | alpha pseudogene 1 |
| 26765 | SNORD12C | small nucleolar RNA, C/D box | 728464 | METTL24 | methyltransferase like 24 |
| | | 12C | | | |
| 26773 | SNORD4A | small nucleolar RNA, C/D box | 728609 | SDHAP3 | succinate dehydrogenase |
| | | 4A | | | complex flavoprotein subunit A |
| | | | | | pseudogene 3 |
| 26774 | SNORD80 | small nucleolar RNA, C/D box | 728640 | FAM133CP | family with sequence similarity |
| | | 80 | | | 133, member A pseudogene |
| 26775 | SNORA72 | small nucleolar RNA, H/ACA | 728747 | ANKRD20A4 | ankyrin repeat domain 20 family |
| | | box 72 | | | member A4 |
| 26776 | SNORA71B | small nucleolar RNA, H/ACA | 729 | C6 | complement C6 |
| | | box 71B | | | |
| 26777 | SNORA71A | small nucleolar RNA, H/ACA | 729162 | FAM239B | zinc finger protein 839 |
| | | box 71A | | | pseudogene |
| 26779 | SNORA69 | small nucleolar RNA, H/ACA | 729171 | ANKRD20A8P | ankyrin repeat domain 20 family |
| | | box 69 | | | member A8, pseudogene |
| 2678 | GGT1 | gamma-glutamyltransferase 1 | 729230 | CCR2 | C-C motif chemokine receptor 2 |
| 26782 | SNORA66 | small nucleolar RNA, H/ACA | 729359 | PLIN4 | perilipin 4 |
| | | box 66 | | | |
| 26783 | SNORA65 | small nucleolar RNA, H/ACA | 729648 | ZNF812P | zinc finger protein 812, |
| | | box 65 | | | pseudogene |
| 26785 | SNORD63 | small nucleolar RNA, C/D box | 729737 | LOC729737 | uncharacterized LOC729737 |
| | | 63 | | | |
| 26787 | SNORD61 | small nucleolar RNA, C/D box | 729970 | LOC729970 | hCG2028352-like |
| | | 61 | | | |
| 26788 | SNORD60 | small nucleolar RNA, C/D box | 729993 | SHISA9 | shisa family member 9 |
| | | 60 | | | |
| 26791 | SNORD58A | small nucleolar RNA, C/D box | 730 | C7 | complement C7 |
| | | 58A | | | |
| 26792 | SNORD57 | small nucleolar RNA, C/D box | 730005 | SEC14L6 | SEC14 like lipid binding 6 |
| | | 57 | | | |
| 26793 | SNORD56 | small nucleolar RNA, C/D box | 730013 | ABCC6P2 | ATP binding cassette subfamily C |
| | | 56 | | | member 6 pseudogene 2 |
| 26795 | SNORD54 | small nucleolar RNA, C/D box | 730087 | ZNF726 | zinc finger protein 726 |
| | | 54 | | | |
| 26796 | SNORD53 | small nucleolar RNA, C/D box | 7305 | TYROBP | TYRO protein tyrosine kinase |
| | | 53 | | | binding protein |
| 26799 | SNORD50A | small nucleolar RNA, C/D box | 731220 | RFX8 | RFX family member 8, lacking |
| | | 50A | | | RFX DNA binding domain |
| 26800 | SNORD49A | small nucleolar RNA, C/D box | 7345 | UCHL1 | ubiquitin C-terminal hydrolase L1 |
| | | 49A | | | |
| 26801 | SNORD48 | small nucleolar RNA, C/D box | 735 | C9 | complement C9 |
| | | 48 | | | |
| 26802 | SNORD47 | small nucleolar RNA, C/D box | 7351 | UCP2 | uncoupling protein 2 |
| | | 47 | | | |
| 26805 | SNORD45A | small nucleolar RNA, C/D box | 7364 | UGT2B7 | UDP glucuronosyltransferase |
| | | 45A | | | family 2 member B7 |
| 2681 | GGTA1P | glycoprotein, alpha- | 7368 | UGT8 | UDP glycosyltransferase 8 |
| | | galactosyltransferase 1 | | | |
| | | pseudogene | | | |
| 26810 | SNORD41 | small nucleolar RNA, C/D box | 7373 | COL14A1 | collagen type XIV alpha 1 chain |
| | | 41 | | | |
| 26811 | SNORD55 | small nucleolar RNA, C/D box | 7388 | UQCRH | ubiquinol-cytochrome c reductase |
| | | 55 | | | hinge protein |
| 26813 | SNORD36C | small nucleolar RNA, C/D box | 7404 | UTY | ubiquitously transcribed |
| | | 36C | | | tetratricopeptide repeat |
| | | | | | containing, Y-linked |
| 26814 | SNORD36B | small nucleolar RNA, C/D box | 7409 | VAV1 | vav guanine nucleotide exchange |
| | | 36B | | | factor 1 |
| 26815 | SNORD36A | small nucleolar RNA, C/D box | 7412 | VCAM1 | vascular cell adhesion molecule 1 |
| | | 36A | | | |
| 26816 | SNORD35A | small nucleolar RNA, C/D box | 7422 | VEGFA | vascular endothelial growth factor |
| | | 35A | | | A |
| 26817 | SNORD34 | small nucleolar RNA, C/D box | 7424 | VEGFC | vascular endothelial growth factor |
| | | 34 | | | C |
| 26818 | SNORD33 | small nucleolar RNA, C/D box | 7429 | VIL1 | villin 1 |
| | | 33 | | | |
| 26819 | SNORD32A | small nucleolar RNA, C/D box | 745 | MYRF | myelin regulatory factor |
| | | 32A | | | |
| 26820 | SNORD24 | small nucleolar RNA, C/D box | 7450 | VWF | von Willebrand factor |
| | | 24 | | | |
| 26822 | SNORD14A | small nucleolar RNA, C/D box | 7482 | WNT2B | Wnt family member 2B |
| | | 14A | | | |
| 26824 | RNU11 | RNA, U11 small nuclear | 7490 | WT1 | Wilms tumor 1 |
| 26829 | RNU5E-1 | RNA, U5E small nuclear 1 | 7498 | XDH | xanthine dehydrogenase |
| 26831 | RNU5A-1 | RNA, U5A small nuclear 1 | 7503 | XIST | X inactive specific transcript (non- |
| | | | | | protein coding) |
| 26832 | RNU5B-1 | RNA, U5B small nuclear 1 | 7512 | XPNPEP2 | X-prolyl aminopeptidase 2 |
| 26834 | RNU4-2 | RNA, U4 small nuclear 2 | 7535 | ZAP70 | zeta chain of T-cell receptor |
| | | | | | associated protein kinase 70 |
| 26835 | RNU4-1 | RNA, U4 small nuclear 1 | 7538 | ZFP36 | ZFP36 ring finger protein |
| 26851 | SNORD3B-1 | small nucleolar RNA, C/D box | 7544 | ZFY | zinc finger protein, Y-linked |
| | | 3B-1 | | | |
| 26855 | RNU2-2P | RNA, U2 small nuclear 2, | 7552 | ZNF711 | zinc finger protein 711 |
| | | pseudogene | | | |
| 26860 | RNU1-13P | RNA, U1 small nuclear 13, | 760 | CA2 | carbonic anhydrase 2 |
| | | pseudogene | | | |
| 2687 | GGT5 | gamma-glutamyltransferase 5 | 762 | CA4 | carbonic anhydrase 4 |
| 26872 | STEAP1 | STEAP family member 1 | 7643 | ZNF90 | zinc finger protein 90 |
| 2690 | GHR | growth hormone receptor | 767 | CA8 | carbonic anhydrase 8 |
| 2697 | GJA1 | gap junction protein alpha 1 | 768 | CA9 | carbonic anhydrase 9 |
| 26996 | GPR160 | G protein-coupled receptor | 768206 | PRCD | photoreceptor disc component |
| | | 160 | | | |
| 270 | AMPD1 | adenosine monophosphate | 7694 | ZNF135 | zinc finger protein 135 |
| | | deaminase 1 | | | |
| 2701 | GJA4 | gap junction protein alpha 4 | 7704 | ZBTB16 | zinc finger and BTB domain |
| | | | | | containing 16 |
| 27019 | DNAI1 | dynein axonemal | 771 | CA12 | carbonic anhydrase 12 |
| | | intermediate chain 1 | | | |
| 2702 | GJA5 | gap junction protein alpha 5 | 7710 | ZNF154 | zinc finger protein 154 |
| 2705 | GJB1 | gap junction protein beta 1 | 775 | CACNA1C | calcium voltage-gated channel |
| | | | | | subunit alpha1 C |
| 2706 | GJB2 | gap junction protein beta 2 | 7754 | ZNF204P | zinc finger protein 204, |
| | | | | | pseudogene |
| 27063 | ANKRD1 | ankyrin repeat domain 1 | 7757 | ZNF208 | zinc finger protein 208 |
| 27074 | LAMP3 | lysosomal associated | 776 | CACNA1D | calcium voltage-gated channel |
| | | membrane protein 3 | | | subunit alpha1 D |
| 27075 | TSPAN13 | tetraspanin 13 | 7772 | ZNF229 | zinc finger protein 229 |
| 27122 | DKK3 | dickkopf WNT signaling | 778 | CACNA1F | calcium voltage-gated channel |
| | | pathway inhibitor 3 | | | subunit alpha1 F |
| 27128 | CYTH4 | cytohesin 4 | 7784 | ZP3 | zona pellucida glycoprotein 3 |
| 27132 | CPNE7 | copine 7 | 7802 | DNALI1 | dynein axonemal light |
| | | | | | intermediate chain 1 |
| 27141 | CIDEB | cell death-inducing DFFA-like | 7804 | LRP8 | LDL receptor related protein 8 |
| | | effector b | | | |
| 27145 | FILIP1 | filamin A interacting protein 1 | 780851 | SNORD3A | small nucleolar RNA, C/D box 3A |
| 27147 | DENND2A | DENN domain containing 2A | 780853 | SNORD3C | small nucleolar RNA, C/D box 3C |
| 27156 | RSPH14 | radial spoke head 14 | 780854 | SNORD3D | small nucleolar RNA, C/D box 3D |
| | | homolog | | | |
| 27181 | SIGLEC8 | sialic acid binding Ig like | 781 | CACNA2D1 | calcium voltage-gated channel |
| | | lectin 8 | | | auxiliary subunit alpha2delta 1 |
| 2719 | GPC3 | glypican 3 | 783 | CACNB2 | calcium voltage-gated channel |
| | | | | | auxiliary subunit beta 2 |
| 27202 | C5AR2 | complement component 5a | 7837 | PXDN | peroxidasin |
| | | receptor 2 | | | |
| 27233 | SULT1C4 | sulfotransferase family 1C | 7849 | PAX8 | paired box 8 |
| | | member 4 | | | |
| 27237 | ARHGEF16 | Rho guanine nucleotide | 7850 | IL1R2 | interleukin 1 receptor type 2 |
| | | exchange factor 16 | | | |
| 27242 | TNFRSF21 | TNF receptor superfamily | 7851 | MALL | mal, T-cell differentiation protein |
| | | member 21 | | | like |
| 27253 | PCDH17 | protocadherin 17 | 7857 | SCG2 | secretogranin II |
| 27283 | TINAG | tubulointerstitial nephritis | 7869 | SEMA3B | semaphorin 3B |
| | | antigen | | | |
| 27285 | TEKT2 | tektin 2 | 7881 | KCNAB1 | potassium voltage-gated channel |
| | | | | | subfamily A member regulatory |
| | | | | | beta subunit 1 |
| 27286 | SRPX2 | sushi repeat containing | 78989 | COLEC11 | collectin subfamily member 11 |
| | | protein, X-linked 2 | | | |
| 27293 | SMPDL3B | sphingomyelin | 79083 | MLPH | melanophilin |
| | | phosphodiesterase acid like | | | |
| | | 3B | | | |
| 27295 | PDLIM3 | PDZ and LIM domain 3 | 79168 | LILRA6 | leukocyte immunoglobulin like |
| | | | | | receptor A6 |
| 27299 | ADAMDEC1 | ADAM like decysin 1 | 79191 | IRX3 | iroquois homeobox 3 |
| 2731 | GLDC | glycine decarboxylase | 79365 | BHLHE41 | basic helix-loop-helix family |
| | | | | | member e41 |
| 27324 | TOX3 | TOX high mobility group box | 79369 | B3GNT4 | UDP-GIcNAc:betaGal beta-1,3-N- |
| | | family member 3 | | | acetylglucosaminyltransferase 4 |
| 27329 | ANGPTL3 | angiopoietin like 3 | 7940 | LST1 | leukocyte specific transcript 1 |
| 27334 | P2RY10 | P2Y receptor family member | 7941 | PLA2G7 | phospholipase A2 group VII |
| | | 10 | | | |
| 27344 | PCSKIN | proprotein convertase | 79411 | GLB1L | galactosidase beta 1 like |
| | | subtilisin/kexin type 1 inhibitor | | | |
| 27347 | STK39 | serine/threonine kinase 39 | 79444 | BIRC7 | baculoviral IAP repeat containing |
| | | | | | 7 |
| 2743 | GLRB | glycine receptor beta | 79589 | RNF128 | ring finger protein 128, E3 |
| | | | | | ubiquitin protein ligase |
| 27445 | PCLO | piccolo presynaptic | 79605 | PGBD5 | piggyBac transposable element |
| | | cytomatrix protein | | | derived 5 |
| 2745 | GLRX | glutaredoxin | 79611 | ACSS3 | acyl-CoA synthetase short chain |
| | | | | | family member 3 |
| 2791 | GNG11 | G protein subunit gamma 11 | 79623 | GALNT14 | polypeptide N- |
| | | | | | acetylgalactosaminyltransferase |
| | | | | | 14 |
| 28 | ABO | ABO, alpha 1-3-N- | 79625 | NDNF | neuron derived neurotrophic factor |
| | | acetylgalactosaminyltransferase | | | |
| | | and alpha 1-3- | | | |
| | | galactosyltransferase | | | |
| 280 | AMY2B | amylase, alpha 2B | 79632 | FAM184A | family with sequence similarity |
| | | (pancreatic) | | | 184 member A |
| 2805 | GOT1 | glutamic-oxaloacetic | 79633 | FAT4 | FAT atypical cadherin 4 |
| | | transaminase 1 | | | |
| 2810 | SFN | stratifin | 79652 | TMEM204 | transmembrane protein 204 |
| 2819 | GPD1 | glycerol-3-phosphate | 79656 | BEND5 | BEN domain containing 5 |
| | | dehydrogenase 1 | | | |
| 2823 | GPM6A | glycoprotein M6A | 79669 | C3orf52 | chromosome 3 open reading |
| | | | | | frame 52 |
| 28231 | SLCO4A1 | solute carrier organic anion | 79674 | VEPH1 | ventricular zone expressed PH |
| | | transporter family member | | | domain containing 1 |
| | | 4A1 | | | |
| 2828 | GPR4 | G protein-coupled receptor 4 | 79689 | STEAP4 | STEAP4 metalloreductase |
| 2829 | XCR1 | X-C motif chemokine receptor | 79729 | SH3D21 | SH3 domain containing 21 |
| | | 1 | | | |
| 282969 | FUOM | fucose mutarotase | 79730 | NSUN7 | NOP2/Sun RNA |
| | | | | | methyltransferase family member |
| | | | | | 7 |
| 282996 | RBM20 | RNA binding motif protein 20 | 79733 | E2F8 | E2F transcription factor 8 |
| 283 | ANG | angiogenin | 79739 | TTLL7 | tubulin tyrosine ligase like 7 |
| 283120 | H19 | H19, imprinted maternally | 79742 | CXorf36 | chromosome X open reading |
| | | expressed transcript (non- | | | frame 36 |
| | | protein coding) | | | |
| 283131 | NEAT1 | nuclear paraspeckle | 79745 | CLIP4 | CAP-Gly domain containing linker |
| | | assembly transcript 1 (non- | | | protein family member 4 |
| | | protein coding) | | | |
| 283208 | P4HA3 | prolyl 4-hydroxylase subunit | 79746 | ECHDC3 | enoyl-CoA hydratase domain |
| | | alpha 3 | | | containing 3 |
| 283316 | CD163L1 | CD163 molecule like 1 | 79750 | ZNF385D | zinc finger protein 385D |
| 283358 | B4GALNT3 | beta-1,4-N-acetyl- | 79774 | GRTP1 | growth hormone regulated TBC |
| | | galactosaminyltransferase 3 | | | protein 1 |
| 283375 | SLC39A5 | solute carrier family 39 | 79776 | ZFHX4 | zinc finger homeobox 4 |
| | | member 5 | | | |
| 283383 | ADGRD1 | adhesion G protein-coupled | 79781 | IQCA1 | IQ motif containing with AAA |
| | | receptor D1 | | | domain 1 |
| 283392 | TRHDE-AS1 | TRHDE antisense RNA 1 | 79783 | SUGCT | succinyl-CoA:glutarate-CoA |
| | | | | | transferase |
| 283417 | DPY19L2 | dpy-19 like 2 | 79784 | MYH14 | myosin heavy chain 14 |
| 283422 | LINC01559 | long intergenic non-protein | 79785 | RERGL | RERG like |
| | | coding RNA 1559 | | | |
| 283431 | GAS2L3 | growth arrest specific 2 like 3 | 79799 | UGT2A3 | UDP glucuronosyltransferase |
| | | | | | family 2 member A3 |
| 283755 | HERC2P3 | hect domain and RLD 2 | 7980 | TFPI2 | tissue factor pathway inhibitor 2 |
| | | pseudogene 3 | | | |
| 283796 | GOLGA8IP | golgin A8 family member I, | 79801 | SHCBP1 | SHC binding and spindle |
| | | pseudogene | | | associated 1 |
| 283848 | CES4A | carboxylesterase 4A | 79812 | MMRN2 | multimerin 2 |
| 283849 | EXOC3L1 | exocyst complex component | 79814 | AGMAT | agmatinase |
| | | 3 like 1 | | | |
| 283971 | CLEC18C | C-type lectin domain family | 79817 | MOB3B | MOB kinase activator 3B |
| | | 18 member C | | | |
| 284 | ANGPT1 | angiopoietin 1 | 79820 | CATSPERB | cation channel sperm associated |
| | | | | | auxiliary subunit beta |
| 284047 | CCDC144B | coiled-coil domain containing | 79822 | ARHGAP28 | Rho GTPase activating protein 28 |
| | | 144B (pseudogene) | | | |
| 284076 | TTLL6 | tubulin tyrosine ligase like 6 | 79827 | CLMP | CXADR like membrane protein |
| 284217 | LAMA1 | laminin subunit alpha 1 | 79839 | CCDC102B | coiled-coil domain containing |
| | | | | | 102B |
| 284297 | SSC5D | scavenger receptor cysteine | 79849 | PDZD3 | PDZ domain containing 3 |
| | | rich family member with 5 | | | |
| | | domains | | | |
| 284339 | TMEM145 | transmembrane protein 145 | 79883 | PODNL1 | podocan like 1 |
| 284422 | SMIM24 | small integral membrane | 79888 | LPCAT1 | lysophosphatidylcholine |
| | | protein 24 | | | acyltransferase 1 |
| 284612 | SYPL2 | synaptophysin like 2 | 799 | CALCR | calcitonin receptor |
| 2847 | MCHR1 | melanin concentrating | 79931 | TNIP3 | TNFAIP3 interacting protein 3 |
| | | hormone receptor 1 | | | |
| 284716 | RIMKLA | ribosomal modification | 79953 | SYNDIG1 | synapse differentiation inducing 1 |
| | | protein rimK like family | | | |
| | | member A | | | |
| 284904 | SEC14L4 | SEC14 like lipid binding 4 | 79971 | WLS | wntless Wnt ligand secretion |
| | | | | | mediator |
| 285 | ANGPT2 | angiopoietin 2 | 79983 | POF1B | POF1B, actin binding protein |
| 285016 | ALKAL2 | ALK and LTK ligand 2 | 79986 | ZNF702P | zinc finger protein 702, |
| | | | | | pseudogene |
| 285025 | CCDC141 | coiled-coil domain containing | 79987 | SVEP1 | sushi, von Willebrand factor type |
| | | 141 | | | A, EGF and pentraxin domain |
| | | | | | containing 1 |
| 28514 | DLL1 | delta like canonical Notch | 79993 | ELOVL7 | ELOVL fatty acid elongase 7 |
| | | ligand 1 | | | |
| 2852 | GPER1 | G protein-coupled estrogen | 80000 | GREB1L | growth regulation by estrogen in |
| | | receptor 1 | | | breast cancer 1 like |
| 285220 | EPHA6 | EPH receptor A6 | 80008 | TMEM156 | transmembrane protein 156 |
| 285498 | RNF212 | ring finger protein 212 | 80022 | MYO15B | myosin XVB |
| 285590 | SH3PXD2B | SH3 and PX domains 2B | 80031 | SEMA6D | semaphorin 6D |
| 285596 | FAM153A | family with sequence | 80036 | TRPM3 | transient receptor potential cation |
| | | similarity 153 member A | | | channel subfamily M member 3 |
| 2857 | GPR34 | G protein-coupled receptor | 80039 | FAM106A | family with sequence similarity |
| | | 34 | | | 106 member A |
| 2859 | GPR35 | G protein-coupled receptor | 80086 | TUBA4B | tubulin alpha 4b |
| | | 35 | | | |
| 286 | ANK1 | ankyrin 1 | 80115 | BAIAP2L2 | BAI1 associated protein 2 like 2 |
| 286077 | FAM83H | family with sequence | 8013 | NR4A3 | nuclear receptor subfamily 4 |
| | | similarity 83 member H | | | group A member 3 |
| 2861 | GPR37 | G protein-coupled receptor | 80144 | FRAS1 | Fraser extracellular matrix |
| | | 37 | | | complex subunit 1 |
| 286464 | CFAP47 | cilia and flagella associated | 80150 | ASRGL1 | asparaginase like 1 |
| | | protein 47 | | | |
| 286676 | ILDR1 | immunoglobulin like domain | 80162 | PGGHG | protein- |
| | | containing receptor 1 | | | glucosylgalactosylhydroxylysine |
| | | | | | glucosidase |
| 287 | ANK2 | ankyrin 2 | 80164 | PRR36 | proline rich 36 |
| 2875 | GPT | glutamic—pyruvic | 80177 | MYCT1 | MYC target 1 |
| | | transaminase | | | |
| 2878 | GPX3 | glutathione peroxidase 3 | 80183 | RUBCNL | RUN and cysteine rich domain |
| | | | | | containing beclin 1 interacting |
| | | | | | protein like |
| 288 | ANK3 | ankyrin 3 | 80201 | HKDC1 | hexokinase domain containing 1 |
| 2886 | GRB7 | growth factor receptor bound | 80217 | CFAP43 | cilia and flagella associated |
| | | protein 7 | | | protein 43 |
| 2888 | GRB14 | growth factor receptor bound | 80221 | ACSF2 | acyl-CoA synthetase family |
| | | protein 14 | | | member 2 |
| 2892 | GRIA3 | glutamate ionotropic receptor | 80243 | PREX2 | phosphatidylinositol-3,4,5- |
| | | AMPA type subunit 3 | | | trisphosphate dependent Rac |
| | | | | | exchange factor 2 |
| 2894 | GRID1 | glutamate ionotropic receptor | 80258 | EFHC2 | EF-hand domain containing 2 |
| | | delta type subunit 1 | | | |
| 28951 | TRIB2 | tribbles pseudokinase 2 | 80270 | HSD3B7 | hydroxy-delta-5-steroid |
| | | | | | dehydrogenase, 3 beta- and |
| | | | | | steroid delta-isomerase 7 |
| 28968 | SLC6A16 | solute carrier family 6 | 8029 | CUBN | cubilin |
| | | member 16 | | | |
| 28970 | C11orf54 | chromosome 11 open | 80303 | EFHD1 | EF-hand domain family member |
| | | reading frame 54 | | | D1 |
| 28984 | RGCC | regulator of cell cycle | 80307 | FER1L4 | fer-1 like family member 4, |
| | | | | | pseudogene |
| 2899 | GRIK3 | glutamate ionotropic receptor | 80310 | PDGFD | platelet derived growth factor D |
| | | kainate type subunit 3 | | | |
| 28999 | KLF15 | Kruppel like factor 15 | 80323 | CCDC68 | coiled-coil domain containing 68 |
| 290 | ANPEP | alanyl aminopeptidase, | 80328 | ULBP2 | UL16 binding protein 2 |
| | | membrane | | | |
| 2903 | GRIN2A | glutamate ionotropic receptor | 80333 | KCNIP4 | potassium voltage-gated channel |
| | | NMDA type subunit 2A | | | interacting protein 4 |
| 2904 | GRIN2B | glutamate ionotropic receptor | 80339 | PNPLA3 | patatin like phospholipase domain |
| | | NMDA type subunit 2B | | | containing 3 |
| 29089 | UBE2T | ubiquitin conjugating enzyme | 80342 | TRAF3IP3 | TRAF3 interacting protein 3 |
| | | E2 T | | | |
| 29108 | PYCARD | PYD and CARD domain | 8038 | ADAM12 | ADAM metallopeptidase domain |
| | | containing | | | 12 |
| 2918 | GRM8 | glutamate metabotropic | 80380 | PDCD1LG2 | programmed cell death 1 ligand 2 |
| | | receptor 8 | | | |
| 2919 | CXCL1 | C-X-C motif chemokine | 8061 | FOSL1 | FOS like 1, AP-1 transcription |
| | | ligand 1 | | | factor subunit |
| 2920 | CXCL2 | C-X-C motif chemokine | 80704 | SLC19A3 | solute carrier family 19 member 3 |
| | | ligand 2 | | | |
| 2938 | GSTA1 | glutathione S-transferase | 80726 | IQCN | IQ motif containing N |
| | | alpha 1 | | | |
| 2939 | GSTA2 | glutathione S-transferase | 80731 | THSD7B | thrombospondin type 1 domain |
| | | alpha 2 | | | containing 7B |
| 2944 | GSTM1 | glutathione S-transferase mu | 80736 | SLC44A4 | solute carrier family 44 member 4 |
| | | 1 | | | |
| 2947 | GSTM3 | glutathione S-transferase mu | 8076 | MFAP5 | microfibril associated protein 5 |
| | | 3 | | | |
| 2949 | GSTM5 | glutathione S-transferase mu | 80760 | ITIH5 | inter-alpha-trypsin inhibitor heavy |
| | | 5 | | | chain family member 5 |
| 29760 | BLNK | B-cell linker | 80763 | SPX | spexin hormone |
| 29763 | PACSIN3 | protein kinase C and casein | 80816 | ASXL3 | additional sex combs like 3, |
| | | kinase substrate in neurons 3 | | | transcriptional regulator |
| 29802 | VPREB3 | V-set pre-B cell surrogate | 80832 | APOL4 | apolipoprotein L4 |
| | | light chain 3 | | | |
| 29851 | ICOS | inducible T-cell costimulator | 80896 | NPL | N-acetylneuraminate pyruvate |
| | | | | | lyase |
| 29909 | GPR171 | G protein-coupled receptor | 81029 | WNT5B | Wnt family member 5B |
| | | 171 | | | |
| 29923 | HILPDA | hypoxia inducible lipid droplet | 81030 | ZBP1 | Z-DNA binding protein 1 |
| | | associated | | | |
| 29943 | PADI1 | peptidyl arginine deiminase 1 | 81031 | SLC2A10 | solute carrier family 2 member 10 |
| 29944 | PNMA3 | PNMA family member 3 | 81035 | COLEC12 | collectin subfamily member 12 |
| 29948 | OSGIN1 | oxidative stress induced | 8120 | AP3B2 | adaptor related protein complex 3 |
| | | growth inhibitor 1 | | | beta 2 subunit |
| 29953 | TRHDE | thyrotropin releasing | 81285 | OR51E2 | olfactory receptor family 51 |
| | | hormone degrading enzyme | | | subfamily E member 2 |
| 29958 | DMGDH | dimethylglycine | 8140 | SLC7A5 | solute carrier family 7 member 5 |
| | | dehydrogenase | | | |
| 29968 | PSAT1 | phosphoserine | 81466 | OR2L5 | olfactory receptor family 2 |
| | | aminotransferase 1 | | | subfamily L member 5 |
| 29974 | A1CF | APOBEC1 complementation | 81575 | APOLD1 | apolipoprotein L domain |
| | | factor | | | containing 1 |
| 2999 | GZMH | granzyme H | 81578 | COL21A1 | collagen type XXI alpha 1 chain |
| 3001 | GZMA | granzyme A | 81615 | TMEM163 | transmembrane protein 163 |
| 3002 | GZMB | granzyme B | 81624 | DIAPH3 | diaphanous related formin 3 |
| 3003 | GZMK | granzyme K | 81693 | AMN | amnion associated |
| | | | | | transmembrane protein |
| 3007 | HIST1H1D | histone cluster 1 H1 family | 81706 | PPP1R14C | protein phosphatase 1 regulatory |
| | | member d | | | inhibitor subunit 14C |
| 3009 | HIST1H1B | histone cluster 1 H1 family | 81792 | ADAMTS12 | ADAM metallopeptidase with |
| | | member b | | | thrombospondin type 1 motif 12 |
| 3012 | HIST1H2AE | histone cluster 1 H2A family | 81794 | ADAMTS10 | ADAM metallopeptidase with |
| | | member e | | | thrombospondin type 1 motif 10 |
| 3013 | HIST1H2AD | histone cluster 1 H2A family | 81831 | NETO2 | neuropilin and tolloid like 2 |
| | | member d | | | |
| 3018 | HIST1H2BB | histone cluster 1 H2B family | 8263 | F8A1 | coagulation factor VIII associated |
| | | member b | | | 1 |
| 3024 | HIST1H1A | histone cluster 1 H1 family | 827 | CAPN6 | calpain 6 |
| | | member a | | | |
| 3026 | HABP2 | hyaluronan binding protein 2 | 8284 | KDM5D | lysine demethylase 5D |
| 3037 | HAS2 | hyaluronan synthase 2 | 8287 | USP9Y | ubiquitin specific peptidase 9, Y- |
| | | | | | linked |
| 3039 | HBA1 | hemoglobin subunit alpha 1 | 8294 | HIST1H41 | histone cluster 1 H4 family |
| | | | | | member i |
| 3040 | HBA2 | hemoglobin subunit alpha 2 | 8302 | KLRC4 | killer cell lectin like receptor C4 |
| 3043 | HBB | hemoglobin subunit beta | 8309 | ACOX2 | acyl-CoA oxidase 2 |
| 3048 | HBG2 | hemoglobin subunit gamma 2 | 8320 | EOMES | eomesodermin |
| 306 | ANXA3 | annexin A3 | 8321 | FZD1 | frizzled class receptor 1 |
| 3067 | HDC | histidine decarboxylase | 8322 | FZD4 | frizzled class receptor 4 |
| 307 | ANXA4 | annexin A4 | 8329 | HIST1H2AI | histone cluster 1 H2A family |
| | | | | | member i |
| 3071 | NCKAP1L | NCK associated protein 1 like | 8331 | HIST1H2AJ | histone cluster 1 H2A family |
| | | | | | member j |
| 3075 | CFH | complement factor H | 8332 | HIST1H2AL | histone cluster 1 H2A family |
| | | | | | member I |
| 3078 | CFHR1 | complement factor H related | 8335 | HIST1H2AB | histone cluster 1 H2A family |
| | | 1 | | | member b |
| 3081 | HGD | homogentisate 1,2- | 8336 | HIST1H2AM | histone cluster 1 H2A family |
| | | dioxygenase | | | member m |
| 30818 | KCNIP3 | potassium voltage-gated | 8339 | HIST1H2BG | histone cluster 1 H2B family |
| | | channel interacting protein 3 | | | member g |
| 3082 | HGF | hepatocyte growth factor | 8340 | HIST1H2BL | histone cluster 1 H2B family |
| | | | | | member I |
| 30832 | ZNF354C | zinc finger protein 354C | 83416 | FCRL5 | Fc receptor like 5 |
| 30835 | CD209 | CD209 molecule | 8342 | HIST1H2BM | histone cluster 1 H2B family |
| | | | | | member m |
| 3084 | NRG1 | neuregulin 1 | 8343 | HIST1H2BF | histone cluster 1 H2B family |
| | | | | | member f |
| 3099 | HK2 | hexokinase 2 | 8345 | HIST1H2BH | histone cluster 1 H2B family |
| | | | | | member h |
| 3101 | HK3 | hexokinase 3 | 83450 | DRC3 | dynein regulatory complex subunit |
| | | | | | 3 |
| 3111 | HLA-DOA | major histocompatibility | 8346 | HIST1H2BI | histone cluster 1 H2B family |
| | | complex, class II, DO alpha | | | member i |
| 3112 | HLA-DOB | major histocompatibility | 83468 | GLT8D2 | glycosyltransferase 8 domain |
| | | complex, class II, DO beta | | | containing 2 |
| 3116 | HLA-DPB2 | major histocompatibility | 83478 | ARHGAP24 | Rho GTPase activating protein 24 |
| | | complex, class II, DP beta 2 | | | |
| | | (pseudogene) | | | |
| 3119 | HLA-DQB1 | major histocompatibility | 8348 | HIST1H2BO | histone cluster 1 H2B family |
| | | complex, class II, DQ beta 1 | | | member o |
| 312 | ANXA13 | annexin A13 | 83481 | EPPK1 | epiplakin 1 |
| 3120 | HLA-DQB2 | major histocompatibility | 83483 | PLVAP | plasmalemma vesicle associated |
| | | complex, class II, DQ beta 2 | | | protein |
| 3123 | HLA-DRB1 | major histocompatibility | 8350 | HIST1H3A | histone cluster 1 H3 family |
| | | complex, class II, DR beta 1 | | | member a |
| 3125 | HLA-DRB3 | major histocompatibility | 8352 | HIST1H3C | histone cluster 1 H3 family |
| | | complex, class II, DR beta 3 | | | member c |
| 3126 | HLA-DRB4 | major histocompatibility | 8353 | HIST1H3E | histone cluster 1 H3 family |
| | | complex, class II, DR beta 4 | | | member e |
| 3128 | HLA-DRB6 | major histocompatibility | 83539 | CHST9 | carbohydrate sulfotransferase 9 |
| | | complex, class II, DR beta 6 | | | |
| | | (pseudogene) | | | |
| 313 | AOAH | acyloxyacyl hydrolase | 8354 | HIST1H31 | histone cluster 1 H3 family |
| | | | | | member i |
| 3131 | HLF | HLF, PAR bZIP transcription | 83540 | NUF2 | NUF2, NDC80 kinetochore |
| | | factor | | | complex component |
| 3134 | HLA-F | major histocompatibility | 83543 | AIF1L | allograft inflammatory factor 1 like |
| | | complex, class I, F | | | |
| 3135 | HLA-G | major histocompatibility | 8355 | HIST1H3G | histone cluster 1 H3 family |
| | | complex, class I, G | | | member g |
| 3136 | HLA-H | major histocompatibility | 8356 | HIST1H3J | histone cluster 1 H3 family |
| | | complex, class I, H | | | member j |
| | | (pseudogene) | | | |
| 3137 | HLA-J | major histocompatibility | 8357 | HIST1H3H | histone cluster 1 H3 family |
| | | complex, class I, J | | | member h |
| | | (pseudogene) | | | |
| 3158 | HMGCS2 | 3-hydroxy-3-methylglutaryl- | 8358 | HIST1H3B | histone cluster 1 H3 family |
| | | CoA synthase 2 | | | member b |
| 3159 | HMGA1 | high mobility group AT-hook | 83592 | AKR1E2 | aldo-keto reductase family 1 |
| | | 1 | | | member E2 |
| 316 | AOX1 | aldehyde oxidase 1 | 8361 | HIST1H4F | histone cluster 1 H4 family |
| | | | | | member f |
| 3161 | HMMR | hyaluronan mediated motility | 83660 | TLN2 | talin 2 |
| | | receptor | | | |
| 3162 | HMOX1 | heme oxygenase 1 | 8368 | HIST1H4L | histone cluster 1 H4 family |
| | | | | | member I |
| 3164 | NR4A1 | nuclear receptor subfamily 4 | 83690 | CRISPLD1 | cysteine rich secretory protein |
| | | group A member 1 | | | LCCL domain containing 1 |
| 3170 | FOXA2 | forkhead box A2 | 83706 | FERMT3 | fermitin family member 3 |
| 3172 | HNF4A | hepatocyte nuclear factor 4 | 83714 | NRIP2 | nuclear receptor interacting |
| | | alpha | | | protein 2 |
| 3174 | HNF4G | hepatocyte nuclear factor 4 | 83715 | ESPN | espin |
| | | gamma | | | |
| 3177 | SLC29A2 | solute carrier family 29 | 83716 | CRISPLD2 | cysteine rich secretory protein |
| | | member 2 | | | LCCL domain containing 2 |
| 319103 | SNORD8 | small nucleolar RNA, C/D box | 83758 | RBP5 | retinol binding protein 5 |
| | | 8 | | | |
| 3199 | HOXA2 | homeobox A2 | 838 | CASP5 | caspase 5 |
| 3201 | HOXA4 | homeobox A4 | 8382 | NME5 | NME/NM23 family member 5 |
| 3203 | HOXA6 | homeobox A6 | 83872 | HMCN1 | hemicentin 1 |
| 3204 | HOXA7 | homeobox A7 | 83879 | CDCA7 | cell division cycle associated 7 |
| 3206 | HOXA10 | homeobox A10 | 83935 | TMEM133 | transmembrane protein 133 |
| 3207 | HOXA11 | homeobox A11 | 8395 | PIP5K1B | phosphatidylinositol-4-phosphate |
| | | | | | 5-kinase type 1 beta |
| 3209 | HOXA13 | homeobox A13 | 83953 | FCAMR | Fc fragment of IgA and IgM |
| | | | | | receptor |
| 321 | APBA2 | amyloid beta precursor | 83987 | CCDC8 | coiled-coil domain containing 8 |
| | | protein binding family A | | | |
| | | member 2 | | | |
| 3226 | HOXC10 | homeobox C10 | 83992 | CTTNBP2 | cortactin binding protein 2 |
| 3233 | HOXD4 | homeobox D4 | 84033 | OBSCN | obscurin, cytoskeletal calmodulin |
| | | | | | and titin-interacting RhoGEF |
| 3235 | HOXD9 | homeobox D9 | 8404 | SPARCL1 | SPARC like 1 |
| 3236 | HOXD10 | homeobox D10 | 84054 | PCDHB19P | protocadherin beta 19 |
| | | | | | pseudogene |
| 3237 | HOXD11 | homeobox D11 | 8406 | SRPX | sushi repeat containing protein, X- |
| | | | | | linked |
| 3240 | HP | haptoglobin | 84109 | QRFPR | pyroglutamylated RFamide |
| | | | | | peptide receptor |
| 3241 | HPCAL1 | hippocalcin like 1 | 84125 | LRRIQ1 | leucine rich repeats and IQ motif |
| | | | | | containing 1 |
| 3242 | HPD | 4-hydroxyphenylpyruvate | 84129 | ACAD11 | acyl-CoA dehydrogenase family |
| | | dioxygenase | | | member 11 |
| 3248 | HPGD | 15-hydroxyprostaglandin | 84144 | SYDE2 | synapse defective Rho GTPase |
| | | dehydrogenase | | | homolog 2 |
| 3249 | HPN | hepsin | 8416 | ANXA9 | annexin A9 |
| 326342 | ADGRE4P | adhesion G protein-coupled | 84166 | NLRC5 | NLR family CARD domain |
| | | receptor E4, pseudogene | | | containing 5 |
| 3270 | HRC | histidine rich calcium binding | 84168 | ANTXR1 | anthrax toxin receptor 1 |
| | | protein | | | |
| 3274 | HRH2 | histamine receptor H2 | 84171 | LOXL4 | lysyl oxidase like 4 |
| 3280 | HES1 | hes family bHLH transcription | 84174 | SLA2 | Src like adaptor 2 |
| | | factor 1 | | | |
| 3284 | HSD3B2 | hydroxy-delta-5-steroid | 84217 | ZMYND12 | zinc finger MYND-type containing |
| | | dehydrogenase, 3 beta- and | | | 12 |
| | | steroid delta-isomerase 2 | | | |
| 3290 | HSD11B1 | hydroxysteroid 11-beta | 84239 | ATP13A4 | ATPase 13A4 |
| | | dehydrogenase 1 | | | |
| 3291 | HSD11B2 | hydroxysteroid 11-beta | 8424 | BBOX1 | gamma-butyrobetaine |
| | | dehydrogenase 2 | | | hydroxylase 1 |
| 3293 | HSD17B3 | hydroxysteroid 17-beta | 8425 | LTBP4 | latent transforming growth factor |
| | | dehydrogenase 3 | | | beta binding protein 4 |
| 3294 | HSD17B2 | hydroxysteroid 17-beta | 84251 | SGIP1 | SH3 domain GRB2 like endophilin |
| | | dehydrogenase 2 | | | interacting protein 1 |
| 3299 | HSF4 | heat shock transcription | 84264 | HAGHL | hydroxyacylglutathione hydrolase |
| | | factor 4 | | | like |
| 33 | ACADL | acyl-CoA dehydrogenase | 84302 | TMEM246 | transmembrane protein 246 |
| | | long chain | | | |
| 330 | BIRC3 | baculoviral IAP repeat | 8436 | CAVIN2 | caveolae associated protein 2 |
| | | containing 3 | | | |
| 3303 | HSPA1A | heat shock protein family A | 8437 | RASAL1 | RAS protein activator like 1 |
| | | (Hsp70) member 1A | | | |
| 3304 | HSPA1B | heat shock protein family A | 8438 | RAD54L | RAD54 like |
| | | (Hsp70) member 1B | | | |
| 3306 | HSPA2 | heat shock protein family A | 84417 | C2orf40 | chromosome 2 open reading |
| | | (Hsp70) member 2 | | | frame 40 |
| 3311 | HSPA7 | heat shock protein family A | 84419 | C15orf48 | chromosome 15 open reading |
| | | (Hsp70) member 7 | | | frame 48 |
| 3316 | HSPB2 | heat shock protein family B | 84433 | CARD11 | caspase recruitment domain |
| | | (small) member 2 | | | family member 11 |
| 332 | BIRC5 | baculoviral IAP repeat | 84451 | MAP3K21 | mitogen-activated protein kinase |
| | | containing 5 | | | kinase kinase 21 |
| 333 | APLP1 | amyloid beta precursor like | 84457 | PHYHIPL | phytanoyl-CoA 2-hydroxylase |
| | | protein 1 | | | interacting protein like |
| 3339 | HSPG2 | heparan sulfate proteoglycan | 84465 | MEGF11 | multiple EGF like domains 11 |
| | | 2 | | | |
| 3357 | HTR2B | 5-hydroxytryptamine receptor | 8447 | DOC2B | double C2 domain beta |
| | | 2B | | | |
| 3371 | TNC | tenascin C | 8448 | DOC2A | double C2 domain alpha |
| 3373 | HYAL1 | hyaluronoglucosaminidase 1 | 845 | CASQ2 | calsequestrin 2 |
| 337875 | HIST2H2BA | histone cluster 2 H2B family | 84546 | SNORD35B | small nucleolar RNA, C/D box 35B |
| | | member a (pseudogene) | | | |
| 338 | APOB | apolipoprotein B | 84561 | SLC12A8 | solute carrier family 12 member 8 |
| 338094 | FAM151A | family with sequence | 84612 | PARD6B | par-6 family cell polarity regulator |
| | | similarity 151 member A | | | beta |
| 3381 | IBSP | integrin binding sialoprotein | 84624 | FNDC1 | fibronectin type III domain |
| | | | | | containing 1 |
| 338328 | GPIHBP1 | glycosylphosphatidylinositol | 84626 | KRBA1 | KRAB-A domain containing 1 |
| | | anchored high density | | | |
| | | lipoprotein binding protein 1 | | | |
| 338440 | ANO9 | anoctamin 9 | 84627 | ZNF469 | zinc finger protein 469 |
| 338442 | HCAR2 | hydroxycarboxylic acid | 84631 | SLITRK2 | SLIT and NTRK like family |
| | | receptor 2 | | | member 2 |
| 338596 | ST8SIA6 | ST8 alpha-N-acetyl- | 84634 | KISS1R | KISS1 receptor |
| | | neuraminide alpha-2,8- | | | |
| | | sialyltransferase 6 | | | |
| 338707 | B4GALNT4 | beta-1,4-N-acetyl- | 84636 | GPR174 | G protein-coupled receptor 174 |
| | | galactosaminyltransferase 4 | | | |
| 338773 | TMEM119 | transmembrane protein 119 | 84647 | PLA2G12B | phospholipase A2 group XIIB |
| 339400 | FLG-AS1 | FLG antisense RNA 1 | 84675 | TRIM55 | tripartite motif containing 55 |
| 3397 | ID1 | inhibitor of DNA binding 1, | 84689 | MS4A14 | membrane spanning 4-domains |
| | | HLH protein | | | A14 |
| 339778 | C2orf70 | chromosome 2 open reading | 84699 | CREB3L3 | CAMP responsive element binding |
| | | frame 70 | | | protein 3 like 3 |
| 3399 | ID3 | inhibitor of DNA binding 3, | 8470 | SORBS2 | sorbin and SH3 domain containing |
| | | HLH protein | | | 2 |
| 339965 | CCDC158 | coiled-coil domain containing | 84701 | COX412 | cytochrome c oxidase subunit 412 |
| | | 158 | | | |
| 3400 | ID4 | inhibitor of DNA binding 4, | 84706 | GPT2 | glutamic—pyruvic transaminase 2 |
| | | HLH protein | | | |
| 340267 | COL28A1 | collagen type XXVIII alpha 1 | 84707 | BEX2 | brain expressed X-linked 2 |
| | | chain | | | |
| 340307 | CTAGE6 | CTAGE family member 6 | 84708 | LNX1 | ligand of numb-protein X 1 |
| 340348 | TSPAN33 | tetraspanin 33 | 84709 | MGARP | mitochondria localized glutamic |
| | | | | | acid rich protein |
| 340351 | AGBL3 | ATP/GTP binding protein like | 8477 | GPR65 | G protein-coupled receptor 65 |
| | | 3 | | | |
| 340542 | BEX5 | brain expressed X-linked 5 | 84803 | GPAT3 | glycerol-3-phosphate |
| | | | | | acyltransferase 3 |
| 340547 | VSIG1 | V-set and immunoglobulin | 84808 | PERM1 | PPARGC1 and ESRR induced |
| | | domain containing 1 | | | regulator, muscle 1 |
| 341 | APOC1 | apolipoprotein C1 | 84812 | PLCD4 | phospholipase C delta 4 |
| 341019 | DCDC1 | doublecortin domain | 84866 | TMEM25 | transmembrane protein 25 |
| | | containing 1 | | | |
| 341640 | FREM2 | FRAS1 related extracellular | 84868 | HAVCR2 | hepatitis A virus cellular receptor 2 |
| | | matrix protein 2 | | | |
| 341676 | NEK5 | NIMA related kinase 5 | 84879 | MFSD2A | major facilitator superfamily |
| | | | | | domain containing 2A |
| 342527 | SMTNL2 | smoothelin like 2 | 84894 | LINGO1 | leucine rich repeat and Ig domain |
| | | | | | containing 1 |
| 3426 | CFI | complement factor I | 8490 | RGS5 | regulator of G protein signaling 5 |
| 3429 | IFI27 | interferon alpha inducible | 8492 | PRSS12 | protease, serine 12 |
| | | protein 27 | | | |
| 342908 | ZNF404 | zinc finger protein 404 | 84935 | MEDAG | mesenteric estrogen dependent |
| | | | | | adipogenesis |
| 342979 | PALM3 | paralemmin 3 | 84952 | CGNL1 | cingulin like 1 |
| 3434 | IFIT1 | interferon induced protein | 84960 | CCDC183 | coiled-coil domain containing 183 |
| | | with tetratricopeptide repeats | | | |
| | | 1 | | | |
| 343413 | FCRL6 | Fc receptor like 6 | 8497 | PPFIA4 | PTPRF interacting protein alpha 4 |
| 343450 | KCNT2 | potassium sodium-activated | 8499 | PPFIA2 | PTPRF interacting protein alpha 2 |
| | | channel subfamily T member | | | |
| | | 2 | | | |
| 345079 | SOWAHB | sosondowah ankyrin repeat | 85004 | RERG | RAS like estrogen regulated |
| | | domain family member B | | | growth inhibitor |
| 346171 | ZFP57 | ZFP57 zinc finger protein | 85016 | C11orf70 | chromosome 11 open reading |
| | | | | | frame 70 |
| 346389 | MACC1 | MACC1, MET transcriptional | 85027 | SMIM3 | small integral membrane protein 3 |
| | | regulator | | | |
| 346606 | MOGAT3 | monoacylglycerol O- | 8503 | PIK3R3 | phosphoinositide-3-kinase |
| | | acyltransferase 3 | | | regulatory subunit 3 |
| 347 | APOD | apolipoprotein D | 8515 | ITGA10 | integrin subunit alpha 10 |
| 347475 | CCDC160 | coiled-coil domain containing | 8516 | ITGA8 | integrin subunit alpha 8 |
| | | 160 | | | |
| 347733 | TUBB2B | tubulin beta 2B class IIb | 85235 | HIST1H2AH | histone cluster 1 H2A family |
| | | | | | member h |
| 347735 | SERINC2 | serine incorporator 2 | 8530 | CST7 | cystatin F |
| 3479 | IGF1 | insulin like growth factor 1 | 8532 | CPZ | carboxypeptidase Z |
| 348 | APOE | apolipoprotein E | 85329 | LGALS12 | galectin 12 |
| 348093 | RBPMS2 | RNA binding protein with | 85358 | SHANK3 | SH3 and multiple ankyrin repeat |
| | | multiple splicing 2 | | | domains 3 |
| 348158 | ACSM2B | acyl-CoA synthetase medium | 8538 | BARX2 | BARX homeobox 2 |
| | | chain family member 2B | | | |
| 348174 | CLEC18A | C-type lectin domain family | 85388 | SNORD14B | small nucleolar RNA, C/D box 14B |
| | | 18 member A | | | |
| 348249 | CCL15-CCL14 | CCL15-CCL14 readthrough | 85389 | SNORD14C | small nucleolar RNA, C/D box |
| | | (NMD candidate) | | | 14C |
| 3484 | IGFBP1 | insulin like growth factor | 85390 | SNORD14D | small nucleolar RNA, C/D box |
| | | binding protein 1 | | | 14D |
| 348487 | FAM131C | family with sequence | 85409 | NKD2 | naked cuticle homolog 2 |
| | | similarity 131 member C | | | |
| 3485 | IGFBP2 | insulin like growth factor | 8542 | APOL1 | apolipoprotein L1 |
| | | binding protein 2 | | | |
| 3486 | IGFBP3 | insulin like growth factor | 85439 | STON2 | stonin 2 |
| | | binding protein 3 | | | |
| 348738 | C2orf48 | chromosome 2 open reading | 85442 | KNDC1 | kinase non-catalytic C-lobe |
| | | frame 48 | | | domain containing 1 |
| 3488 | IGFBP5 | insulin like growth factor | 85453 | TSPYL5 | TSPY like 5 |
| | | binding protein 5 | | | |
| 3489 | IGFBP6 | insulin like growth factor | 8547 | FCN3 | ficolin 3 |
| | | binding protein 6 | | | |
| 3491 | CYR61 | cysteine rich angiogenic | 85477 | SCIN | scinderin |
| | | inducer 61 | | | |
| 349152 | DPY19L2P2 | DPY19L2 pseudogene 2 | 85479 | DNAJC5B | DnaJ heat shock protein family |
| | | | | | (Hsp40) member C5 beta |
| 350 | APOH | apolipoprotein H | 85495 | RPPH1 | ribonuclease P RNA component |
| | | | | | H1 |
| 3512 | JCHAIN | joining chain of multimeric | 8564 | KMO | kynurenine 3-monooxygenase |
| | | IgA and IgM | | | |
| 353189 | SLCO4C1 | solute carrier organic anion | 8572 | PDLIM4 | PDZ and LIM domain 4 |
| | | transporter family member | | | |
| | | 4C1 | | | |
| 353514 | LILRA5 | leukocyte immunoglobulin | 860 | RUNX2 | runt related transcription factor 2 |
| | | like receptor A5 | | | |
| 3549 | IHH | indian hedgehog | 8611 | PLPP1 | phospholipid phosphatase 1 |
| 3553 | IL1B | interleukin 1 beta | 8612 | PLPP2 | phospholipid phosphatase 2 |
| 3557 | IL1RN | interleukin 1 receptor | 8613 | PLPP3 | phospholipid phosphatase 3 |
| | | antagonist | | | |
| 3559 | IL2RA | interleukin 2 receptor subunit | 8614 | STC2 | stanniocalcin 2 |
| | | alpha | | | |
| 356 | FASLG | Fas ligand | 8622 | PDE8B | phosphodiesterase 8B |
| 3560 | IL2RB | interleukin 2 receptor subunit | 8635 | RNASET2 | ribonuclease T2 |
| | | beta | | | |
| 3561 | IL2RG | interleukin 2 receptor subunit | 8638 | OASL | 2′-5′-oligoadenylate synthetase |
| | | gamma | | | like |
| 3563 | IL3RA | interleukin 3 receptor subunit | 8639 | AOC3 | amine oxidase, copper containing |
| | | alpha | | | 3 |
| 3569 | IL6 | interleukin 6 | 8641 | PCDHGB4 | protocadherin gamma subfamily |
| | | | | | B, 4 |
| 3575 | IL7R | interleukin 7 receptor | 8642 | DCHS1 | dachsous cadherin-related 1 |
| 3576 | CXCL8 | C-X-C motif chemokine | 8645 | KCNK5 | potassium two pore domain |
| | | ligand 8 | | | channel subfamily K member 5 |
| 358 | AQP1 | aquaporin 1 (Colton blood | 8646 | CHRD | chordin |
| | | group) | | | |
| 3580 | CXCR2P1 | C-X-C motif chemokine | 8653 | DDX3Y | DEAD-box helicase 3, Y-linked |
| | | receptor 2 pseudogene 1 | | | |
| 3586 | IL10 | interleukin 10 | 8659 | ALDH4A1 | aldehyde dehydrogenase 4 family |
| | | | | | member A1 |
| 3587 | IL10RA | interleukin 10 receptor | 866 | SERPINA6 | serpin family A member 6 |
| | | subunit alpha | | | |
| 3594 | IL12RB1 | interleukin 12 receptor | 8660 | IRS2 | insulin receptor substrate 2 |
| | | subunit beta 1 | | | |
| 3595 | IL12RB2 | interleukin 12 receptor | 8671 | SLC4A4 | solute carrier family 4 member 4 |
| | | subunit beta 2 | | | |
| 3598 | IL13RA2 | interleukin 13 receptor | 8685 | MARCO | macrophage receptor with |
| | | subunit alpha 2 | | | collagenous structure |
| 360 | AQP3 | aquaporin 3 (Gill blood group) | 8701 | DNAH11 | dynein axonemal heavy chain 11 |
| 3604 | TNFRSF9 | TNF receptor superfamily | 8722 | CTSF | cathepsin F |
| | | member 9 | | | |
| 3606 | IL18 | interleukin 18 | 8736 | MYOM1 | myomesin 1 |
| 361 | AQP4 | aquaporin 4 | 8743 | TNFSF10 | TNF superfamily member 10 |
| 3613 | IMPA2 | inositol monophosphatase 2 | 8787 | RGS9 | regulator of G protein signaling 9 |
| 3620 | IDO1 | indoleamine 2,3-dioxygenase | 8792 | TNFRSF11A | TNF receptor superfamily member |
| | | 1 | | | 11a |
| 3623 | INHA | inhibin alpha subunit | 8794 | TNFRSF10C | TNF receptor superfamily member |
| | | | | | 10c |
| 3624 | INHBA | inhibin beta A subunit | 88 | ACTN2 | actinin alpha 2 |
| 3625 | INHBB | inhibin beta B subunit | 8808 | IL1RL2 | interleukin 1 receptor like 2 |
| 3627 | CXCL10 | C-X-C motif chemokine | 8824 | CES2 | carboxylesterase 2 |
| | | ligand 10 | | | |
| 364 | AQP7 | aquaporin 7 | 8825 | LIN7A | lin-7 homolog A, crumbs cell |
| | | | | | polarity complex component |
| 3643 | INSR | insulin receptor | 8839 | WISP2 | WNT1 inducible signaling pathway |
| | | | | | protein 2 |
| 3659 | TRF | interferon regulatory factor 1 | 8842 | PROM1 | prominin 1 |
| 366 | AQP9 | aquaporin 9 | 8854 | ALDH1A2 | aldehyde dehydrogenase 1 family |
| | | | | | member A2 |
| 3662 | IRF4 | interferon regulatory factor 4 | 8857 | FCGBP | Fc fragment of IgG binding protein |
| 3664 | IRF6 | interferon regulatory factor 6 | 8862 | APLN | apelin |
| 3667 | IRS1 | insulin receptor substrate 1 | 8863 | PER3 | period circadian regulator 3 |
| 367 | AR | androgen receptor | 8870 | IER3 | immediate early response 3 |
| 3671 | ISLR | immunoglobulin superfamily | 8875 | VNN2 | vanin 2 |
| | | containing leucine rich repeat | | | |
| 3675 | ITGA3 | integrin subunit alpha 3 | 8876 | VNN1 | vanin 1 |
| 3679 | ITGA7 | integrin subunit alpha 7 | 890 | CCNA2 | cyclin A2 |
| 368 | ABCC6 | ATP binding cassette | 891 | CCNB1 | cyclin B1 |
| | | subfamily C member 6 | | | |
| 3681 | ITGAD | integrin subunit alpha D | 8912 | CACNA1H | calcium voltage-gated channel |
| | | | | | subunit alpha1 H |
| 3683 | ITGAL | integrin subunit alpha L | 8942 | KYNU | kynureninase |
| 3687 | ITGAX | integrin subunit alpha X | 8968 | HIST1H3F | histone cluster 1 H3 family |
| | | | | | member f |
| 3690 | ITGB3 | integrin subunit beta 3 | 8969 | HIST1H2AG | histone cluster 1 H2A family |
| | | | | | member g |
| 3691 | ITGB4 | integrin subunit beta 4 | 8970 | HIST1H2BJ | histone cluster 1 H2B family |
| | | | | | member j |
| 3694 | ITGB6 | integrin subunit beta 6 | 8972 | MGAM | maltase-glucoamylase |
| 3696 | ITGB8 | integrin subunit beta 8 | 89765 | RSPH1 | radial spoke head 1 homolog |
| 3699 | ITIH3 | inter-alpha-trypsin inhibitor | 89790 | SIGLEC10 | sialic acid binding Ig like lectin 10 |
| | | heavy chain 3 | | | |
| 3700 | ITIH4 | inter-alpha-trypsin inhibitor | 89795 | NAV3 | neuron navigator 3 |
| | | heavy chain family member 4 | | | |
| 3702 | ITK | IL2 inducible T-cell kinase | 89858 | SIGLEC12 | sialic acid binding Ig like lectin 12 |
| | | | | | (gene/pseudogene) |
| 3710 | ITPR3 | inositol 1,4,5-trisphosphate | 89870 | TRIM15 | tripartite motif containing 15 |
| | | receptor type 3 | | | |
| 3714 | JAG2 | jagged 2 | 89876 | MAATS1 | MYCBP associated and testis |
| | | | | | expressed 1 |
| 3718 | JAK3 | Janus kinase 3 | 8989 | TRPA1 | transient receptor potential cation |
| | | | | | channel subfamily A member 1 |
| 3725 | JUN | Jun proto-oncogene, AP-1 | 89932 | PAPLN | papilin, proteoglycan like sulfated |
| | | transcription factor subunit | | | glycoprotein |
| 3726 | JUNB | JunB proto-oncogene, AP-1 | 89944 | GLB1L2 | galactosidase beta 1 like 2 |
| | | transcription factor subunit | | | |
| 3730 | ANOS1 | anosmin 1 | 8996 | NOL3 | nucleolar protein 3 |
| 3732 | CD82 | CD82 molecule | 8999 | CDKL2 | cyclin dependent kinase like 2 |
| 374 | AREG | amphiregulin | 9002 | F2RL3 | F2R like thrombin or trypsin |
| | | | | | receptor 3 |
| 3741 | KCNA5 | potassium voltage-gated | 90139 | TSPAN18 | tetraspanin 18 |
| | | channel subfamily A member | | | |
| | | 5 | | | |
| 374383 | NCR3LG1 | natural killer cell cytotoxicity | 9021 | SOCS3 | suppressor of cytokine signaling 3 |
| | | receptor 3 ligand 1 | | | |
| 374407 | DNAJB13 | DnaJ heat shock protein | 9027 | NAT8 | N-acetyltransferase 8 (putative) |
| | | family (Hsp40) member B13 | | | |
| 374618 | TEX9 | testis expressed 9 | 9032 | TM4SF5 | transmembrane 4 L six family |
| | | | | | member 5 |
| 374666 | WASH3P | WAS protein family homolog | 90332 | EXOC3L2 | exocyst complex component 3 like |
| | | 3 pseudogene | | | 2 |
| 3748 | KCNC3 | potassium voltage-gated | 90381 | TICRR | TOPBP1 interacting checkpoint |
| | | channel subfamily C member | | | and replication regulator |
| | | 3 | | | |
| 374864 | CCDC178 | coiled-coil domain containing | 9047 | SH2D2A | SH2 domain containing 2A |
| | | 178 | | | |
| 375033 | PEAR1 | platelet endothelial | 9051 | PSTPIP1 | proline-serine-threonine |
| | | aggregation receptor 1 | | | phosphatase interacting protein 1 |
| 3752 | KCND3 | potassium voltage-gated | 9052 | GPRC5A | G protein-coupled receptor class |
| | | channel subfamily D member | | | C group 5 member A |
| | | 3 | | | |
| 375298 | CERKL | ceramide kinase like | 9053 | MAP7 | microtubule associated protein 7 |
| 375449 | MAST4 | microtubule associated | 9056 | SLC7A7 | solute carrier family 7 member 7 |
| | | serine/threonine kinase family | | | |
| | | member 4 | | | |
| 375616 | KCP | kielin/chordin-like protein | 90649 | ZNF486 | zinc finger protein 486 |
| 375775 | PNPLA7 | patatin like phospholipase | 9071 | CLDN10 | claudin 10 |
| | | domain containing 7 | | | |
| 3759 | KCNJ2 | potassium voltage-gated | 9075 | CLDN2 | claudin 2 |
| | | channel subfamily J member | | | |
| | | 2 | | | |
| 3760 | KCNJ3 | potassium voltage-gated | 9076 | CLDN1 | claudin 1 |
| | | channel subfamily J member | | | |
| | | 3 | | | |
| 3764 | KCNJ8 | potassium voltage-gated | 9079 | LDB2 | LIM domain binding 2 |
| | | channel subfamily J member | | | |
| | | 8 | | | |
| 3772 | KCNJ15 | potassium voltage-gated | 9086 | EIF1AY | eukaryotic translation initiation |
| | | channel subfamily J member | | | factor 1A, Y-linked |
| | | 15 | | | |
| 3773 | KCNJ16 | potassium voltage-gated | 90865 | IL33 | interleukin 33 |
| | | channel subfamily J member | | | |
| | | 16 | | | |
| 3776 | KCNK2 | potassium two pore domain | 90952 | ESAM | endothelial cell adhesion molecule |
| | | channel subfamily K member | | | |
| | | 2 | | | |
| 3777 | KCNK3 | potassium two pore domain | 9099 | USP2 | ubiquitin specific peptidase 2 |
| | | channel subfamily K member | | | |
| | | 3 | | | |
| 3778 | KCNMA1 | potassium calcium-activated | 90993 | CREB3L1 | CAMP responsive element binding |
| | | channel subfamily M alpha 1 | | | protein 3 like 1 |
| 3782 | KCNN3 | potassium calcium-activated | 9103 | FCGR2C | Fc fragment of IgG receptor IIc |
| | | channel subfamily N member | | | (gene/pseudogene) |
| | | 3 | | | |
| 378706 | RN7SL2 | RNA, 7SL, cytoplasmic 2 | 9104 | RGN | regucalcin |
| 379 | ARL4D | ADP ribosylation factor like | 91156 | IGFN1 | immunoglobulin-like and |
| | | GTPase 4D | | | fibronectin type III domain |
| | | | | | containing 1 |
| 3791 | KDR | kinase insert domain receptor | 9122 | SLC16A4 | solute carrier family 16 member 4 |
| 3795 | KHK | ketohexokinase | 91316 | GUSBP11 | glucuronidase, beta pseudogene |
| | | | | | 11 |
| 3796 | KIF2A | kinesin family member 2A | 9133 | CCNB2 | cyclin B2 |
| 38 | ACAT1 | acetyl-CoA acetyltransferase | 914 | CD2 | CD2 molecule |
| | | 1 | | | |
| 3805 | KIR2DL4 | killer cell immunoglobulin like | 915 | CD3D | CD3d molecule |
| | | receptor, two lg domains and | | | |
| | | long cytoplasmic tail 4 | | | |
| 3815 | KIT | KIT proto-oncogene receptor | 91522 | COL23A1 | collagen type XXIII alpha 1 chain |
| | | tyrosine kinase | | | |
| 3818 | KLKB1 | kallikrein B1 | 9154 | SLC28A1 | solute carrier family 28 member 1 |
| 3820 | KLRB1 | killer cell lectin like receptor | 9156 | EXO1 | exonuclease 1 |
| | | B1 | | | |
| 3821 | KLRC1 | killer cell lectin like receptor | 916 | CD3E | CD3e molecule |
| | | C1 | | | |
| 3822 | KLRC2 | killer cell lectin like receptor | 91614 | DEPDC7 | DEP domain containing 7 |
| | | C2 | | | |
| 3823 | KLRC3 | killer cell lectin like receptor | 9162 | DGKI | diacylglycerol kinase iota |
| | | C3 | | | |
| 3833 | KIFC1 | kinesin family member C1 | 91624 | NEXN | nexilin F-actin binding protein |
| 384 | ARG2 | arginase 2 | 91683 | SYT12 | synaptotagmin 12 |
| 3846 | KRTAP5-9 | keratin associated protein 5-9 | 917 | CD3G | CD3g molecule |
| 3855 | KRT7 | keratin 7 | 91703 | ACY3 | aminoacylase 3 |
| 3856 | KRT8 | keratin 8 | 9173 | IL1RL1 | interleukin 1 receptor like 1 |
| 3872 | KRT17 | keratin 17 | 91768 | CABLES1 | Cdk5 and Abl enzyme substrate 1 |
| 387357 | THEMIS | thymocyte selection | 9182 | RASSF9 | Ras association domain family |
| | | associated | | | member 9 |
| 387496 | RASL11A | RAS like family 11 member A | 91828 | EXOC3L4 | exocyst complex component 3 like |
| | | | | | 4 |
| 3875 | KRT18 | keratin 18 | 9185 | REPS2 | RALBP1 associated Eps domain |
| | | | | | containing 2 |
| 387597 | ILDR2 | immunoglobulin like domain | 91851 | CHRDL1 | chordin like 1 |
| | | containing receptor 2 | | | |
| 387638 | C10orf113 | chromosome 10 open | 919 | CD247 | CD247 molecule |
| | | reading frame 113 | | | |
| 387646 | LRRC37A6P | leucine rich repeat containing | 91937 | TIMD4 | T-cell immunoglobulin and mucin |
| | | 37 member A6, pseudogene | | | domain containing 4 |
| 387695 | C10orf99 | chromosome 10 open | 91975 | ZNF300 | zinc finger protein 300 |
| | | reading frame 99 | | | |
| 387700 | SLC16A12 | solute carrier family 16 | 9201 | DCLK1 | doublecortin like kinase 1 |
| | | member 12 | | | |
| 387748 | OR56B1 | olfactory receptor family 56 | 921 | CD5 | CD5 molecule |
| | | subfamily B member 1 | | | |
| 387751 | GVINP1 | GTPase, very large interferon | 9212 | AURKB | aurora kinase B |
| | | inducible pseudogene 1 | | | |
| 387763 | C11orf96 | chromosome 11 open | 92126 | DSEL | dermatan sulfate epimerase-like |
| | | reading frame 96 | | | |
| 387804 | VSTM5 | V-set and transmembrane | 9214 | FCMR | Fc fragment of IgM receptor |
| | | domain containing 5 | | | |
| 387882 | C12orf75 | chromosome 12 open | 92162 | TMEM88 | transmembrane protein 88 |
| | | reading frame 75 | | | |
| 388 | RHOB | ras homolog family member | 92211 | CDHR1 | cadherin related family member 1 |
| | | B | | | |
| 3880 | KRT19 | keratin 19 | 92291 | CAPN13 | calpain 13 |
| 388011 | LINC01550 | long intergenic non-protein | 92292 | GLYATL1 | glycine-N-acyltransferase like 1 |
| | | coding RNA 1550 | | | |
| 388335 | TMEM220 | transmembrane protein 220 | 923 | CD6 | CD6 molecule |
| 388372 | CCL4L1 | C-C motif chemokine ligand 4 | 9232 | PTTG1 | pituitary tumor-transforming 1 |
| | | like 1 | | | |
| 388512 | CLEC17A | C-type lectin domain | 92359 | CRB3 | crumbs 3, cell polarity complex |
| | | containing 17A | | | component |
| 388559 | ZNF888 | zinc finger protein 888 | 924 | CD7 | CD7 molecule |
| 388630 | TRABD2B | TraB domain containing 2B | 9242 | MSC | musculin |
| 388886 | LRRC75B | leucine rich repeat containing | 9244 | CRLF1 | cytokine receptor like factor 1 |
| | | 75B | | | |
| 389336 | C5orf46 | chromosome 5 open reading | 9245 | GCNT3 | glucosaminyl (N-acetyl) |
| | | frame 46 | | | transferase 3, mucin type |
| 389337 | ARHGEF37 | Rho guanine nucleotide | 925 | CD8A | CD8a molecule |
| | | exchange factor 37 | | | |
| 389643 | NUGGC | nuclear GTPase, germinal | 92558 | BICDL1 | BICD family like cargo adaptor 1 |
| | | center associated | | | |
| 389668 | XKR9 | XK related 9 | 92737 | DNER | delta/notch like EGF repeat |
| | | | | | containing |
| 3897 | L1CAM | L1 cell adhesion molecule | 92745 | SLC38A5 | solute carrier family 38 member 5 |
| 3898 | LAD1 | ladinin 1 | 92815 | HIST3H2A | histone cluster 3 H2A |
| 389840 | MAP3K15 | mitogen-activated protein | 9289 | ADGRG1 | adhesion G protein-coupled |
| | | kinase kinase kinase 15 | | | receptor G1 |
| 389860 | PAGE2B | PAGE family member 2B | 929 | SNORD29 | small nucleolar RNA, C/D box 29 |
| 3899 | AFF3 | AF4/FMR2 family member 3 | 930 | CD19 | CD19 molecule |
| 390072 | OR52N4 | olfactory receptor family 52 | 9300 | SNORD28 | small nucleolar RNA, C/D box 28 |
| | | subfamily N member 4 | | | |
| | | (gene/pseudogene) | | | |
| 3902 | LAG3 | lymphocyte activating 3 | 9301 | SNORD27 | small nucleolar RNA, C/D box 27 |
| 390649 | OR4F15 | olfactory receptor family 4 | 9302 | SNORD26 | small nucleolar RNA, C/D box 26 |
| | | subfamily F member 15 | | | |
| 390651 | OR4F13P | olfactory receptor family 4 | 9308 | CD83 | CD83 molecule |
| | | subfamily F member 13 | | | |
| | | pseudogene | | | |
| 3908 | LAMA2 | laminin subunit alpha 2 | 93099 | DMKN | dermokine |
| 3909 | LAMA3 | laminin subunit alpha 3 | 931 | MS4A1 | membrane spanning 4-domains |
| | | | | | A1 |
| 391190 | OR2L8 | olfactory receptor family 2 | 9314 | KLF4 | Kruppel like factor 4 |
| | | subfamily L member 8 | | | |
| | | (gene/pseudogene) | | | |
| 391267 | ANKRD20A11P | ankyrin repeat domain 20 | 93145 | OLFM2 | olfactomedin 2 |
| | | family member A11, | | | |
| | | pseudogene | | | |
| 3914 | LAMB3 | laminin subunit beta 3 | 9317 | PTER | phosphotriesterase related |
| 3918 | LAMC2 | laminin subunit gamma 2 | 933 | CD22 | CD22 molecule |
| 392255 | GDF6 | growth differentiation factor 6 | 9332 | CD163 | CD163 molecule |
| 392360 | CTSL3P | cathepsin L family member 3, | 93432 | MGAM2 | maltase-glucoamylase 2 (putative) |
| | | pseudogene | | | |
| 392364 | LOC392364 | nuclear pore associated | 9351 | SLC9A3R2 | SLC9A3 regulator 2 |
| | | protein 1 pseudogene | | | |
| 392636 | AGMO | alkylglycerol monooxygenase | 93517 | SDR42E1 | short chain |
| | | | | | dehydrogenase/reductase family |
| | | | | | 42E, member 1 |
| 3929 | LBP | lipopolysaccharide binding | 9353 | SLIT2 | slit guidance ligand 2 |
| | | protein | | | |
| 3932 | LCK | LCK proto-oncogene, Src | 9358 | ITGBL1 | integrin subunit beta like 1 |
| | | family tyrosine kinase | | | |
| 3934 | LCN2 | lipocalin 2 | 9365 | KL | klotho |
| 3936 | LCP1 | lymphocyte cytosolic protein | 9379 | NRXN2 | neurexin 2 |
| | | 1 | | | |
| 3949 | LDLR | low density lipoprotein | 9388 | LIPG | lipase G, endothelial type |
| | | receptor | | | |
| 3957 | LGALS2 | galectin 2 | 939 | CD27 | CD27 molecule |
| 3958 | LGALS3 | galectin 3 | 93953 | GCNA | germ cell nuclear acidic peptidase |
| 3960 | LGALS4 | galectin 4 | 93986 | FOXP2 | forkhead box P2 |
| 3976 | LIF | LIF, interleukin 6 family | 94 | ACVRL1 | activin A receptor like type 1 |
| | | cytokine | | | |
| 3977 | LIFR | LIF receptor alpha | 94031 | HTRA3 | HtrA serine peptidase 3 |
| 3984 | LIMK1 | LIM domain kinase 1 | 9415 | FADS2 | fatty acid desaturase 2 |
| 3990 | LIPC | lipase C, hepatic type | 94161 | SNORD46 | small nucleolar RNA, C/D box 46 |
| 400566 | C17orf97 | chromosome 17 open | 94162 | SNORD38A | small nucleolar RNA, C/D box 38A |
| | | reading frame 97 | | | |
| 400759 | GBP1P1 | guanylate binding protein 1 | 942 | CD86 | CD86 molecule |
| | | pseudogene 1 | | | |
| 400916 | CHCHD10 | coiled-coil-helix-coiled-coil- | 94234 | FOXQ1 | forkhead box Q1 |
| | | helix domain containing 10 | | | |
| 401124 | DTHD1 | death domain containing 1 | 94240 | EPSTI1 | epithelial stromal interaction 1 |
| 401190 | RGS7BP | regulator of G protein | 94274 | PPP1R14A | protein phosphatase 1 regulatory |
| | | signaling 7 binding protein | | | inhibitor subunit 14A |
| 401409 | RAB19 | RAB19, member RAS | 9429 | ABCG2 | ATP binding cassette subfamily G |
| | | oncogene family | | | member 2 (Junior blood group) |
| 401427 | OR2A7 | olfactory receptor family 2 | 9437 | NCR1 | natural cytotoxicity triggering |
| | | subfamily A member 7 | | | receptor 1 |
| 4015 | LOX | lysyl oxidase | 9447 | AIM2 | absent in melanoma 2 |
| 4016 | LOXL1 | lysyl oxidase like 1 | 9450 | LY86 | lymphocyte antigen 86 |
| 4017 | LOXL2 | lysyl oxidase like 2 | 9452 | ITM2A | integral membrane protein 2A |
| 4023 | LPL | lipoprotein lipase | 9457 | FHL5 | four and a half LIM domains 5 |
| 4033 | LRMP | lymphoid restricted | 947 | CD34 | CD34 molecule |
| | | membrane protein | | | |
| 4036 | LRP2 | LDL receptor related protein | 9472 | AKAP6 | A-kinase anchoring protein 6 |
| | | 2 | | | |
| 4038 | LRP4 | LDL receptor related protein | 9476 | NAPSA | napsin A aspartic peptidase |
| | | 4 | | | |
| 404550 | C16orf74 | chromosome 16 open | 9478 | CABP1 | calcium binding protein 1 |
| | | reading frame 74 | | | |
| 4046 | LSP1 | lymphocyte-specific protein 1 | 9479 | MAPK8IP1 | mitogen-activated protein kinase 8 |
| | | | | | interacting protein 1 |
| 4050 | LTB | lymphotoxin beta | 948 | CD36 | CD36 molecule |
| 4051 | CYP4F3 | cytochrome P450 family 4 | 9481 | SLC25A27 | solute carrier family 25 member |
| | | subfamily F member 3 | | | 27 |
| 4052 | LTBP1 | latent transforming growth | 949 | SCARB1 | scavenger receptor class B |
| | | factor beta binding protein 1 | | | member 1 |
| 4053 | LTBP2 | latent transforming growth | 9493 | KIF23 | kinesin family member 23 |
| | | factor beta binding protein 2 | | | |
| 4056 | LTC4S | leukotriene C4 synthase | 9499 | MYOT | myotilin |
| 4057 | LTF | lactotransferrin | 9507 | ADAMTS4 | ADAM metallopeptidase with |
| | | | | | thrombospondin type 1 motif 4 |
| 4059 | BCAM | basal cell adhesion molecule | 9509 | ADAMTS2 | ADAM metallopeptidase with |
| | | (Lutheran blood group) | | | thrombospondin type 1 motif 2 |
| 4060 | LUM | lumican | 9510 | ADAMTS1 | ADAM metallopeptidase with |
| | | | | | thrombospondin type 1 motif 1 |
| 4061 | LY6E | lymphocyte antigen 6 family | 9514 | GAL3ST1 | galactose-3-O-sulfotransferase 1 |
| | | member E | | | |
| 4063 | LY9 | lymphocyte antigen 9 | 9518 | GDF15 | growth differentiation factor 15 |
| 4064 | CD180 | CD180 molecule | 952 | CD38 | CD38 molecule |
| 4068 | SH2D1A | SH2 domain containing 1A | 9536 | PTGES | prostaglandin E synthase |
| 4069 | LYZ | lysozyme | 954 | ENTPD2 | ectonucleoside triphosphate |
| | | | | | diphosphohydrolase 2 |
| 4070 | TACSTD2 | tumor associated calcium | 9547 | CXCL14 | C-X-C motif chemokine ligand 14 |
| | | signal transducer 2 | | | |
| 4071 | TM4SF1 | transmembrane 4 L six family | 9560 | CCL4L2 | C-C motif chemokine ligand 4 like |
| | | member 1 | | | 2 |
| 4072 | EPCAM | epithelial cell adhesion | 957 | ENTPD5 | ectonucleoside triphosphate |
| | | molecule | | | diphosphohydrolase 5 |
| 408186 | OVOS | ovostatin | 9572 | NR1D1 | nuclear receptor subfamily 1 |
| | | | | | group D member 1 |
| 4093 | SMAD9 | SMAD family member 9 | 9580 | SOX13 | SRY-box 13 |
| 4118 | MAL | mal, T-cell differentiation | 9582 | APOBEC3B | apolipoprotein B mRNA editing |
| | | protein | | | enzyme catalytic subunit 3B |
| 4128 | MAOA | monoamine oxidase A | 9586 | CREB5 | CAMP responsive element binding |
| | | | | | protein 5 |
| 4129 | MAOB | monoamine oxidase B | 959 | CD40LG | CD40 ligand |
| 4133 | MAP2 | microtubule associated | 9590 | AKAP12 | A-kinase anchoring protein 12 |
| | | protein 2 | | | |
| 4137 | MAPT | microtubule associated | 9595 | CYTIP | cytohesin 1 interacting protein |
| | | protein tau | | | |
| 414157 | C10orf62 | chromosome 10 open | 960 | CD44 | CD44 molecule (Indian blood |
| | | reading frame 62 | | | group) |
| 414194 | CCNYL2 | cyclin Y-like 2 (pseudogene) | 9615 | GDA | guanine deaminase |
| 414224 | AGAP12P | ArfGAP with GTPase domain, | 962 | CD48 | CD48 molecule |
| | | ankyrin repeat and PH | | | |
| | | domain 12, pseudogene | | | |
| 414235 | PRR26 | proline rich 26 | 969 | CD69 | CD69 molecule |
| 4143 | MAT1A | methionine | 970 | CD70 | CD70 molecule |
| | | adenosyltransferase 1A | | | |
| 4147 | MATN2 | matrilin 2 | 9708 | PCDHGA8 | protocadherin gamma subfamily |
| | | | | | A, 8 |
| 4148 | MATN3 | matrilin 3 | 971 | CD72 | CD72 molecule |
| 415 | ARSE | arylsulfatase E | 9719 | ADAMTSL2 | ADAMTS like 2 |
| | | (chondrodysplasia punctata | | | |
| | | 1) | | | |
| 4162 | MCAM | melanoma cell adhesion | 9720 | CCDC144A | coiled-coil domain containing |
| | | molecule | | | 144A |
| 4192 | MDK | midkine | 9727 | RAB11FIP3 | RAB11 family interacting protein 3 |
| 420 | ART4 | ADP-ribosyltransferase 4 | 973 | CD79A | CD79a molecule |
| | | (Dombrock blood group) | | | |
| 4210 | MEFV | MEFV, pyrin innate immunity | 974 | CD79B | CD79b molecule |
| | | regulator | | | |
| 4223 | MEOX2 | mesenchyme homeobox 2 | 9744 | ACAP1 | ArfGAP with coiled-coil, ankyrin |
| | | | | | repeat and PH domains 1 |
| 4232 | MEST | mesoderm specific transcript | 9750 | RIPOR2 | RHO family interacting cell |
| | | | | | polarization regulator 2 |
| 4237 | MFAP2 | microfibril associated protein | 9760 | TOX | thymocyte selection associated |
| | | 2 | | | high mobility group box |
| 4239 | MFAP4 | microfibril associated protein | 978 | CDA | cytidine deaminase |
| | | 4 | | | |
| 4241 | MELTF | melanotransferrin | 9787 | DLGAP5 | DLG associated protein 5 |
| 4248 | MGAT3 | mannosyl (beta-1,4-)- | 9805 | SCRN1 | secernin 1 |
| | | glycoprotein beta-1,4-N- | | | |
| | | acetylglucosaminyltransferase | | | |
| 4254 | KITLG | KIT ligand | 9828 | ARHGEF17 | Rho guanine nucleotide exchange |
| | | | | | factor 17 |
| 4256 | MGP | matrix Gla protein | 983 | CDK1 | cyclin dependent kinase 1 |
| 4257 | MGST1 | microsomal glutathione S- | 9833 | MELK | maternal embryonic leucine zipper |
| | | transferase 1 | | | kinase |
| 4261 | CIITA | class II major | 9848 | MFAP3L | microfibril associated protein 3 like |
| | | histocompatibility complex | | | |
| | | transactivator | | | |
| 4281 | MID1 | midline 1 | 9886 | RHOBTB1 | Rho related BTB domain |
| | | | | | containing 1 |
| 4283 | CXCL9 | C-X-C motif chemokine | 990 | CDC6 | cell division cycle 6 |
| | | ligand 9 | | | |
| 4288 | MKI67 | marker of proliferation Ki-67 | 9902 | MRC2 | mannose receptor C type 2 |
| 4306 | NR3C2 | nuclear receptor subfamily 3 | 991 | CDC20 | cell division cycle 20 |
| | | group C member 2 | | | |
| 4311 | MME | membrane | 9915 | ARNT2 | aryl hydrocarbon receptor nuclear |
| | | metalloendopeptidase | | | translocator 2 |
| 4312 | MMP1 | matrix metallopeptidase 1 | 9928 | KIF14 | kinesin family member 14 |
| 4313 | MMP2 | matrix metallopeptidase 2 | 9934 | P2RY14 | purinergic receptor P2Y14 |
| 4316 | MMP7 | matrix metallopeptidase 7 | 9945 | GFPT2 | glutamine-fructose-6-phosphate |
| | | | | | transaminase 2 |
| 4318 | MMP9 | matrix metallopeptidase 9 | 9956 | HS3ST2 | heparan sulfate-glucosamine 3- |
| | | | | | sulfotransferase 2 |
| 4320 | MMP11 | matrix metallopeptidase 11 | 9963 | SLC23A1 | solute carrier family 23 member 1 |
| 4321 | MMP12 | matrix metallopeptidase 12 | 9971 | NR1H4 | nuclear receptor subfamily 1 |
| | | | | | group H member 4 |
| 4325 | MMP16 | matrix metallopeptidase 16 | 9976 | CLEC2B | C-type lectin domain family 2 |
| | | | | | member B |
| 4327 | MMP19 | matrix metallopeptidase 19 | 999 | CDH1 | cadherin 1 |

Any of the methods described herein may include classification of a patient's sample into a cluster, e.g., any cluster identified herein. For example, machine learning algorithms can be used to develop a classifier from gene expression data. Any suitable machine learning algorithm can be used, including supervised learning (e.g., decision tree, random forest, gradient boost machine (GBM), CATBOOST, XGBOOST, support vector machine (SVM), PCA, K-nearest neighbor, and naïve Bayes) and unsupervised learning approaches. In particular instances, the machine learning algorithm is a random forest algorithm, as described, e.g., in Examples 1 and 2. For example, a classifier can be developed using the random forest machine learning algorithm (e.g., using the R package randomForest). The random forest classifier can be learned on a training gene set and then used to predict the cluster (e.g., NMF classes) in a second gene set. In other instances, K-means clustering, K-mediods clustering, or PAM can be used for classification.

Any of the methods disclosed herein may further include determining the expression level (e.g., the mRNA expression level) of one or more genes or gene signatures.

In some examples, the method further comprises determining the mRNA expression level of one or more of the following gene signatures in the tumor sample from the patient: (a) a T-effector signature comprising one or more (e.g., one, two, three, or four), or all, of CD8A, IFNG, EOMES, PRF1, and PD-L1; (b) an angiogenesis signature comprising one or more (e.g., one, two, three, four, or five), or all, of VEGFA, KDR, ESM1, CD34, PECAM1, and ANGPTL4; (c) a fatty acid oxidation (FAO)/AMPK signature comprising one or more (e.g., one, two, three, four, or five), or all, of CPT2, PPARA, CPT1A, PRKAA2, PDK2, and PRKAB1; (d) a cell cycle signature comprising one or more (e.g., one, two, three, four, five, six, seven, eight, or nine), or all, of CDK2, CDK4, CDK6, BUB1, BUB1B, CCNE1, POLQ, AURKA, MKI67, and CCNB2; (e) a fatty acid synthesis (FAS)/pentose phosphate signature comprising one or more (e.g., one, two, three, four, five, or six), or all, of FASN, PARP1, ACACA, G6PD, TKT, TALDO1, and PGD; (f) a stroma signature comprising one or more (e.g., one, two, three, four, five, six, or seven), or all, of FAP, FN1, COL5A1, COL5A2, POSTN, COL1A1, COL1 A2, and MMP2; (g) a myeloid inflammation signature comprising one or more (e.g., one, two, three, four, or five), or all, of CXCL1, CXCL2, CXCL3, CXCL8, IL6, and PTGS2; (h) a complement cascade signature comprising one or more (e.g., one, two, three, four, or five), or all, of F2, C1S, C9, C1R, CFB, and C3; (i) an Ω-oxidation signature comprising one or more (e.g., one, two, three, four, five, six, or seven), or all, of CYP4F3, CYP8B1, NNMT, MGST1, MAOA, CYP4F11, CYP4F2, CYP4F12; and/or (j) a snoRNA signature comprising one or more (e.g., one, two, three, four, or five), or all, of SNORD38A, SNORD104, SNORD32A, SNORD68, SNORD66, and SNORD100.

In some examples, the patient's tumor sample is assigned into the angiogenic/stromal cluster, and the patient's tumor sample has increased expression levels, relative to reference expression levels, of the angiogenesis signature and the stroma signature, optionally wherein the patient's tumor sample has decreased expression levels, relative to reference expression levels, of the T-effector signature, the cell cycle signature, and/or the FAS/pentose phosphate signature.

In some examples, the patient's tumor sample is assigned into the angiogenic cluster, and the patient's tumor sample has increased expression levels, relative to a reference expression levels, of the angiogenesis signature and the FAO/AMPK signature, optionally wherein the patient's tumor has decreased expression levels, relative to reference expression levels, of the cell cycle signature, the FAS/pentose phosphate signature, the stroma signature, the myeloid inflammation signature, and/or the complement cascade signature.

In some examples, the patient's tumor sample is assigned into the complement/Ω-oxidation cluster, and the patient's tumor sample has increased expression levels, relative to reference expression levels, of the complement cascade signature and the Ω-oxidation signature, optionally wherein the patient's tumor sample has an increased expression level, relative to a reference expression level, of the myeloid inflammation signature, and/or decreased expression levels, relative to reference expression levels, of the angiogenesis signature and/or the T-effector signature.

In some examples, the patient's tumor sample is assigned into the T-effector/proliferative cluster, and the patient's tumor sample has increased expression levels, relative to reference expression levels, of the cell cycle signature and the T-effector signature, optionally wherein the patient's tumor sample has increased expression levels, relative to reference expression levels, of the FAS/pentose phosphate signature, the myeloid inflammation signature, and/or the complement cascade signature, and/or decreased expression levels, relative to reference expression levels, of the angiogenesis signature, the FAO/AMP signature, and/or the snoRNA signature.

In some examples, the patient's tumor sample is assigned into the proliferative cluster, and the patient's tumor sample has increased expression levels, relative to reference expression levels, of the cell cycle signature and the FAS/pentose phosphate signature, optionally wherein the patient's tumor sample has increased expression levels, relative to reference expression levels, of the myeloid inflammation signature and/or the FAO/AMPK signature, and/or decreased expression levels, relative to reference expression levels, of the angiogenesis signature, the T-effector signature, the stroma signature, the complement cascade signature, the Ω-oxidation signature, and/or the snoRNA signature.

In some examples, the patient's tumor sample is assigned into the stromal/proliferative cluster, and the patient's tumor sample has increased expression levels, relative to reference expression levels, of the cell cycle signature and the stromal signature, optionally wherein the patient's tumor sample has increased expression levels, relative to reference expression levels, of the FAS/pentose phosphate signature and/or the myeloid inflammation signature, and/or decreased expression levels, relative to reference expression levels, of the angiogenesis signature, the FAO/AMPK signature, the complement cascade signature, the Ω-oxidation signature, and/or the snoRNA signature.

In some examples, the patient's tumor sample is assigned into the snoRNA cluster, and the patient's tumor sample has an increased expression level, relative to a reference expression level, of the snoRNA signature, optionally wherein the patient's tumor sample has decreased expression levels, relative to reference expression levels, of the FOA/AMPK signature, the cell cycle signature, and the FAS/pentose phosphate signature.

Any suitable reference expression level for a signature may be used. In some examples, the reference expression level is determined from a population of patients having a previously untreated kidney cancer (e.g., an inoperable, locally advanced, or metastatic RCC). In some examples, the reference expression level of a signature is the median Z-score of the signature in a population of patients having a previously untreated inoperable, locally advanced, or metastatic RCC.

In some examples, assignment of the patient's tumor sample into one of the following clusters: (4) T-effector/proliferative; (5) proliferative; or (7) snoRNA indicates that the patient is likely to have an increased clinical benefit from treatment with an anti-cancer therapy comprising a PD-1 axis binding antagonist (e.g., atezolizumab or avelumab) and a VEGF antagonist (e.g., bevacizumab or axitinib) compared to treatment with a tyrosine kinase inhibitor (e.g., sunitinib). In some examples, assignment of the patient's tumor sample into one of the following clusters: (4) T-effector/proliferative; (5) proliferative; or (7) snoRNA indicates that the patient is likely to have an increased clinical benefit from treatment with an anti-cancer therapy comprising atezolizumab and bevacizumab compared to treatment with sunitinib. In some examples, assignment of the patient's tumor sample into one of the following clusters: (4) T-effector/proliferative; (5) proliferative; or (7) snoRNA indicates that the patient is likely to have an increased clinical benefit from treatment with an anti-cancer therapy comprising avelumab and axitinib compared to treatment with sunitinib. In some examples, the patient's tumor sample is assigned into cluster (4). In other examples, the patient's tumor is assigned into cluster (5). In yet other examples, the patient's tumor sample is assigned into cluster (7). In some examples, increased clinical benefit comprises a relative increase in one or more of the following: objective response rate (ORR), overall survival (OS), progression-free survival (PFS), compete response (CR), partial response (PR), or a combination thereof. In some examples, increased clinical benefit comprises a relative increase in ORR or PFS.

In some examples, the patient's tumor sample is assigned into one of the following clusters: (4) T-effector/proliferative; (5) proliferative; or (7) snoRNA, and the method further comprises selecting an anti-cancer therapy comprising a PD-1 axis binding antagonist (e.g., atezolizumab or avelumab) and a VEGF antagonist (e.g., bevacizumab or axitinib) for the patient. In some examples, the method further comprises selecting an anti-cancer therapy comprising atezolizumab and bevacizumab. In other examples, the method further comprises selecting an anti-cancer therapy comprising avelumab and axitinib.

In some examples, the patient's tumor sample is assigned into one of the following clusters: (4) T-effector/proliferative; (5) proliferative; or (7) snoRNA, and the method further comprises treating the patient by administering an anti-cancer therapy comprising a PD-1 axis binding antagonist (e.g., atezolizumab or avelumab) and a VEGF antagonist (e.g., bevacizumab or axitinib) to the patient. In some examples, the method further comprises administering an anti-cancer therapy comprising atezolizumab and bevacizumab to the patient. In other examples, the method further comprises administering an anti-cancer therapy comprising avelumab and axitinib to the patient.

In some examples, the patient's tumor is assigned into one of the following clusters: (1) angiogenic/stromal; or (2) angiogenic, and the method further comprises selecting an anti-cancer therapy comprising a PD-1 axis binding antagonist (e.g., atezolizumab or avelumab) and a next-generation anti-angiogenic agent (e.g., XL092 (a next generation tyrosine kinase inhibitor from Exilixis, which targets VEGF receptors; MET, TYRO3, AXL and MERTK (TAM) kinases; and other kinases implicated in cancer's growth and spread) or a HIF2A inhibitor (e.g., belzutifan (also known as MK-6482) or PT2385)) for the patient.

In some examples, the patient's tumor is assigned into one of the following clusters: (1) angiogenic/stromal; or (2) angiogenic, and the method further comprises treating the patient by administering an anti-cancer therapy comprising a PD-1 axis binding antagonist (e.g., atezolizumab or avelumab) and a next-generation anti-angiogenic agent (e.g., XL092 or a HIF2A inhibitor (e.g., belzutifan (also known as MK-6482) or PT2385)).

In some examples, the patient's tumor is assigned into one of the following clusters: (2) angiogenic; or (3) complement/Ω-oxidation, and the method further comprises selecting an anti-cancer therapy comprising an AMP-activated protein kinase (AMPK) inhibitor (e.g., SBI-0206965, 5′-hydroxy-staurosporine, or compound C (also known as dorsomorphin)) for the patient. Exemplary AMPK inhibitors are described, e.g., in Das et al. Sci. Rep. 8:3770, 2018; Vara-Ciruelos et al. Open Biol. 9(7):190099, 2019; Scott et al. Chem. Biol. 22:705-711, 2015; and Dite et al. J. Biol. Chem. 293:8874-8885, 2018..

In some examples, the patient's tumor is assigned into one of the following clusters: (2) angiogenic; or (3) complement/Ω-oxidation, and the method further comprises treating the patient by administering an anti-cancer therapy comprising an AMPK inhibitor (e.g., SBI-0206965, 5′-hydroxy-staurosporine, or compound C (also known as dorsomorphin)) to the patient.

In some examples, the patient's tumor is assigned into the following cluster: (4) T-effector/proliferative, and the method further comprises selecting an anti-cancer therapy comprising an immunotherapy (e.g., an anti-TIGIT antibody (e.g., tiragolumab), PD1-IL2v (a fusion of an anti-PD-1 antibody and modified IL-2), PD1-LAG3, IL-15, anti-CCR8 (e.g., an anti-CCR8 antibody, e.g., FPA157), FAP-4-1 BBL (fibroblast activation protein-targeted 4-1 BBL agonist), or a combination thereof for the patient.

In some examples, the patient's tumor is assigned into the following cluster: (4) T-effector/proliferative, and the method further comprises treating the patient by administering an anti-cancer therapy comprising an immunotherapy (e.g., an anti-TIGIT antibody (e.g., tiragolumab), PD1-IL2v, PD1-LAG3, IL-15, anti-CCR8 (e.g., an anti-CCR8 antibody, e.g., FPA157 or HBM1022), FAP-4-1BBL, or a combination thereof to the patient.

In some examples, the immunotherapy agent is an immune checkpoint inhibitor. In some examples, the immunotherapy agent is a CD28, OX40, GITR, CD137, CD27, ICOS, HVEM, NKG2D, MICA, or 2B4 agonist or a CTLA-4, PD-1 axis, TIM-3, BTLA, VISTA, LAG-3, B7H4, CD96, TIGIT, or CD226 antagonist. Other particular immunotherapy agents that may be used include anti-CTLA-4 antibodies or antigen-binding fragments thereof, anti-CD27 antibodies or antigen-binding fragments thereof, anti-CD30 antibodies or antigen-binding fragments thereof, anti-CD40 antibodies or antigen-binding fragments thereof, anti-4-1 BB antibodies or antigen-binding fragments thereof, anti-GITR antibodies or antigen-binding fragments thereof, anti-OX40 antibodies or antigen-binding fragments thereof, anti-TRAILR1 antibodies or antigen-binding fragments thereof, anti-TRAILR2 antibodies or antigen-binding fragments thereof, anti-TWEAK antibodies or antigen-binding fragments thereof, anti-TWEAKR antibodies or antigen-binding fragments thereof, anti-BRAF antibodies or antigen-binding fragments thereof, anti-MEK antibodies or antigen-binding fragments thereof, anti-CD33 antibodies or antigen-binding fragments thereof, anti-CD20 antibodies or antigen-binding fragments thereof, anti-CD52 antibodies or antigen-binding fragments thereof, anti-A33 antibodies or antigen-binding fragments thereof, anti-GD3 antibodies or antigen-binding fragments thereof, anti-PSMA antibodies or antigen-binding fragments thereof, anti-Ceacan 1 antibodies or antigen-binding fragments thereof, anti-Galedin 9 antibodies or antigen-binding fragments thereof, anti-HVEM antibodies or antigen-binding fragments thereof, anti-VISTA antibodies or antigen-binding fragments thereof, anti-B7 H4 antibodies or antigen-binding fragments thereof, anti-HHLA2 antibodies or antigen-binding fragments thereof, anti-CD155 antibodies or antigen-binding fragments thereof, anti-CD80 antibodies or antigen-binding fragments thereof, anti-BTLA antibodies or antigen-binding fragments thereof, anti-CD160 antibodies or antigen-binding fragments thereof, anti-CD28 antibodies or antigen-binding fragments thereof, anti-CD226 antibodies or antigen-binding fragments thereof, anti-CEACAM1 antibodies or antigen-binding fragments thereof, anti-TIM3 antibodies or antigen-binding fragments thereof, anti-CD96 antibodies or antigen-binding fragments thereof, anti-CD70 antibodies or antigen-binding fragments thereof, anti-CD27 antibodies or antigen-binding fragments thereof, anti-LIGHT antibodies or antigen-binding fragments thereof, anti-CD137 antibodies or antigen-binding fragments thereof, anti-DR4 antibodies or antigen-binding fragments thereof, anti-CR5 antibodies or antigen-binding fragments thereof, anti-FAS antibodies or antigen-binding fragments thereof, anti-CD95 antibodies or antigen-binding fragments thereof, anti-TRAIL antibodies or antigen-binding fragments thereof, anti-DR6 antibodies or antigen-binding fragments thereof, anti-EDAR antibodies or antigen-binding fragments thereof, anti-NGFR antibodies or antigen-binding fragments thereof, anti-OPG antibodies or antigen-binding fragments thereof, anti-RANKL antibodies or antigen-binding fragments thereof, anti-LTPR antibodies or antigen-binding fragments thereof, anti-BCMA antibodies or antigen-binding fragments thereof, anti-TACI antibodies or antigen-binding fragments thereof, anti-BAFFR antibodies or antigen-binding fragments thereof, anti-EDAR2 antibodies or antigen-binding fragments thereof, anti-TROY antibodies or antigen-binding fragments thereof, and anti-RELT antibodies or antigen-binding fragments thereof.

In some examples, the patient's tumor is assigned into one of the following clusters: (4) T-effector/proliferative; (5) proliferative; or (6) stromal/proliferative, and the method further comprises selecting an anti-cancer therapy comprising an anti-proliferative agent or a growth inhibitory agent (e.g., a CDK4/6 inhibitor (e.g., palbociclib, ribociclib, or abemaciclib)) for the patient.

In some examples, the patient's tumor is assigned into one of the following clusters: (4) T-effector/proliferative; (5) proliferative; or (6) stromal/proliferative, and the method further comprises treating the patient by administering an anti-cancer therapy comprising an anti-proliferative agent or a growth inhibitory agent (e.g., a cyclin dependent kinase (CDK)4/6 inhibitor (e.g., palbociclib, ribociclib, or abemaciclib)) to the patient.

In some examples, the patient's tumor is assigned into the following cluster: (3) complement/Q-oxidation, and the method further comprises selecting an anti-cancer therapy comprising a complement antagonist (e.g., a C1 inhibitor (e.g., CINRYZE® C1 esterase inhibitor)), a C3 inhibitor (e.g., a PEGylated pentadecapeptide (e.g., pegcetacoplan) or an anti-C3 antibody (e.g., H17)), a C5 inhibitor (e.g., an anti-C5 antibody (e.g., eculizumab, ABP959, ALXN1210, ALXN5500, SKY59, or LFG 316), an anti-C5 antibody fragment (e.g., MUBODINA®, a neutralizing mini antibody against C5), an siRNA (e.g., ALNCC5), a recombinant protein (e.g., coversin), or a small molecule (e.g., RA101348)), a C5a receptor antagonist (e.g., PMX53, CCX168, or MP-435)), an FD inhibitor (e.g., an anti-FD antibody (e.g., lampalizumab) or a small molecule (e.g., ACH-3856, ACH-4100, or ACH-4471)), an FB inhibitor (e.g., an anti-FB antibody, e.g., TA106), a small molecule (e.g., LNP023), an siRNA (e.g., anti-FB siRNA, Alnylam), or an antisense (e.g., Ionis-FB-L_{Rx})), a properdin inhibitor (e.g., an anti-properdin antibody (e.g., NM9401)), a C3 convertase (C3bBb) inhibitor (e.g., an FFH-based protein such as TT30 (CR2/CFH) or mini-FH (Amyndas)), or a C3 convertase (C4bC3B and C3bBb) inhibitor (e.g., mirococept (APT070)) for the patient. Other exemplary complement antagonists are described, e.g., in Risitano et al. Am. J. Hematol. 93:564-577, 2018.

In some examples, the patient's tumor is assigned into the following cluster: (3) complement/Q-oxidation, and the method further comprises treating the patient by administering an anti-cancer therapy a complement antagonist (e.g., a C1 inhibitor (e.g., CINRYZE® C1 esterase inhibitor)), a C3 inhibitor (e.g., a PEGylated pentadecapeptide (e.g., pegcetacoplan) or an anti-C3 antibody (e.g., H17)), a C5 inhibitor (e.g., an anti-C5 antibody (e.g., eculizumab, ABP959, ALXN1210, ALXN5500, SKY59, or LFG 316), an anti-C5 antibody fragment (e.g., MUBODINA®, a neutralizing mini antibody against C5), an siRNA (e.g., ALNCC5), a recombinant protein (e.g., coversin), or a small molecule (e.g., RA101348)), a C5a receptor antagonist (e.g., PMX53, CCX168, or MP-435)), an FD inhibitor (e.g., an anti-FD antibody (e.g., lampalizumab) or a small molecule (e.g., ACH-3856, ACH-4100, or ACH-4471)), an FB inhibitor (e.g., an anti-FB antibody, e.g., TA106), a small molecule (e.g., LNP023), an siRNA (e.g., anti-FB siRNA, Alnylam), or an antisense (e.g., Ionis-FB-L_{Rx})), a properdin inhibitor (e.g., an anti-properdin antibody (e.g., NM9401)), a C3 convertase (C3bBb) inhibitor (e.g., an FFH-based protein such as TT30 (CR2/CFH) or mini-FH (Amyndas)), or a C3 convertase (C4bC3B and C3bBb) inhibitor (e.g., mirococept (APT070)) to the patient.

In some examples, the patient's tumor is assigned into one of the following clusters: (3) complement/Ω-oxidation; (4) T-effector/proliferative; (5) proliferative; or (6) stromal/proliferative, and the method further comprises selecting an anti-cancer therapy comprising a metabolism inhibitor (e.g., a proprotein convertase subtilisin/kexin type 9 serine protease (PCSK9) inhibitor (e.g., an anti-PCSK9 antibody, e.g., alirocumab or evolocumab) or a fatty acid synthase (FAS) inhibitor (e.g., cerulenin, C75, isoniazid, or orlistat (tetrahydrolipstatin)) for the patient.

In some examples, the patient's tumor is assigned into one of the following clusters: (3) complement/Ω-oxidation; (4) T-effector/proliferative; (5) proliferative; or (6) stromal/proliferative, and the method further comprises treating the patient by administering an anti-cancer therapy comprising a metabolism inhibitor (e.g., a proprotein convertase subtilisin/kexin type 9 serine protease (PCSK9) inhibitor (e.g., an anti-PCSK9 antibody, e.g., alirocumab or evolocumab) or a fatty acid synthase (FAS) inhibitor (e.g., cerulenin, C75, isoniazid, or orlistat (tetrahydrolipstatin)) to the patient.

In some examples, the patient's tumor is assigned into one of the following clusters: (1) angiogenic/stromal; or (6) stromal/proliferative, and the method further comprises selecting an anti-cancer therapy comprising a stromal inhibitor (e.g., a transforming growth factor beta (TGF-β), podoplanin (PDPN), leukocyte-associated immunoglobulin-like receptor 1 (LAIR1), SMAD, anaplastic lymphoma kinase (ALK), connective tissue growth factor (CTGF/CCN2), endothelial-1 (ET-1), AP-1, interleukin (IL)-13, lysyl oxidase homolog 2 (LOXL2), endoglin (CD105), fibroblast activation protein (FAP), vascular cell adhesion protein 1 (CD106), thymocyte antigen 1 (THY1), beta 1 integrin (CD29), platelet-derived growth factor (PDGF), PDGF receptor A (PDGFRα), PDGF receptor B (PDGFRβ), vimentin, smooth muscle actin alpha (ACTA2), desmin, endosialin (CD248), or S100 calcium-binding protein A4 (S100A4) antagonist) for the patient. In some examples, the stromal inhibitor is a TGF-β antagonist (e.g., an anti-TGF-β antibody, e.g., any anti-TGF-β antibody disclosed herein).

In some examples, the patient's tumor is assigned into one of the following clusters: (1) angiogenic/stromal; or (6) stromal/proliferative, and the method further comprises treating the patient by administering an anti-cancer therapy comprising a stromal inhibitor (e.ga transforming growth factor beta (TGF-β), podoplanin (PDPN), leukocyte-associated immunoglobulin-like receptor 1 (LAIR1), SMAD, anaplastic lymphoma kinase (ALK), connective tissue growth factor (CTGF/CCN2), endothelial-1 (ET-1), AP-1, interleukin (IL)-13, lysyl oxidase homolog 2 (LOXL2), endoglin (CD105), fibroblast activation protein (FAP), vascular cell adhesion protein 1 (CD106), thymocyte antigen 1 (THY1), beta 1 integrin (CD29), platelet-derived growth factor (PDGF), PDGF receptor A (PDGFRα), PDGF receptor B (PDGFRβ), vimentin, smooth muscle actin alpha (ACTA2), desmin, endosialin (CD248), or S100 calcium-binding protein A4 (S100A4) antagonist) to the patient. In some examples, the stromal inhibitor is a TGF-β antagonist (e.g., an anti-TGF-β antibody, e.g., any anti-TGF-β antibody disclosed herein).

Any of the methods disclosed herein may comprise assaying for somatic alterations in the patient's genotype in the tumor sample obtained from the patient. Any suitable somatic alterations may be assayed. In some examples, the method comprises assaying for somatic alterations in PBRM1, CDKN2A, CDK2NB, TP53, ARID1A, and/or KMT2C.

In some examples, (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1 indicates that the patient is likely to have an increased clinical benefit from treatment with an anti-cancer therapy comprising a PD-1 axis binding antagonist (e.g., atezolizumab) and a VEGF antagonist (e.g., bevacizumab) compared to treatment with a tyrosine kinase inhibitor (e.g., sunitinib).

In some examples, the patient's genotype is determined to comprise a somatic alteration in one or more of the following genes: CDKN2A, CDK2NB, TP53, ARID1A, and KMT2C, and the method further comprises selecting an anti-cancer therapy comprising a PD-1 axis binding antagonist (e.g., atezolizumab) and a VEGF antagonist (e.g., bevacizumab) for the patient.

In some examples, the patient's genotype is determined to comprise a somatic alteration in one or more of the following genes: CDKN2A, CDK2NB, TP53, ARID1A, and KMT2C, and the method further comprises administering to the patient an anti-cancer therapy comprising a PD-1 axis binding antagonist (e.g., atezolizumab) and a VEGF antagonist (e.g., bevacizumab).

In some examples, the presence of a somatic alteration in the patient's genotype in PBRM1 indicates that the patient is likely to have an increased clinical benefit from treatment with sunitinib compared a patient whose genotype lacks a somatic alteration in PBRM1.

In some examples, the patient's genotype is determined to comprise a somatic alteration in PBRM1, and the method further comprises administering a tyrosine kinase inhibitor (e.g., sunitinib) to the patient.

Any suitable somatic alterations may be assessed. In some examples, the somatic alteration is a short variant, a loss, an amplification, a deletion, a duplication, a rearrangement, or a truncation.

Any suitable sample may be used for patient classification in the methods described herein. In some examples, the sample is a tumor sample. In some examples, the tumor sample is a formalin-fixed and paraffin-embedded (FFPE) sample, an archival sample, a fresh sample, or a frozen sample. In some examples, the tumor sample is a pre-treatment tumor sample. In some examples, the tumor sample from the patient has a clear cell histology. In other examples, the tumor sample from the patient has a non-clear cell histology. In some examples, the tumor sample from the patient has a sarcomatoid component. In some examples, the tumor sample lacks a sarcomatoid component.

In some examples, the method further comprises determining the patient's Memorial Sloan Kettering Cancer Center (MSKCC) risk score.

In some examples, the method further comprises selecting an additional therapeutic agent to the patient.

In some examples, the method further comprises administering an additional therapeutic agent to the patient.

In some examples, the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof. In some examples, the growth inhibitory agent is a CDK4/6 inhibitor (e.g., palbociclib, ribociclib, or abemaciclib). In some examples, the anti-angiogenic agent is a VEGF antagonist (e.g., any VEGF antagonist disclosed herein, e.g., an anti-VEGF antibody (e.g., bevacizumab) or a tyrosine kinase inhibitor (e.g., sunitinib or axitinib)) or a HIF2A inhibitor (e.g., belzutifan (also known as MK-6482) or PT2385). In some examples, the stromal inhibitor is a TGF-β antagonist (e.g., an anti-TGF-β antibody, e.g., any anti-TGF-β antibody disclosed herein). In some examples, the metabolism inhibitor is a PCSK9 inhibitor (e.g., an anti-PCSK9 antibody, e.g., alirocumab or evolocumab), a FAS inhibitor (e.g., cerulenin, C75, isoniazid, or orlistat (tetrahydrolipstatin)), or an AMPK inhibitor (e.g., SBI-0206965, 5′-hydroxy-staurosporine, or compound C (also known as dorsomorphin)). In some embodiments, the complement antagonist is a C1 inhibitor (e.g., CINRYZE® C1 esterase inhibitor), a C3 inhibitor (e.g., a PEGylated pentadecapeptide (e.g., pegcetacoplan) or an anti-C3 antibody (e.g., H17)), a C5 inhibitor (e.g., an anti-C5 antibody (e.g., eculizumab, ABP959, ALXN1210, ALXN5500, SKY59, or LFG 316), an anti-C5 antibody fragment (e.g., MUBODINA®, a neutralizing mini antibody against C5), an siRNA (e.g., ALNCC5), a recombinant protein (e.g., coversin), or a small molecule (e.g., RA101348)), a C5a receptor antagonist (e.g., PMX53, CCX168, or MP-435), an FD inhibitor (e.g., an anti-FD antibody (e.g., lampalizumab) or a small molecule (e.g., ACH-3856, ACH-4100, or ACH-4471)), an FB inhibitor (e.g., an anti-FB antibody, e.g., TA106), a small molecule (e.g., LNP023), an siRNA (e.g., anti-FB siRNA, Alnylam), or an antisense (e.g., Ionis-FB-L_{Rx})), a properdin inhibitor (e.g., an anti-properdin antibody (e.g., NM9401)), a C3 convertase (C3bBb) inhibitor (e.g., an FFH-based protein such as TT30 (CR2/CFH) or mini-FH (Amyndas)), or a C3 convertase (C4bC3B and C3bBb) inhibitor (e.g., mirococept (APT070)).

Any of the methods of classifying a kidney cancer in a patient may further include treating the patient, e.g., using any approach described below in Section III.

### III. Therapeutic Methods, Compositions, and Uses for Kidney Cancer

In one example, provided herein is a method of treating a kidney cancer (e.g., RCC, e.g., an inoperable, locally advanced, or metastatic RCC) in a human patient, the method comprising: classifying the cancer in the patient according to any one of the methods disclosed herein; and administering an anti-cancer therapy to the patient based on the classification.

In another example, provided herein is an anti-cancer therapy for use in treating a kidney cancer (e.g., RCC, e.g., an inoperable, locally advanced, or metastatic RCC) in a human patient, wherein the kidney cancer in the patient has been classified according to any one of the methods disclosed herein.

In another example, provided herein is the use of an anti-cancer therapy in the preparation of a medicament for treating a kidney cancer (e.g., RCC, e.g., an inoperable, locally advanced, or metastatic RCC) in a human patient, wherein the kidney cancer in the patient has been classified according to any one of the methods disclosed herein.

In some examples, the kidney cancer is previously untreated.

For example, provided herein is a method of treating a kidney cancer (e.g., RCC, e.g., an inoperable, locally advanced, or metastatic RCC) in a human patient, wherein the kidney cancer is untreated, the method comprising: classifying the cancer in the patient according to any one of the methods disclosed herein; and administering an anti-cancer therapy to the patient based on the classification.

In another example, provided herein is an anti-cancer therapy for use in treating a kidney cancer (e.g., RCC, e.g., an inoperable, locally advanced, or metastatic RCC) in a human patient, wherein the kidney cancer is untreated, wherein the kidney cancer in the patient has been classified according to any one of the methods disclosed herein.

In another example, provided herein is the use of an anti-cancer therapy in the preparation of a medicament for treating a kidney cancer (e.g., RCC, e.g., an inoperable, locally advanced, or metastatic RCC) in a human patient, wherein the kidney cancer is previously untreated, wherein the kidney cancer in the patient has been classified according to any one of the methods disclosed herein.

In one example, provided herein is a method of treating an inoperable, locally advanced, or metastatic RCC in a human patient, the method comprising: classifying the previously untreated inoperable, locally advanced, or metastatic RCC in the patient according to any one of the methods disclosed herein; and administering an anti-cancer therapy to the patient based on the classification.

In another example, provided herein is an anti-cancer therapy for use in treating an inoperable, locally advanced, or metastatic RCC in a human patient, wherein the previously untreated inoperable, locally advanced, or metastatic RCC in the patient has been classified according to any one of the methods disclosed herein.

In another example, provided herein is the use of an anti-cancer therapy in the preparation of a medicament for treating an inoperable, locally advanced, or metastatic RCC in a human patient, wherein the previously untreated inoperable, locally advanced, or metastatic RCC in the patient has been classified according to any one of the methods disclosed herein.

Any suitable anti-cancer therapy may be administered to the patient based on the classification. For example, in some embodiments, a PD-1 axis binding antagonist (e.g., an anti-PD-L1 antibody, e.g., atezolizumab or avelumab) is administered to the patient. In some examples, a VEGF antagonist (e.g., an anti-VEGF antibody (e.g., bevacizumab) or a tyrosine kinase inhibitor (e.g., sunitinib or axitinib) is administered to the patient. In some examples, the anti-cancer therapy comprises atezolizumab and bevacizumab. In other examples, the anti-cancer therapy comprises avelumab and axitinib. In some examples, the method further comprises administering an additional therapeutic agent to the patient.

In another example, provided herein is a method of treating a kidney cancer (e.g., RCC, e.g., an inoperable, locally advanced, or metastatic RCC) in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, the method comprising administering to the patient an anti-cancer therapy comprising a PD-1 axis binding antagonist (e.g., atezolizumab or avelumab) and a VEGF antagonist (e.g., bevacizumab or axitinib).

In another example, provided herein is a PD-1 axis binding antagonist (e.g., atezolizumab or axitinib) for use in treating a kidney cancer (e.g., RCC, e.g., an inoperable, locally advanced, or metastatic RCC) in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, wherein the PD-1 axis binding antagonist is administered in combination with a VEGF antagonist (e.g., bevacizumab or axitinib).

In another example, provided herein is the use of a PD-1 axis binding antagonist (e.g., atezolizumab or avelumab) in the preparation of a medicament for treating a kidney cancer (e.g., RCC, e.g., an inoperable, locally advanced, or metastatic RCC) in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, wherein the medicament is administered in combination with a VEGF antagonist (e.g., bevacizumab or axitinib).

In some examples, the kidney cancer is previously untreated.

For example, provided herein is a method of treating a previously untreated kidney cancer (e.g., RCC, e.g., an inoperable, locally advanced, or metastatic RCC) in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, the method comprising administering to the patient an anti-cancer therapy comprising a PD-1 axis binding antagonist (e.g., atezolizumab or avelumab) and a VEGF antagonist (e.g., bevacizumab or axitinib).

In another example, provided herein is a PD-1 axis binding antagonist (e.g., atezolizumab or avelumab) for use in treating a previously untreated kidney cancer (e.g., RCC, e.g., an inoperable, locally advanced, or metastatic RCC) in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, wherein the PD-1 axis binding antagonist is administered in combination with a VEGF antagonist (e.g., bevacizumab or axitinib).

In another example, provided herein is the use of a PD-1 axis binding antagonist (e.g., atezolizumab or avelumab) in the preparation of a medicament for treating a previously untreated kidney cancer (e.g., RCC, e.g., an inoperable, locally advanced, or metastatic RCC) in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, wherein the medicament is administered in combination with a VEGF antagonist (e.g., bevacizumab or axitinib).

In some examples, the kidney cancer is RCC. In some examples, the kidney cancer is an inoperable, locally advanced, or metastatic RCC.

In another example, provided herein is a method of treating a previously untreated inoperable, locally advanced, or metastatic RCC in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, the method comprising administering to the patient an anti-cancer therapy comprising atezolizumab or bevacizumab.

In another example, provided herein is atezolizumab for use in treating a previously untreated inoperable, locally advanced, or metastatic RCC in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, wherein the atezolizumab is administered in combination with bevacizumab.

In another example, provided herein is the use of atezolizumab in the preparation of a medicament for treating a previously untreated inoperable, locally advanced, or metastatic RCC in a patient whose genotype has been determined to comprise a somatic alteration in one or more of the following genes: CDKN2A, CDK2NB, TP53, ARID1A, and KMT2C, wherein the medicament is administered in combination bevacizumab.

In some examples, the PD-1 axis binding antagonist and/or the VEGF antagonist is administered in combination with an effective amount of one or more additional therapeutic agents. In some examples, the PD-1 axis binding antagonist is administered in combination with an effective amount of a VEGF antagonist. In some examples, the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof. In some examples, the growth inhibitory agent is a CDK4/6 inhibitor (e.g., palbociclib, ribociclib, or abemaciclib). In some examples, the anti-angiogenic agent is a VEGF antagonist (e.g., any VEGF antagonist disclosed herein, e.g., an anti-VEGF antibody (e.g., bevacizumab) or a tyrosine kinase inhibitor (e.g., sunitinib or axitinib)) or a HIF2A inhibitor (e.g., belzutifan (also known as MK-6482) or PT2385). In some examples, the stromal inhibitor is a TGF-β antagonist (e.g., an anti-TGF-β antibody, e.g., any anti-TGF-β antibody disclosed herein). In some examples, the metabolism inhibitor is a PCSK9 inhibitor (e.g., an anti-PCSK9 antibody, e.g., alirocumab or evolocumab), a FAS inhibitor (e.g., cerulenin, C75, isoniazid, or orlistat (tetrahydrolipstatin)), or an AMPK inhibitor (e.g., SBI-0206965, 5′-hydroxy-staurosporine, or compound C (also known as dorsomorphin)). In some embodiments, the complement antagonist is a C1 inhibitor (e.g., CINRYZE® C1 esterase inhibitor), a C3 inhibitor (e.g., a PEGylated pentadecapeptide (e.g., pegcetacoplan) or an anti-C3 antibody (e.g., H17)), a C5 inhibitor (e.g., an anti-C5 antibody (e.g., eculizumab, ABP959, ALXN1210, ALXN5500, SKY59, or LFG 316), an anti-C5 antibody fragment (e.g., MUBODINA®, a neutralizing mini antibody against C5), an siRNA (e.g., ALNCC5), a recombinant protein (e.g., coversin), or a small molecule (e.g., RA101348)), a C5a receptor antagonist (e.g., PMX53, CCX168, or MP-435), an FD inhibitor (e.g., an anti-FD antibody (e.g., lampalizumab) or a small molecule (e.g., ACH-3856, ACH-4100, or ACH-4471)), an FB inhibitor (e.g., an anti-FB antibody, e.g., TA106), a small molecule (e.g., LNP023), an siRNA (e.g., anti-FB siRNA, Alnylam), or an antisense (e.g., Ionis-FB-L_{Rx})), a properdin inhibitor (e.g., an anti-properdin antibody (e.g., NM9401)), a C3 convertase (C3bBb) inhibitor (e.g., an FFH-based protein such as TT30 (CR2/CFH) or mini-FH (Amyndas)), or a C3 convertase (C4bC3B and C3bBb) inhibitor (e.g., mirococept (APT070)).

In any of the preceding examples, each dosing cycle may have any suitable length, e.g., about 7 days, about 14 days, about 21 days, about 28 days, about 35 days, about 42 days, or longer. In some instances, each dosing cycle is about 21 days. In some instances, each dosing cycle is about 42 days.

As a general proposition, the therapeutically effective amount of a PD-1 axis binding antagonist (e.g., atezolizumab) administered to a human will be in the range of about 0.01 to about 50 mg/kg of patient body weight, whether by one or more administrations.

In some exemplary embodiments, the PD-1 axis binding antagonist is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example.

In one instance, a PD-1 axis binding antagonist is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some instances, the PD-1 axis binding antagonist may be administered at a dose of about 1000 mg to about 1400 mg every three weeks (e.g., about 1100 mg to about 1300 mg every three weeks, e.g., about 1150 mg to about 1250 mg every three weeks). In some instances, the PD-1 axis binding antagonist may be administered at a dose of 1200 mg every three weeks.

In some instances, a patient is administered a total of 1 to 50 doses of a PD-1 axis binding antagonist, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

In some instances, atezolizumab is administered to the patient intravenously at a dose of about 840 mg every 2 weeks, about 1200 mg every 3 weeks, or about 1680 mg of every 4 weeks.

In some instances, atezolizumab is administered at a fixed dose of 1200 mg via intravenous infusion on Days 1 and 22 of each 42-day cycle.

In some instances, atezolizumab is administered at a fixed dose of 1200 mg via intravenous (IV) infusion on Days 1 and 22 of each 42-day cycle, and bevacizumab is administered at a dose of 15 mg/kg via IV infusion on Days 1 and 22 of each 42-day cycle.

In some instances, avelumab is administered at a dose of 10 mg/kg IV every two weeks.

In some instances, axitinib is administered at a dose of 5 mg orally twice a day (PO BID).

In some instances, avelumab is administered at a dose of 10 mg/kg IV every two weeks, and axitinib is administered at a dose of 5 mg PO BID for a 6-week cycle.

In some instances, sunitinib is administered at a dose of 50 mg PO every day (QD). The PD-1 axis binding antagonist, the VEGF antagonist, and/or any additional therapeutic agent(s), including an immunotherapy agent, a cytotoxic agent, a growth inhibitory agent, a stromal inhibitor, a metabolism inhibitor, a complement antagonist, a radiation therapy agent, an anti-angiogenic agent (e.g., a VEGF antagonist), or a combination thereof, may be administered in any suitable manner known in the art.

For example, the PD-1 axis binding antagonist, the VEGF antagonist, and/or any additional therapeutic agent(s) may be administered sequentially (on different days) or concurrently (on the same day or during the same treatment cycle). In some instances, the PD-1 axis binding antagonist is administered prior to the additional therapeutic agent. In other instances, the PD-1 axis binding antagonist is administered after the additional therapeutic agent. In some instances, the PD-1 axis binding antagonist and/or any additional therapeutic agent(s) may be administered on the same day. In some instances, the PD-1 axis binding antagonist may be administered prior to an additional therapeutic agent that is administered on the same day. For example, the PD-1 axis binding antagonist may be administered prior to chemotherapy on the same day. In another example, the PD-1 axis binding antagonist may be administered prior to both chemotherapy and another drug (e.g., bevacizumab) on the same day. In other instances, the PD-1 axis binding antagonist may be administered after an additional therapeutic agent that is administered on the same day. In yet other instances, the PD-1 axis binding antagonist is administered at the same time as the additional therapeutic agent. In some instances, the PD-1 axis binding antagonist is in a separate composition as the additional therapeutic agent. In some instances, the PD-1 axis binding antagonist is in the same composition as the additional therapeutic agent. In some instances, the PD-1 axis binding antagonist is administered through a separate intravenous line from any other therapeutic agent administered to the patient on the same day.

The PD-1 axis binding antagonist, the VEGF antagonist, and any additional therapeutic agent(s) may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred embodiment, the PD-1 axis binding antagonist is administered intravenously. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus.

Also provided herein are methods for treating kidney cancer (e.g., RCC, e.g., an inoperable, locally advanced, or metastatic RCC) in a patient comprising administering to the patient a treatment regimen comprising an effective amount of a PD-1 axis binding antagonist (e.g., atezolizumab) and/or a VEGF antagonist (e.g., bevacizumab) in combination with another anti-cancer agent or cancer therapy. For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof. For example, the PD-1 axis binding antagonist may be administered in combination with bevacizumab, paclitaxel, paclitaxel protein-bound (e.g., nab-paclitaxel), carboplatin, cisplatin, pemetrexed, gemcitabine, etoposide, cobimetinib, vemurafenib, or a combination thereof. The PD-1 axis binding antagonist may be an anti-PD-L1 antibody (e.g., atezolizumab) or an anti-PD-1 antibody.

For example, when administering with chemotherapy with or without bevacizumab, atezolizumab may be administered at a dose of 1200 mg every 3 weeks prior to chemotherapy and bevacizumab. In another example, following completion of 4-6 cycles of chemotherapy, and if bevacizumab is discontinued, atezolizumab may be administered at a dose of 840 mg every 2 weeks, 1200 mg every 3 weeks, or 1680 mg every four weeks. In another example, atezolizumab may be administered at a dose of 840 mg, followed by 100 mg/m² of paclitaxel protein-bound (e.g., nab-paclitaxel); for each 28 day cycle, atezolizumab is administered on days 1 and 15, and paclitaxel protein-bound is administered on days 1, 8, and 15. In another example, when administering with carboplatin and etoposide, atezolizumab can be administered at a dose of 1200 mg every 3 weeks prior to chemotherapy. In yet another example, following completion of 4 cycles of carboplatin and etoposide, atezolizumab may be administered at a dose of 840 mg every 2 weeks, 1200 mg every 3 weeks, or 1680 mg every 4 weeks. In another example, following completion of a 28-day cycle of cobimenitib and vemurafenib, atezolizumab may be administered at a dose of 840 mg every 2 weeks with cobimetinib at a dose of 60 mg orally once daily (21 days on, 7 days off) and vemurafenib at a dose of 720 mg orally twice daily.

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

### IV. Assessment of PD-L1 Expression

The expression of PD-L1 may be assessed in a patient treated according to any of the methods, compositions for use, and uses described herein. The methods, compositions for use, and uses may include determining the expression level of PD-L1 in a biological sample (e.g., a tumor sample) obtained from the patient. In other examples, the expression level of PD-L1 in a biological sample (e.g., a tumor sample) obtained from the patient has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1 L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the patient has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the patient has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some examples, tumor samples may be scored for PD-L1 positivity in tumor-infiltrating immune cells and/or in tumor cells according to the criteria for diagnostic assessment shown in Table 2 and/or Table 3, respectively.

TABLE 2
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by | |
| tumor cells, associated intratumoral stroma, | |
| and contiguous peri-tumoral desmoplastic | |
| stroma | |
| Presence of discernible PD-L1 staining of any | IC1 |
| intensity in tumor-infiltrating immune cells | |
| covering ≥1% to <5% of tumor area occupied | |
| by tumor cells, associated intratumoral stroma, | |
| and contiguous peri-tumoral desmoplastic | |
| stroma | |
| Presence of discernible PD-L1 staining of any | IC2 |
| intensity in tumor-infiltrating immune cells | |
| covering ≥5% to <10% of tumor area occupied | |
| by tumor cells, associated intratumoral stroma, | |
| and contiguous peri-tumoral desmoplastic | |
| stroma | |
| Presence of discernible PD-L1 staining of any | IC3 |
| intensity in tumor-infiltrating immune cells | |
| covering ≥10% of tumor area occupied by | |
| tumor cells, associated intratumoral stroma, | |
| and contiguous peri-tumoral desmoplastic | |
| stroma | |

TABLE 3
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### V. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:

(a) an HVR-H1, HVR-H2, and HVR-H3 sequence of
(SEQ ID NO: 3)
GFTFSDSWIH,
(SEQ ID NO: 4)
AWISPYGGSTYYADSVKG
and
(SEQ ID NO: 5)
RHWPGGFDY,
respectively, and
(b) an HVR-L1, HVR-L2, and HVR-L3 sequence of
(SEQ ID NO: 6)
RASQDVSTAVA,
(SEQ ID NO: 7)
SASFLYS
and
(SEQ ID NO: 8)
QQYLYHPAT,
respectively.

In one embodiment, the anti-PD-L1 antibody comprises: PGP-30 DNA

(a) a heavy chain variable region (VH) comprising
the amino acid sequence:
(SEQ ID NO: 9)
EVQLVESGGGLVQPGGSLRLSCAASGFTFSDSWIHWVRQAPGKGLEW
VAWISPYGGSTYYADSVKGRFTISADTSKNTAYLQMNSLRAEDTAVY
YCARRHWPGGFDYWGQGTLVTVSS,
and
(b) the light chain variable region (VL)
comprising the amino acid sequence:
(SEQ ID NO: 10)
DIQMTQSPSSLSASVGDRVTITCRASQDVSTAVAWYQQKPGKAPKLL
IYSASFLYSGVPSRFSGSGSGTDFTLTISSLQPEDFATYYCQQYLYH
PATFGQGTKVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQAPGKGLEW
VAWISPYGGSTYYADSVKGRFTISADTSKNTAYLQMNSLRAEDTAVY
YCARRHWPGGFDYWGQGTLVTVSSASTKGPSVFPLAPSSKSTSGGTA
ALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDG
VEVHNAKTKPREEQYASTYRVVSVLTVLHQDWLNGKEYKCKVSNKAL
PAPIEKTISKAKGQPREPQVYTLPPSREEMTKNQVSLTCLVKGFYPS
DIAVEWESNGQPENNYKTTPPVLDSDGSFFLYSKLTVDKSRWQQGNV
FSCSVMHEALHNHYTQKSLSLSPG,
and
(b) the light chain amino acid sequence:
(SEQ ID NO: 2)
DIQMTQSPSSLSASVGDRVTITCRASQDVSTAVAWYQQKPGKAPKLL
IYSASFLYSGVPSRFSGSGSGTDFTLTISSLQPEDFATYYCQQYLYH
PATFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances the anti-PD-1 antibody is a monoclonal antibody. In some instances, the anti-PD-1 antibody is an antibody fragment selected from the group consisting of Fab, Fab′, Fab′-SH, Fv, scFv, and (Fab′)2 fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)2 fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### VI. VEGF Antagonists

Provided herein are methods for treating kidney cancer (e.g., RCC, e.g., an inoperable, locally advanced, or metastatic RCC) in a patient comprising administering to the patient a treatment regimen comprising a PD-1 axis binding antagonist (e.g., atezolizumab) and a VEGF antagonist (e.g., bevacizumab). Also provided are related compositions (e.g., pharmaceutical compositions) for use, kits, and articles of manufacture. Any of the methods, compositions for use, kits, or articles of manufacture described herein may include or involve any of the agents described below.

VEGF antagonists include any molecule capable of binding VEGF, reducing VEGF expression levels, or neutralizing, blocking, inhibiting, abrogating, reducing, or interfering with VEGF biological activities. An exemplary human VEGF is shown under UniProtKB/Swiss-Prot Accession No. P15692, Gene ID (NCBI): 7422.

In some instances, the VEGF antagonist is an anti-VEGF antibody. In some embodiments, the anti-VEGF antibody is bevacizumab, also known as “rhuMab VEGF” or “AVASTIN®.” Bevacizumab is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al. (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

In other instances, the VEGF antagonist is an anti-VEGFR2 antibody or related molecule (e.g., ramucirumab, tanibirumab, aflibercept); an anti-VEGFR1 antibody or related molecules (e.g., icrucumab, aflibercept (VEGF Trap-Eye; EYLEA®), or ziv-aflibercept (VEGF Trap; ZALTRAP®)); a bispecific VEGF antibody (e.g., MP-0250, vanucizumab (VEGF-ANG2), or bispecific antibodies disclosed in US 2001/0236388); a bispecific antibody including a combination of two of anti-VEGF, anti-VEGFR1, and anti-VEGFR2 arms; an anti-VEGFA antibody (e.g., bevacizumab, sevacizumab); an anti-VEGFB antibody; an anti-VEGFC antibody (e.g., VGX-100), an anti-VEGFD antibody; or a nonpeptide small molecule VEGF antagonist (e.g., pazopanib, axitinib, vandetanib, stivarga, cabozantinib, lenvatinib, nintedanib, orantinib, telatinib, dovitinib, cediranib, motesanib, sulfatinib, apatinib, foretinib, famitinib, or tivozanib). In some examples, the VEGF antagonist may be a tyrosine kinase inhibitor, including a receptor tyrosine kinase inhibitors (e.g., a multi-targeted receptor tyrosine kinase inhibitor such as sunitinib or axitinib).

### VII. Pharmaceutical Compositions and Formulations

Also provided herein are pharmaceutical compositions and formulations comprising a PD-1 axis binding antagonist (e.g., atezolizumab) and, optionally, a pharmaceutically acceptable carrier. The disclosure also provides pharmaceutical compositions and formulations comprising a VEGF antagonist (e.g., bevacizumab), and optionally, a pharmaceutically acceptable carrier. Any of the additional therapeutic agents described herein may also be included in a pharmaceutical composition or formulation.

Pharmaceutical compositions and formulations as described herein can be prepared by mixing the active ingredients (e.g., a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

### VIII. Articles of Manufacture or Kits

Also provided herein are articles of manufacture and kits, which may be used for classifying a patient according to any of the methods disclosed herein.

In one example, provided herein is a kit for classifying a kidney cancer (e.g., RCC, e.g., an inoperable, locally advanced, or metastatic RCC) in a human patient, wherein the kidney cancer is previously untreated, the kit comprising: (a) reagents for assaying mRNA in a tumor sample from the patient to provide a transcriptional profile of the patient's tumor; and (b) instructions for assigning the patient's tumor sample into one of the following seven clusters based on the transcriptional profile of the patient's tumor: (1) angiogenic/stromal; (2) angiogenic; (3) complement/Ω-oxidation; (4) T-effector/proliferative; (5) proliferative; (6) stromal/proliferative; and (7) snoRNA, thereby classifying the kidney cancer in the patient. Any suitable reagents for assaying mRNA may be included in the kit, e.g., nucleic acids, enzymes, buffers, and the like.

In one example, provided herein is a kit for identifying a human patient suffering from an kidney cancer (e.g., RCC, e.g., an inoperable, locally advanced, or metastatic RCC) who may benefit from treatment with an anti-cancer therapy comprising a PD-1 axis binding antagonist (e.g., atezolizumab) and a VEGF antagonist (e.g., bevacizumab), wherein the kidney cancer is previously untreated, the kit comprising: (a) reagents for determining the presence of a somatic alteration in one or more of the following genes: PBRM1, CDKN2A, CDK2NB, TP53, ARID1A, and KMT2C in a tumor sample obtained from the patient; and (b) instructions for using the reagents to identify the patient as one who may benefit from a treatment with an anti-cancer therapy comprising a PD-1 axis binding antagonist and a VEGF antagonist. In some examples, (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1 indicates that the patient is likely to have an increased clinical benefit from treatment with an anti-cancer therapy comprising a PD-1 axis binding antagonist (e.g., atezolizumab) and a VEGF antagonist (e.g., bevacizumab) compared to treatment with a tyrosine kinase inhibitor (e.g., sunitinib).

In another aspect, provided herein is an article of manufacture or a kit comprising a PD-1 axis binding antagonist (e.g., atezolizumab) and/or a VEGF antagonist (e.g., bevacizumab). In some instances, the article of manufacture or kit further comprises package insert comprising instructions for using the PD-1 axis binding antagonist to treat or delay progression of kidney cancer (e.g., RCC, e.g., an inoperable, locally advanced, or metastatic RCC) in a patient, e.g., for a patient who has been classified according to any of the methods disclosed herein. In some instances, the article of manufacture or kit further comprises package insert comprising instructions for using the PD-1 axis binding antagonist in combination with a VEGF antagonist to treat or delay progression of kidney cancer (e.g., RCC, e.g., an inoperable, locally advanced, or metastatic RCC) in a patient. Any of the PD-1 axis binding antagonists, VEGF antagonists, and/or any additional therapeutic agents described herein may be included in the article of manufacture or kits.

In some instances, the PD-1 axis binding antagonist, the VEGF antagonist, and/or any additional therapeutic agent are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags, and syringes.

Any of the articles of manufacture or kits may include instructions to administer a PD-1 axis binding antagonist and/or a VEGF antagonist, or another anti-cancer therapy, to a patient in accordance with any of the methods described herein, e.g., any of the methods set forth in Section III above.

### EXAMPLES

### Example 1: Molecular Subsets in Renal Cancer Determine Outcome to Checkpoint and Angiogenesis Blockade

This Example describes integrated multi-omics analyses that led to identification of robust molecular subtypes in 823 tumors from patients with advanced renal cell carcinoma (RCC), including 134 tumors with sarcomatoid features, from a randomized, global Phase III trial (IMmotion151). These molecular subgroups were associated with differential clinical outcomes of the combination of an anti-angiogenesis agent (i.e., bevacizumab, anti-VEGF) and a checkpoint inhibitor (CPI; i.e., atezolizumab, anti-PD-L1) versus a VEGF receptor tyrosine kinase inhibitor (TKI; i.e., sunitinib). The biological and clinical insights gained from this study inform biomarker strategies for personalized treatment and guide future therapeutic development in RCC and other cancers.

### A. Study Design

IMmotion151 (NCT02420821) was a multicenter, open-label, Phase 3, randomized controlled trial of atezolizumab plus bevacizumab (n=454) versus sunitinib (n=461) in patients with previously untreated advanced RCC (Rini et al. Lancet. 393: 2404-2415 (2019)). The study design, methods, and primary clinical findings from IMmotion151 have been reported previously (Rini et al. Lancet. 393: 2404-2415 (2019)).

Briefly, previously untreated patients with unresectable locally advanced or metastatic renal cell carcinoma with any component of clear-cell or sarcomatoid histology were randomized to receive atezolizumab 1200 mg+bevacizumab 15 mg/kg (atezolizumab+bevacizumab) once every 3 weeks (n=454) or sunitinib 50 mg once daily (n=461; 4 weeks on, 2 weeks off). The co-primary endpoints were investigator-assessed progression-free survival (PFS) in patients with ≥1% expressing PD-L1 on immune cells (IC, PD-L1⁺) and overall survival (OS) in the intent-to-treat (ITT) population. Patients with PD-L1⁺ tumors who received atezolizumab+bevacizumab showed improved PFS vs. sunitinib (Hazard ratio, HR 0.74, 95% CI: 0.57-0.96; p=0.0217, median PFS (mPFS) 11.2 vs 7.7 months; Rini et al. Lancet. 393: 2404-2415 (2019)).

In the present study, pre-treatment tumors from 823/915 (90%) patients were transcriptionally profiled by RNA-seq. This subset comprised of 198 metastatic and 625 primary tumors, all of which were collected no longer than 2 years prior to enrollment in this study. In this biomarker evaluable tumor collection, 688 tumors were of clear cell histology without a sarcomatoid component, 110 tumors were of clear cell histology with any sarcomatoid component, 1 tumor was of clear cell histology with unknown sarcomatoid component, and 24 tumors were of non-clear cell histology with any sarcomatoid component. Pre-treatment tumors from 715 patients were assessed for somatic mutations and alterations using the FOUNDATIONONE® assay (Foundation Medicine, MA). Overall, tumors from 702 patients were profiled both by RNA-seq and the FOUNDATIONONE® assay, representing the largest genomic biomarker dataset to date in a randomized trial in untreated advanced RCC. Validation of molecular classification was conducted in tumors collected from patients in the randomized Phase II trial, IMmotion150.

### B. Materials and Methods

i. Patients

IMmotion151 (NCT02420821) was a multicenter, open-label, Phase 3, randomized controlled trial of atezolizumab plus bevacizumab (n=454) vs. sunitinib (n=461) in patients with previously untreated advanced renal cell carcinoma (Rini et al. Lancet. 393: 2404-2415 (2019)).

ii. PD-L1 Immunohistochemistry and Scoring

PD-L1 expression was assessed by immunohistochemistry using the SP142 assay (Ventana, AZ). Tumors were characterized as PD-L1+ if PD-L1 staining of any intensity on immune cells covered 21% of tumor area occupied by tumor cells, associated intratumoral, and contiguous peri-tumoral desmoplastic stroma.

iii. RNA Processing

Formalin-fixed paraffin-embedded (FFPE) tissue was macro-dissected for tumor area using hematoxylin and eosin (H&E) staining as a guide. RNA was extracted using the High Pure FFPET RNA Isolation Kit (Roche) and assessed by QUBIT™ (Thermo Fisher Scientific) and Agilent Bioanalyzer for quantity and quality. First-strand cDNA synthesis was primed from total RNA using random primers, followed by the generation of second strand cDNA with dUTP in place of dTTP in the master mix to facilitate preservation of strand information. Libraries were enriched for the mRNA fraction by positive selection using a cocktail of biotinylated oligos corresponding to coding regions of the genome. Libraries were sequenced using the Illumina sequencing method.

iv. RNA-seq Data Generation and Processing

Whole-transcriptome profiles were generated using TruSeq RNA Access technology (Illumina). RNA-seq reads were first aligned to ribosomal RNA sequences to remove ribosomal reads. The remaining reads were aligned to the human reference genome (NCBI Build 38) using GSNAP (Wu and Nacu. Bioinformatics. 26(7): 873-881 (2010); Wu et al. Methods Mol Biol. 1418: 283-334 (2016)) version 2013-10-10, allowing a maximum of two mismatches per 75 base sequence (parameters: ‘-M 2-n 10-B 2-i 1-N 1-w 200000-E 1-pairmax-rna=200000-clip-overlap). To quantify gene expression levels, the number of reads mapped to the exons of each RefSeq gene was calculated using the functionality provided by the R/Bioconductor package GenomicAlignments. Raw counts were adjusted for gene length using transcript-per-million (TPM) normalization, and subsequently log 2-transformed.

v. DNA Mutation and Copy-Number Profiling by FOUNDATIONONE® Assay

Comprehensive genomic profiling (CGP) was carried out using the FOUNDATIONONE® T7 assay (Foundation Medicine Inc., Cambridge, MA) in a Clinical Laboratory Improvement Amendments (CLIA)-certified, College of American Pathologists (CAP)-accredited laboratory. Hybrid capture was carried out for all coding exons from up to 395 cancer-related genes plus select introns from up to 31 genes frequently rearranged in cancer. All classes of genomic alterations (GA) were assessed, including short variant (missense, stop, nonstart, splice site point mutations as well as short indels), biallelic deletions, amplifications and rearrangement alterations, as previously described (Frampton et al. Nat Biotechnol. 31: 1023-1031 (2013)). Shallow copy-number loss (CN=1) was called using similar methodology to arm-level calling. Normalized coverage data for exonic, intronic, and SNP targets accounting for stromal admixture were plotted on a logarithmic scale and minor allele SNP frequencies were concordantly plotted. Custom circular binary segmentation further clustered targets and minor allele SNPs to define upper and lower bounds of genomic segments. Signal-to-noise ratios for each segment were used to determine whether the segment was gained or lost. The sum of those segment sizes determined the fraction of each segment gained or lost. For gene alteration analyses described herein, position-level information was leveraged to define per-gene alteration profiles, and every gene's mutational profile was dichotomized as altered (including copy-number loss or gain) or non-altered.

vi. Fusion Detection

Paired trimmed/clipped and de-duplicated RNA-seq reads were used to identify gene fusion events. Reads were aligned using STAR v 2.7.2b with default parameters to the GRCh38 genome. This aligned output was used as input to STAR-Fusion v 1.9.1 (Haas et al. Genome Biol. 20: 213 (2019)) using the developer-supplied gencode v 33 CTAT library from Apr. 6, 2020. Each fusion gene was required to be supported by at least two reads.

vii. T-effector and Angiogenesis Gene Signature Threshold Definition and Validation

RNA-seq data from the randomized Phase II trial IMmotion150 were processed as described above. Transcriptional signature scores were derived from T-effector and angiogenesis signatures (McDermott et al. Nat Med. 24: 749-757 (2018)) for each sample, and hazard ratios were calculated at various gene expression scores. Gene expression score cutoffs of 2.93 (40% prevalence) and 5.82 (50% prevalence) were defined for the T-effector and angiogenesis signatures in IMmotion150 based on a combination of prevalence and hazard ratio plateauing. These absolute thresholds were prospectively applied to the IMmotion151 data to classify tumors with high and low T-effector and angiogenesis signatures. Cox-proportional hazard regression models were fit to compare PFS in atezolizumab+bevacizumab or sunitinib-treated patients in gene expression high and low subsets.

viii. Non-negative Matrix Factorization (NMF)

Using Median Absolute Deviation (MAD) analysis, 3072 genes (top 10%) were selected with the highest variability across patients. Subclasses were then computed by reducing the dimensionality of the expression data from thousands of genes to a few metagenes using consensus NMF clustering (CRAN. R package version 0.22.0, Brunet et al. Proc Natl Acad Sci USA. 101: 4164-4169 (2004)). This method computes multiple k-factor factorization decompositions of the expression matrix and evaluates the stability of the solutions using a cophenetic coefficient. The most robust consensus NMF clustering of 823 patient samples using the 3072 most variable genes selected and testing k=2 to k=8 was identified as k=7.

ix. Validation of NMF Clustering in IMmotion150

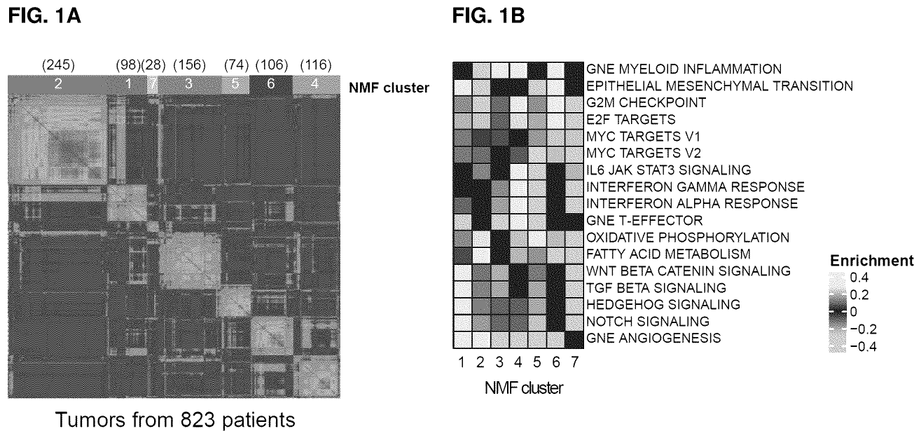

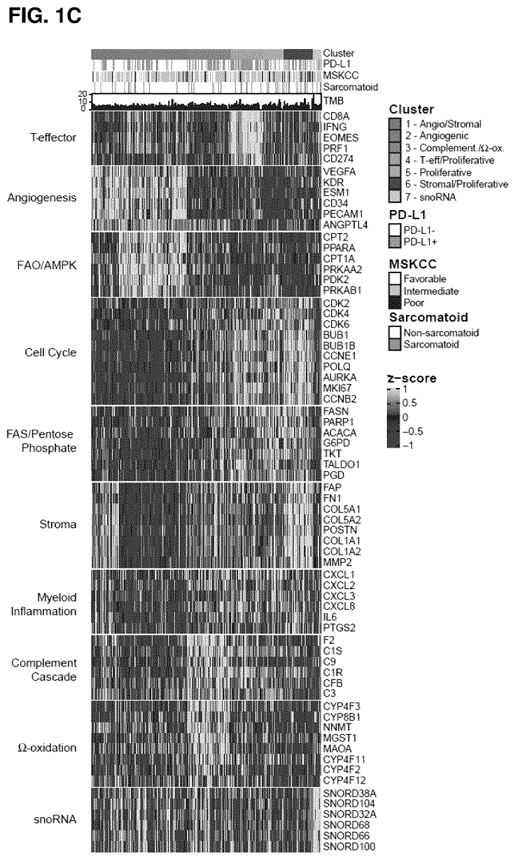

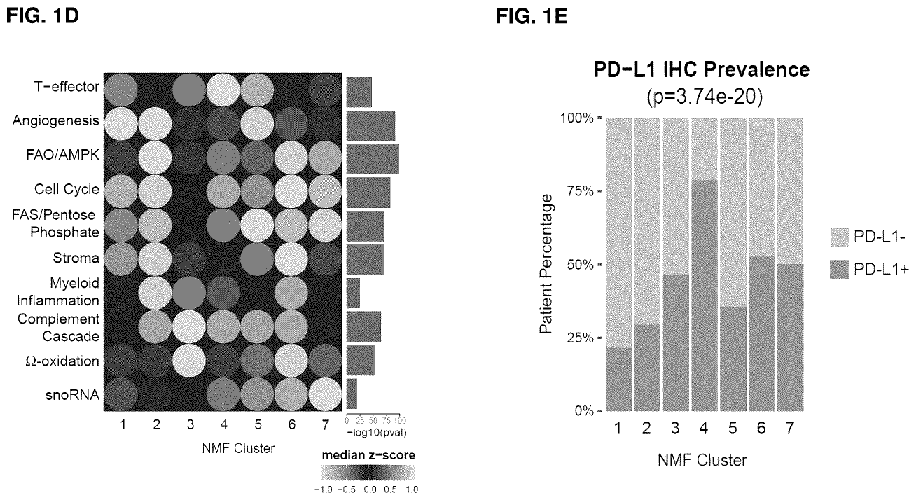

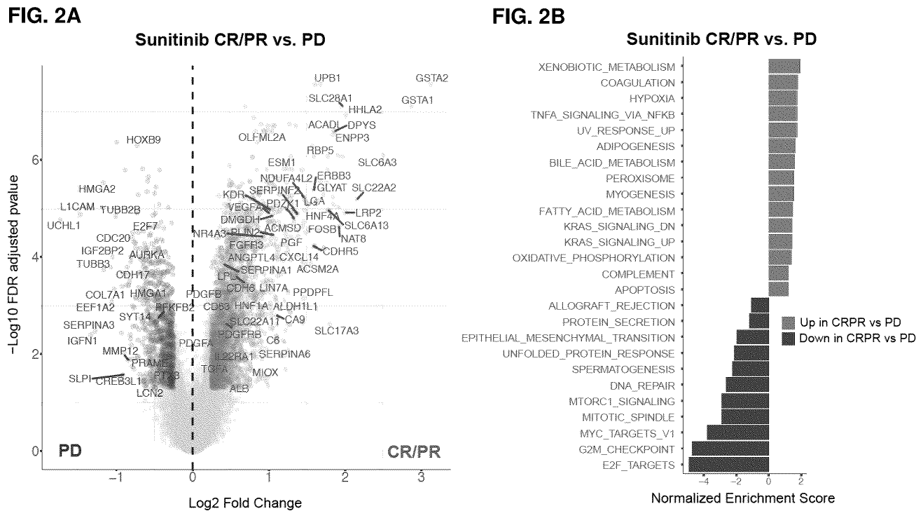

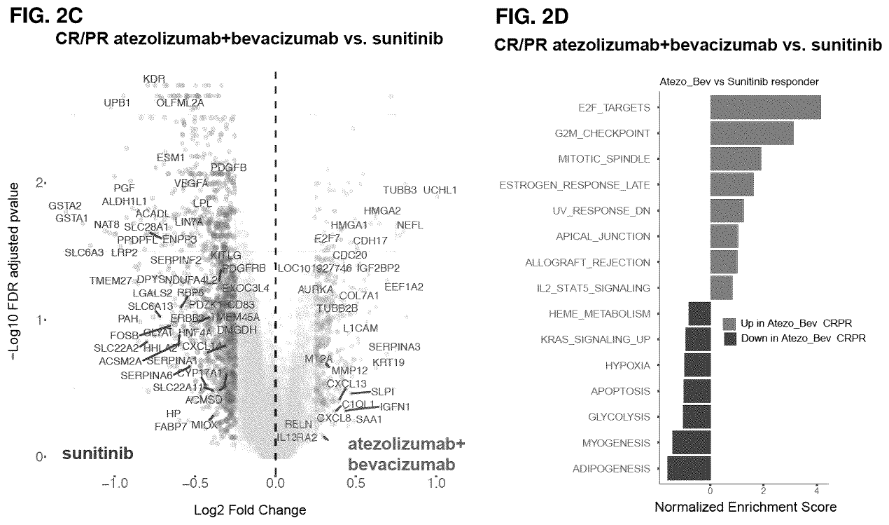

To validate molecular subtypes derived in IMmotion151, the random forest machine learning algorithm (R package randomForest) was used to derive a classifier and then predict the NMF clusters in an independent data set (IMmotion150). A random forest classifier involves learning a large number of binary decision trees from random subsets of a training set. These trees in the classifier can then be used in a predication algorithm to identify the similarity of a given sample to a given class in the training set. Before learning the random forest classifier, the data was preprocessed to generate the training set. First, the gene expression matrix in the test and training set was limited to the top 10% most variable genes in IMmotion151 (n=3,072), from which the initial NMF classification was derived. The gene expression values were normalized (z-score transformed) in each set to ensure that the test and training set were on the same scale. Finally, the random forest classifier was learned on the IMmotion151-derived trained data and then the classifier was utilized to predict the NMF classes in IMmotion150. Subsequently, the expression of gene expression signatures assessed in IMmotion151 was evaluated (FIG. 1C) in the NMF clusters identified in IMmotion150 (FIGS. 2A-2D).

x. Quantitative Set Analysis for Gene Expression (QuSAGE)

To understand biological pathways underlying NMF clustering, QuSAGE analysis (R/Bionconductor qusage v 2.18.0) was conducted to compare each cluster to all others, leveraging MSigDb hallmark gene sets to identify enriched pathways within each cluster. Enrichment scores were represented as a heatmap (FIG. 1B).

xi. Gene Signatures and Scores

Gene signatures were defined as follows: Angiogenesis: VEGFA, KDR, ESM1, PECAM1, ANGPTL4, CD34; T-effector: CD8A, EOMES, PRF1, IFNG, and CD274; Fatty Acid Oxidation/AMP-activated protein kinase (FAO/AMPK): CPT2, PPARA, CPT1A, PRKAA2, PDK2, PRKAB1; Cell cycle: CDK2, CDK4, CDK6, BUB1B, CCNE1, POLQ, AURKA, MKI67, CCNB2; Fatty Acid Synthesis (FAS)/Pentose Phosphate: FASN, PARP1, ACACA, G6PD, TKT, TALDO1, PGD; Stroma: FAP, FN1, COL5A1, COL5A2, POSTN, COL1A1, COL1A2, MMP2; Myeloid Inflammation: CXCL1, CXCL2, CXCL3, CXCL8, IL6, PTGS2; Complement Cascade: F2, C1 S, C1R, CFB, C3; Omega Oxidation: CYP4F3, CYP8B1, NNMT, MGST1, MAOA, CYP4F11, CYP4F2, CYP4F12; snoRNA: SNORD38A, SNORD104, SNORD32A, SNORD68, SNORD66, SNORD100. Signature scores were calculated as the median z-score of genes included in each signature for each sample. When summarized by patient group, as in FIG. 1D, log 2-transformed expression data were first aggregated by patient group using the mean, and subsequently converted to a group z-score.

xii. Quantification and Statistical Analysis

All analyses were conducted using Rv3.6.1. Unless otherwise stated, all comparisons for continuous variables use the two-sided Mann-Whitney test (R function wilcox.test) for two groups and the Kruskal-Wallis test (R function kruskal.test) for more than two groups. Dunn's post-hoc test was applied with Benjamini-Hochberg multiple testing correction for pairwise comparisons. For categorical variables, Pearson's Chi-squared test with continuity correction was used (R function chisq.test). Unless otherwise stated, false discovery rate (FDR)-adjusted p-values are reported. *: p<0.05; **: p<0.01; ***: p<0.001. Survival analyses were conducted using Cox-proportional hazard models using the R survival package (v 3.1.7). Log-rank p-values were reported for survival analyses including more than two groups. For all boxplots, the horizontal line represents the median. The lower and upper hinges correspond to the first and third quartiles. The upper whisker extends from the hinge to the largest value no further than 1.5*IQR from the hinge (where IQR is the inter-quartile range, or distance between the first and third quartiles). The lower whisker extends from the hinge to the smallest value at most 1.5*IQR of the hinge.

### C. Results

i. Patient Cohorts, Biomarker Collection and Validation of Initial Biomarker Findings

The study design and primary clinical findings from IMmotion151 were reported previously (Rini et al. Lancet. 393: 2404-2415 (2019)). Here, integrated RNA-seq and targeted somatic variant analysis using pre-treatment tumor samples from this study are reported. Baseline tumors from 823/915 (90%) patients were available for biomarker evaluation (Table 4). This subset comprised 625 primary and 198 metastatic tumors, all of which were collected no longer than two years prior to enrollment in the study. Of these, 688 tumors were of clear cell histology without a sarcomatoid component, 110 tumors were of clear cell histology with any sarcomatoid component, 1 tumor was of clear cell histology with unknown sarcomatoid component, and 24 tumors were of non-clear cell histology with any sarcomatoid component. In these exploratory analyses, biomarker associations with objective response (OR) and progression free survival (PFS) were evaluated, as these clinical outcomes capture the immediate effect of therapeutic intervention and are less affected than OS by subsequent treatments.

TABLE 4
Patient Characteristics

| | | | RNAseq | | RNAseq/ | | |
|---|---|---|---|---|---|---|---|
| | ITT | | BEP | | FMI BEP | | |
| Variable | n (%) | | n (%) | | n (%) | | p-value |
| All Patients | 915 | | 823 | | 702 | | N/A |
| Age | | | | | | | |
| Median age | 61 | (18-88) | 61 | (18-88) | 61 | (18-84) | N/A |
| (years, range) | | | | | | | |
| Sex | | | | | | | |
| Male | 669 | (73) | 594 | (72) | 513 | (73) | >0.05 |
| Female | 246 | (27) | 229 | (28) | 189 | (27) | |
| Race | | | | | | | |
| White | 660 | (72) | 596 | (72) | 516 | (73) | >0.05 |
| Black | 5 | (1) | 5 | (1) | 4 | (1) | |
| Asian | 171 | (19) | 157 | (19) | 128 | (18) | |
| Other | 79 | (8) | 65 | (8) | 54 | (8) | |
| Liver Metastasis | | | | | | | |
| Yes | 169 | (18) | 154 | (19) | 131 | (19) | >0.05 |
| No | 746 | (82) | 669 | (81) | 571 | (81) | |
| MSKCC Risk Score | | | | | | | |
| Favorable | 179 | (19) | 156 | (19) | 134 | (19) | >0.05 |
| Intermediate | 629 | (69) | 573 | (70) | 498 | (71) | |
| Poor | 107 | (12) | 94 | (11) | 70 | (10) | |
| IMDC Risk Score | | | | | | | |
| Favorable | 202 | (22) | 176 | (21) | 151 | (22) | >0.05 |
| Intermediate | 560 | (61) | 513 | (62) | 444 | (63) | |
| Poor | 153 | (17) | 134 | (17) | 107 | (15) | |
| Sarcomatoid component | | | | | | | |
| Yes | 142 | (16) | 134 | (16) | 120 | (17) | >0.05 |
| No | 772 | (84) | 688 | (84) | 581 | (83) | |

ITT, intent to treat;
BEP, biomarker evaluable population;
N/A, not applicable;
MSKCC, Memorial Sloan Kettering Cancer Center;
IMDC, International Metastatic Renal Cell Carcinoma Database Consortium.

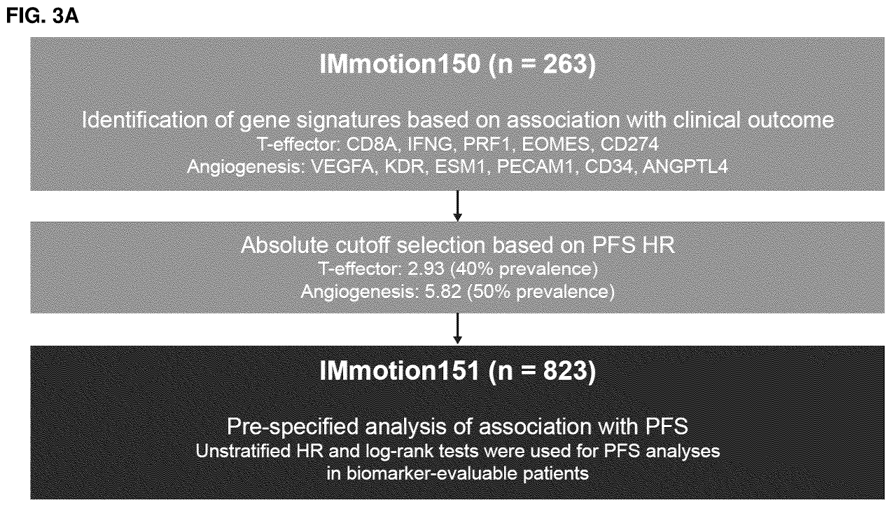

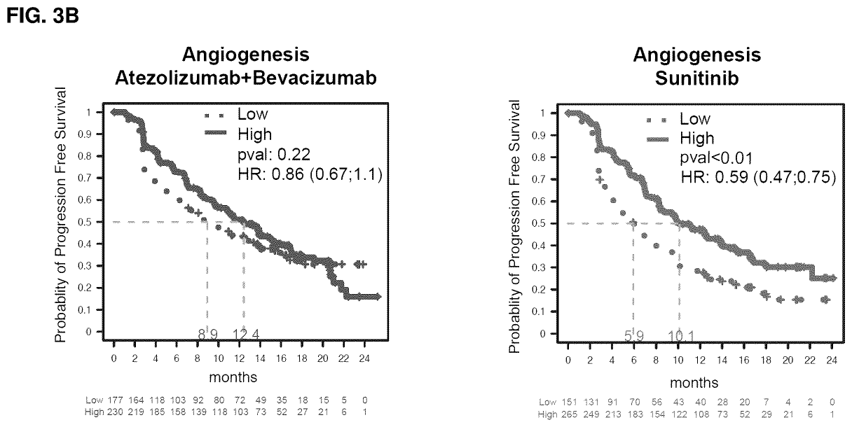

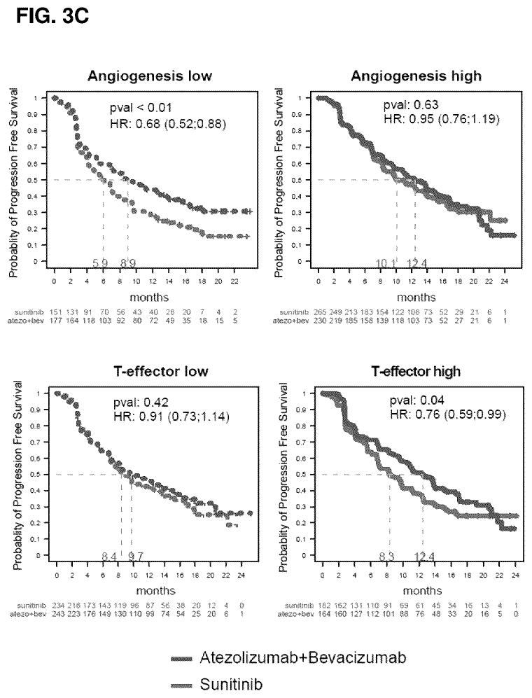

Previous reports describe the associations between Angiogenesis and T-effector gene expression signatures and clinical outcome to treatment with atezolizumab+bevacizumab or sunitinib in the randomized Phase II trial IMmotion150 (McDermott et al. Nat Med. 24: 749-757 (2018)). The association of these signatures with clinical outcomes in IMmotion151 were evaluated by pre-determining transcriptional cutoffs for both signatures in IMmotion150 and retrospectively applying them in IMmotion151 to define high and low expression patient subsets (FIG. 3A). Supporting observations in IMmotion150, high expression of the Angiogenesis signature was associated with improved PFS in the sunitinib treatment arm (HR=0.59, 95% CI 0.47, 0.75, FIG. 3B). When compared across treatment arms, no difference in PFS was observed in the Angiogenesis^{high} or T-effector^{low} tumors. Atezolizumab+bevacizumab improved PFS vs. sunitinib in T-effector^{high} (HR=0.76, 95% CI 0.59-0.99) and in Angiogenesis^{low} (HR=0.68, 95% CI 0.52-0.88) tumors (FIG. 3C). These findings underscore the relevance of immune and angiogenesis biology as reproducible biomarkers of differential clinical outcomes to checkpoint and angiogenesis blockade in independent advanced RCC cohorts.

ii. Identification and Characterization of Seven Molecular Subtypes of Clear Cell Renal Cell Carcinoma (ccRCC) Tumors

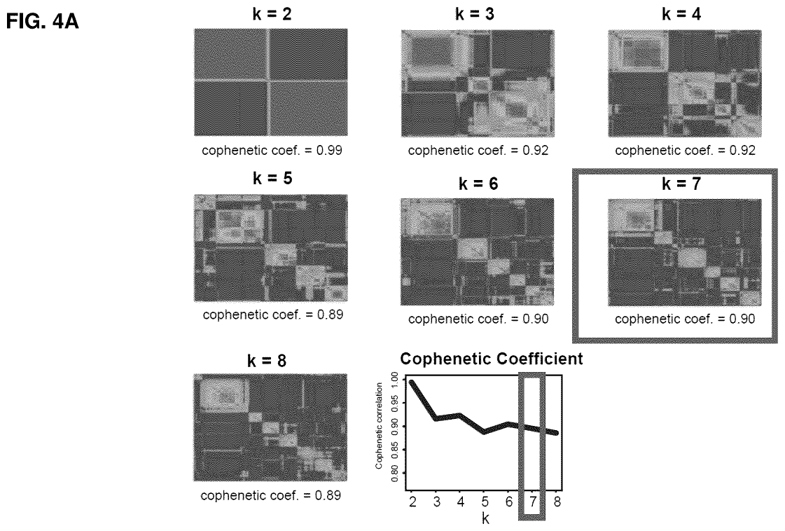

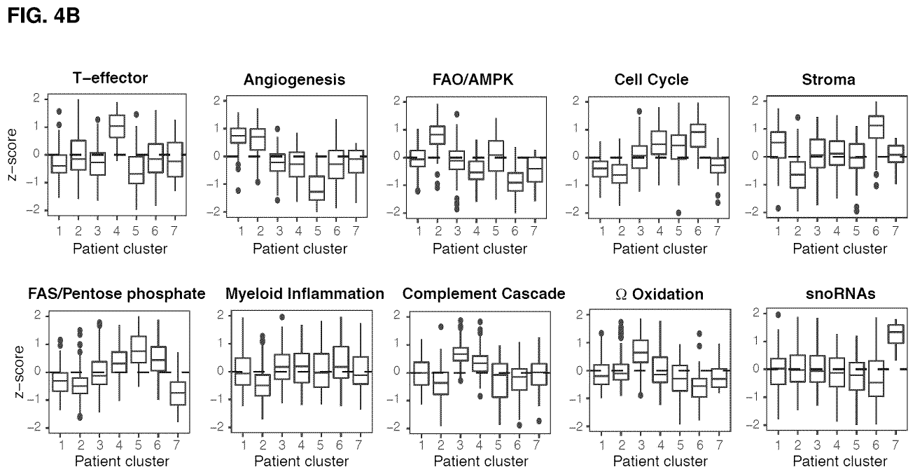

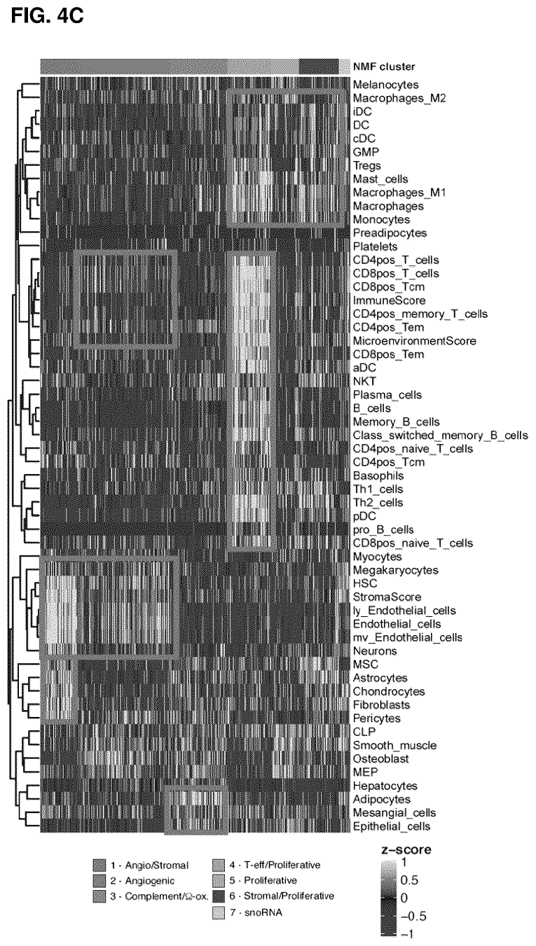

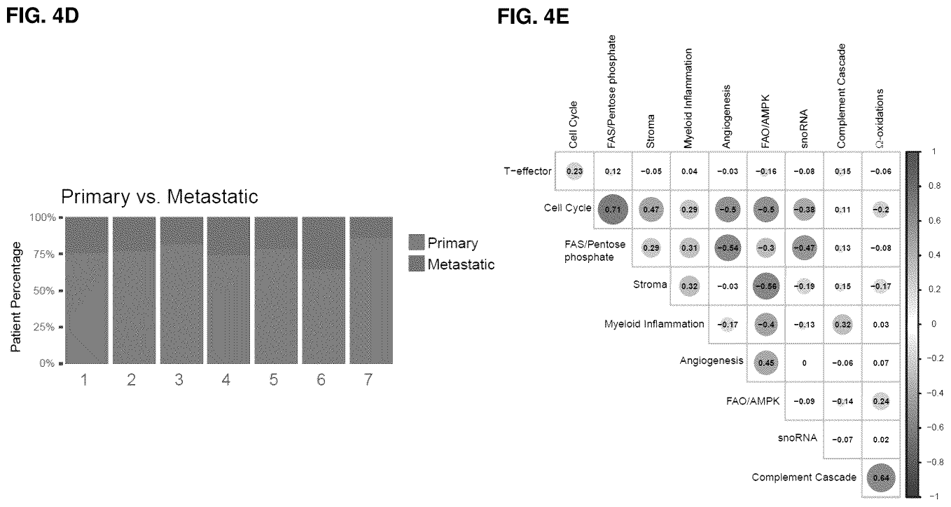

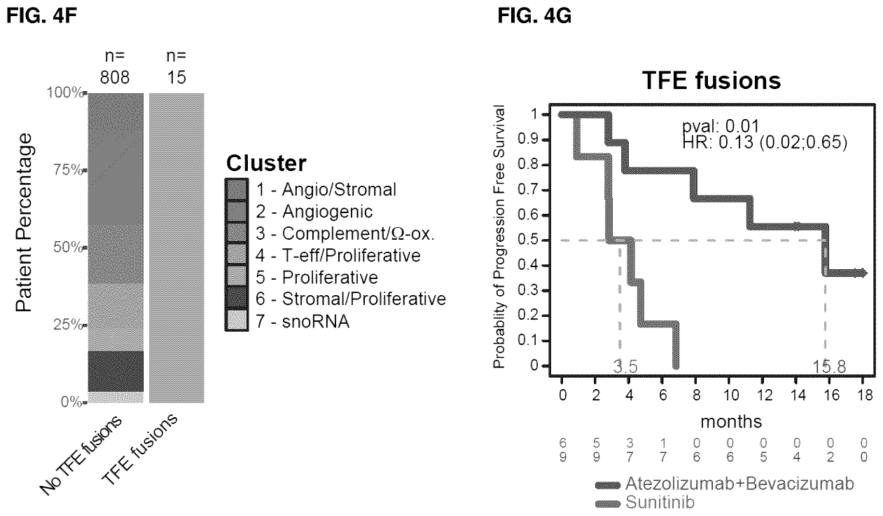

To expand the understanding of the biology of RCC, the large IMmotion151 RNA-seq data set was leveraged to further identify and refine transcriptionally-defined subgroups of patients in an unbiased manner by utilizing non-negative matrix factorization (NMF). NMF is an unsupervised clustering algorithm that iteratively selects the most robust clustering pattern within a given dataset (Brunet et al. Proc Natl Acad Sci USA. 101: 4164-4169 (2004)). Here, NMF identified seven clusters of patients based on the top 10% (3074) most variable genes in the IMmotion151 cohort (FIGS. 1A and 4A).

To understand the main biological features driving these clusters, the clusters were compared individually to all others using quantitative set analysis for gene expression (QuSAGE) (Yaari et al. Nucleic Acids Res. 41: e170 (2013)), leveraging hallmark gene sets from the Molecular Signatures Database (MSigDb) (Liberzon et al. Cell Syst. 1: 417-425 (2015)) combined with the previously described angiogenesis, T-effector, and myeloid inflammation signatures (McDermott et al. Nat Med. 24: 749-757 (2018)) (FIG. 1B). This analysis was complemented with differential gene expression (DGE) analysis, again contrasting each cluster to all others, and conducting pathway enrichment analysis using gene sets from the Reactome database (Fabregat et al. Nucleic Acids Res. 46: D649-D655 (2018)). To summarize these pathway-level analyses and further refine discriminatory transcriptomic profiles, simplified signatures were derived consisting of representative genes associated with cell cycle, stroma, the complement cascade, small nucleolar RNAs (snoRNAs), and metabolism-related pathways including fatty acid oxidation (FAO)/AMPK signaling, fatty acid synthesis (FAS)/pentose phosphate and biological oxidation pathways that complemented the initial T-effector, angiogenesis and myeloid inflammation signatures. These transcriptional programs were summarized across patient clusters both at the gene-(FIG. 1C) and signature-levels (FIGS. 1D and 4B). In addition, xCell (Aran et al. Genome Biol. 18: 220 (2017)) was applied to infer relative frequency of immune and stromal cell types across the tumor transcriptomes (FIG. 4C).

Patient tumors in NMF-derived clusters 1 (n=98, 12%) and 2 (n=245, 30%) were primarily characterized as highly angiogenic, with enrichment of vascular and VEGF pathway-related genes (FIGS. 1B-1D) as well as inferred endothelial cell presence (FIG. 4C). These clusters also exhibited high expression of TGF-β, WNT, hedgehog and NOTCH signaling modules (FIG. 1B). Cluster 1 differentiated from cluster 2 by higher stroma-specific expression (FIGS. 1C, 1D, and 4C), exemplified by high degree of fibroblast-derived gene expression (FIG. 4C), and elevated expression of collagens and activated stroma-associated genes (FAP, FN1, POSTN, MMP2). Cluster 2 additionally showed moderate T-effector gene signature expression, low cell cycle-associated genes, and higher expression of genes associated with catabolic metabolism, including those in fatty acid oxidation (CPT2, PPARA, CPT1A) and AMPK (PRKAA2, PDK2, PRKAB1) pathways. Thus, cluster 1 was labeled as Angiogenic/Stromal, and cluster 2 was labeled as Angiogenic.

Tumors in cluster 3 (n=156, 19%) were characterized by relatively lower expression of both angiogenesis and immune genes and moderate expression of cell cycle genes. These tumors showed elevated expression of genes associated with the complement cascade (C3, CIS, C1R), which has been associated with poor prognosis in the ccRCC TCGA cohort (Roumenina et al. Nat Rev Cancer. 19: 698-715 (2019)), as well as genes associated with the cytochrome P450 family, which is involved in omega oxidation. This cluster was labeled as the Complement/n-oxidation cluster.

Tumors in clusters 4 (n=116, 14%), 5 (n=74, 9%), and 6 (n=106, 13%) were characterized by enrichment of cell cycle transcriptional programs (G2M, E2F targets, MYC targets), and lower expression of angiogenesis-related genes. Mutual exclusion was observed between the angiogenesis signature enriched in clusters 1 and 2 and the cell cycle signature (including the cyclin-dependent kinases CDK2, CDK4, CDK6) enriched in clusters 4, 5 and 6 (FIGS. 1C and 1D), which was confirmed by correlation analysis (R=−0.50, p<0.001; FIG. 4E). Clusters 4, 5, and 6 also exhibited an anabolic metabolism transcriptomic profile, with higher expression of genes associated with FAS (FASN, PARP1, ACACA) and the pentose phosphate pathway (TKT, TALDO1, PGD), which may be related to the proliferative nature of these tumors. Tumors in cluster 4 were additionally characterized as highly immunogenic, exhibiting strong enrichment in T-effector, JAK/STAT, and interferon-α and -γ gene expression modules (FIGS. 1B and 1C). These tumors also showed the highest expression of PD-L1 by IHC (FIG. 1E) and highest infiltration of both adaptive and innate immune cell subsets, including CD8+, CD4+, and regulatory T cells, B cells, macrophages, and dendritic cells (FIG. 4C). In contrast, while tumors in clusters 5 and 6 showed enrichment of the myeloid gene signature and innate immune cell presence as inferred from xCell, they exhibited lower expression of T-effector gene signature and inferred T cell presence (FIG. 4C). The expression of FAS/Pentose phosphate pathway-associated genes was highest in cluster 5. Moreover, Cluster 5 included 15 tumors that contained TFE-fusions (12 tumors with TFE3 fusions and 3 tumors with TFEB fusions, FIG. 4F), which have been implicated in mTORC1 signaling, upregulation of cyclin proteins, dysregulation of metabolic pathways, and increased tumor aggressiveness (Brady et al. Elife. 7 (2018); Kauffman et al. Nat Rev Urol. 11: 465-475 (2014)). Cluster 6 showed high expression of the epithelial-mesenchymal transition (EMT) transcriptional module and enrichment of collagen- and fibroblast-associated stromal genes. Cluster 4 was termed as T-effector/Proliferative, cluster 5 as Proliferative, and cluster 6 as Stromal/Proliferative.

Finally, cluster 7 (n=28, 3%) was characterized by enrichment of expression of snoRNA, especially, C/D box snoRNAs (SNORDs). SNORDs have been implicated in alterations of epigenetic and translation programs and have been linked to carcinogenesis (Gong et al. Cell Rep. 21: 1968-1981 (2017)). For example, SNORD66, which was upregulated in this cluster, has been reported to be associated with lung cancer tumorigenesis (Braicu et al. Cancers (Basel). 11 (2019)). The precise role of the overexpressed SNORDs in RCC tumors remains to be characterized. This small cluster was labeled as the snoRNA cluster.

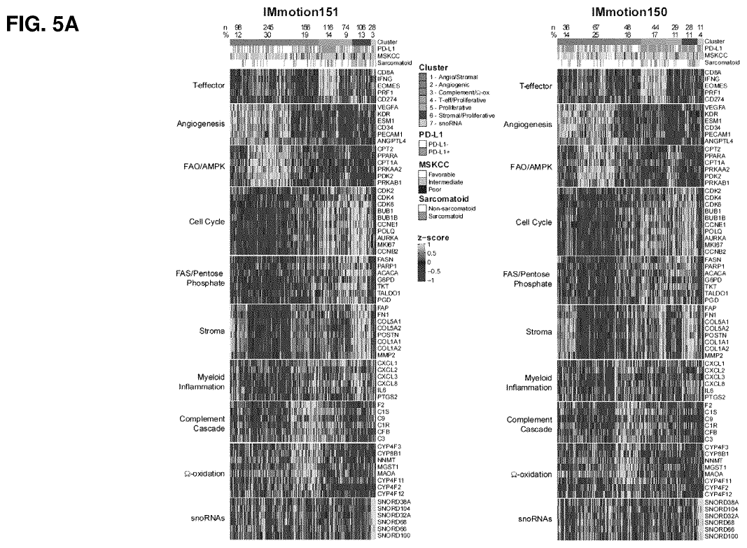

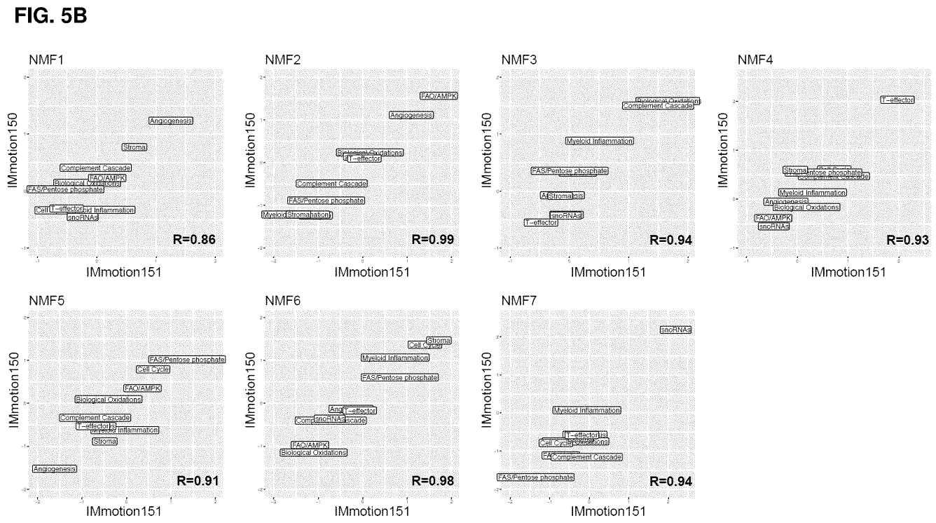

Overall, molecular stratification of 823 RCC tumors identified seven groups of patients with biologically distinct transcriptomes. Given that the tumors in IMmotion151 included both primary and metastatic collections, the prevalence of each was evaluated across the seven NMF subsets. As shown in FIG. 4D, metastatic tumors were distributed across all clusters, suggesting that the transcriptional stratification scheme is not primarily driven by the primary or metastatic origin of tumors. To validate these molecular subgroups in an independent cohort, a random forest classifier was trained from the RNA-seq data in IMmotion151 and was used to predict the NMF class of tumors from patients in the IMmotion150 randomized Phase II trial. The observed distribution of the NMF clusters and the transcriptional expression profile of these clusters in IMmotion150 were highly concordant with those in IMmotion151 (FIGS. 5A and 5B), confirming the robustness of these molecular subtypes.

iii. RCC Molecular Subtypes Associate with Prognostic Risk Categories and Differential Clinical Outcomes to Atezolizumab+Bevacizumab and Sunitinib

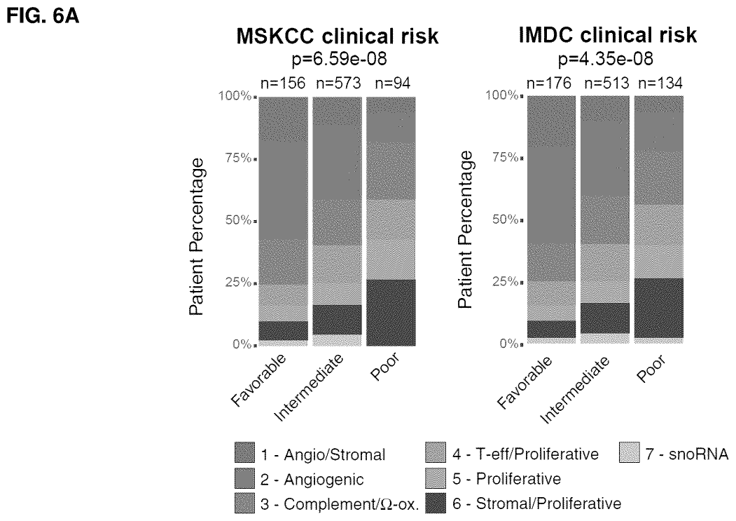

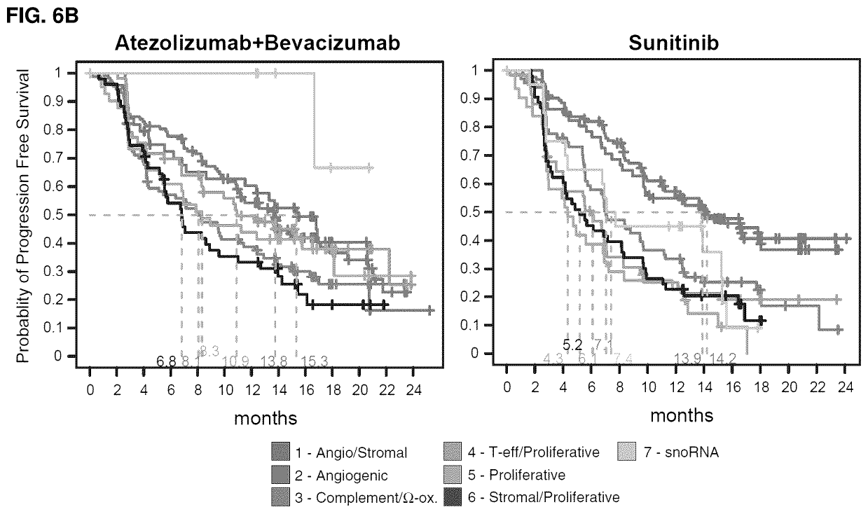

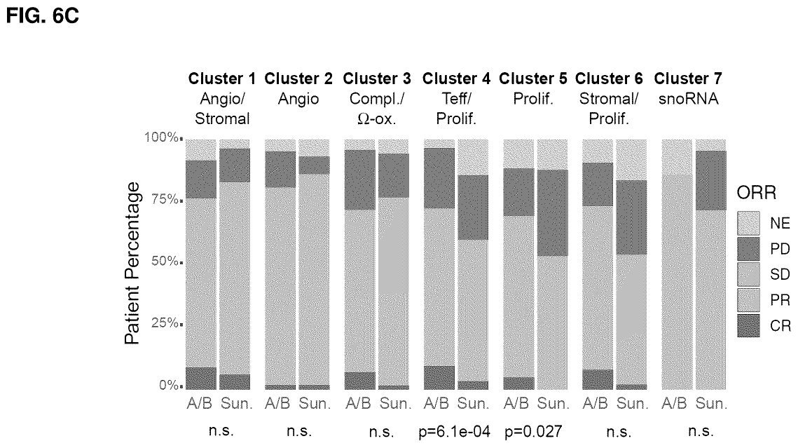

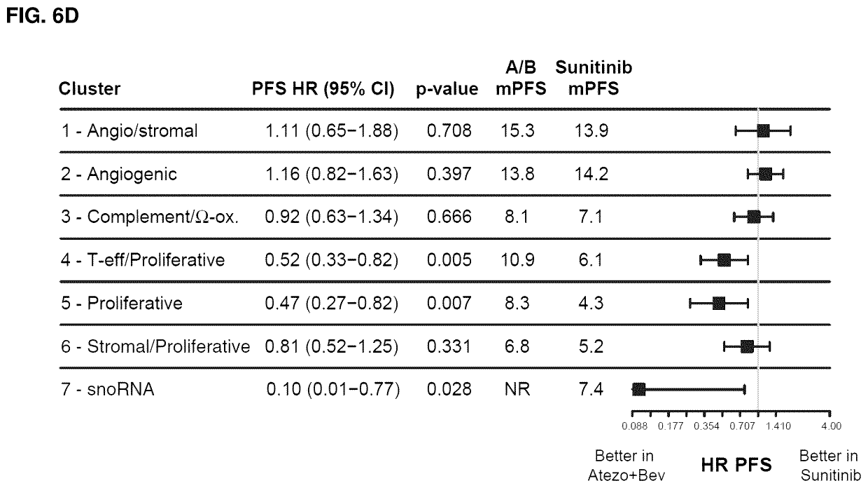

The Memorial Sloan Kettering Cancer Center (MSKCC) and the International Metastatic Renal Cell Carcinoma Database Consortium (IMDC) models are frequently applied in advanced RCC for patient prognostication (Heng et al. J Clin Oncol. 27: 5794-5799 (2009); Motzer et al. J Clin Oncol. 17, 2530-2540 (1999)). These models utilize clinical and laboratory parameters to stratify patients into favorable, intermediate, and poor risk categories. However, the molecular features of tumors associated with these risk categories are incompletely understood. The distribution of the NMF molecular clusters across MSKCC and IMDC risk categories was evaluated, and enrichment of the Angiogenic/Stromal (#1) and Angiogenic (#2) clusters in the favorable risk groups in both classifications was observed. Conversely, the T-effector/Proliferative (#4), Proliferative (#5) and Stromal/Proliferative (#6) clusters were enriched in the poor risk groups (FIG. 6A).

Subsequently, clinical outcomes to atezolizumab+bevacizumab and sunitinib treatment in each cluster were evaluated. Patients in the Angiogenic/Stromal (#1) and Angiogenic (#2) clusters demonstrated longer PFS in both treatment arms, suggesting better outcome regardless of treatment, while those in the Stromal/Proliferative cluster (#5) had relatively shorter PFS (atezolizumab+bevacizumab mPFS: 6.8 months; sunitinib mPFS: 5.2 months), suggesting poor prognostic association of proliferative/stromal biology with clinical outcomes (FIG. 6B).

When evaluated across treatment arms, no apparent difference in clinical outcomes was observed between atezolizumab+bevacizumab and sunitinib arms in the Angiogenic/Stromal (#1), Angiogenic (#2) and Complement/Ω-oxidation (#3) clusters (FIGS. 6C and 6D). Atezolizumab+bevacizumab demonstrated improved objective response rate (ORR, 52.0% vs 19.4%, p<0.001) and PFS (hazard ratio (HR) 0.52, 95% CI 0.33-0.82) vs. sunitinib (FIGS. 6C and 6D) in the T-effector/Proliferative cluster (#4), confirming the contribution of pre-existing intratumoral adaptive immune presence in determining benefit to immunotherapy containing regimens. In addition, atezolizumab+bevacizumab showed improved ORR (26.2% vs 3.1%, p<0.001, FIG. 6C) and PFS (HR 0.47, 95% CI 0.27-0.82, FIG. 6D) in the Proliferative cluster (#5), including in tumors that harbored TFE-fusions (FIG. 4G), implicating the relevance of PD-L1 blockade in this low angiogenesis, but high proliferative subgroup. Atezolizumab+bevacizumab also showed improved PFS (HR 0.1, 95% CI 0.01-0.77) in the snoRNA cluster (#7); however, the biological basis of this effect in this small cluster of patients remains to be elucidated.

Subsequently, the HRs obtained above using cox proportional hazard model that only tests treatment arm in each NMF subgroup were compared against a model that included treatment arm, PD-L1 IHC, and MSKCC clinical risk score. These multivariate analyses confirmed that the differential clinical benefit observed in these NMF clusters is independent of PD-L1 expression and MSKCC prognostic risk (Table 5).

TABLE 5
Univariate vs. Multivariate PFS Hazard Ratios (HR) Comparing
Atezolizumab + Bevacizumab vs. Sunitinib in NMF Clusters

| | | | Multivariate | |
|---|---|---|---|---|
| | Univariate | | Treatment arm + | |
| | Treatment arm | | PD-L1 + MSKCC | |
| | PFS HR | p-value | PFS HR | p-value |
| Stromal/Angiogenic | 1.110 | 0.708 | 1.174 | 0.562 |
| (Cluster 1) | | | | |
| Angiogenic (Cluster 2) | 1.160 | 0.397 | 1.092 | 0.613 |
| Complement/Ω-oxidation | 0.920 | 0.666 | 0.894 | 0.558 |
| (Cluster 3) | | | | |
| T-effector/Proliferative | 0.520 | 0.005 | 0.515 | 0.005 |
| (Cluster 4) | | | | |
| Proliferative (Cluster 5) | 0.470 | 0.007 | 0.467 | 0.007 |
| Stromal/Proliferative | 0.810 | 0.331 | 0.847 | 0.457 |
| (Cluster 6) | | | | |
| snoRNA (Cluster 7) | 0.100 | 0.028 | 0.088 | 0.025 |

Finally, differentially expressed genes between responders (complete or partial objective response, CR/PR) and non-responders (progressive disease, PD) within and across treatment arms were additionally evaluated. In sunitinib-treated patients, linear modeling complemented with MSigDb hallmark gene set enrichment analysis revealed higher expression of genes associated with VEGF pathway in tumors from responders and higher expression of cell cycle-associated pathways in tumors from non-responders (FIGS. 2A and 2B). Comparison of gene expression in responders with non-responders treated with atezolizumab+bevacizumab did not identify any significantly differentially expressed genes (FDR<0.05). Within responders across treatment arms, genes associated with proliferation and immune pathways were enriched in patients responding to atezolizumab+bevacizumab, while genes associated with VEGF signaling (hypoxia) were enriched in patients responding to sunitinib (FIGS. 2C and 2D). No differentially expressed genes (FDR<0.05) were observed in non-responders treated with atezolizumab+bevacizumab vs. sunitinib. These data confirm and support the findings from the unbiased NMF classification.

iv. Somatic Alterations Associate with Tumor Intrinsic and Extrinsic Transcriptional Profiles

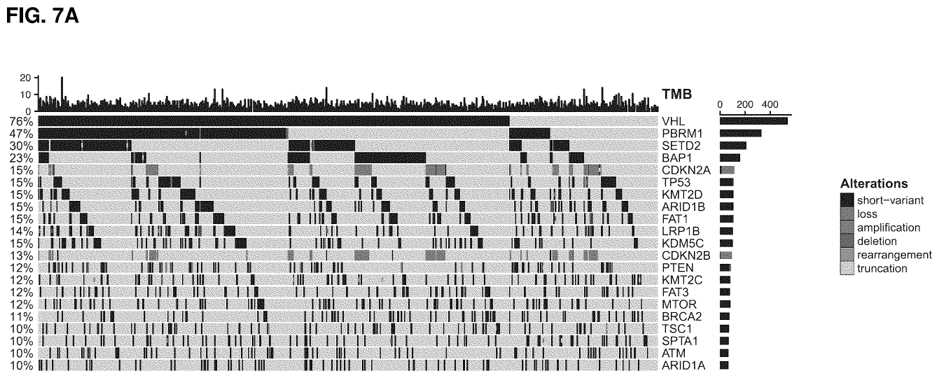

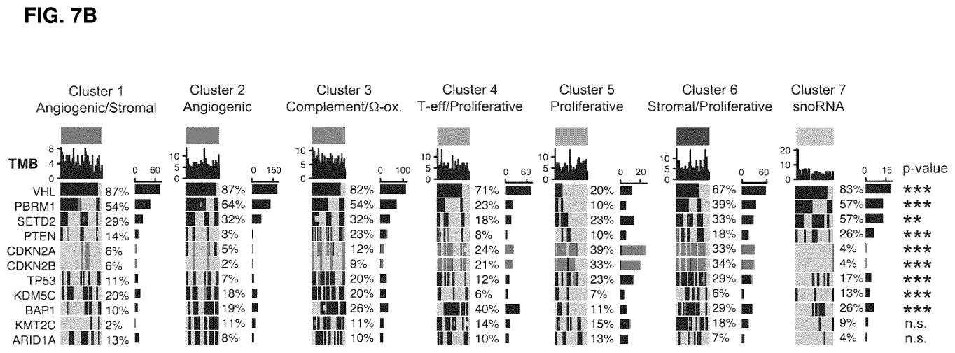

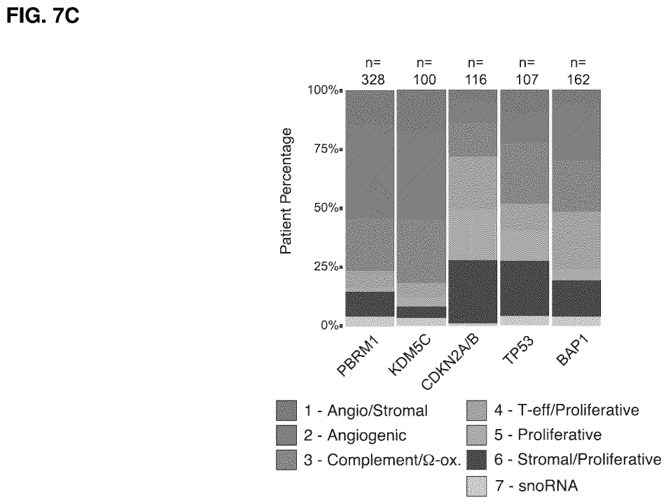

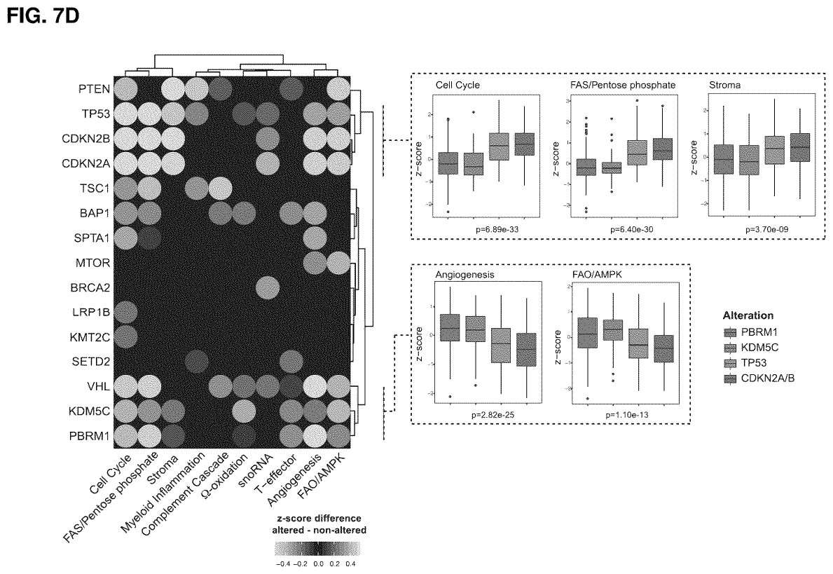

Transcriptional profiling was complemented with evaluation of somatic alterations in tumors from 715 patients. The pattern and prevalence of somatic alterations in this cohort were broadly in alignment with prior reports of recurrent gene alterations in RCC tumors (FIGS. 7A and BA) (Cancer Genome Atlas Research. Nature. 499: 43-49 (2013); Chen et al. Cell Rep. 14:2476-2489(2016); Ricketts et al. Cell Rep. 23: 3698 (2018)).

Previous studies have reported differences in genomic alteration profiles between primary and metastatic tumors, including enrichment of loss of chromosome 9p21.3 in metastatic lesions compared to primary tumors (Turajlic et al. Cell. 173: 581-594, e52 (2018)). In the IMmotion051 cohort, while no genes were exclusively expressed in metastatic tumors, the frequency of genomic alterations in 12 genes, including CDKN2A1B (23.8% vs 14.6%, p=0.011), BRCA2 (15.7% vs 9.2%, p=0.034), ZNF216 (12.2% vs 6.3%, p=0.025) and NF2 (10.9% vs 5.6%, p=0.036) was increased in metastatic tumors compared to primary tumors (Table 6).

TABLE 6
Genomic Alterations in Primary vs. Metastatic Tumors

| | Primary | Primary | | Metastasis | Metastasis | | chi- | chi- |
|---|---|---|---|---|---|---|---|---|
| | non-altered | altered | Primary | non-altered | altered | Metastasis | square | square |
| Gene | (n) | (n) | % | (n) | (n) | % | statistic | p-value |
| CDKN2A/B | 474 | 81 | 14.59 | 112 | 35 | 23.81 | 6.5 | 0.011 |
| EGFR | 544 | 11 | 1.98 | 138 | 9 | 6.12 | 5.78 | 0.016 |
| NTRK2 | 548 | 7 | 1.26 | 140 | 7 | 4.76 | 5.61 | 0.018 |
| TIPARP | 553 | 2 | 0.36 | 143 | 4 | 2.72 | 5.11 | 0.024 |
| ZNF217 | 520 | 35 | 6.31 | 129 | 18 | 12.24 | 5.05 | 0.025 |
| STAT4 | 551 | 4 | 0.72 | 142 | 5 | 3.40 | 4.65 | 0.031 |
| MAP2K4 | 551 | 4 | 0.72 | 142 | 5 | 3.40 | 4.65 | 0.031 |
| MEN1 | 549 | 6 | 1.08 | 141 | 6 | 4.08 | 4.57 | 0.033 |
| BRCA2 | 504 | 51 | 9.19 | 124 | 23 | 15.65 | 4.48 | 0.034 |
| NF2 | 524 | 31 | 5.59 | 131 | 16 | 10.88 | 4.41 | 0.036 |
| ZNRF3 | 542 | 13 | 2.34 | 138 | 9 | 6.12 | 4.3 | 0.038 |
| ERCC4 | 544 | 11 | 1.98 | 139 | 8 | 5.44 | 4.05 | 0.044 |

Alterations that showed statistically different prevalence (Chi square test, p<0.05) are shown.

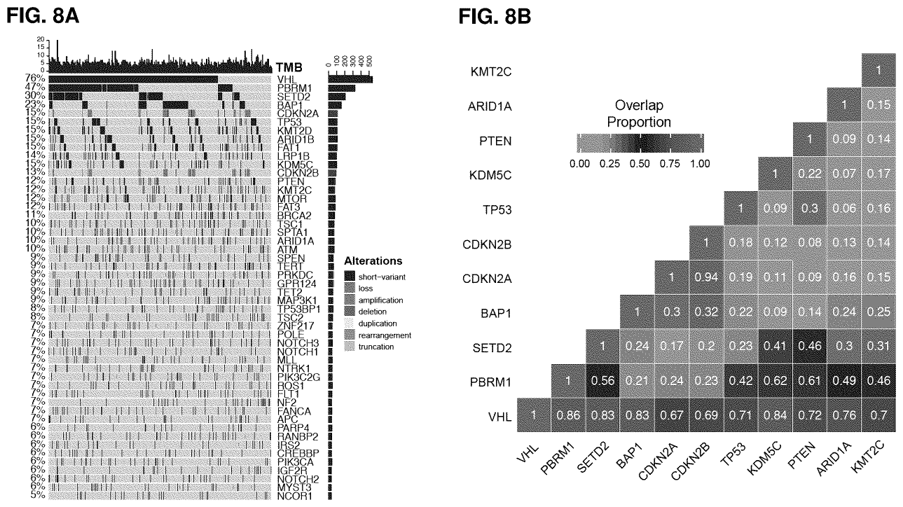

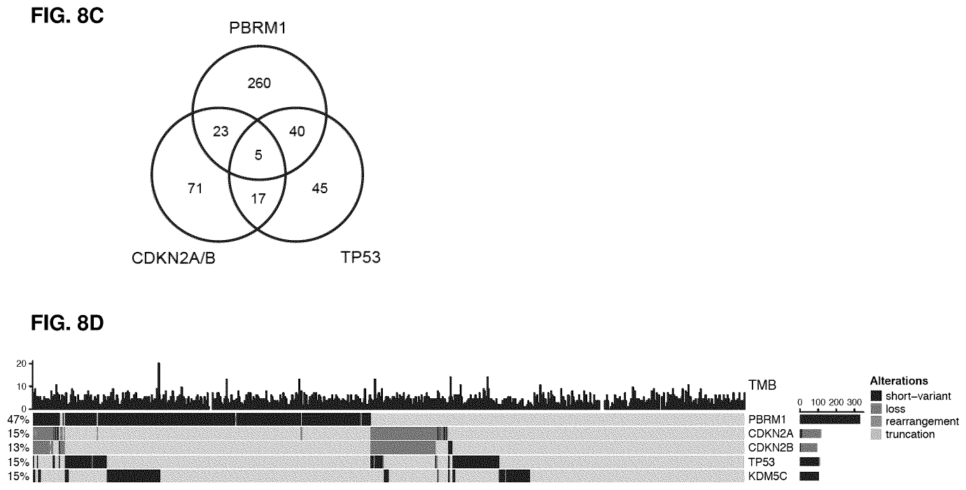

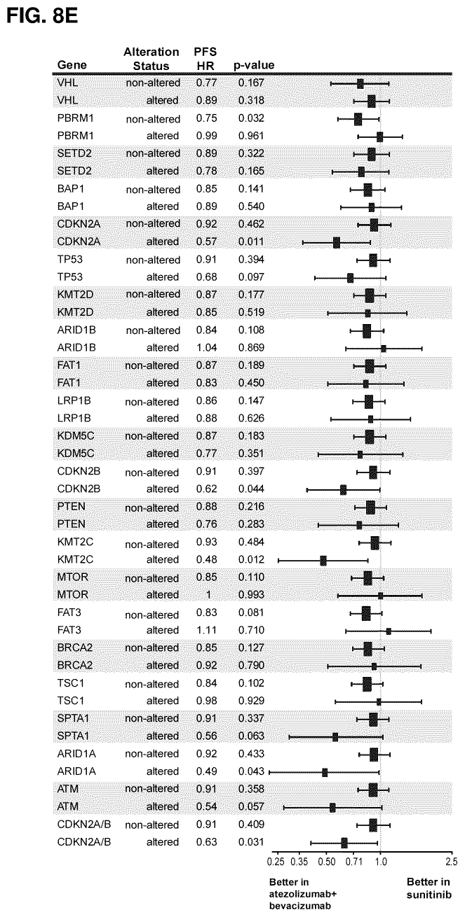

Co-occurrence analysis showed >50% overlap of SETD2, KDM5C, or PTEN alterations with PBRM1 mutations (FIG. 8B1). Conversely, mutations in PBRM1, BAP1, and CDKN2A/B were largely non-overlapping (<25% overlap, hypergeometric p=9.5e-09, FIGS. 8B1-8D), supporting models of distinct tumor lineages associated with PBRM1 vs. BAP1 mutations (Kapur et al. Lancet Oncol. 14: 159-167 (2013); Pena-Llopis et al. Nat Genet. 44: 751-759 (2012)) and further suggesting evolutionary distinctions between tumors harboring 3p associated aberrations only versus those that also have 9p arm level or focal copy number alterations (Turajlic et al. Cell. 173, 595-610, e511 (2018)). Additionally, CDKN2A/B alterations were non-overlapping with TP53 mutations (<20% overlap, FIGS. 8B and 8C).

The prevalence of the top altered genes in each NMF cluster was further characterized, and the observations showed lower prevalence of PBRM1 mutations (p<0.001) and enrichment of CDKN2A/B alterations (p<0.001) in the T-effector/Proliferative (#4), Proliferative (#5) and Stromal/Proliferative (#6) clusters (FIG. 7B). The prevalence of TP53 mutations was highest in the Proliferative (#5) and Stromal/Proliferative (#6) clusters (p<0.001) and that of BAP1 mutations was highest in the T-effector/Proliferative cluster (#4) (p<0.01) (FIG. 7B). When analyzing cluster distribution by mutation status, the Angiogenic cluster (#2) was enriched in PBRM1 and KDMSC mutants, while the Proliferative (#5) and Stromal/Proliferative (#6) clusters were enriched in CDKN2A/B mutants (FIG. 7C).

Subsequently, evaluations were conducted on the association of somatic alterations present in at least 10% of the tumors with transcriptomic signatures discussed above (FIG. 7D). Compared to non-mutants, tumors with mutations in PBRM1 or KDMSC exhibited higher expression of angiogenesis (PBRM1 p=3.46e-20; KDMSC p=0.001) and FAO/AMPK (PBRM1 p=4.59e-17; KDMSC p=3.79e-05) associated gene signatures, and reduced expression of the cell cycle gene signature (PBRM1 p=7.74e-12; KDMSC p=1.09e-04). In contrast, tumors harboring TP53, CDKN2A/B, and PTEN alterations showed upregulation of cell cycle (TP53 p=1.22e-13; CDKN2A/B p=5.00e-18; PTEN p=3.71e-04), FAS/pentose phosphate pathway (TP53 p=2.52e-09; CDKN2A/B p=1.97e-14), and stromal gene expression (TP53 p=4.69e-04; CDKN2A/B p=8.35e-06; PTEN p=2.46e-07). KMT2C mutations also showed higher expression of cell cycle genes (p=0.022). PTEN alterations were associated with higher myeloid inflammation (p=0.03). BAP1 mutations showed elevated expression of cell cycle (p=0.0028) and T-effector (p=8.64e-04) gene signatures, the latter supporting previously described association of BAP1 mutations with IFN-γ signaling (Clark et al. Cell. 179: 964-983, e931 (2019); Wang et al. Cancer Discov. 8: 1142-1155 (2018)).

Overall, somatic alteration profiles suggest a genetic basis for the distinct transcriptomic profiles in advanced RCC. Functional depletion of PBRM1 and/or KDMSC associate with a subtype typified by angiogenic features, whereas functional depletions of tumor suppressor genes including CDKN2A/B and TP53, associate with high proliferation, anabolic metabolism, and stromal biology (FIG. 7D).

v. Associations Between Somatic Alterations and Clinical Outcome

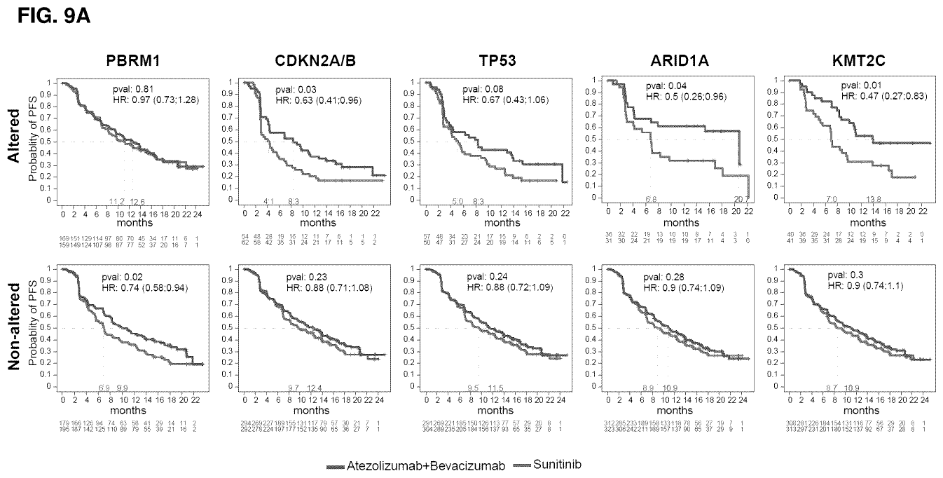

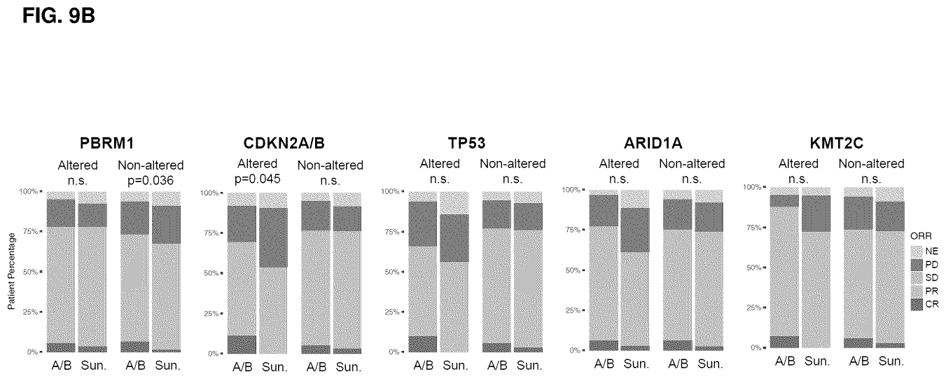

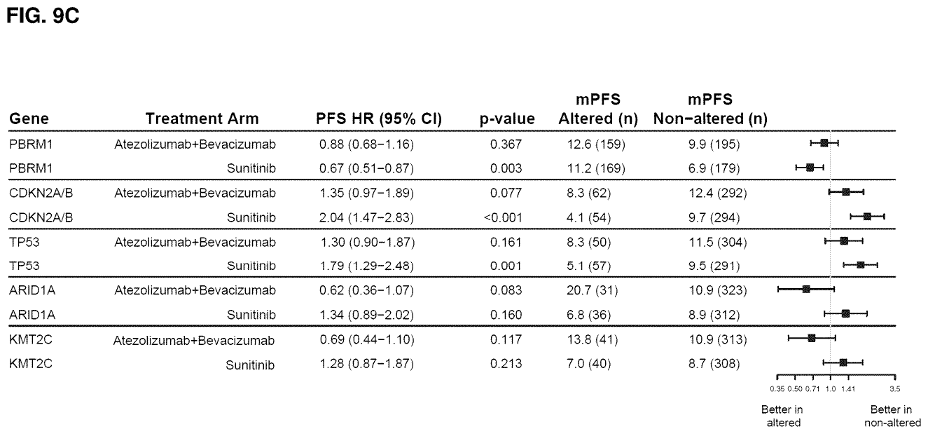

Evaluation of clinical outcomes in somatic alteration subgroups showed that PBRM1 mutations conferred overall better prognosis, regardless of treatment arm (FIGS. 8E, 9A, and 9C). Sunitinib-treated patients whose tumors harbored PBRM1 mutations showed longer PFS compared to those with non-mutant PBRM1 (HR=0.67; 95% CI: 0.51, 0.87; mPFS: 11.2 months vs. 6.9 months). This trend of longer PFS in PBRM1 mutant tumors was also observed in atezolizumab+bevacizumab-treated patients, but did not reach statistical significance. When compared across treatment arms, there was no difference in PFS or ORR in PBRM1 mutated tumors. In patients with PBRM1 non-mutant tumors, atezolizumab+bevacizumab improved PFS (HR=0.74; 95% CI: 0.58-0.94; mPFS atezolizumab+bevacizumab: 9.9 months; mPFS sunitinib: 6.9 months) (FIGS. 8E and 9A) and ORR (40% vs. 27%, p=0.036) (FIG. 9B) vs. sunitinib.

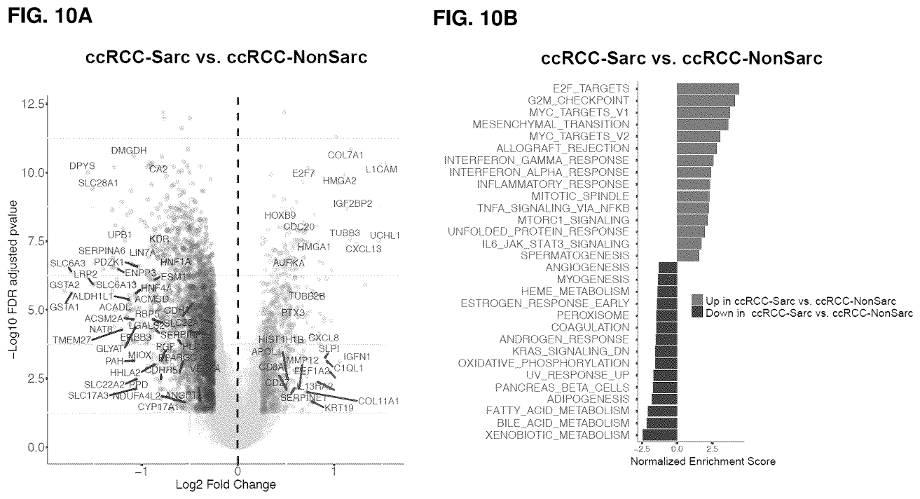

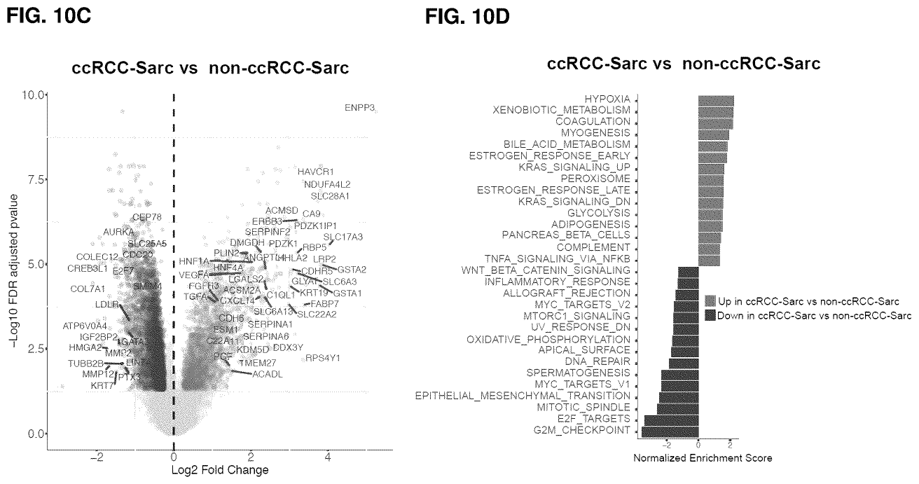

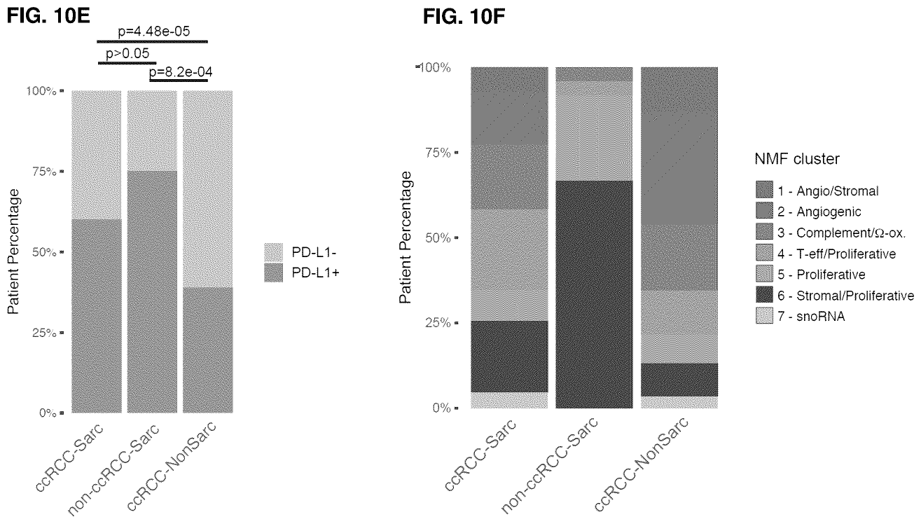

Conversely, CDKN2A/B alterations conferred worse prognosis when compared to non-altered tumors (FIGS. 9A and 9C). When compared across treatment arms, patients whose tumors had CDKN2A/B alterations showed longer PFS (HR=0.63; 95% CI: 0.41-0.96, mPFS: 8.3 months vs. 4.1 months) (FIG. 9A) and higher ORR (42% vs. 20%, p=0.045) (FIG. 9B), including complete responses (11% vs. 0%) when treated with atezolizumab+bevacizumab vs. sunitinib. Patients with TP53 mutant tumors, which were largely non-overlapping with CDKN2A/B altered tumors (FIGS. 10C and 10D), also showed a statistically non-significant trend toward improved clinical benefit with atezolizumab+bevacizumab vs. sunitinib (FIGS. 9A and 9B).

Finally, this analysis revealed that patients with tumors harboring loss-of-function mutations in ARID1A and/or KMT2C had significantly better PFS when treated with atezolizumab+bevacizumab vs. sunitinib (ARID1A HR=0.50; 95% CI: 0.26-0.96; mPFS: 20.7 vs. 6.8 months; KMT2C HR=0.47; 95% CI: 0.27-0.83; mPFS: 13.8 months vs. 7.0 months) (FIGS. 8E, 9A, and 9B).

Overall, five genes were identified with frequent loss-of-function alterations that associate with distinct clinical outcomes to atezolizumab+bevacizumab vs. sunitinib, suggesting that targeted somatic mutation profiling in advanced RCC could help guide treatment selection.

vi. Molecular Characterization of Sarcomatoid RCC Tumors

RCC tumors that include a sarcomatoid component (sRCC) associate with poor prognosis and show limited response to standard-of-care treatment with VEGF pathway inhibitors (Golshayan et al. J Clin Oncol. 27: 235-241 (2009)). Therefore, the molecular characteristics of sRCC tumors that distinguish it from non-sarcomatoid RCC (non-sRCC) tumors were subsequently examined.

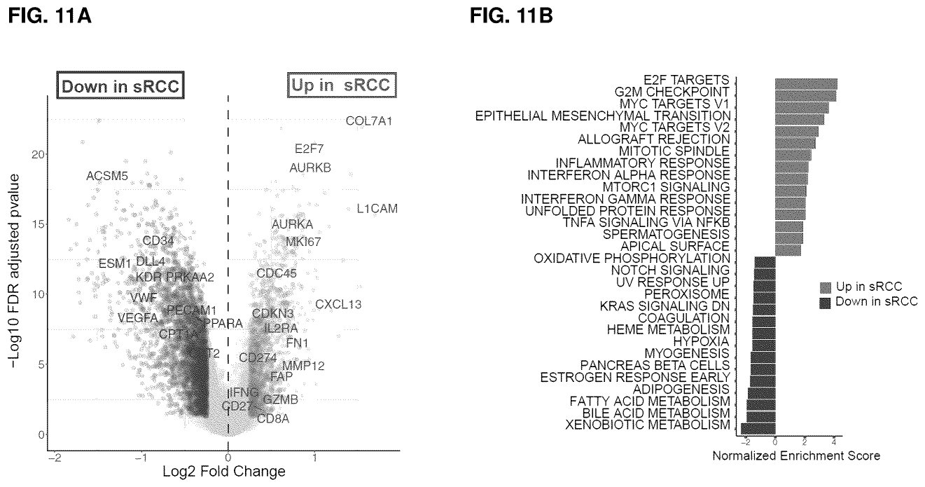

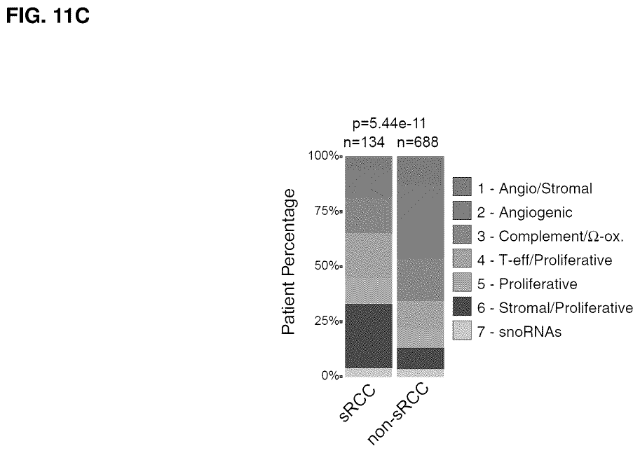

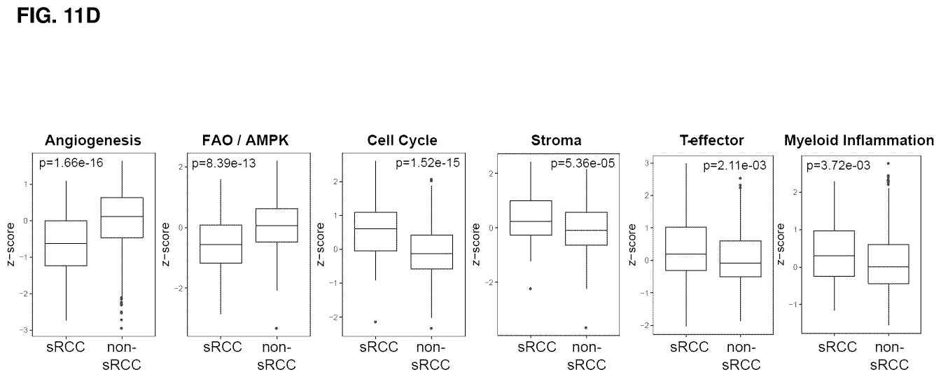

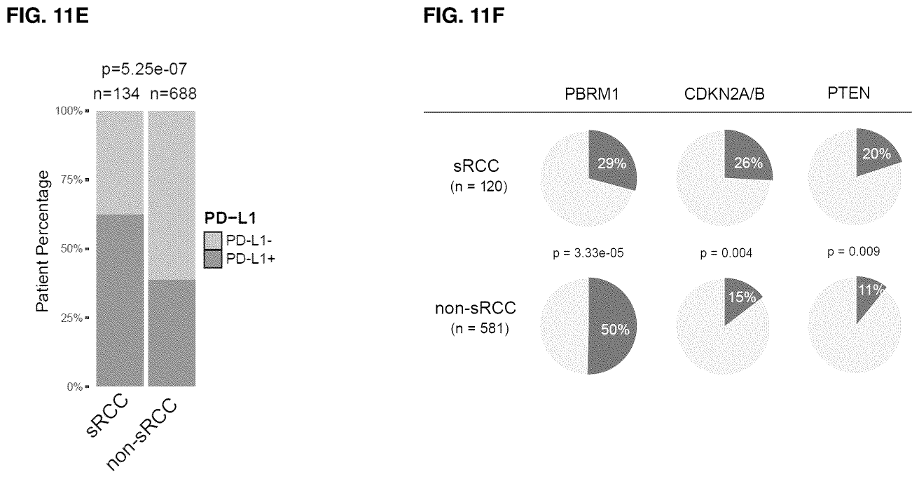

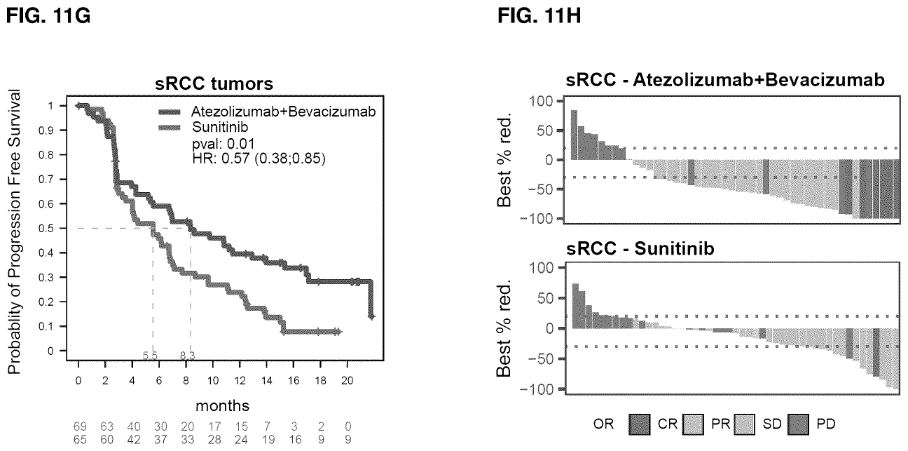

DGE analysis (FDR<0.05) identified 2917 overexpressed and 6309 under expressed genes in sRCC compared to non-sRCC tumors (FIG. 11A). Gene set enrichment analysis demonstrated enrichment of transcriptional pathways involved in cell cycle/proliferation (E2F targets, G2M checkpoints, MYC targets, EMT and immune response (Allograft rejection, Interferon gamma response, Inflammatory response) and lower expression of genes involved in the VEGF pathway (Angiogenesis, Hypoxia) (FIG. 11B) in sRCC. The distribution of sRCC and non-sRCC tumors in the transcriptomic NMF clusters were further compared, and it was observed that sRCC tumors were enriched in the T-effector/Proliferative (#4), Proliferative (#5) and Stromal/Proliferative (#6) clusters, and were less prevalent in the Angiogenic/Stromal (#1) and Angiogenic (#2) clusters (FIG. 11C). Moreover, evaluation of gene expression signatures confirmed lower expression of angiogenesis and FAO/AMPK signatures and higher expression of cell cycle, stromal, T-effector, and myeloid signatures in sRCC tumors compared to non-sRCC tumors (FIG. 11D).

PD-L1 protein prevalence was significantly higher in sRCC vs. non-sRCC (63% vs 39%, p<0.001, FIG. 11E), confirming the increased presence of IFN-γ response observed by gene expression analysis, and reflective of adaptive upregulation of PD-L1 by IFN-γ in sRCC.

Somatic alteration analysis revealed lower prevalence of PBRM1 (29% vs 50%, p=3.33e-05) mutations in sRCC, which suggests a genomic basis for the observed lower angiogenesis gene expression in these tumors. Conversely, the prevalence of CDKN2A/B (26% vs 15%, p=0.004), and PTEN (20% vs 11%, p=0.009) alterations was significantly higher in sRCC, suggesting that somatic loss-of-function in these genes may contribute to the aggressive phenotype of sarcomatoid tumors (FIG. 11F).

Given the differences in etiology between ccRCC and non-ccRCC, molecular features between ccRCC non-sarcomatoid (ccRCC-NonSarc), ccRCC-Sarc, and non-ccRCC-Sarc tumors were compared. ccRCC-Sarc tumors showed enrichment of pathways associated with cell cycle/proliferation and immune response, and lower expression of genes associated with angiogenesis and hypoxia compared to ccRCC-NonSarc tumors (FIGS. 10A and 10B). This is noteworthy, as it confirms that the downregulation of angiogenesis pathways in the overall sarcomatoid subset (sRCC) is independent of non-ccRCC-Sarc tumors.

DGE analysis (FDR<0.05) comparing the two subsets of sarcomatoid tumors (ccRCC-Sarc vs. non-ccRCC-Sarc) (FIGS. 10C and 10D) showed upregulation of VEGF pathway-associated genes (hypoxia) in ccRCC-Sarc tumors and higher expression of cell cycle/proliferation pathways (G2M, E2F targets, EMT, MYC targets) in non-ccRCC-Sarc tumors. Compared with ccRCC-NonSarc tumors, PD-L1 expression was enriched in both ccRCC-Sarc and non-ccRCC-Sarc tumors (FIG. 10E).

Comparison of the distribution of NMF clusters in the histological subtypes showed that ccRCC-Sarc tumors were enriched in T-effector/Proliferative (#4) and Stromal/Proliferative (#5) clusters, and non-ccRCC-Sarc tumors were enriched in Proliferative (#5) and Stromal/Proliferative (#6) clusters (FIG. 10F).

Evaluation of somatic alterations across the three histological subtypes (Table 7) confirmed higher prevalence of VHL mutations in ccRCC subtypes reported in previous studies. The prevalence of PBRM1 mutations was lower and that of CDKN2A/2B and PTEN alterations was higher in ccRCC-Sarc and non-ccRCC-Sarc tumors compared to ccRCC-NonSarc tumors. Prevalence of BAP1 mutations was highest in ccRCC-Sarc, whereas non-ccRCC-Sarc showed enrichment in TP53 and RB1 alterations.

TABLE 7
Genomic Alterations in Sarcomatoid Subsets

| | ccRCC-NonSarc | | | ccRCC-Sarc | | | Non-ccRCC-Sarc | | | p-value | p-value | |
|---|---|---|---|---|---|---|---|---|---|---|---|---|
| | Non- | | | Non- | | | Non- | | | ccRCC_{—} | ccRCC_Sarc | |
| | altered | Altered | % | altered | Altered | % | altered | Altered | % | nonSarc vs. | vs. non- | |
| Gene | (n) | (n) | Altered | (n) | (n) | Altered | (n) | (n) | Altered | ccRCC_Sarc | ccRCC_Sarc | all p-value |
| VHL | 124 | 457 | 78.7% | 29 | 70 | 70.7% | 18 | 3 | 14.3% | 1.05E−01 | 4.99E−06 | 6.11E−11 |
| PBRM1 | 288 | 293 | 50.4% | 65 | 34 | 34.3% | 20 | 1 | 4.8% | 4.34E−03 | 1.45E−02 | 5.70E−06 |
| BAP1 | 461 | 120 | 20.7% | 58 | 41 | 41.4% | 20 | 1 | 4.8% | 1.28E−05 | 3.21E−03 | 4.51E−06 |
| CDKN2A/2B | 496 | 85 | 14.6% | 73 | 26 | 26.3% | 16 | 5 | 23.8% | 6.00E−03 | 1.00E+00 | 1.05E−02 |
| TP53 | 500 | 81 | 13.9% | 84 | 15 | 15.2% | 10 | 11 | 52.4% | 8.70E−01 | 5.21E−04 | 9.38E−06 |
| FAT3 | 517 | 64 | 11.0% | 87 | 12 | 12.1% | 15 | 6 | 28.6% | 8.81E−01 | 1.14E−01 | 4.81E−02 |
| PTEN | 518 | 63 | 10.8% | 80 | 19 | 19.2% | 16 | 5 | 23.8% | 2.85E−02 | 8.57E−01 | 1.82E−02 |
| SPTA1 | 529 | 52 | 9.0% | 82 | 17 | 17.2% | 17 | 4 | 19.0% | 2.01E−02 | 1.00E+00 | 1.97E−02 |
| TERT | 533 | 48 | 8.3% | 87 | 12 | 12.1% | 15 | 6 | 28.6% | 2.89E−01 | 1.14E−01 | 4.53E−03 |
| MAP3K1 | 539 | 42 | 7.2% | 85 | 14 | 14.1% | 17 | 4 | 19.0% | 3.44E−02 | 8.14E−01 | 1.65E−02 |
| RANBP2 | 549 | 32 | 5.5% | 93 | 6 | 6.1% | 17 | 4 | 19.0% | 1.00E+00 | 1.28E−01 | 3.69E−02 |
| TRRAP | 549 | 32 | 5.5% | 98 | 1 | 1.0% | 18 | 3 | 14.3% | 9.45E−02 | 1.60E−02 | 2.69E−02 |
| NF2 | 550 | 31 | 5.3% | 86 | 13 | 13.1% | 19 | 2 | 9.5% | 7.07E−03 | 9.28E−01 | 1.29E−02 |
| ASXL1 | 558 | 23 | 4.0% | 92 | 7 | 7.1% | 18 | 3 | 14.3% | 2.59E−01 | 5.14E−01 | 4.39E−02 |
| SH2B3 | 563 | 18 | 3.1% | 97 | 2 | 2.0% | 18 | 3 | 14.3% | 7.91E−01 | 5.07E−02 | 1.38E−02 |
| FANCF | 564 | 17 | 2.9% | 89 | 10 | 10.1% | 21 | 0 | 0.0% | 1.93E−03 | 2.77E−01 | 1.81E−03 |
| RPTOR | 566 | 15 | 2.6% | 97 | 2 | 2.0% | 17 | 4 | 19.0% | 1.00E+00 | 6.92E−03 | 6.49E−05 |
| CDH20 | 567 | 14 | 2.4% | 98 | 1 | 1.0% | 18 | 3 | 14.3% | 6.13E−01 | 1.60E−02 | 1.89E−03 |
| RB1 | 574 | 7 | 1.2% | 98 | 1 | 1.0% | 15 | 6 | 28.6% | 1.00E+00 | 1.17E−05 | 1.08E−17 |
| CDH2 | 574 | 7 | 1.2% | 97 | 2 | 2.0% | 18 | 3 | 14.3% | 8.57E−01 | 5.07E−02 | 3.24E−05 |

ccRCC-NonSarc = clear cell RCC, non-sarcomatoid tumors; ccRCC-Sarc = clear cell RCC, sarcomatoid tumors; Non-ccRCC-Sarc = non-clear cell RCC, sarcomatoid tumors. Genes with at least 10% alterations in either of the three subsets are included in this table.

Overall, these analyses show that sRCC tumors exhibit a highly proliferative molecular phenotype, characterized by relatively low angiogenesis, and accompanied with high immune presence and PD-L1 expression, which may explain the increased sensitivity of sarcomatoid tumors to therapeutic intervention with atezolizumab+bevacizumab vs. sunitinib (FIGS. 11G and 11H; Rini et al. Lancet. 393: 2404-2415 (2019)).

vii. Discussion

This Example presents comprehensive molecular analyses of 823 tumors from advanced RCC patients treated with atezolizumab+bevacizumab or sunitinib, representing the largest set of integrated multi-omics characterization of advanced RCC in a randomized global Phase III clinical trial. The findings provide important new insights into key biological pathways underlying RCC progression, validate for the first time the prognostic and predictive capability of transcriptional signatures identified in a Phase II cohort in a randomized Phase III trial, describe distinct molecular subtypes that associate with differential overall outcome to antiangiogenics alone or combined with checkpoint blockade, and identify additional targets for future therapeutic development.

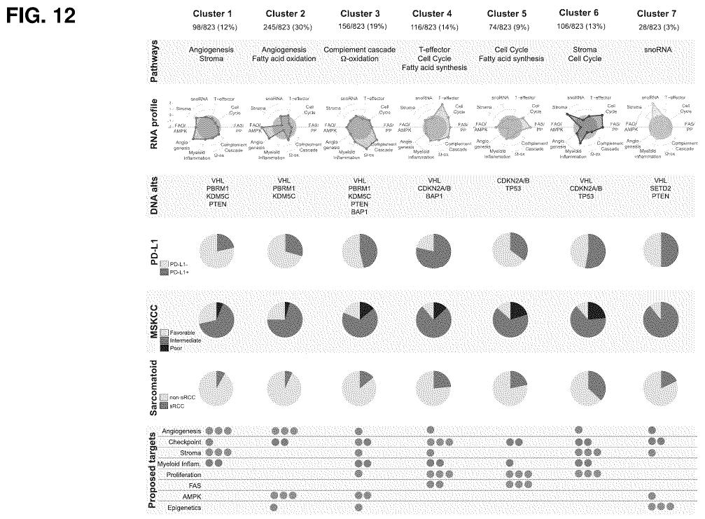

The unsupervised transcriptomic analysis identified seven robust tumor subsets (summarized in FIG. 12). This subtyping scheme corroborates and significantly expands on recent reports on gene expression-based subgrouping in smaller RCC data sets (Beuselinck et al. Clin Cancer Res. 21, 1329-1339, 2015; Brannon et al. Genes Cancer. 1, 152-163, 2010; Clark et al. Cell. 179, 964-983 e931, 2019; Hakimi et al. Cancer Discov. 9, 510-525, 2019). The substantially larger number of samples in the present data set resulted in increased resolution and detection of additional transcriptomic features associated with these subsets, such as differential metabolic profiles. Importantly, the clustering scheme was validated using an independent transcriptomic data set from IMmotion150 (McDermott et al. Nat Med. 24, 749-757, 2018), which also enrolled patients with untreated advanced RCC. Overall, the concordance of molecular subtypes across these different studies strengthens the case for a unified molecular classification in advanced RCC and its utility in understanding differential prognosis and sensitivity to therapeutics, including antiangiogenics, CPIs, and their combinations, which are now standards of care in untreated advanced RCC.

Indeed, RCC molecular subgroups could be reproducibly associated with differential clinical responses to anti-angiogenics and a CPI. Patients in angiogenesis enriched clusters 1 and 2 demonstrated superior prognosis in both atezolizumab+bevacizumab and sunitinib-treated patients, with no significant difference in PFS between the two treatment arms, likely as a result of both treatment arms containing an angiogenesis inhibitor. In contrast, sunitinib showed worse clinical outcomes in the angiogenesis poor, but immune rich, and cell cycle enriched clusters 4 and 5, and atezolizumab+bevacizumab significantly improved ORR and PFS vs sunitinib in these subsets, consistent with the inclusion of an immunotherapeutic in the combination regimen.

The dual CPI combination of nivolumab plus ipilimumab showed improved OS and ORR in patients with intermediate and poor prognostic risk as assessed by the IMDC score, whereas patients with favorable risk showed numerically superior results for OS, PFS, and ORR with sunitinib (Motzer et al. N Engl J Med. 378, 1277-1290, 2018). In contrast, combined VEGF and checkpoint inhibition by atezolizumab+bevacizumab, avelumab+axitinib, and pembrolizumab+axitinib (Motzer et al. N Engl J Med. 378, 1277-1290 (2019); Rini et al. N Engl J Med. 380, 1116-1127 (2019); Rini et al. Lancet. 393, 2404-2415 (2019)) showed PFS benefit across clinical risk groups, including in patients with favorable prognostic risk. In this study, tumors from favorable risk patients were enriched in the Angiogenic/Stromal (#1) and the Angiogenic (#2) clusters, which exhibited higher expression of genes associated with the VEGF pathway. These findings provide a molecular explanation for improved clinical outcomes to combined CPI+VEGF inhibition vs. CPI only therapy across clinical risk categories and support treatment of favorable risk patients with therapeutic regimens that include VEGF pathway inhibitors. Moving forward, treatment of patients based on transcriptomic profiling of tumors, and independent of IMDC risk categorization, if prospectively validated, could allow for a more personalized, biology-based approach to treatment selection.

Integration of gene expression profiles with somatic alterations provided further insights into the molecular underpinnings of the transcriptomic subgroups. PBRM1 mutant tumors associated with higher expression of the angiogenesis gene signature, and in agreement with previous clinical findings (Carlo et al. Kidney Cancer. 1, 49-56, 2017; Hakimi et al. Cancer Discov. 9, 510-525, 2019; McDermott et al. Nat Med. 24, 749-757, 2018; Voss et al. Lancet Oncol. 19, 1688-1698, 2018), showed improved clinical outcomes to sunitinib vs. PBRM1 non-mutants. Recent preclinical studies have shown that PBRM1 loss in VHL deficient cell lines and mouse models induced amplification of HIF-1A/HIF-2A mediated hypoxia response (Gao et al. Proc Natl Acad Sci USA. 114:1027-1032 (2017); Nargund et al. Cell Rep. 18: 2893-2906 (2017)). Thus, evaluation of clinical activity of novel agents targeting hypoxia and angiogenesis, such as HIF-2A inhibitors (Jonasch et al. Ann Oncol. 30(suppl_5): v 356-v 402 (2019)), is especially warranted in PBRM1 mutant tumors.

Tumors harboring CDKN2A/2B alterations were more prevalent in T-effector/Proliferative (#4), Proliferative (#5), and Stromal/Proliferative (#6) clusters; and TP53 mutations were more prevalent in Proliferative (#5), and Stromal/Proliferative (#6) clusters. Atezolizumab+bevacizumab improved clinical outcomes vs. sunitinib in these highly proliferative and aggressive tumors. Importantly, patients whose tumors harbored CDKN2A/B loss and/or TP53 mutations showed overall worse prognosis and may additionally benefit from therapeutic approaches that target these specific aberrations, such as stromal disruptors, cytotoxic agents, or CDK4/6 inhibitors. Preclinical studies have demonstrated immunomodulatory effects of CDK4/6 inhibition in tumor models, such as increase in antigen presentation by tumor cells, upregulation of PD-L1 expression, reduction in intratumoral regulatory T cells, and activation of CD8⁺ T cells, as well as enhancement of anti-tumor efficacy in combination with PD-L1 blockade (Deng et al. Cancer Discov. 8: 216-233 (2018); Goel et al. Nature. 548: 471-475 (2017); Schaer et al. Cell Rep. 22: 2978-2994 (2018)). Collectively, these data support clinical investigation of CDK4/6 inhibitors in combination with CPI in RCC.

Intriguingly, loss-of-function mutations in ARID1A and KMT2C associated with improved PFS in atezolizumab+bevacizumab vs. sunitinib-treated patients, in the absence of clear associations with transcriptional signatures. Alterations in ARID1A, a component of the chromatin remodeling SWI/SNF complex, and KMT2C, a histone methyl transferase, have been implicated in epigenetic dysregulation and DNA damage repair deficiency (Rampias et al. EMBO Rep. 20(3): e46821 (2019); Shen et al. Nat Med. 24: 556-562 (2018)). While the mechanistic basis for the differential clinical outcome in patients with either mutation remains to be elucidated in RCC, these observations support combining epigenetic regulators with CPI in subsets of patients with RCC.

Sarcomatoid dedifferentiation in RCC has been historically associated with poor outcomes to VEGF inhibition (Golshayan et al. J Clin Oncol. 27: 235-241 (2009)). In contrast, atezolizumab+bevacizumab, as well as other CPI-based therapies, have demonstrated substantial efficacy, including complete responses, in patients whose tumors include a sarcomatoid component (Choueiri et al. Ann Oncol. 30(Supp. 5): v 361 (2019); McDermott et al. J Clin Oncol. 37(15_suppl): 4513 (2019); Rini et al. J Clin Oncol. 37(15_suppl): 4500 (2019); Rini et al. Lancet. 393: 2404-2415 (2019)). The distinct genomic features of sarcomatoid tumors identified in this study suggest a molecular basis for the aggressive phenotype of sarcomatoid tumors, and provide a biological rationale for prioritizing checkpoint blockade-based therapy in patients with sarcomatoid RCC.

Overall, findings from this randomized Phase III study expand our understanding of RCC biology and provide a molecular basis for differential clinical outcomes and resistance mechanisms associated with angiogenesis blockade, checkpoint inhibition and their combinations in patients with untreated advanced RCC. Given that these combinations are under clinical evaluation and have shown promising activity in additional indications, such as hepatocellular carcinoma, non-small cell lung cancer, and endometrial cancer, the findings from this study may be applicable in interpreting clinical outcomes and developing personalized therapies across many cancers.

### Example 2: Evaluation of IMmotion151 Molecular Subtypes in JAVELIN 101 Data Set

This Example describes a study that validated the IMmotion151 molecular subtypes identified in Example 1 using an independent data set obtained from the JAVELIN 101 study. Briefly, the IMmotion151 gene set was used as a training set to develop a transcriptional classifier model. The model was then applied to predict NMF clusters in the JAVELIN 101 data set (n=724). Comparisons of the transcriptional signatures from the IMmotion151 and JAVELIN 101 data sets indicated that the biological pathways and distribution of the NMF subtypes among patients was similar. In addition, NMF subtypes were associated with similar prognostic and predictive clinical effects in the IMmotion151 and JAVELIN 101 data. In summary, these findings demonstrate the identification and reproducibility of the first transcriptomic classifier in advanced RCC across multiple data sets.

### A. Study Design

JAVELIN 101 (NCT02684006) was a multicenter, randomized, open-label, Phase 3 trial comparing avelumab in combination with axitinib versus sunitinib monotherapy in the first-line treatment of patients with advanced RCC. The study design, methods, and primary clinical findings from JAVELIN 101 have been reported previously (Motzer et al. N Engl J Med. 380: 1103-1115 (2019)).

Key inclusion criteria of patients for entry into the JAVELIN 101 study:
Previously untreated advanced RCC with a clear cell component
At least one measurable lesion as defined by RECIST, version 1.1
Tumor tissue available for PD-L1 staining
Eastern Cooperative Oncology Group performance-status score (ECOG PS) of 0 or 1

Randomization in a 1:1 ratio was stratified according to ECOG PS (0 vs. 1) and geographic region (United States vs. Canada and Western Europe vs. rest of the world).

Patients were randomly assigned in a 1:1 ratio to receive avelumab (10 mg per kg of body weight) intravenously every 2 weeks plus axitinib (5 mg) orally twice daily or sunitinib (50 mg) orally once daily for 4 weeks of a 6-week cycle (4 weeks on, 2 weeks off). The two independent primary efficacy endpoints were PFS and OS among patients with PD-L1-positive tumors (21% of immune cells staining positive within the tumor area of the tested tissue sample). A key secondary efficacy endpoint was PFS in the overall population; other endpoints included objective response rate and tumor-tissue biomarkers.

### B. Materials and Methods

Method details are described in the Validation of NMF Clustering in IMmotion150 section in Example 1. Similar to Example 1, a classifier was developed using the random forest machine learning algorithm (R package randomForest). The random forest classifier was learned on the IMmotion151-derived training gene set and then the classifier was used to predict the NMF classes in the JAVELIN data set. Each gene was normalized by z-score, and downsampling was also performed.

### C. Results

i. Similar Biological Pathways and Distribution of NMF Subtypes in IMmotion151 and JAVELIN 101 Data Sets

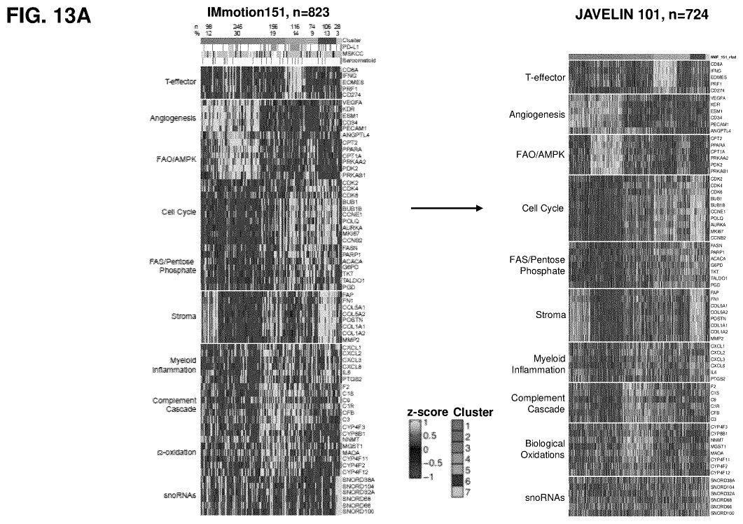

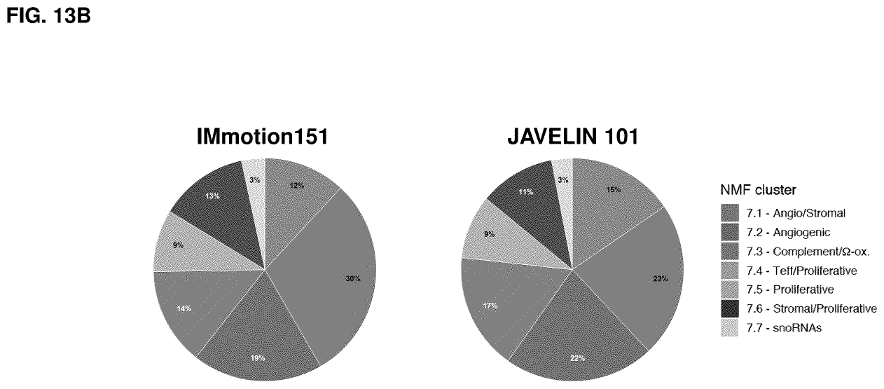

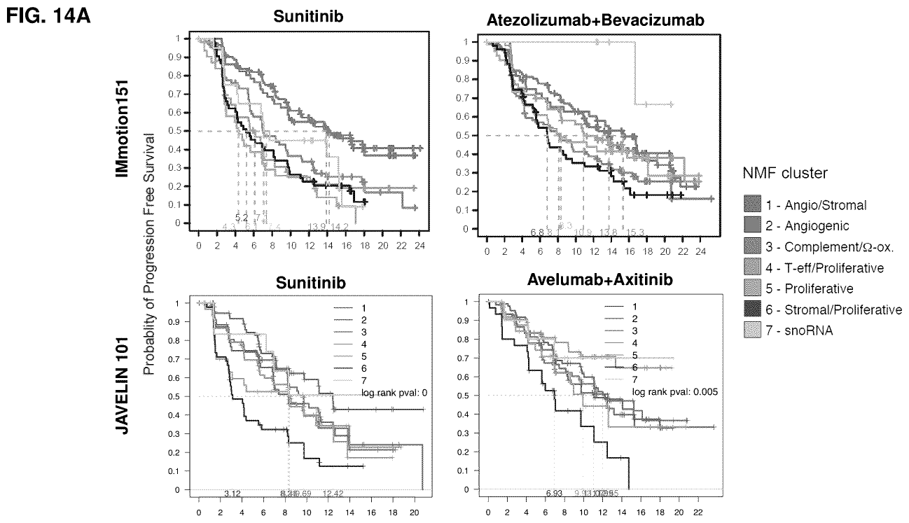

To validate the IMmotion151 molecular subtypes identified in Example 1, gene expression data from patient tumors (n=724) was obtained and a random forest model trained on the IMmotion151 data set was applied to predict the NMF subtypes in the JAVELIN 101 samples. A comparison of the IMmotion151 and JAVELIN 101 transcriptional signatures indicated that the biological pathways of the NMF clusters was similar between the two studies (FIG. 13A). Also similar between the IMmotion151 and JAVELIN 101 studies was the distribution of the NMF clusters among patients (FIG. 13B). These results indicate that this transcriptomic classifier for advanced RCC molecular biology is highly reproducible across multiple, independent data sets.

ii. NMF Subtypes are Associated with Similar Prognostic and Predictive Clinical Outcomes in IMmotion151 and JAVELIN 101 Data Sets

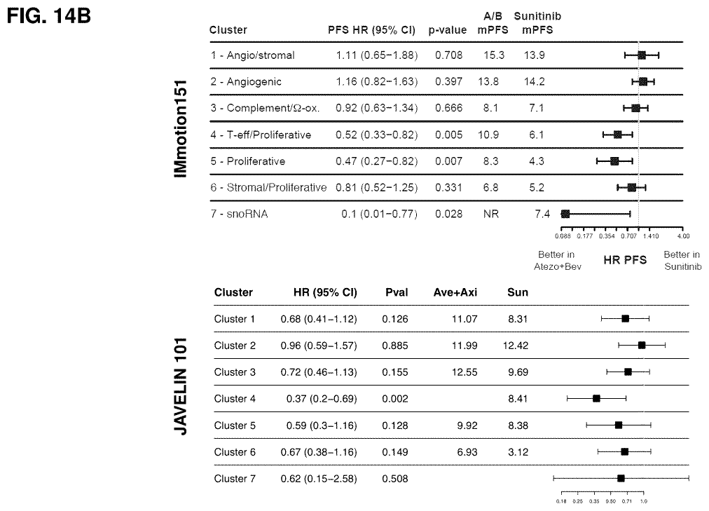

To characterize the clinical outcomes in the IMmotion151 and JAVELIN 101 studies by NMF molecular subtypes, the PFS of the treatment groups was compared for each NMF cluster. The NMF clusters were associated with similar clinical outcomes in the IMmotion151 and JAVELIN 101 data sets (FIGS. 14A and 14B). For the T-effector/Proliferative cluster (#4) in both the IMmotion151 and JAVELIN 101 data sets, the clinical benefit was significantly enriched in atezolizumab+bevacizumab versus sunitib and avelumab+axinitinib versus sunitinib, respectively. In contrast, for the Stromal/Proliferative cluster (#6), the clinical outcome was the lowest (as measured by lowest PFS) to atezolizumab+bevacizumab versus sunitinib and avelumab+axitinib versus sunitinib for IMmotion151 and JAVELIN 101, respectively. Angiogenesis-enriched subtypes (clusters #1 and 2) exhibited similar PFS outcomes to atezolizumab+bevacizumab, sunitinib, and avelumab+axitinib. Immune and/or proliferative subtypes (clusters #4, 5, and 6) show improved outcomes to atezolizumab+bevacizumab versus sunitinib and avelumab+axitinib versus sunitinib.

In summary, this analysis of the JAVELIN 101 data set provides confirmation of the prevalence, biology, and differential clinical outcomes associated with molecular subtypes identified in Example 1. These integrative biomarker analyses improve understanding of RCC biology and identify molecular bases for differential clinical outcomes to VEGF inhibition, checkpoint inhibitors, and combination therapies thereof in advanced RCC.

### OTHER EMBODIMENTS

Although the foregoing invention has been described in some detail by way of illustration and example for purposes of clarity of understanding, the descriptions and examples should not be construed as limiting the scope of the invention.

1. A method of treating an inoperable, locally advanced, or metastatic renal cell carcinoma (RCC) in a human patient, wherein the inoperable, locally advanced, or metastatic RCC is previously untreated, the method comprising:
(a) assaying mRNA in a tumor sample from the patient to provide a transcriptional profile of the patient's tumor;
(b) assigning the patient's tumor sample into one of the following seven clusters based on the transcriptional profile of the patient's tumor:
(1) angiogenic/stromal;
(2) angiogenic;
(3) complement/Ω-oxidation;
(4) T-effector/proliferative;
(5) proliferative;
(6) stromal/proliferative; and
(7) snoRNA,
thereby classifying the previously untreated inoperable, locally advanced, or metastatic RCC in the patient; and
(c) administering an anti-cancer therapy to the patient based on the classification.

2-3. (canceled)

4. The method of claim 1, wherein:
(a) assaying mRNA in the tumor sample from the patient comprises RNA sequencing (RNA-seq), reverse transcription-quantitative polymerase chain reaction (RT-qPCR), qPCR, multiplex qPCR or RT-qPCR, microarray analysis, serial analysis of gene expression (SAGE), MassARRAY technique, in situ hybridization (ISH), or a combination thereof;
(b) the seven clusters are identified by non-negative matrix factorization (NMF);
(c) the tumor sample is a formalin-fixed and paraffin-embedded (FFPE) sample, an archival sample, a fresh sample, or a frozen sample;
(d) the tumor sample is a pre-treatment tumor sample;
(e) the tumor sample from the patient has a clear cell histology or a non-clear cell histology;
(f) the tumor sample from the patient has a sarcomatoid component or lacks a sarcomatoid component;
(q) the method further comprises determining the patient's Memorial Sloan Kettering Cancer Center (MSKCC) risk score;
(h) assignment of the patient's tumor sample into one of the following clusters:
(4) T-effector/proliferative;
(5) proliferative; or
(7) snoRNA,
indicates that the patient is likely to have an increased clinical benefit from treatment with an anti-cancer therapy comprising atezolizumab and bevacizumab compared to treatment with sunitinib;
(i) the patient's tumor sample is assigned into one of the following clusters:
(4) T-effector/proliferative;
(5) proliferative; or
(7) snoRNA,
and the method further comprises treating the patient by administering an anti-cancer therapy comprising atezolizumab and bevacizumab to the patient;
(j) the anti-cancer therapy comprises atezolizumab and bevacizumab; and/or
(k) the method further comprises administering an additional therapeutic agent to the patient.

5. The method of claim 4, wherein:
(a) assaying mRNA in the tumor sample from the patient comprises RNA-seq;
(b) the seven clusters identified by NMF are based on a set of genes representing the top 10% most variable genes in a population of patients having previously untreated inoperable, locally advanced, or metastatic RCC; and/or
(c) increased clinical benefit comprises a relative increase in one or more of the following: objective response rate (ORR), overall survival (OS), progression-free survival (PFS), compete response (CR), partial response (PR), or a combination thereof.

6-7. (canceled)

8. The method of claim 5, wherein:
(a) the set of genes is set forth in Table 1; and/or
(b) increased clinical benefit comprises a relative increase in ORR or PFS.

9. The method of claim 1, wherein the method further comprises determining the mRNA expression level of one or more of the following gene signatures in the tumor sample from the patient:
(a) a T-effector signature comprising CD8A, IFNG, EOMES, PRF1, and PD-L1;
(b) an angiogenesis signature comprising VEGFA, KDR, ESM1, CD34, PECAM1, and ANGPTL4;
(c) a fatty acid oxidation (FAO)/AMPK signature comprising CPT2, PPARA, CPT1A, PRKAA2, PDK2, and PRKAB1;
(d) a cell cycle signature comprising CDK2, CDK4, CDK6, BUB1, BUB1B, CCNE1, POLQ, AURKA, MKI67, and CCNB2;
(e) a fatty acid synthesis (FAS)/pentose phosphate signature comprising FASN, PARP1, ACACA, G6PD, TKT, TALDO1, and PGD;
(f) a stroma signature comprising FAP, FN1, COL5A1, COL5A2, POSTN, COL1 A1, COL1 A2, and MMP2;
(g) a myeloid inflammation signature comprising CXCL1, CXCL2, CXCL3, CXCL8, IL6, and PTGS2;
(h) a complement cascade signature comprising F2, C1S, C9, C1R, CFB, and C3;
(i) an Ω-oxidation signature comprising CYP4F3, CYP8B1, NNMT, MGST1, MAOA, CYP4F11, CYP4F2, CYP4F12; and/or
(j) a snoRNA signature comprising SNORD38A, SNORD104, SNORD32A, SNORD68, SNORD66, and SNORD100.

10. The method of claim 9, wherein;
(a) the patient's tumor sample is assigned into the angiogenic/stromal cluster, and the patient's tumor sample has increased expression levels, relative to reference expression levels, of the angiogenesis signature and the stroma signature;
(b) the patient's tumor sample is assigned into the angiogenic cluster, and the patient's tumor sample has increased expression levels, relative to a reference expression levels, of the angiogenesis signature and the FAO/AMPK signature;
(c) the patient's tumor sample is assigned into the complement/Ω-oxidation cluster, and the patient's tumor sample has increased expression levels, relative to reference expression levels, of the complement cascade signature and the Ω-oxidation signature;
(d) the patient's tumor sample is assigned into the T-effector/proliferative cluster, and the patient's tumor sample has increased expression levels, relative to reference expression levels, of the cell cycle signature and the T-effector signature;
(e) the patient's tumor sample is assigned into the proliferative cluster, and the patient's tumor sample has increased expression levels, relative to reference expression levels, of the cell cycle signature and the FAS/pentose phosphate signature;
(f) the patient's tumor sample is assigned into the stromal/proliferative cluster, and the patient's tumor sample has increased expression levels, relative to reference expression levels, of the cell cycle signature and the stromal signature; or
(g) the patient's tumor sample is assigned into the snoRNA cluster, and the patient's tumor sample has an increased expression level, relative to a reference expression level, of the snoRNA signature.

11-16. (canceled)

17. The method of claim 9, wherein:
(a) the reference expression level of a signature is the median Z-score of the signature in a population of patients having a previously untreated inoperable, locally advanced, or metastatic RCC; and/or
(b)
(i) the patient's tumor sample is assigned into the angiogenic/stromal cluster, and the patient's tumor sample has decreased expression levels, relative to reference expression levels, of the T-effector signature, the cell cycle signature, and/or the FAS/pentose phosphate signature;
(ii) the patient's tumor sample is assigned into the angiogenic cluster, and the patient's tumor has decreased expression levels, relative to reference expression levels, of the cell cycle signature, the FAS/pentose phosphate signature, the stroma signature, the myeloid inflammation signature, and/or the complement cascade signature;
(iii) the patient's tumor sample is assigned into the complement/Ω-oxidation cluster, and the patient's tumor sample has an increased expression level, relative to a reference expression level, of the myeloid inflammation signature, and/or decreased expression levels, relative to reference expression levels, of the angiogenesis signature and/or the T-effector signature;
(iv) the patient's tumor sample is assigned into the T-effector/proliferative cluster, and the patient's tumor sample has increased expression levels, relative to reference expression levels, of the FAS/pentose phosphate signature, the myeloid inflammation signature, and/or the complement cascade signature, and/or decreased expression levels, relative to reference expression levels, of the angiogenesis signature, the FAO/AMP signature, and/or the snoRNA signature;
(v) the patient's tumor sample is assigned into the proliferative cluster, and the patient's tumor sample has increased expression levels, relative to reference expression levels, of the myeloid inflammation signature and/or the FAO/AMPK signature, and/or decreased expression levels, relative to reference expression levels, of the angiogenesis signature, the T-effector signature, the stroma signature, the complement cascade signature, the Ω-oxidation signature, and/or the snoRNA signature;
(vi) the patient's tumor sample is assigned into the stromal/proliferative cluster, and the patient's tumor sample has increased expression levels, relative to reference expression levels, of the FAS/pentose phosphate signature and/or the myeloid inflammation signature, and/or decreased expression levels, relative to reference expression levels, of the angiogenesis signature, the FAO/AMPK signature, the complement cascade signature, the Ω-oxidation signature, and/or the snoRNA signature; or
(vii) the patient's tumor sample is assigned into the snoRNA cluster, and the patient's tumor sample has decreased expression levels, relative to reference expression levels, of the FOA/AMPK signature, the cell cycle signature, and the FAS/pentose phosphate signature.

18-21. (canceled)

22. The method of claim 1, further comprising assaying for somatic alterations in the patient's genotype in the tumor sample obtained from the patient.

23. The method of claim 22, wherein:
(a) the method comprises assaying for somatic alterations in PBRM1, CDKN2A, CDK2NB, TP53, ARID1A, and/or KMT2C;
(b) the patient's genotype is determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, and the method further comprises administering to the patient an anti-cancer therapy comprising atezolizumab and bevacizumab; and/or
(c) the somatic alteration is a short variant, a loss, an amplification, a deletion, a duplication, a rearrangement, or a truncation.

24. The method of claim 23, wherein:
(a) (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1 indicates that the patient is likely to have an increased clinical benefit from treatment with an anti-cancer therapy comprising atezolizumab and bevacizumab compared to treatment with sunitinib; or
(b) the presence of a somatic alteration in the patient's genotype in PBRM1 indicates that the patient is likely to have an increased clinical benefit from treatment with sunitinib compared with a patient whose genotype lacks a somatic alteration in PBRM1.

25. (canceled)

26. A method of treating a previously untreated inoperable, locally advanced, or metastatic RCC in a patient whose genotype has been determined to comprise (i) the presence of a somatic alteration in the patient's genotype in one or more of the following genes: CDKN2A, CDK2NB, TP53, ARID1A, and KMT2C or (ii) the absence of a somatic alteration in the patient's genotype in PBRM1, the method comprising administering to the patient an anti-cancer therapy comprising atezolizumab and bevacizumab.

27. (canceled)

28. The method of claim 24, wherein the patient's genotype is determined to comprise a somatic alteration in PBRM1, and the method further comprises administering sunitinib to the patient.

29-37. (canceled)

38. The method of claim 4, wherein the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof.

39. The method of claim 38, wherein:
(a) the growth inhibitory agent is a CDK4/6 inhibitor;
(b) the anti-angiogenic agent is a VEGF antagonist or a HIF2A inhibitor;
(c) the stromal inhibitor is a TGF-β antagonist; or
(d) the metabolism inhibitor is a PCSK9 inhibitor or a FAS inhibitor.

40. The method of claim 39, wherein the CDK4/6 inhibitor is palbociclib, ribociclib, or abemaciclib.

41-43. (canceled)

44. A kit for classifying an inoperable, locally advanced, or metastatic RCC in a human patient, wherein the inoperable, locally advanced, or metastatic RCC is previously untreated, the kit comprising:
(a) reagents for assaying mRNA in a tumor sample from the patient to provide a transcriptional profile of the patient's tumor; and
(b) instructions for assigning the patient's tumor sample into one of the following seven clusters based on the transcriptional profile of the patient's tumor:
(1) angiogenic/stromal;
(2) angiogenic;
(3) complement/Ω-oxidation;
(4) T-effector/proliferative;
(5) proliferative;
(6) stromal/proliferative; and
(7) snoRNA,
thereby classifying the previously untreated inoperable, locally advanced, or metastatic RCC in the patient.

45. A kit for identifying a human patient suffering from an inoperable, locally advanced, or metastatic RCC who may benefit from treatment with an anti-cancer therapy comprising atezolizumab and bevacizumab, wherein the inoperable, locally advanced, or metastatic RCC is previously untreated, the kit comprising:
(a) reagents for determining the presence of a somatic alteration in one or more of the following genes: PBRM1, CDKN2A, CDK2NB, TP53, ARID1A, and KMT2C in a tumor sample obtained from the patient; and
(b) instructions for using the reagents to identify the patient as one who may benefit from a treatment with an anti-cancer therapy comprising atezolizumab and bevacizumab.

46-56. (canceled)
